SEQUENCE LISTING (266 sequences)

SEQ ID NO: 1 - protein, 22 aa, synthetic construct
  REGION 1..22: note = CCL1 N-loop peptide
GGKFSFAEQEIPLRAILCYRNT

SEQ ID NO: 2 - protein, 23 aa, synthetic construct
  REGION 1..23: note = CCL2 N-loop peptide
GGKYNFTNRKISVQRLASYRRIT

SEQ ID NO: 3 - protein, 25 aa, synthetic construct
  REGION 1..25: note = CCL3 N-loop peptide
GGSFSYTSRQIPQNFIADYFETSSQ

SEQ ID NO: 4 - protein, 25 aa, synthetic construct
  REGION 1..25: note = CCL4 N-loop peptide
GGSFSYTARKLPRNFVVDYYETSSL

SEQ ID NO: 5 - protein, 25 aa, synthetic construct
  REGION 1..25: note = CCL5 N-loop peptide
GGSFAYIARPLPRAHIKEYFYTSGK

SEQ ID NO: 6 - protein, 25 aa, synthetic construct
  REGION 1..25: note = CCL7 N-loop peptide
GGSYRFINKKIPKQRLESYRRTTSS

SEQ ID NO: 7 - protein, 25 aa, synthetic construct
  REGION 1..25: note = CCL8 N-loop peptide
GGSFNVINRKIPIQRLESYTRITNI

SEQ ID NO: 8 - protein, 25 aa, synthetic construct
  REGION 1..25: note = CCL11 N-loop peptide
GGSFNLANRKIPLQRLESYRRITSG

SEQ ID NO: 9 - protein, 25 aa, synthetic construct
  REGION 1..25: note = CCL13 N-loop peptide
GGSFTFSSKKISLQRLKSYVITTSR

SEQ ID NO: 10 - protein, 25 aa, synthetic construct
  REGION 1..25: note = CCL14 N-loop peptide
GGSFTYTTYKIPRQRIMDYYETNSQ

SEQ ID NO: 11 - protein, 25 aa, synthetic construct
  REGION 1..25: note = CCL15 N-loop peptide
GGSTSYISQSIPCSLMKSYFETSSE

SEQ ID NO: 12 - protein, 25 aa, synthetic construct
  REGION 1..25: note = CCL16 N-loop peptide
GGGSLKYYEKVLPRRLVVGYRKALN

SEQ ID NO: 13 - protein, 25 aa, synthetic construct
  REGION 1..25: note = CCL17 N-loop peptide
GGSLEYFKGAIPLRKLKTWYQTSED

SEQ ID NO: 14 - protein, 25 aa, synthetic construct
  REGION 1..25: note = CCL18 N-loop peptide
GGSLVYTSWQIPQKFIVDYSETSPQ

SEQ ID NO: 15 - protein, 25 aa, synthetic construct
  REGION 1..25: note = CCL19 N-loop peptide
GGSLSVTQKPIPGYIVRNFHYLLIK

SEQ ID NO: 16 - protein, 25 aa, synthetic construct
  REGION 1..25: note = CCL20 N-loop peptide
GGSLGYTDRILHPKFIVGFTRQLAN

SEQ ID NO: 17 - protein, 25 aa, synthetic construct
  REGION 1..25: note = CCL21 N-loop peptide
GGSLKYSQRKIPAKVVRSYRKQEPS

SEQ ID NO: 18 - protein, 25 aa, synthetic construct
  REGION 1..25: note = CCL22 N-loop peptide
GGSRDYVRYRLPLRVVKHFYWTSDS

SEQ ID NO: 19 - protein, 25 aa, synthetic construct
  REGION 1..25: note = CCL23 N-loop peptide
GGSISYTPRSIPCSLLESYFETNSE

SEQ ID NO: 20 - protein, 25 aa, synthetic construct
  REGION 1..25: note = CCL24 N-loop peptide
GGSMFFVSKRIPENRVVSYQLSSRS

SEQ ID NO: 21 - protein, 25 aa, synthetic construct
  REGION 1..25: note = CCL25 N-loop peptide
GGSLAYHYPIGWAVLRRAWTYRIQE

SEQ ID NO: 22 - protein, 25 aa, synthetic construct
  REGION 1..25: note = CCL26 N-loop peptide
GGSFQYSHKPLPWTWVRSYEFTSNS

SEQ ID NO: 23 - protein, 25 aa, synthetic construct
  REGION 1..25: note = CCL27 N-loop peptide
GGSTQLYRKPLSDKLLRKVIQVELQ

SEQ ID NO: 24 - protein, 25 aa, synthetic construct
  REGION 1..25: note = CCL28 N-loop peptide
GGSTEVSHHISRRLLERVNMCRIQR

SEQ ID NO: 25 - protein, 25 aa, synthetic construct
  REGION 1..25: note = CXCL1 N-loop peptide
GGSLQTLQGIHPKNIQSVNVKSPGP

SEQ ID NO: 26 - protein, 25 aa, synthetic construct
  REGION 1..25: note = CXCL2 N-loop peptide
GGSLQTLQGIHLKNIQSVKVKSPGP

SEQ ID NO: 27 - protein, 25 aa, synthetic construct
  REGION 1..25: note = CXCL3 N-loop peptide
GSLQTLQGIHLKNIQSVNVRSPGPH

SEQ ID NO: 28 - protein, 25 aa, synthetic construct
  REGION 1..25: note = CXCL4 N-loop peptide
GGSVKTTSQVRPRHITSLEVIKAGP

SEQ ID NO: 29 - protein, 25 aa, synthetic construct
  REGION 1..25: note = CXCL5 N-loop peptide
GGSLQTTQGVHPKMISNLQVFAIGP

SEQ ID NO: 30 - protein, 25 aa, synthetic construct
  REGION 1..25: note = CXCL6 N-loop peptide
GGSLRVTLRVNPKTIGKLQVFPAGP

SEQ ID NO: 31 - protein, 25 aa, synthetic construct
  REGION 1..25: note = CXCL7 N-loop peptide
GGSIKTTSGIHPKNIQSLEVIGKGT

SEQ ID NO: 32 - protein, 25 aa, synthetic construct
  REGION 1..25: note = CXCL8 N-loop peptide
GGSIKTYSKPFHPKFIKELRVIESG

SEQ ID NO: 33 - protein, 25 aa, synthetic construct
  REGION 1..25: note = CXCL9 N-loop peptide
GGSISTNQGTIHLQSLKDLKQFAPS

SEQ ID NO: 34 - protein, 25 aa, synthetic construct
  REGION 1..25: note = CXCL10 N-loop peptide
GGSISISNQPVNPRSLEKLEIIPAS

SEQ ID NO: 35 - protein, 25 aa, synthetic construct
  REGION 1..25: note = CXCL11 N-loop peptide
GGSIGPGVKAVKVADIEKASIMYPS

SEQ ID NO: 36 - protein, 25 aa, synthetic construct
  REGION 1..25: note = CXCL12 N-loop peptide
GGSRFFESHVARANVKHLKILNTPN

SEQ ID NO: 37 - protein, 25 aa, synthetic construct
  REGION 1..25: note = CXCL13 N-loop peptide
GGSVQESSVFIPRRFIDRIQILPRG

SEQ ID NO: 38 - protein, 25 aa, synthetic construct
  REGION 1..25: note = CXCL14 N-loop peptide
GGSSRKGPKIRYSDVKKLEMKPKYP

SEQ ID NO: 39 - protein, 25 aa, synthetic construct
  REGION 1..25: note = CXCL16 N-loop peptide
GGSGKRISSDSPPSVQFMNRLRKHL

SEQ ID NO: 40 - protein, 25 aa, synthetic construct
  REGION 1..25: note = CXCL17 N-loop peptide
GGSKDWFLRAPRRKFMTVSGLPKKQ

SEQ ID NO: 41 - protein, 25 aa, synthetic construct
  REGION 1..25: note = XCL1/XCL2 N-loop peptide
GGSVSLTTQRLPVSRIKTYTITEGS

SEQ ID NO: 42 - protein, 25 aa, synthetic construct
  REGION 1..25: note = CX3CL1 N-loop peptide
GGGSSKMTSKIPVALLIHYQQNQAS

SEQ ID NO: 43 - protein, 25 aa, synthetic construct
  REGION 1..25: note = CCL8 C-terminal peptide
GGGSKERWVRDSMKHLDQIFQNLKP

SEQ ID NO: 44 - protein, 25 aa, synthetic construct
  REGION 1..25: note = CCL16 C-terminal peptide
GGSNDDWVQEYIKDPNLPLLPTRNL

SEQ ID NO: 45 - protein, 25 aa, synthetic construct
  REGION 1..25: note = CCL26 C-terminal peptide
GGGSTHPRKKWVQKYISLLKTPKQL

SEQ ID NO: 46 - protein, 25 aa, synthetic construct
  REGION 1..25: note = CXCL1 C-terminal peptide
GGSLNPASPIVKKIIEKMLNSDKSN

SEQ ID NO: 47 - protein, 25 aa, synthetic construct
  REGION 1..25: note = CXCL12 C-terminal peptide
GGSIDPKLKWIQEYLEKALNKRFKM

SEQ ID NO: 48 - protein, 25 aa, synthetic construct
  REGION 1..25: note = CXCL13 C-terminal peptide
GGSQAEWIQRMMEVLRKRSSSTLPV

SEQ ID NO: 49 - protein, 25 aa, synthetic construct
  REGION 1..25: note = CXCL16 C-terminal peptide
GGSKDPWVQELMSCLDLKECGHAYS

SEQ ID NO: 50 - protein, 25 aa, synthetic construct
  REGION 1..25: note = IFN-a2 (aa 7-28) peptide
GGSHSLGSRRTLMLLAQMRKISLFS

SEQ ID NO: 51 - protein, 25 aa, synthetic construct
  REGION 1..25: note = SARS-CoV-2 Nucleocapsid (aa 157-178) peptide
GGSIVLQLPQGTTLPKGFYAEGSRG

SEQ ID NO: 52 - protein, 19 aa, Homo sapiens
FSFAEQEIPLRAILCYRNT

SEQ ID NO: 53 - protein, 20 aa, Homo sapiens
YNFTNRKISVQRLASYRRIT

SEQ ID NO: 54 - protein, 22 aa, Homo sapiens
FSYTSRQIPQNFIADYFETSSQ

SEQ ID NO: 55 - protein, 22 aa, Homo sapiens
FSYTARKLPRNFVVDYYETSSL

SEQ ID NO: 56 - protein, 22 aa, Homo sapiens
FAYIARPLPRAHIKEYFYTSGK

SEQ ID NO: 57 - protein, 22 aa, Homo sapiens
YRFINKKIPKQRLESYRRTTSS

SEQ ID NO: 58 - protein, 22 aa, Homo sapiens
FNVINRKIPIQRLESYTRITNI

SEQ ID NO: 59 - protein, 22 aa, Homo sapiens
FNLANRKIPLQRLESYRRITSG

SEQ ID NO: 60 - protein, 22 aa, Homo sapiens
FTFSSKKISLQRLKSYVITTSR

SEQ ID NO: 61 - protein, 22 aa, Homo sapiens
FTYTTYKIPRQRIMDYYETNSQ

SEQ ID NO: 62 - protein, 22 aa, Homo sapiens
TSYISQSIPCSLMKSYFETSSE

SEQ ID NO: 63 - protein, 21 aa, Homo sapiens
LKYYEKVLPRRLVVGYRKALN

SEQ ID NO: 64 - protein, 22 aa, Homo sapiens
LEYFKGAIPLRKLKTWYQTSED

SEQ ID NO: 65 - protein, 22 aa, Homo sapiens
LVYTSWQIPQKFIVDYSETSPQ

SEQ ID NO: 66 - protein, 22 aa, Homo sapiens
LSVTQKPIPGYIVRNFHYLLIK

SEQ ID NO: 67 - protein, 22 aa, Homo sapiens
LGYTDRILHPKFIVGFTRQLAN

SEQ ID NO: 68 - protein, 22 aa, Homo sapiens
LKYSQRKIPAKVVRSYRKQEPS

SEQ ID NO: 69 - protein, 22 aa, Homo sapiens
RDYVRYRLPLRVVKHFYWTSDS

SEQ ID NO: 70 - protein, 22 aa, Homo sapiens
ISYTPRSIPCSLLESYFETNSE

SEQ ID NO: 71 - protein, 22 aa, Homo sapiens
MFFVSKRIPENRVVSYQLSSRS

SEQ ID NO: 72 - protein, 22 aa, Homo sapiens
LAYHYPIGWAVLRRAWTYRIQE

SEQ ID NO: 73 - protein, 22 aa, Homo sapiens
FQYSHKPLPWTWVRSYEFTSNS

SEQ ID NO: 74 - protein, 22 aa, Homo sapiens
TQLYRKPLSDKLLRKVIQVELQ

SEQ ID NO: 75 - protein, 22 aa, Homo sapiens
TEVSHHISRRLLERVNMCRIQR

SEQ ID NO: 76 - protein, 22 aa, Homo sapiens
LQTLQGIHPKNIQSVNVKSPGP

SEQ ID NO: 77 - protein, 22 aa, Homo sapiens
LQTLQGIHLKNIQSVKVKSPGP

SEQ ID NO: 78 - protein, 23 aa, Homo sapiens
LQTLQGIHLKNIQSVNVRSPGPH

SEQ ID NO: 79 - protein, 22 aa, Homo sapiens
VKTTSQVRPRHITSLEVIKAGP

SEQ ID NO: 80 - protein, 22 aa, Homo sapiens
LQTTQGVHPKMISNLQVFAIGP

SEQ ID NO: 81 - protein, 22 aa, Homo sapiens
LRVTLRVNPKTIGKLQVFPAGP

SEQ ID NO: 82 - protein, 22 aa, Homo sapiens
IKTTSGIHPKNIQSLEVIGKGT

SEQ ID NO: 83 - protein, 22 aa, Homo sapiens
IKTYSKPFHPKFIKELRVIESG

SEQ ID NO: 84 - protein, 22 aa, Homo sapiens
ISTNQGTIHLQSLKDLKQFAPS

SEQ ID NO: 85 - protein, 22 aa, Homo sapiens
ISISNQPVNPRSLEKLEIIPAS

SEQ ID NO: 86 - protein, 22 aa, Homo sapiens
IGPGVKAVKVADIEKASIMYPS

SEQ ID NO: 87 - protein, 22 aa, Homo sapiens
RFFESHVARANVKHLKILNTPN

SEQ ID NO: 88 - protein, 22 aa, Homo sapiens
VQESSVFIPRRFIDRIQILPRG

SEQ ID NO: 89 - protein, 22 aa, Homo sapiens
SRKGPKIRYSDVKKLEMKPKYP

SEQ ID NO: 90 - protein, 22 aa, Homo sapiens
GKRISSDSPPSVQFMNRLRKHL

SEQ ID NO: 91 - protein, 22 aa, Homo sapiens
KDWFLRAPRRKFMTVSGLPKKQ

SEQ ID NO: 92 - protein, 22 aa, Homo sapiens
VSLTTQRLPVSRIKTYTITEGS

SEQ ID NO: 93 - protein, 21 aa, Homo sapiens
SKMTSKIPVALLIHYQQNQAS

SEQ ID NO: 94 - protein, 4 aa, synthetic construct
  REGION 1..4: note = linker
GGGS

SEQ ID NO: 95 - protein, 10 aa, Homo sapiens
GDSVSSESYF

SEQ ID NO: 96 - protein, 7 aa, Homo sapiens
VWYNGDT

SEQ ID NO: 97 - protein, 16 aa, Homo sapiens
ATEHCIDEICSPASDS

SEQ ID NO: 98 - protein, 12 aa, Homo sapiens
QSVFFSSNNENC

SEQ ID NO: 99 - skipped

SEQ ID NO: 100 - protein, 9 aa, Homo sapiens
LIHWASTRA

SEQ ID NO: 101 - protein, 9 aa, Homo sapiens
HQYLKAPRT

SEQ ID NO: 102 - protein, 124 aa, Homo sapiens
QVQLQESGPGLVKPAETLSLTCTVSGDSVSSESYFWGWIRQSPRKGLEWLGAVWYNGDTYYNPSVGGRVTISMDTSMNEISLRLSSVTAADTAVYFCATEHCIDEICSPASDSWGQGTLVTVSS

SEQ ID NO: 103 - protein, 113 aa, synthetic construct
  REGION 1..113: note = aCCL8.001 VL
DIVMTQSPDSLAVSLGERATINCKSSQSVFFSSNNENCLAWFQQKPGQPPKLLIHWASTRASGVPDRFSGSGSGTDFTLTISSLQAEDVAVYYCHQYLKAPRTFGQGTKVEIK

SEQ ID NO: 104 - protein, 112 aa, Homo sapiens
DIVMTQSPDSLAVSLGERATINCKSSQSVFFSSNNENCLAWFQQKPGQPPKLLIHWASTRASGVPDRFSGSGSGTDFTLTISSLQAEDVAVYYCHQYLKAPRTFGQGTKVEI

SEQ ID NO: 105 - protein, 10 aa, Homo sapiens
GDSVSSSFYF

SEQ ID NO: 106 - protein, 7 aa, Homo sapiens
IYYTGDT

SEQ ID NO: 107 - protein, 16 aa, Homo sapiens
ATEFCTSGICHPASDS

SEQ ID NO: 108 - protein, 12 aa, Homo sapiens
QSVLLSANNKNC

SEQ ID NO: 109 - protein, 9 aa, Homo sapiens
LIYWASTRE

SEQ ID NO: 110 - protein, 9 aa, Homo sapiens
QQYYKAPRT

SEQ ID NO: 111 - protein, 124 aa, Homo sapiens
QLQLQESGPGLVKPSETLSLTCTVSGDSVSSSFYFWGWVRQPPGKGLEWIGSIYYTGDTYYKPSLKSRVTISMNTSKNQFSLRLSSVTAADTAVYYCATEFCTSGICHPASDSWGQGTLVTVSS

SEQ ID NO: 112 - protein, 113 aa, Homo sapiens
DIVMTQSPDSLAVSLGERATINCKSSQSVLLSANNKNCLAWFQQKPGQPPKLLIYWASTRESGVPDRFSGSGSGTDFTLTISSLQAEDVAIYYCQQYYKAPRTFGQGTKVEIK

SEQ ID NO: 113 - protein, 10 aa, Homo sapiens
GDSVSSPSYF

SEQ ID NO: 114 - protein, 7 aa, Homo sapiens
TWFNGDT

SEQ ID NO: 115 - protein, 16 aa, Homo sapiens
ATEHCFDEICSPASDS

SEQ ID NO: 116 - protein, 9 aa, Homo sapiens
LIYWASTRA

SEQ ID NO: 117 - protein, 9 aa, Homo sapiens
QQYLKAPRT

SEQ ID NO: 118 - protein, 124 aa, synthetic construct
  REGION 1..124: note = aCCL8.004 VH
QVQLQESGPGLVKPAETLSLTCIVSGDSVSSPSYFWGWIRQPPGKGLEWIGATWFNGDTYYNPSLQSRVSISMDMSLNRIYLSLNSVTAADTAVYFCATEHCFDEICSPASDSWGQGTLVTVSS

SEQ ID NO: 119 - protein, 113 aa, synthetic construct
  REGION 1..113: note = aCCL8.004 VL
DIVMTQSPDSLAVSLGERATISCMSSQSVFFSSNNENCLAWFQQKPGQPPKLLIYWASTRASGVPDRFSGSGSGTDFTLTISSLQAEDVAVYYCQQYLKAPRTFGQGTKVEIK

SEQ ID NO: 120 - protein, 124 aa, Homo sapiens
QLQLQESGPGLVKPAETLSLTCIVSGDSVSSPSYFWGWIRQPPGKGLEWIGATWFNGDTYYNPSLQSRVSISMDMSLNRIYLSLNSVTAADTAVYFCATEHCFDEICSPASDSWGQGTLVTVSS

SEQ ID NO: 121 - protein, 113 aa, Homo sapiens
DIVMTQSPDSLAVSLGERATISCMSSQSVFFSSNNENCLAWFQQKPGQPPKLLIYWASTRASGVPDRFSGSGSGTDFTLTISSLQAEDVAVYYCQQYLKAPRTFGQGTKVELK

SEQ ID NO: 122 - protein, 7 aa, Homo sapiens
TYYTGDT

SEQ ID NO: 123 - protein, 16 aa, Homo sapiens
ATEFCINGICRPASDS

SEQ ID NO: 124 - protein, 12 aa, Homo sapiens
QSVLLSSNNKNC

SEQ ID NO: 125 - protein, 124 aa, Homo sapiens
QLQLQESGPGLVKPSETLSLTCTVSGDSVSSSFYFWGWVRQPPGKGLEWIGGTYYTGDTYHNPSLKSRLTISMDTSKNQFSLRLSSVTAADTAVYYCATEFCINGICRPASDSWGQGTLVTVSS

SEQ ID NO: 126 - protein, 113 aa, Homo sapiens
DIVMTQSPDSLAVSLGERATINCKSSQSVLLSSNNKNCLAWFQQRPGQPPKLLIYWASTRESGVPDRFSGSGSGTDFTLTISSLQAEDVAIYYCQQYYKAPRTFGQGTKVEIK

SEQ ID NO: 127 - protein, 8 aa, Homo sapiens
GYQFTDYC

SEQ ID NO: 128 - protein, 8 aa, Homo sapiens
IFPDTFDI

SEQ ID NO: 129 - protein, 21 aa, Homo sapiens
ARIERVPYGSEGEYLNVGCDS

SEQ ID NO: 130 - protein, 6 aa, Homo sapiens
QNINSW

SEQ ID NO: 131 - skipped

SEQ ID NO: 132 - protein, 9 aa, Homo sapiens
LIYMASTLQ

SEQ ID NO: 133 - protein, 9 aa, Homo sapiens
QHFNKSPYT

SEQ ID NO: 134 - protein, 128 aa, synthetic construct
  REGION 1..128: note = aCCL8.008 VH
EVQLVQSRAEVKKPGQSLTISCEASGYQFTDYCVAWVRQVPGKGLEFVAIIFPDTFDIKYSPSFQGHVTISADKSITTVFLHWTSLETSDTAMYFCARIERVPYGSEGEYLNVGCDSWGQGTLVTVSS

SEQ ID NO: 135 - protein, 107 aa, synthetic construct
  REGION 1..107: note = aCCL8.008 VL
DIQMTQSPSSLSASVGGRVTITCRASQNINSWLAWYQQKPGKTPKLLIYMASTLQPGVPSRFSGSGFGTDFTLTISGLQPDDFATYYCQHFNKSPYTFGQGTKVEIK

SEQ ID NO: 136 - protein, 128 aa, Homo sapiens
EVQLVQSGAEVKKPGQSLTISCEASGYQFTDYCVAWVRQVPGKGLEFVAIIFPDTFDIKYSPSFQGHVTISADKSITTVFLHWTSLETSDTAMYFCARIERVPYGSEGEYLNVGCDSWGQGTQVIVSS

SEQ ID NO: 137 - protein, 107 aa, Homo sapiens
DIQMTQSPSTLSASVGGRVTITCRASQNINSWLAWYQQKPGKTPKLLIYMASTLQPGVPSRFSGSGFGTDFTLTISGLQPDDFATYYCQHFNKSPYTFGQGTLVEVK

SEQ ID NO: 138 - protein, 10 aa, Homo sapiens
GDSVSSSSYF

SEQ ID NO: 139 - protein, 124 aa, Homo sapiens
QLQLQESGPGLVKPSETLSLTCTVSGDSVSSSSYFWGWVRQPPGKGLEWIGSIYYTGDTYYKPSLKSRVTISMNTSKNQISLRLSSVTAADTAVYYCATEFCTSGICHPASDSWGQGTLVTVSS

SEQ ID NO: 140 - protein, 113 aa, Homo sapiens
DIVMTQSPDSLAMSLGERATINCKSSQSVLLSANNKNCLAWIQQKPGQPPKVLIYWASTRESGVPDRFSGSGSGTDFTLTISSLQAEDVAIYYCQQYYKAPRTFGQGTKVEIK

SEQ ID NO: 141 - protein, 10 aa, Homo sapiens
GDSVSTSSYF

SEQ ID NO: 142 - protein, 7 aa, Homo sapiens
IFYTGDT

SEQ ID NO: 143 - protein, 16 aa, Homo sapiens
ATEFCTSGICHPASDS

SEQ ID NO: 144 - protein, 124 aa, Homo sapiens
QLQLQESGPGLLKPSETLSLTCTVSGDSVSTSSYFWGWVRQPPGKGLEWIGSIFYTGDTYYKPSLKSRVTISMNTSKNQISLRLSSVTAADTAVYYCATEFCTSGICHPASDSWGQGTLVTVSS

SEQ ID NO: 145 - protein, 113 aa, Homo sapiens
DIVMTQSPDSLAVSLGERATINCKSSQSVLLSANNKNCLAWIQQKPGQPPKVLIYWASTRESGVPDRFSGSGSGTDFTLTISSLQAEDVAIYYCQQYYKAPRTFGQGTKVEIK

SEQ ID NO: 146 - protein, 124 aa, Homo sapiens
QLQLQESGPGLLKPSETLSLTCTVSGDSVSSSSYFWGWVRQPPGKGLEWIGSIFYTGDTYYKPSLKSRVTISMNTSKNQISLRLSSVTAADTAVYYCATEFCTSGICHPASDSWGQGTLVTVSS

SEQ ID NO: 147 - protein, 113 aa, Homo sapiens; identical to SEQ ID NO: 145

SEQ ID NO: 148 - protein, 8 aa, Homo sapiens
GSRVTEYY

SEQ ID NO: 149 - protein, 8 aa, Homo sapiens
IRPATLDI

SEQ ID NO: 150 - protein, 21 aa, Homo sapiens
ARIKVMPYGSEGNYWNVGCDS

SEQ ID NO: 151 - protein, 6 aa, Homo sapiens
QNIYSW

SEQ ID NO: 152 - skipped

SEQ ID NO: 153 - protein, 9 aa, Homo sapiens
LIYLASDLQ

SEQ ID NO: 154 - protein, 9 aa, Homo sapiens
QHYNKSPYT

SEQ ID NO: 155 - protein, 136 aa, Homo sapiens
EVQLVQSGPEVKKPGESLTISCEASGSRVTEYYVAWVRQMPGRGLEYVGIIRPATLDIRYDPSFQGHVTMSADESSTTATDRSRTTVFLHWTSLETSDTAIYYCARIKVMPYGSEGNYWNVGCDSWGQGTQVIVSS

SEQ ID NO: 156 - protein, 106 aa, Homo sapiens
DIQMTQSPSTLSASVGDRVTITCRASQNIYSWLAWYQQKPGKTPKLLIYLASDLQPAVPSRFSGSGFGTYFTLTINDLQPDDFATYYCQHYNKSPYTFGQGTRVEI

SEQ ID NO: 157 - protein, 8 aa, Homo sapiens
GFIFSNYA

SEQ ID NO: 158 - protein, 8 aa, Homo sapiens
ISFDARNK

SEQ ID NO: 159 - protein, 23 aa, Homo sapiens
ARDLFLSDYDNGVYDPTRGGFDY

SEQ ID NO: 160 - protein, 6 aa, Homo sapiens
QSVSDW

SEQ ID NO: 161 - skipped

SEQ ID NO: 162 - protein, 9 aa, Homo sapiens
LIYKASILE

SEQ ID NO: 163 - protein, 9 aa, Homo sapiens
QKYNKSPYT

SEQ ID NO: 164 - protein, 130 aa, Homo sapiens
QVQLVESGGGVVQPGRPLRLSCSGSGFIFSNYAMHWVRQAPGKGLEWVAIISFDARNKRYADSVEGRFTISRDNSKNILYLQMNSLRLEDSAVYFCARDLFLSDYDNGVYDPTRGGFDYWGQGTLVTVSS

SEQ ID NO: 165 - protein, 106 aa, Homo sapiens
  SITE 9: note = misc_feature - Xaa can be any naturally occurring amino acid
DIQMTQSPXSLSASVGDRVIITCRTSQSVSDWLAWYQQKPGRTPKLLIYKASILEAGVPSRFRGSGYGTEFTLTINSLQPDDFATYYCQKYNKSPYTFGQGTRLEL

SEQ ID NO: 166 - protein, 12 aa, Homo sapiens
QSVLFGSNNENC

SEQ ID NO: 167 - protein, 9 aa, Homo sapiens
LISWASTRA

SEQ ID NO: 168 - protein, 9 aa, Homo sapiens
QQYRKAPRT

SEQ ID NO: 169 - protein, 130 aa, Homo sapiens; identical to SEQ ID NO: 164

SEQ ID NO: 170 - protein, 113 aa, Homo sapiens
DIVMTQSPDSLAVPLGERATISCKSSQSVLFGSNNENCLAWFQQRPGQPPKLLISWASTRASGVPDRFSGSGSETDFTLTISSLQPEDVAIYYCQQYRKAPRTFGQGTRLEIK

SEQ ID NO: 171 - protein, 10 aa, Homo sapiens
GASISRGDYA

SEQ ID NO: 172 - protein, 7 aa, Homo sapiens
VYHSGTT

SEQ ID NO: 173 - protein, 17 aa, Homo sapiens
AREGRATAWEKNNFFDS

SEQ ID NO: 174 - protein, 12 aa, Homo sapiens
QSVLFGSNNENC

SEQ ID NO: 175 - protein, 124 aa, Homo sapiens
QLQLQESGSGLVKPSQTLSLTCIVSGASISRGDYAWSWFRQPPGKGLEWIGYVYHSGTTYYSPSLKNRVTISVDRSRNQFSVRLTSVTAADTAVYYCAREGRATAWEKNNFFDSWGQGTLVTVS

SEQ ID NO: 176 - protein, 113 aa, Homo sapiens
DIVMTQSPDSLAVSLGERATISCRSSQSVLFGSNNENCLAWFQQRPGQPPKLLISWASTRASGVPDRFSGSGSETDFTLTISSLQPEDVAIYYCQQYRKAPRTFGQGTRLEIK

SEQ ID NO: 177 - protein, 10 aa, Homo sapiens
GASVNSDAYF

SEQ ID NO: 178 - protein, 7 aa, Homo sapiens
IYYTGET

SEQ ID NO: 179 - protein, 16 aa, Homo sapiens
ATESCIDGLCSPASDS

SEQ ID NO: 180 - protein, 14 aa, Homo sapiens
QSVLFSSDNDNENC

SEQ ID NO: 181 - protein, 124 aa, Homo sapiens
QLQLQESGPGLVKPWETLSLTCSVSGASVNSDAYFWGWVRQPPGQGLEWIGSIYYTGETHYNPSLKSRVSISMDTSENQLSLILNSVTAADTAVYYCATESCIDGLCSPASDSWGQGTLVTVSA

SEQ ID NO: 182 - protein, 115 aa, Homo sapiens
  SITE 9: note = misc_feature - Xaa can be any naturally occurring amino acid
DIVMTQSPXSLAVSLGERATISCKSSQSVLFSSDNDNENCLAWFQQKPGQPPKLLIYWASTRASGVPDRFSGSGSGTDFTLTISSLQPEDVAVYYCQQYRKAPRTFGQGTRLEIK

SEQ ID NO: 183 - protein, 8 aa, Homo sapiens
GYTFTNHY

SEQ ID NO: 184 - protein, 8 aa, Homo sapiens
VNPSGGNT

SEQ ID NO: 185 - protein, 20 aa, Homo sapiens
ARDGSHYDDDGGYYGDAFDI

SEQ ID NO: 186 - protein, 12 aa, Homo sapiens
QSVLYNSNNKNF

SEQ ID NO: 187 - protein, 9 aa, Homo sapiens
LMYWASTRE

SEQ ID NO: 188 - protein, 9 aa, Homo sapiens
QQYYSSPFT

SEQ ID NO: 189 - protein, 127 aa, synthetic construct
  REGION 1..127: note = aCCL20.001 VH
QVQLVQSGAEVKKPGASVKISCKASGYTFTNHYMHWVRQAPGVGLEWMGIVNPSGGNTSYARNFQGRIIMTRDMSTRTIYMELGSLRSEDTAVYYCARDGSHYDDDGGYYGDAFDIWGQGTMVTVSS

SEQ ID NO: 190 - protein, 113 aa, synthetic construct
  REGION 1..113: note = aCCL20.001 VL
DIVMTQSPDSLAVSLGERATINCKSSQSVLYNSNNKNFLAWYQQRPGQPPKLLMYWASTRESGVPDRFSGSGSATDFTLTISSLQAEDVAVYYCQQYYSSPFTFGPGTKVDIK

SEQ ID NO: 191 - protein, 127 aa, Homo sapiens
QVQLVQSGAEVKKPGASVKISCKASGYTFTNHYMHWVRQAPGVGLEWMGIVNPSGGNTSYARNFQGRIIMTRDMSTRTIYMELGSLRSEDTAVYYCARDGSHYDDDGGYYGDAFDIWGQGTVVTVSS

SEQ ID NO: 192 - protein, 113 aa, Homo sapiens
DIVMTQSPDSLAVSLGERATINCKSSQSVLYNSNNKNFLAWYQQRPGQPPKLLMYWASTRESGVPDRFSGSGSATDFTLTISSLQAEDVAVYYCQQYYSSPFTFGPGTKVDLK

SEQ ID NO: 193 - protein, 8 aa, Homo sapiens
GFTFSNYS

SEQ ID NO: 194 - protein, 8 aa, Homo sapiens
ISSRSSYI

SEQ ID NO: 195 - protein, 13 aa, Homo sapiens
ARVEVIRGVRFDY

SEQ ID NO: 196 - protein, 6 aa, Homo sapiens
QGISSY

SEQ ID NO: 197 - skipped

SEQ ID NO: 198 - protein, 9 aa, Homo sapiens
LIYGASTLQ

SEQ ID NO: 199 - protein, 9 aa, Homo sapiens
QQLHSSPPS

SEQ ID NO: 200 - protein, 120 aa, synthetic construct
  REGION 1..120: note = aCCL23.001 VH
QVQLVESGGGLVKPGGSLRLSCAASGFTFSNYSMNWVRQAPGKGLEWVSSISSRSSYIYYADSVKGRFTISRDNAKKSLFLQMNSLRVEDTAVYYCARVEVIRGVRFDYWGQGTLVTVSS

SEQ ID NO: 201 - protein, 107 aa, Homo sapiens
DIQLTQSPSFLSASVGDRVTITCRASQGISSYLAWYQQKPGKAPKLLIYGASTLQGGVPSTFSGSGSGTDFTLTISSLQPEDSATYYCQQLHSSPPSFGQGTKLEIK

SEQ ID NO: 202 - protein, 120 aa, Homo sapiens
EVQLVESGGGLVKPGGSLRLSCAASGFTFSNYSMNWVRQAPGKGLEWVSSISSRSSYIYYADSVKGRFTISRDNAKKSLFLQMNSLRVEDTAVYYCARVEVIRGVRFDYWGQGTLVTVSS

SEQ ID NO: 203 - protein, 8 aa, Homo sapiens
GFTFSSYA

SEQ ID NO: 204 - protein, 8 aa, Homo sapiens
ISGSGGGT

SEQ ID NO: 205 - protein, 16 aa, Homo sapiens
ARVKGEVGGVIYGMDV

SEQ ID NO: 206 - protein, 7 aa, Homo sapiens
QSVTSSY

SEQ ID NO: 207 - protein, 9 aa, Homo sapiens
LIYGASTRA

SEQ ID NO: 208 - protein, 6 aa, Homo sapiens
QHRGFT

SEQ ID NO: 209 - protein, 123 aa, Homo sapiens
EVQLLESGGGLVQPGGSLRVSCAASGFTFSSYAMSWVRQAPGKGLEWVSAISGSGGGTYYAHSVKGRITISRDNSKNTLYLQMNSLRAEDTAVYHCARVKGEVGGVIYGMDVWGQGTTVTVSS

SEQ ID NO: 210 - protein, 105 aa, Homo sapiens
EIVLTQSPGTLSLSPGERATLSCRASQSVTSSYLAWYQQKPGQAPRLLIYGASTRATGIPDRFSGSGSGTDFTLTISRLEPEDFAVYYCQHRGFTFGPGTKVDIK

SEQ ID NO: 211 - protein, 8 aa, Homo sapiens
GFMFSDYY

SEQ ID NO: 212 - protein, 8 aa, Homo sapiens
IGNSDAST

SEQ ID NO: 213 - protein, 21 aa, Homo sapiens
ARDGYSHSRGYSYGRKDAFDI

SEQ ID NO: 214 - protein, 6 aa, Homo sapiens
QSVSSY

SEQ ID NO: 215 - skipped

SEQ ID NO: 216 - protein, 9 aa, Homo sapiens
LIYAASQRA

SEQ ID NO: 217 - protein, 9 aa, Homo sapiens
QQRGNWPGT

SEQ ID NO: 218 - protein, 128 aa, Homo sapiens
QVQLVESGGGLVKPGGSLRLSCAASGFMFSDYYMSWIRQAPGKGLEWISYIGNSDASTFYADSVKGRFAISRDNAKNLLFLQMNSLRAEDTAVYYCARDGYSHSRGYSYGRKDAFDIWGRGTMVTVSS

SEQ ID NO: 219 - protein, 107 aa, synthetic construct
  REGION 1..107: note = aCXCL13.001 VL
EIVLTQSPATLSLSPGERATLSCRASQSVSSYLAWYQQKPGQAPRLLIYAASQRATGIPARFSGTGSGTDFTLTISSLEPEDSAVYYCQQRGNWPGTFGQGTKVEIK

SEQ ID NO: 220 - protein, 107 aa, Homo sapiens
  SITE 67: note = misc_feature - Xaa can be any naturally occurring amino acid
  SITE 83: note = misc_feature - Xaa can be any naturally occurring amino acid
EIVLTQSPATLSLSPGERATLSCRASQSVSSYLAWYQQKPGQAPRLLIYAASQRATGIPARFSGTGXGTDFTLTISSLEPEDXAVYYCQQRGNWPGTFGQGTKLEIK

SEQ ID NO: 221 - protein, 8 aa, Homo sapiens
GFTFSSYS

SEQ ID NO: 222 - protein, 8 aa, Homo sapiens
ISSSSSYI

SEQ ID NO: 223 - protein, 13 aa, Homo sapiens
ARDGIYDYAPVDY

SEQ ID NO: 224 - protein, 12 aa, Homo sapiens
QSVLYSSNNKNY

SEQ ID NO: 225 - protein, 9 aa, Homo sapiens
QQYYSTPFT

SEQ ID NO: 226 - protein, 120 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDGIYDYAPVDYWGQGTLVTVSS

SEQ ID NO: 227 - protein, 113 aa, synthetic construct
  REGION 1..113: note = aCXCL13.002 VL
DILMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPFTFGGGTKVEIK

SEQ ID NO: 228 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPFTFGGGTKVEIK

SEQ ID NO: 229 - protein, 8 aa, Homo sapiens
GGSFSGYY

SEQ ID NO: 230 - protein, 7 aa, Homo sapiens
INHSGST

SEQ ID NO: 231 - protein, 15 aa, Homo sapiens
ARAGRSYGDYGAFDI

SEQ ID NO: 232 - protein, 121 aa, synthetic construct
  REGION 1..121: note = aCXCL13.003 VH
QVQLQEWGAGLLKPSETLSLTCAVYGGSFSGYYWSWIRQPPGKGLEWIGEINHSGSTNYNPSLKSRVTISVDTSKNQFSLKLSSVTAADTAVYYCARAGRSYGDYGAFDIWGQGTMVTVSS

SEQ ID NO: 233 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPFTFGPGTKVDIK

SEQ ID NO: 234 - protein, 121 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARAGRSYGDYGAFDIWGQGTMVTVSS

SEQ ID NO: 235 - protein, 8 aa, Homo sapiens
GYPFANAW

SEQ ID NO: 236 - protein, 10 aa, Homo sapiens
IKTKSEGETT

SEQ ID NO: 237 - protein, 5 aa, Homo sapiens
TTVFF

SEQ ID NO: 238 - protein, 6 aa, Homo sapiens
ALSQQY

SEQ ID NO: 239 - skipped

SEQ ID NO: 240 - protein, 9 aa, Homo sapiens
VISKDTERP

SEQ ID NO: 241 - protein, 11 aa, Homo sapiens
HSTDISDSYWV

SEQ ID NO: 242 - protein, 114 aa, synthetic construct
  REGION 1..114: note = aCXCL16.001 VH
EVQLVESGGGLVKPGESLTLSCVASGYPFANAWMSWVRQAPGKGLEWVGRIKTKSEGETTDYAPLVRGRFTISRDDSKTTVFLQMNSLKTEDTGMYYCTTVFFWGRGTLVTVSS

SEQ ID NO: 243 - protein, 108 aa, synthetic construct
  REGION 1..108: note = aCXCL16.001 VL
SYELTQPPSVSVSPGQTARITCSGDALSQQYLFWHQQRPGQAPKVVISKDTERPSGIPERFSGSISGTTGTLTISGVQTEDEADYYCHSTDISDSYWVFGGGTKLTVL

SEQ ID NO: 244 - protein, 114 aa, Homo sapiens
EVQLVESGGGLVKPGESLTLSCVASGYPFANAWMSWVRQAPGKGLEWVGRIKTKSEGETTDYAPLVRGRFTISRDDSKTTVFLQMNSLKTEDTGMYYCTTVFFWGRGTLVGVSS

SEQ ID NO: 245 - protein, 106 aa, Homo sapiens
SYELTQPPSVSVSPGQTARITCSGDALSQQYLFWHQQRPGQAPKVVISKDTERPSGIPERFSGSISGTTGTLTISGVQTEDEADYYCHSTDISDSYWVFGGGTKLT

SEQ ID NO: 246 - protein, 8 aa, Homo sapiens
GLTVTRNY

SEQ ID NO: 247 - protein, 7 aa, Homo sapiens
LWTDGQK

SEQ ID NO: 248 - protein, 21 aa, Homo sapiens
TRDKVVPVRITPDYHYYGMDV

SEQ ID NO: 249 - protein, 6 aa, Homo sapiens
QGVRRY

SEQ ID NO: 250 - protein, 9 aa, synthetic construct
  REGION 1..9: note = aCXCL16.002 CDRL2 long
LIYGASSLH

SEQ ID NO: 251 - protein, 9 aa, Homo sapiens
QQLDTYPLT

SEQ ID NO: 252 - protein, 127 aa, synthetic construct
  REGION 1..127: note = aCXCL16.002 VH
EVQLVESGGGLSQPGWSLRLSCAASGLTVTRNYMSWVRQAPGKGLEWVSTLWTDGQKFYADSVKGRFTVSRDSYTNTLNLEMSGLRAEDTAVYFCTRDKVVPVRITPDYHYYGMDVWGQGTTVTVSS

SEQ ID NO: 253 - protein, 107 aa, synthetic construct
  REGION 1..107: note = aCXCL16.002 VL
DIQLTQSPSFLSASVGDRVTITCRASQGVRRYLAWYQQKPGKAPKFLIYGASSLHTGVPSRFSGGGSDTEFTLTISNLQTEDFATYYCQQLDTYPLTFGGGTKVEIK

SEQ ID NO: 254 - protein, 9 aa, Homo sapiens
  SITE 1: note = misc_feature - Xaa can be any naturally occurring amino acid
XIYGASSLH

SEQ ID NO: 255 - protein, 127 aa, Homo sapiens
EVQLVESGGGLSQPGWSLRLSCAASGLTVTRNYMSWVRQAPGKGLEWVSTLWTDGQKFYADSVKGRFTVSRDSYTNTLNLEMSGLRAEDTAVYFCTRDKVVPVRITPDYHYYGMDVWGQGTTVIVSS

SEQ ID NO: 256 - protein, 107 aa, Homo sapiens
  SITE 47: note = misc_feature - Xaa can be any naturally occurring amino acid
  SITE 67: note = misc_feature - Xaa can be any naturally occurring amino acid
DIQLTQSPSFLSASVGDRVTITCRASQGVRRYLAWYQQKPGKAPKFXIYGASSLHTGVPSRFSGGGXDTEFTLTISNLQTEDFATYYCQQLDTYPLTFGGGTKVEIK

SEQ ID NO: 257 - protein, 8 aa, Homo sapiens
GYSFTSYW

SEQ ID NO: 258 - protein, 8 aa, Homo sapiens
IYPGDSDT

SEQ ID NO: 259 - protein, 12 aa, Homo sapiens
ARLHGDLYYFDY

SEQ ID NO: 260 - protein, 8 aa, Homo sapiens
SNNVGSYA

SEQ ID NO: 261 - skipped

SEQ ID NO: 262 - protein, 9 aa, Homo sapiens
VMFGNSLPS

SEQ ID NO: 263 - protein, 11 aa, Homo sapiens
STWDYSLSAQV

SEQ ID NO: 264 - protein, 119 aa, synthetic construct
  REGION 1..119: note = aCXCL16.003 VH
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLHGDLYYFDYWGQGTLVTVSS

SEQ ID NO: 265 - protein, 109 aa, Homo sapiens
QSVLTQEASVSGTVGQKVTLSCTGNSNNVGSYAVGWYQQISHGAPKTVMFGNSLPSGIPDRFSGSKSGTTASLTISGLQPEDEADYYCSTWDYSLSAQVFGGGTKLTVL

SEQ ID NO: 266 - protein, 116 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLHGDLYYFDYWGQGTLVT

(19) United States
(12) Patent Application Publication
(10) Pub. No.: US 2025/0298032 A1
(43) Pub. Date: Sep. 25, 2025

(54) ANTIBODIES AGAINST CHEMOKINES, METHOD FOR IDENTIFYING SAID ANTIBODIES AND USES THEREOF

(71) Applicant: INSTITUTE FOR RESEARCH IN BIOMEDICINE, Bellinzona (CH)

(72) Inventors: Davide ROBBIANI, Massagno (CH); Mariagrazia UGUCCIONI, Bellinzona (CH); Andrea CAVALLI, Zurich (CH); Jonathan MURI, Bellinzona (CH); Valentina CECCHINATO, Cermenate (IT)

(21) Appl. No.: 18/861,629

(22) PCT Filed: May 3, 2023
(86) PCT No.: PCT/EP2023/061726

§ 371 Date: Oct. 30, 2024

(30) Foreign Application Priority Data
May 5, 2022 (WO) PCT/EP2022/062147

(51) Int. Cl. G01N 33/68; C07K 16/24

(52) U.S. Cl. CPC G01N 33/6854; C07K 16/24; G01N 33/6863; C07K 2317/33; C07K 2317/92; G01N 2333/521; G01N 2800/26

(57) ABSTRACT

The present disclosure provides antibodies against chemokines, in particular to auto-antibodies against chemokines, nucleic acids encoding such antibodies and compositions comprising such antibodies. The present disclosure also provides a method for identifying (auto-)antibodies against chemokines, antibodies identified by said method and to the use of antibodies against chemokines as biomarkers, and for the treatment and diagnosis of diseases, such as COVID-19.

The present invention relates to the field of antibodies against chemokines, in particular to auto-antibodies against chemokines. In particular, the present invention relates to a method for identifying (auto-)antibodies against chemokines, to antibodies identified by said method and to the use of such antibodies as biomarkers, for the treatment and diagnosis of diseases, such as COVID-19.

The spectrum of disease manifestations upon Severe Acute Respiratory Syndrome Coronavirus 2 (SARS-CoV-2) infection is broad. Some of the factors that predispose to a higher risk of hospitalization and death include age, gender, ethnicity, obesity, genetic predisposition, autoantibodies against interferon, and comorbidities such as hypertension, diabetes, and coronary heart disease. A large fraction of COVID-19 convalescent individuals laments protracted symptoms over months, a condition referred to as long COVID or PASC (Post-Acute Sequelae of COVID), and infection increases the risk of cardiovascular events after 1 year (Blomberg, B., Mohn, K. G., Brokstad, K. A., Zhou, F., Linchausen, D. W., Hansen, B. A., Lartey, S., Onyango, T. B., Kuwelker, K., Saevik, M., et al. (2021). Long COVID in a prospective cohort of home-isolated patients. Nat Med 27, 1607-1613; Davis, H. E., Assaf, G. S., McCorkell, L., Wei, H., Low, R. J., Re'em, Y., Redfield, S., Austin, J. P., and Akrami, A. (2021). Characterizing long COVID in an international cohort: 7 months of symptoms and their impact. EClinicalMedicine 38, 101019). Some evidence points to a role for immune dysregulation and autoimmunity as contributors to long COVID, although virus persistence has also been proposed (Cervia, C., Zurbuchen, Y., Taeschler, P., Ballouz, T., Menges, D., Hasler, S., Adamo, S., Raeber, M. E., Bachli, E., Rudiger, A., et al. (2022). Immunoglobulin signature predicts risk of post-acute COVID-19 syndrome. Nat Commun 13, 446; Phetsouphanh, C., Darley, D. R., Wilson, D. B., Howe, A., Munier, C. M. L., Patel, S. K., Juno, J. A., Burrell, L. M., Kent, S. J., Dore, G. J., et al. (2022). Immunological dysfunction persists for 8 months following initial mild-to-moderate SARS-CoV-2 infection. Nat Immunol 23, 210-216). Overall, there is however little understanding of the causes of different COVID-19 severities and of the biology underlying long COVID.

Chemokines are chemotactic cytokines that mediate leukocyte trafficking and activity by binding to seven-transmembrane G protein-coupled receptors. Chemokines play a fundamental role in health and disease, and the proper trafficking of leukocyte subsets is governed by the combinatorial diversity of their responsiveness to chemokines. In addition to elevated levels of pro-inflammatory cytokines (e.g., IL-6, TNF, and IL1β), higher levels of certain chemokines are observed in COVID-19 (e.g., CCL2, CCL3, CCL4, CCL7, CCL8, CCL19, CXCL2, CXCL5, CXCL8, CXCL9, CXCL10, CXCL13, CXCL16 and CXCL17). Accordingly, neutrophils and monocytes are recruited to sites of infection, where they play a key role in the pathophysiology of COVID-19 by sustaining inflammation and causing tissue damage and fibrosis also in the inflammatory phase of COVID-19 that follows virus clearance. Anti-inflammatory treatment, such as steroids or IL-6 blockade, are efficacious in hospitalized COVID-19 patients, while therapies targeting the chemokine system are under development for immunological disorders and cancers.

Similar to earlier work linking anti-cytokine antibodies to mycobacterial, staphylococcal and fungal diseases, autoantibodies against cytokines have been described in COVID-19. In particular, anti-type I Interferon antibodies were observed in about 10% of life-threatening pneumonia and in about 20% of deaths from COVID-19 (Bastard, P., Gervais, A., Le Voyer, T., Rosain, J., Philippot, Q., Manry, J., Michailidis, E., Hoffmann, H. H., Eto, S., Garcia-Prat, M., et al. (2021). Autoantibodies neutralizing type I IFNs are present in ˜4% of uninfected individuals over 70 years old and account for ˜20% of COVID-19 deaths. Sci Immunol 6; Bastard, P., Rosen, L. B., Zhang, Q., Michailidis, E., Hoffmann, H. H., Zhang, Y., Dorgham, K., Philippot, Q., Rosain, J., Beziat, V., et al. (2020). Autoantibodies against type I IFNs in patients with life-threatening COVID-19. Science 370; Damoiseaux, J., Dotan, A., Fritzler, M. J., Bogdanos, D. P., Meroni, P. L., Roggenbuck, D., Goldman, M., Landegren, N., Bastard, P., Shoenfeld, Y., et al. (2021). Autoantibodies and SARS-CoV2 infection: The spectrum from association to clinical implication: Report of the 15th Dresden Symposium on Autoantibodies. Autoimmun Rev 21, 103012). Moreover, autoantibodies characteristic of systemic autoimmune disorders, such as anti-phospholipid antibodies, anti-nuclear antibodies and rheumatoid factor, have also been reported in COVID-19 (Chang, S. E., Feng, A., Meng, W., Apostolidis, S. A., Mack, E., Artandi, M., Barman, L., Bennett, K., Chakraborty, S., Chang, I., et al. (2021). New-onset IgG autoantibodies in hospitalized patients with COVID-19. Nat Commun 12, 5417; van der Wijst, M. G. P., Vazquez, S. E., Hartoularos, G. C., Bastard, P., Grant, T., Bueno, R., Lee, D. S., Greenland, J. R., Sun, Y., Perez, R., et al. (2021). Type I interferon autoantibodies are associated with systemic immune alterations in patients with COVID-19. Sci Transl Med 13, eabh2624; Woodruff, M. C., Ramonell, R. P., Saini, A. S., Haddad, N. S., Anam, F. A., Rudolph, M. E., Bugrovsky, R., Hom, J., Cashman, K. S., Nguyen, D. C., et al. (2021). Relaxed peripheral tolerance drives broad de novo autoreactivity in severe COVID-19. medRxiv; Zhou, Y., Han, T., Chen, J., Hou, C., Hua, L., He, S., Guo, Y., Zhang, S., Wang, Y., Yuan, J., et al. (2020). Clinical and Autoimmune Characteristics of Severe and Critical Cases of COVID-19. Clin Transl Sci 13, 1077-1086; Zuo, Y., Estes, S. K., Ali, R. A., Gandhi, A. A., Yalavarthi, S., Shi, H., Sule, G., Gockman, K., Madison, J. A., Zuo, M., et al. (2020). Prothrombotic autoantibodies in serum from patients hospitalized with COVID-19. Sci Transl Med 12). A recent high-throughput screening by yeast-display of the secretome further revealed the presence of autoantibodies against a number of immune factors, including chemokines (Wang, E. Y., Mao, T., Klein, J., Dai, Y., Huck, J. D., Jaycox, J. R., Liu, F., Zhou, T., Israelow, B., Wong, P., et al. (2021). Diverse functional autoantibodies in patients with COVID-19. Nature 595, 283-288). However, anti-chemokine antibodies were infrequent by this method, and there was neither correlation with disease severity or long COVID, nor information about the persistence of such autoantibodies over time. Moreover, Wang et al. did not clone any antibodies targeting chemokines.

In view of the above, it is the object of the present invention to provide a novel method for identifying autoantibodies against chemokines, which is based on a set of peptides designed to obtain antibodies that bind to a functional region of each of the 43 human chemokines. It is also an object of the present invention to provide anti-chemokine antibodies identified with said method. It is also an object of the present invention to provide the use of anti-chemokine antibodies in the diagnosis and/or treatment of diseases, and/or as biomarkers.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%, for example, x±5%, or x±7%, or x±10%, or x±12%, or x±15%, or x±20%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention, prophylaxis, attenuation, amelioration and therapy. The terms “subject” or “patient” are used interchangeably herein to mean all mammals including humans. Examples of subjects include humans, cows, dogs, cats, horses, goats, sheep, pigs, and rabbits. In some embodiments, the subject or patient is a human.

Doses are often expressed in relation to the bodyweight. Thus, a dose which is expressed as [g, mg, or other unit]/kg (or g, mg etc.) usually refers to [g, mg, or other unit]“per kg (or g, mg etc.) bodyweight”, even if the term “bodyweight” is not explicitly mentioned.

The term “binding” and similar reference usually means “specifically binding”, which does not encompass non-specific sticking.

As used herein, the term “antibody” encompasses various forms of antibodies including, without being limited to, whole antibodies, antibody fragments (such as antigen binding fragments), human antibodies, chimeric antibodies, humanized antibodies, recombinant antibodies and genetically engineered antibodies (variant or mutant antibodies) as long as the characteristic properties according to the invention are retained. In some embodiments, the antibody is a human antibody. In some embodiments, the antibody is a monoclonal antibody. For example, the antibody is a human monoclonal antibody.

As described above, the term “antibody” generally also includes antibody fragments. Fragments of the antibodies may retain the antigen-binding activity of the antibodies. Such fragments are referred to as “antigen-binding fragments”. Antigen-binding fragments include, but are not limited to, single chain antibodies, Fab, Fab′, F(ab′)2, Fv or scFv. Fragments of the antibodies can be obtained from the antibodies by methods that include digestion with enzymes, such as pepsin or papain, and/or by cleavage of disulfide bonds by chemical reduction. Alternatively, fragments of the antibodies can be obtained by recombinant means, for example by cloning and expressing a part (fragment) of the sequences of the heavy and/or light chain. The invention also encompasses single-chain Fv fragments (scFv) derived from the heavy and light chains of an antibody of the invention. For example, the invention includes a scFv comprising the CDRs from an antibody of the invention. Also included are heavy or light chain monomers and dimers, single domain heavy chain antibodies, single domain light chain antibodies, as well as single chain antibodies, e.g., single chain Fv in which the heavy and light chain variable domains are joined by a peptide linker. Antibody fragments of the invention may be contained in a variety of structures known to the person skilled in the art. In addition, the sequences of the invention may be a component of multispecific molecules in which the sequences of the invention target the epitopes of the invention and other regions of the molecule bind to other targets. Although the specification, including the claims, may, in some places, refer explicitly to antigen binding fragment(s), antibody fragment(s), variant(s) and/or derivative(s) of antibodies, it is understood that the term “antibody” includes all categories of antibodies, namely, antigen binding fragment(s), antibody fragment(s), variant(s) and derivative(s) of antibodies.

Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., j. Immunol. 147 (1991) 86-95). In some embodiments, human monoclonal antibodies are prepared by using improved EBV-B cell immortalization as described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5. As used herein, the term “variable region” (variable region of a light chain (V_{L}), variable region of a heavy chain (V_{H})) denotes each of the pair of light and heavy chains which is involved directly in binding the antibody to the antigen.

Antibodies of the invention can be of any isotype (e.g., IgA, IgG, IgM i.e. an α, γ or μ heavy chain). For example, the antibody is of the IgG type. Within the IgG isotype, antibodies may be IgG1, IgG2, IgG3 or IgG4 subclass, for example IgG1. Antibodies of the invention may have a κ or a λ light chain. In some embodiments, the antibody is of IgG1 type and has a κ light chain.

Antibodies according to the present invention may be provided in purified form. Typically, the antibody will be present in a composition that is substantially free of other polypeptides e.g., where less than 90% (by weight), usually less than 60% and more usually less than 50% of the composition is made up of other polypeptides.

Antibodies according to the present invention may be immunogenic in human and/or in non-human (or heterologous) hosts e.g., in mice. For example, the antibodies may have an idiotope that is immunogenic in non-human hosts, but not in a human host. Antibodies of the invention for human use include those that cannot be easily isolated from hosts such as mice, goats, rabbits, rats, non-primate mammals, etc. and cannot generally be obtained by humanization or from xeno-mice.

As used herein, the term “antigen” refers to any structural substance which serves as a target for the receptors of an adaptive immune response, in particular as a target for antibodies, T cell receptors, and/or B cell receptors. An “epitope”, also known as “antigenic determinant”, is the part (or fragment) of an antigen that is recognized by the immune system, in particular by antibodies, T cell receptors, and/or B cell receptors. Thus, one antigen has at least one epitope, i.e. a single antigen has one or more epitopes. An antigen may be (i) a peptide, a polypeptide, or a protein, (ii) a polysaccharide, (iii) a lipid, (iv) a lipoprotein or a lipopeptide, (v) a glycolipid, (vi) a nucleic acid, or (vii) a small molecule drug or a toxin. Thus, an antigen may be a peptide, a protein, a polysaccharide, a lipid, a combination thereof including lipoproteins and glycolipids, a nucleic acid (e.g. DNA, siRNA, shRNA, antisense oligonucleotides, decoy DNA, plasmid), or a small molecule drug (e.g. cyclosporine A, paclitaxel, doxorubicin, methotrexate, 5-aminolevulinic acid), or any combination thereof. Preferably, the antigen is selected from (i) a peptide, a polypeptide, or a protein, (ii) a polysaccharide, (iii) a lipid, (iv) a lipoprotein or a lipopeptide and (v) a glycolipid; more preferably, the antigen is a peptide, a polypeptide, or a protein.

As used herein, the term “mutation” relates to a change in the nucleic acid sequence and/or in the amino acid sequence in comparison to a reference sequence, e.g. a corresponding genomic sequence. A mutation, e.g. in comparison to a genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence. A mutation includes substitution, deletion and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

As used herein (i.e. throughout the present specification), the term “sequence variant” refers to any alteration in comparison to a reference sequence. The term “sequence variant” includes nucleotide sequence variants and amino acid sequence variants. Preferably, a reference sequence is any of the sequences listed in the “Table of Sequences and SEQ ID Numbers” (Sequence listing), i.e. SEQ ID NO: 1 to SEQ ID NO: 266. In particular, a sequence variant shares (over the whole length of the sequence) at least 70% or at least 75%, preferably at least 80% or at least 85%, more preferably at least 90% or at least 93%, even more preferably at least 95% or at least 96%, still more preferably at least 97% or at least 98%, particularly preferably at least 99% sequence identity with its reference sequence. In some embodiments, the sequence variant shares at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity. Thereby, the higher the %-identity of a sequence variant, the more it is preferred. For example, a sequence variant having at least 84% sequence identity with a reference sequence is more preferred than a sequence variant having at least 75% sequence identity, but less than 84% sequence identity, with a reference sequence.

Sequence identity may be calculated as described below. Usually a sequence variant may preserve the specific function of the reference sequence. In some embodiments, an amino acid sequence variant has an altered sequence in which one or more (e.g., 1, 2, 3, 4, 5, 6, 7, 8, 9, 10 or more) of the amino acids in the reference sequence is deleted or substituted, or one or more (e.g., 1, 2, 3, 4, 5, 6, 7, 8, 9, 10 or more) amino acids are inserted into or added to the sequence of the reference amino acid sequence. As a result of the alterations, the amino acid sequence variant has an amino acid sequence which is at least 70% or at least 75%, preferably at least 80% or at least 85%, more preferably at least 90% or at least 93%, even more preferably at least 95% or at least 96%, still more preferably at least 97% or at least 98%, particularly preferably at least 99% identical to the reference sequence. For example, variant sequences which are at least 90% identical have no more than 10 alterations, i.e., any combination of deletions, insertions or substitutions, per 100 amino acids of the reference sequence. The same, of course, also applies similarly to nucleic acid sequences.

The “% identity” of the sequence variant is usually determined with respect to the reference sequence. It is usually calculated with regard to the full length of the reference sequence (i.e. the sequence recited in the application). Percentage identity, as referred to herein, can be determined, for example, by methods known in the art, such as BLAST using the default parameters specified by the NCBI (the National Center for Biotechnology Information; http://www.ncbi.nlm.nih.gov/) [Blosum 62 matrix; gap open penalty=11 and gap extension penalty=1].

In general, while it is possible to have non-conservative amino acid substitutions, the substitutions are preferably conservative amino acid substitutions, wherein the substituted amino acid has similar structural or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, cysteine, and glycine, with another.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

### Chemokine Peptides and Related Methods

In a first aspect the present invention provides a (in-vitro) method for determining binding of an antibody, or an antigen binding fragment thereof, to (the N-loop of) a human chemokine, the method comprising the step of determining binding of the antibody, or the antigen binding fragment thereof, to a peptide comprising an amino acid sequence according to any one of SEQ ID NOs 52-93; or to a sequence variant thereof.

The present inventors identified a peptide-based strategy to determine binding of antibodies to a functional region of each of the 43 human chemokines. Based on this method, biologically active, human-derived monoclonal antibodies against chemokines, such as CCL8, CCL20, CCL23, CXCL13 and CXCL16 were obtained. By screening diverse cohorts of individuals using this peptide-based method, the present inventors surprisingly found that auto-antibodies against different sets of chemokines are associated with the status of various diseases, including COVID-19, HIV infection and autoimmune disorders, such as ankylosing spondylitis, rheumatoid arthritis and Sjögren syndrome.

The peptides used in the method as described herein are based on the N-terminal loop (“N-loop”) of the 43 human chemokines. Accordingly, the human chemokine may be selected from the group consisting of CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL16, CXCL17, XCL1, XCL2 and CX3CL1, or any combination thereof.

In some embodiments, the method is used for determining binding of an antibody, or an antigen binding fragment thereof, to CCL1, wherein the binding of the antibody, or the antigen binding fragment thereof, to the peptide comprising the amino acid sequence according to SEQ ID NO: 52 is determined.

In some embodiments, the method is used for determining binding of an antibody, or an antigen binding fragment thereof, to CCL2, wherein the binding of the antibody, or the antigen binding fragment thereof, to the peptide comprising the amino acid sequence according to SEQ ID NO: 53 is determined.

In some embodiments, the method is used for determining binding of an antibody, or an antigen binding fragment thereof, to CCL3, wherein the binding of the antibody, or the antigen binding fragment thereof, to the peptide comprising the amino acid sequence according to SEQ ID NO: 54 is determined.

In some embodiments, the method is used for determining binding of an antibody, or an antigen binding fragment thereof, to CCL4, wherein the binding of the antibody, or the antigen binding fragment thereof, to the peptide comprising an amino acid sequence according to SEQ ID NO: 55 is determined.

In some embodiments, the method is used for determining binding of an antibody, or an antigen binding fragment thereof, to CCL5, wherein the binding of the antibody, or the antigen binding fragment thereof, to the peptide comprising the amino acid sequence according to SEQ ID NO: 56 is determined.

In some embodiments, the method is used for determining binding of an antibody, or an antigen binding fragment thereof, to CCL7, wherein the binding of the antibody, or the antigen binding fragment thereof, to the peptide comprising the amino acid sequence according to SEQ ID NO: 57 is determined.

In some embodiments, the method is used for determining binding of an antibody, or an antigen binding fragment thereof, to CCL8, wherein the binding of the antibody, or the antigen binding fragment thereof, to the peptide comprising the amino acid sequence according to SEQ ID NO: 58 is determined.

In some embodiments, the method is used for determining binding of an antibody, or an antigen binding fragment thereof, to CCL11, wherein the binding of the antibody, or the antigen binding fragment thereof, to the peptide comprising the amino acid sequence according to SEQ ID NO: 59 is determined.

In some embodiments, the method is used for determining binding of an antibody, or an antigen binding fragment thereof, to CCL13, wherein the binding of the antibody, or the antigen binding fragment thereof, to the peptide comprising the amino acid sequence according to SEQ ID NO: 60 is determined.

In some embodiments, the method is used for determining binding of an antibody, or an antigen binding fragment thereof, to CCL14, wherein the binding of the antibody, or the antigen binding fragment thereof, to the peptide comprising the amino acid sequence according to SEQ ID NO: 61 is determined.

In some embodiments, the method is used for determining binding of an antibody, or an antigen binding fragment thereof, to CCL15, wherein the binding of the antibody, or the antigen binding fragment thereof, to the peptide comprising the amino acid sequence according to SEQ ID NO: 62 is determined.

In some embodiments, the method is used for determining binding of an antibody, or an antigen binding fragment thereof, to CCL16, wherein the binding of the antibody, or the antigen binding fragment thereof, to the peptide comprising the amino acid sequence according to SEQ ID NO: 63 is determined.

In some embodiments, the method is used for determining binding of an antibody, or an antigen binding fragment thereof, to CCL17, wherein the binding of the antibody, or the antigen binding fragment thereof, to the peptide comprising the amino acid sequence according to SEQ ID NO: 64 is determined.

In some embodiments, the method is used for determining binding of an antibody, or an antigen binding fragment thereof, to CCL18, wherein the binding of the antibody, or the antigen binding fragment thereof, to the peptide comprising the amino acid sequence according to SEQ ID NO: 65 is determined.

In some embodiments, the method is used for determining binding of an antibody, or an antigen binding fragment thereof, to CCL19, wherein the binding of the antibody, or the antigen binding fragment thereof, to the peptide comprising the amino acid sequence according to SEQ ID NO: 66 is determined.

In some embodiments, the method is used for determining binding of an antibody, or an antigen binding fragment thereof, to CCL20, wherein the binding of the antibody, or the antigen binding fragment thereof, to the peptide comprising the amino acid sequence according to SEQ ID NO: 67 is determined.

In some embodiments, the method is used for determining binding of an antibody, or an antigen binding fragment thereof, to CCL21, wherein the binding of the antibody, or the antigen binding fragment thereof, to the peptide comprising the amino acid sequence according to SEQ ID NO: 68 is determined.

In some embodiments, the method is used for determining binding of an antibody, or an antigen binding fragment thereof, to CCL22, wherein the binding of the antibody, or the antigen binding fragment thereof, to the peptide comprising the amino acid sequence according to SEQ ID NO: 69 is determined.

In some embodiments, the method is used for determining binding of an antibody, or an antigen binding fragment thereof, to CCL23, wherein the binding of the antibody, or the antigen binding fragment thereof, to the peptide comprising the amino acid sequence according to SEQ ID NO: 70 is determined.

In some embodiments, the method is used for determining binding of an antibody, or an antigen binding fragment thereof, to CCL24, wherein the binding of the antibody, or the antigen binding fragment thereof, to the peptide comprising the amino acid sequence according to SEQ ID NO: 71 is determined.

In some embodiments, the method is used for determining binding of an antibody, or an antigen binding fragment thereof, to CCL25, wherein the binding of the antibody, or the antigen binding fragment thereof, to the peptide comprising the amino acid sequence according to SEQ ID NO: 72 is determined.

In some embodiments, the method is used for determining binding of an antibody, or an antigen binding fragment thereof, to CCL26, wherein the binding of the antibody, or the antigen binding fragment thereof, to the peptide comprising the amino acid sequence according to SEQ ID NO: 73 is determined.

In some embodiments, the method is used for determining binding of an antibody, or an antigen binding fragment thereof, to CCL27, wherein the binding of the antibody, or the antigen binding fragment thereof, to the peptide comprising the amino acid sequence according to SEQ ID NO: 74 is determined.

In some embodiments, the method is used for determining binding of an antibody, or an antigen binding fragment thereof, to CCL28, wherein the binding of the antibody, or the antigen binding fragment thereof, to the peptide comprising the amino acid sequence according to SEQ ID NO: 75 is determined.

In some embodiments, the method is used for determining binding of an antibody, or an antigen binding fragment thereof, to CXCL1, wherein the binding of the antibody, or the antigen binding fragment thereof, to the peptide comprising the amino acid sequence according to SEQ ID NO: 76 is determined.

In some embodiments, the method is used for determining binding of an antibody, or an antigen binding fragment thereof, to CXCL2, wherein the binding of the antibody, or the antigen binding fragment thereof, to the peptide comprising the amino acid sequence according to SEQ ID NO: 77 is determined.

In some embodiments, the method is used for determining binding of an antibody, or an antigen binding fragment thereof, to CXCL3, wherein the binding of the antibody, or the antigen binding fragment thereof, to the peptide comprising the amino acid sequence according to SEQ ID NO: 78 is determined.

In some embodiments, the method is used for determining binding of an antibody, or an antigen binding fragment thereof, to CXCL4, wherein the binding of the antibody, or the antigen binding fragment thereof, to the peptide comprising the amino acid sequence according to SEQ ID NO: 79 is determined.

In some embodiments, the method is used for determining binding of an antibody, or an antigen binding fragment thereof, to CXCL5, wherein the binding of the antibody, or the antigen binding fragment thereof, to the peptide comprising the amino acid sequence according to SEQ ID NO: 80 is determined.

In some embodiments, the method is used for determining binding of an antibody, or an antigen binding fragment thereof, to CXCL6, wherein the binding of the antibody, or the antigen binding fragment thereof, to the peptide comprising the amino acid sequence according to SEQ ID NO: 81 is determined.

In some embodiments, the method is used for determining binding of an antibody, or an antigen binding fragment thereof, to CXCL7, wherein the binding of the antibody, or the antigen binding fragment thereof, to the peptide comprising the amino acid sequence according to SEQ ID NO: 82 is determined.

In some embodiments, the method is used for determining binding of an antibody, or an antigen binding fragment thereof, to CXCL8, wherein the binding of the antibody, or the antigen binding fragment thereof, to the peptide comprising the amino acid sequence according to SEQ ID NO: 83 is determined.

In some embodiments, the method is used for determining binding of an antibody, or an antigen binding fragment thereof, to CXCL9, wherein the binding of the antibody, or the antigen binding fragment thereof, to the peptide comprising the amino acid sequence according to SEQ ID NO: 84 is determined.

In some embodiments, the method is used for determining binding of an antibody, or an antigen binding fragment thereof, to CXCL10, wherein the binding of the antibody, or the antigen binding fragment thereof, to the peptide comprising the amino acid sequence according to SEQ ID NO: 85 is determined.

In some embodiments, the method is used for determining binding of an antibody, or an antigen binding fragment thereof, to CXCL11, wherein the binding of the antibody, or the antigen binding fragment thereof, to the peptide comprising the amino acid sequence according to SEQ ID NO: 86 is determined.

In some embodiments, the method is used for determining binding of an antibody, or an antigen binding fragment thereof, to CXCL12, wherein the binding of the antibody, or the antigen binding fragment thereof, to the peptide comprising the amino acid sequence according to SEQ ID NO: 87 is determined.

In some embodiments, the method is used for determining binding of an antibody, or an antigen binding fragment thereof, to CXCL13, wherein the binding of the antibody, or the antigen binding fragment thereof, to the peptide comprising the amino acid sequence according to SEQ ID NO: 88 is determined.

In some embodiments, the method is used for determining binding of an antibody, or an antigen binding fragment thereof, to CXCL14, wherein the binding of the antibody, or the antigen binding fragment thereof, to the peptide comprising the amino acid sequence according to SEQ ID NO: 89 is determined.

In some embodiments, the method is used for determining binding of an antibody, or an antigen binding fragment thereof, to CXCL16, wherein the binding of the antibody, or the antigen binding fragment thereof, to the peptide comprising the amino acid sequence according to SEQ ID NO: 90 is determined.

In some embodiments, the method is used for determining binding of an antibody, or an antigen binding fragment thereof, to CXCL17, wherein the binding of the antibody, or the antigen binding fragment thereof, to the peptide comprising the amino acid sequence according to SEQ ID NO: 91 is determined.

In some embodiments, the method is used for determining binding of an antibody, or an antigen binding fragment thereof, to XCL1 and/or XCL2, wherein the binding of the antibody, or the antigen binding fragment thereof, to the peptide comprising the amino acid sequence according to SEQ ID NO: 92 is determined,

In some embodiments, the method is used for determining binding of an antibody, or an antigen binding fragment thereof, to CX3CL1, wherein the binding of the antibody, or the antigen binding fragment thereof, to the peptide comprising the amino acid sequence according to SEQ ID NO: 93 is determined.

The peptides, which may be used for binding to antibodies against the specific human chemokines, are shown in Table 1 below.

TABLE 1

| | SEQ ID NO | SEQ ID NO N-loop |
|---|---|---|
| Chemokine | N-loop peptide | peptide + linker |
| CCL1 | SEQ ID NO: 52 | SEQ ID NO: 1 |
| CCL2 | SEQ ID NO: 53 | SEQ ID NO: 2 |
| CCL3 | SEQ ID NO: 54 | SEQ ID NO: 3 |
| CCL4 | SEQ ID NO: 55 | SEQ ID NO: 4 |
| CCL5 | SEQ ID NO: 56 | SEQ ID NO: 5 |
| CCL7 | SEQ ID NO: 57 | SEQ ID NO: 6 |
| CCL8 | SEQ ID NO: 58 | SEQ ID NO: 7 |
| CCL11 | SEQ ID NO: 59 | SEQ ID NO: 8 |
| CCL13 | SEQ ID NO: 60 | SEQ ID NO: 9 |
| CCL14 | SEQ ID NO: 61 | SEQ ID NO: 10 |
| CCL15 | SEQ ID NO: 62 | SEQ ID NO: 11 |
| CCL16 | SEQ ID NO: 63 | SEQ ID NO: 12 |
| CCL17 | SEQ ID NO: 64 | SEQ ID NO: 13 |
| CCL18 | SEQ ID NO: 65 | SEQ ID NO: 14 |
| CCL19 | SEQ ID NO: 66 | SEQ ID NO: 15 |
| CCL20 | SEQ ID NO: 67 | SEQ ID NO: 16 |
| CCL21 | SEQ ID NO: 68 | SEQ ID NO: 17 |
| CCL22 | SEQ ID NO: 69 | SEQ ID NO: 18 |
| CCL23 | SEQ ID NO: 70 | SEQ ID NO: 19 |
| CCL24 | SEQ ID NO: 71 | SEQ ID NO: 20 |
| CCL25 | SEQ ID NO: 72 | SEQ ID NO: 21 |
| CCL26 | SEQ ID NO: 73 | SEQ ID NO: 22 |
| CCL27 | SEQ ID NO: 74 | SEQ ID NO: 23 |
| CCL28 | SEQ ID NO: 75 | SEQ ID NO: 24 |
| CXCL1 | SEQ ID NO: 76 | SEQ ID NO: 25 |
| CXCL2 | SEQ ID NO: 77 | SEQ ID NO: 26 |
| CXCL3 | SEQ ID NO: 78 | SEQ ID NO: 27 |
| CXCL4 | SEQ ID NO: 79 | SEQ ID NO: 28 |
| CXCL5 | SEQ ID NO: 80 | SEQ ID NO: 29 |
| CXCL6 | SEQ ID NO: 81 | SEQ ID NO: 30 |
| CXCL7 | SEQ ID NO: 82 | SEQ ID NO: 31 |
| CXCL8 | SEQ ID NO: 83 | SEQ ID NO: 32 |
| CXCL9 | SEQ ID NO: 84 | SEQ ID NO: 33 |
| CXCL10 | SEQ ID NO: 85 | SEQ ID NO: 34 |
| CXCL11 | SEQ ID NO: 86 | SEQ ID NO: 35 |
| CXCL12 | SEQ ID NO: 87 | SEQ ID NO: 36 |
| CXCL13 | SEQ ID NO: 88 | SEQ ID NO: 37 |
| CXCL14 | SEQ ID NO: 89 | SEQ ID NO: 38 |
| CXCL16 | SEQ ID NO: 90 | SEQ ID NO: 39 |
| CXCL17 | SEQ ID NO: 91 | SEQ ID NO: 40 |
| XCL1/XCL2 | SEQ ID NO: 92 | SEQ ID NO: 41 |
| CX3CL1 | SEQ ID NO: 93 | SEQ ID NO: 42 |

In view of Table 1, for each human chemokine, the appropriate peptide to determine antibody binding may be selected. It is understood, that the method may also involve testing for antibodies to different combinations of chemokines (e.g., all chemokines or different subsets), wherein the peptides can be selected based on Table 1 above.

In general, the peptides used in the method of the invention may be of any length. Preferably, the length of the peptides used in the method of the invention does not exceed 100 amino acids. More preferably, the maximum length of the peptides used in the method of the invention does not exceed 50 amino acids, e.g., not more than 50, 49, 48, 47, 46, 45, 44, 43, 42, 41, 40, 39, 38, 37, 36, 35, 34, 33, 32, 31, 30, 29, 28, 27, 26 or 25 amino acids. In particular, the length of the peptides used in the method of the invention is preferably between and 25 amino acids, e.g., 30, 29, 28, 27, 26 or 25 amino acids, with smaller molecules being even more preferred. Even more preferably, the length of the peptides used in the method of the invention is about 25 amino acids.

The peptides used in the method of the invention preferably do not include the C-terminus of the chemokines, i.e. the peptides may not include the C-terminal 1, 2, 3, 4, 5, 6, 7, 8, 9, 10 or more amino acids of the chemokines. As described above, the peptides are based on the N-loop of the chemokines. Without being bound to any theory, the present inventors assume that antibodies impacting cell migration target the N-loop of the chemokine, because this region is required for receptor binding. While the use of full-length chemokines would bind antibodies targeting both, the N-loop as well as the C-terminus, it is preferred to not include the C-terminus in order to specifically identify antibodies targeting the N-loop, which are expected to reduce or inhibit cell migration.

Typically, the peptides used in the method of the invention are recombinant peptides, which may comprise or consist of (i) the N-loop of the (human) chemokine, in particular an amino acid sequence according to any one of SEQ ID NOs 52-93, and (ii) an amino acid sequence unrelated to the human chemokine (from which the other part of the peptide is derived). Accordingly, the peptide may be heterologous, i.e. containing sequences of different origin. Preferably, the additional sequence (other than the chemokine N-loop) is useful in the binding assay of the method of the invention, for example to attach the peptide to a support or to provide a label. In some embodiments, the peptides used in the method of the invention comprise or consist of (i) the N-loop of the (human) chemokine, in particular an amino acid sequence according to any one of SEQ ID NOs 52-93, and (ii) a linker, a tag or a label.

As used herein, a “tag” is a peptide sequence grafted onto a recombinant peptide. Examples of tags include affinity tags, solubilization tags, chromatography tags, epitope tags, fluorescence tags and protein tags. Affinity tags may be used in an affinity technique. Examples of affinity tags include chitin binding protein (CBP), maltose binding protein (MBP), and glutathione-S-transferase (GST). A further example is the poly(His) tag which binds to metal matrices. Solubilization tags may be used, especially for recombinant peptides or proteins expressed in chaperone-deficient species such as E. coli, to assist in the proper folding and to keep them from precipitating. Examples of solubilization tags include thioredoxin (TRX) and poly(NANP). Chromatography tags may be used to alter chromatographic properties of the peptide to afford different resolution across a particular separation technique. Chromatography tags often comprise or consist of polyanionic amino acids, such as FLAG-tag. Epitope tags are short peptide sequences, which are usually derived from viral genes. Epitope tags include V5-tag, Myc-tag, HA-tag and NE-tag. These tags are particularly useful for western blotting, immunofluorescence and immunoprecipitation experiments. Fluorescence tags may be used to give visual readout. Green fluorescent protein (GFP) and its variants are the most commonly used fluorescence tags. Protein tags may allow specific enzymatic modification (such as biotinylation by biotin ligase) or chemical modification (such as reaction with FIAsH-EDT2 for fluorescence imaging). Tags may be combined. Tags may be removable by chemical agents or by enzymatic means, such as proteolysis. To this end, the peptide may (further) include a cleavage site.

A “label”, as used herein, refers to a detectable peptide or protein, for example to provide measurability, e.g. for quantification or to facilitate imaging. Non-limiting examples of labels include enzymes, coenzymes, fluorescers, bioluminescers, chromogens, enzyme substrates or co-factors, enzyme inhibitors, and the like. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; and examples of bioluminescent materials include luciferase, luciferin, and aequorin. Such labels may be used in a variety of well-known assays, such as radioimmunoassays, enzyme immunoassays, e.g., ELISA, fluorescent immunoassays, and the like. In some embodiments, the label may comprise or consist of an amino acid sequence of a “reporter” peptide or protein, such as GFP/EGFP, YFP, RFP, CFP, luciferase, beta-galactosidase, or peroxidase. Preferred labels include (i) enzymes as described above, e.g. horseradish peroxidase (HRP) or alkaline phosphatase; (ii) prosthetic group complexes as described above, e.g. streptavidin/biotin and avidin/biotin, in particular in ELISA and immunohistochemistry; and (iii) fluorescers as described above, such as fluorescent dyes and fluorescent proteins (e.g., (enhanced) green fluorescent protein (EGFP); TagBFP, Turquoise, Venus, KO2, Cherry, Apple, Kate2), in particular in immunofluorescence and flow cytometry.

As used herein, the term “linker” (also referred to as “spacer”), as used herein, refers to a peptide, which may be used to elongate a peptide of interest, such as the N-loop of a chemokine, (e.g. to improve results in an assay) and/or to connect a peptide or protein of interest to a distinct moiety, e.g. a substrate or another peptide. Linkers are known in the art and described in detail, e.g. in Reddy Chichili V P, Kumar V, Sivaraman J. Linkers in the structural biology of protein-protein interactions. Protein Science: A Publication of the Protein Society. 2013; 22(2):153-167). Typically, linkers are designed such that they do not affect functionality. In particular, a linker does not specifically bind to a target. A linker may contain any amino acids, the amino acids glycine (G), serine (S) and lysine (K) may be preferred. Preferably, the linker is composed of the amino acids glycine (G) and serine (S) (“GS-linker”) or glycine (G) and lysine (K) (“GK-linker”). If two or more linkers occur in one peptide, the linkers may be equal or differ from each other. Furthermore, the linker may have a length of 1, 2, 3, 4, 5, 6, 7, 8, 9, 10, 11, 12, 13, 14, 15, 16, 17, 18, 19 or 20 amino acids. Preferably, a linker consists of up to 20 amino acids, such as 1, 2, 3, 4, 5, 6, 7, 8, 9, 10, 11, 12, 13, 14, 15, 16, 17, 18, 19 or 20 amino acids, more preferably of up to 15 amino acids, such as 1, 2, 3, 4, 5, 6, 7, 8, 9, 10, 11, 12, 13, 14 or 15 amino acids, even more preferably of up to 10 amino acids, such as 1, 2, 3, 4, 5, 6, 7, 8, 9, or 10 amino acids, and still more preferably of up to 5 amino acids, such as 1, 2, 3, 4, or 5 amino acids. For example, the linker may have a length of (no more than) 1-10 amino acids, preferably (no more than) 2-7 amino acids, more preferably (no more than) 2-5 amino acids, and still more preferably (no more than) 2-4 amino acids. The linker may be derived from naturally occurring amino acid sequences, other than the human chemokine, or from non-natural amino acid sequences. In some embodiments, the linker does not contain any Cys (C) residues. In some embodiments, the linker is selected from GS, GGS, GGGS (SEQ ID NO: 94), or GGK.

Preferably, the peptides used in the method of the invention consist of (i) the N-loop of the (human) chemokine, in particular an amino acid sequence according to any one of SEQ ID NOs 52-93, and (ii) a linker. Furthermore, it is preferred that the linker is positioned at the N-terminus of the N-loop of the (human) chemokine, in particular the amino acid sequence according to any one of SEQ ID NOs 52-93.

Specific exemplified peptides, which are particularly useful in the method of the invention are the peptides consisting of an amino acid sequence according to any one of SEQ ID NOs 1 to 42. These peptides include the N-loop of the human chemokines and a linker. The length of the linker may depend on the length of the N-loop (in particular on the length of the amino acid sequences according to SEQ ID NOs 52-93), such that each resulting peptide (N-loop and linker) may have the same length. In some embodiments, the peptide used in the method of the invention may have a length of 20 to 30 amino acids, preferably of 21 to 29 amino acids, more preferably of 22 to 28 amino acids, even more preferably of 23 to 27 amino acids, still more preferably of 24 to 26 amino acids, and particularly preferably of about 25 amino acids.

The peptides used in the method of the invention may be modified, in particular at their N-terminus and/or at their C-terminus. The specific modification may depend on the specific method for assessing antibody binding to the peptide. In general, peptide modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, biotinylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature (Proteins Structure and Molecular Properties (1993) 2nd Ed., T. E. Creighton, New York; Post-translational Covalent Modifications of Proteins (1983) B. C. Johnson, Ed., Academic Press, New York; Seifter et al. (1990) Analysis for protein modifications and nonprotein cofactors, Meth. Enzymol. 182: 626-646 and Rattan et al., (1992) Protein Synthesis: Post-translational Modifications and Aging, Ann NY Acad Sci, 663: 48-62). In some embodiments, the peptide is biotinylated at its N-terminus and/or amidated at its C-terminus.

The method of the invention is based on the assessment of binding of an antibody to the peptide as described herein, i.e. the peptide comprising an amino acid sequence according to any one of SEQ ID NOs 52-93; or to a sequence variant thereof. Typically, antibody binding to the peptide is assessed in vitro. Accordingly, the method of the invention is usually an in-vitro method.

In order to assess antibody binding to the peptide as described herein, the skilled artisan is aware of various assays for suitably testing antibody binding. The peptide(s) as described herein may be used in any binding assay. It is understood that the peptides may be modified or adapted in view of the selected binding assay. Examples of assays for testing binding of an antibody to the peptide(s) as described herein include, but are not limited to, enzyme-linked immunoassay (ELISA), surface plasmon resonance (SPR) immunoassay, mesoscale discovery electro-chemi luminescence method (MSD-ELC) via, radioimmunoassay (RIA), fluorescence immunoassay (FIA), thermal shift assay, liquid chromatography-mass spectrometry (LC-MS) detection, bio-layer interferometry (BLI) and Tag-lite assay.

Preferably, a ligand binding assay (LBA) is used to determine binding of the antibody, or the antigen binding fragment thereof, to the peptide as described herein, i.e. the peptide comprising an amino acid sequence according to any one of SEQ ID NOs 52-93; or to a sequence variant thereof. In this context, the peptide as described herein is usually the “ligand” and its binding to an antibody is determined. The ligand binding assay may be a radioligand assay or a non-radioactive binding assay. In radioactive assays, radioligands are typically used to assess binding of the antibody. To this end, the peptide may be radioactively labelled. Examples of non-radioactive binding assays include, but are not limited to, fluorescence polarization (FP), fluorescence resonance energy transfer (FRET) and surface plasmon resonance (SPR) assays. For fluorescence polarization (FP) and fluorescence resonance energy transfer (FRET), the peptide as described herein may be fluorescent-labelled. SPR does not require labelling of the peptide. In some embodiments, the ligand binding assay is a liquid phase binding assay or a solid phase binding assay. Non-limiting examples of liquid phase and solid phase binding assays include immunoprecipitation and ELISA. For solid phase binding assays, multiwell-plates, on-bead binding or on-column binding may be used. To this end, the peptides may be modified or adapted such that they can be linked or bound to the selected support (multiwell-plate, bead, column).

Accordingly, different assays can be used in the step of determining binding of the antibody, or the antigen binding fragment thereof, to the peptide as described herein, i.e. the peptide comprising an amino acid sequence according to any one of SEQ ID NOs 52-93; or to a sequence variant thereof.

Preferably, in the method of the invention, the binding of the antibody, or the antigen binding fragment thereof, to the peptide is determined in an enzyme-linked immunoassay (ELISA). ELISAs are well-known in the art and described, for example, in Voller, A, ‘The Enzyme Linked Immunosorbent Assay (ELISA)” Microbiological Associates Quarterly Publication, Walkersville, Md., Diagnostic Horizons 2 (1978), 1-7; Voller et al, J. Clin. Pathol. 31 (1978), 507-520; Butler, Meth. Enzymol. 73 (1981), 482-523; Maggio, E. (ed.). Enzyme Immunoassay, CRC Press, Boca Raton, Fla., (1980); and Ishikawa, E. et al, (eds), Enzyme Immunoassay, Kgaku Shoin, Tokyo (1981)). To this end, the peptide(s) as described herein may be immobilized, e.g. coated onto the support, such as the bottom and/or side wall of a well in a multiwell plate. For immobilization, the peptide(s) may be modified such that they can be linked/attached to the support. To this end, tags may be used. In some embodiments, the peptide(s) may be biotinylated, e.g. at the N-terminus of the peptide(s). The support (e.g., the bottom and/or side wall of a well in a multiwell plate) may be coated with avidin, streptavidin or neutravidin for binding (and thereby immobilizing) the biotinylated peptide(s). To test the binding of the antibody, or the antigen binding fragment thereof, to the peptide(s) as described herein, the antibody, or the antigen binding fragment thereof, is usually provided in a liquid sample, e.g. in a buffer, such as PBS, which is added to the support (e.g. the multiwell plate) with the immobilized peptide(s) for incubation (in order to enable binding of the antibody, or the antigen binding fragment thereof, to the peptide(s). After incubation, the support (e.g. the plate) may be washed (e.g. with washing buffer) to remove excess sample, which is not specifically bound to the peptide(s). For detection of antibodies bound to the peptide(s), e.g. a secondary antibody against the antibody, or the antigen binding fragment thereof, may be used; for example an anti-human IgG antibody (if the antibody to be tested is a human IgG antibody). The secondary antibody may be labelled for detection. To this end, for example fluorescent labels or enzymatic labels, such as horseradish peroxidase, may be used (e.g. linked to the secondary antibody). Binding may then be detected with an appropriate detection method. It is understood that the label and the detection method are usually suitably selected. For example, if horseradish peroxidase (HRP) is used at a label, the HRP substrate (e.g., tetramethylbenzidine) is applied for development and absorbance may be measured at 450 nm. For other enzymatic labels, the respective substrates are applied. For fluorescent labels, detection is performed at the wavelength as required for the specific fluorescent label.

An exemplary standard ELISA may be performed as follows: ELISA plates may be coated with a sufficient amount of the peptide(s) as described herein, to which binding of the antibody is to be tested (e.g., by coating the plates with avidin, streptavidin or neutravidin and adding (and thereby immobilizing) the biotinylated peptide(s)). Plates may then be incubated with the antibody to be tested. After washing, antibody binding can be revealed, e.g. using a labelled secondary antibody recognizing the test antibody, such as anti-human IgG coupled to HRP (or another label). Plates may then be washed, the required substrate (e.g., a tetramethylbenzidine substrate solution) may be added and plates may be read, e.g. at 450 nm. A specific example of an ELISA, which may be used in the method of the invention, is described in the example section of this specification.

To determine relative affinities of antibody binding, serial dilutions of the sample containing the antibody may be tested, e.g. in an ELISA. The relative affinities of antibody binding may then be obtained by determining the half-maximum effective concentration (EC₅₀) of the antibody, i.e. the concentration of the antibody required to achieve 50% maximal binding at saturation. The EC₅₀ values may be calculated by interpolation of binding curves fitted, for example, with nonlinear regression (with a variable slope). To compare the binding affinity of different antibodies, their EC₅₀ values may be compared.

In a further aspect, the present invention also provides a method for identifying an antibody, or an antigen-binding fragment thereof, which binds to (the N-loop of) a human chemokine in a sample, the method comprising:
performing the method as described above, wherein the sample is tested for binding to the peptide as described above.

The sample may be any composition, which may contain or is assumed to contain an antibody, or an antigen-binding fragment thereof, which may bind to a human chemokine. The sample is usually not part of the human or animal body. In some embodiments, the sample may be a composition, which does not occur in nature. Preferably, the sample is a (isolated) body fluid or derived from a body fluid of a human or animal subject. Body fluids, such as blood and mucous secretions, are well-known to contain antibodies. In some embodiments, the sample (to be tested) is (isolated) blood or derived from blood, such as whole blood, plasma or serum. The method is preferably an in-vitro method.

The method for identifying an antibody, or an antigen-binding fragment thereof, according to the invention corresponds essentially to the method for determining binding of an antibody, or an antigen binding fragment thereof, of the invention as described above. It is based on the very same peptide-based binding test, which may be used
(i) to determine whether or not (or to what extend) an antibody, or an antigen binding fragment thereof, binds to (the N-loop of) a human chemokine (in the method of the invention as described above);
(ii) to determine whether or not a sample contains an antibody, or an antigen binding fragment thereof, binding to (the N-loop of) a human chemokine; and/or
(iii) to identify an antibody, or an antigen-binding fragment thereof, which binds to (the N-loop of) a human chemokine in a sample.

If a sample is found to contain an antibody, or an antigen-binding fragment thereof, which binds to (the N-loop of) a human chemokine, by using the method as described above, the antibody can be readily isolated, sequenced and cloned by methods well known to the skilled person. In particular, heavy and light chain variable region (VH/VL) of an antibody, which binds to (the N-loop of) a human chemokine may be obtained by identifying B cells capable of producing the antibody. To this end, B cells may be sorted and B cells capable of producing the antibody may be obtained by using methods known in the art (using B cell markers and the respective peptide(s) as described herein). For example, B cells may be enriched from peripheral blood mononuclear cells (PBMC), e.g. by using a pan-B-cell isolation kit. Thereafter, they may be stained with cell-lineage specific markers and, e.g., fluorophore-labeled N-loop peptides, and single-cell sorted by flow-cytometry, e.g. as live single Zombie-NIR-CD14-CD16-CD3-CD8-CD20+Ova-N-loop-PE+N-loop-AF647+B cells. The sequences of the heavy and light chain variable region (VH/VL) genes of the B cell may then be identified. Suitable methods for obtaining B cells and the VH/VL sequences from B cells are described, for example, in
Robbiani, D. F., Bozzacco, L., Keeffe, J. R., Khouri, R., Olsen, P. C., Gazumyan, A., Schaefer-Babajew, D., Avila-Rios, S., Nogueira, L., Patel, R., et al. (2017). Recurrent Potent Human Neutralizing Antibodies to Zika Virus in Brazil and Mexico. Cell 169, 597-609 e511;
Tiller, T., Meffre, E., Yurasov, S., Tsuiji, M., Nussenzweig, M. C., and Wardemann, H. (2008). Efficient generation of monoclonal antibodies from single human B cells by single cell RT-PCR and expression vector cloning. J Immunol Methods 329, 112-124; and
von Boehmer, L., Liu, C., Ackerman, S., Gitlin, A. D., Wang, Q., Gazumyan, A., and Nussenzweig, M. C. (2016). Sequencing and cloning of antigen-specific antibodies from mouse memory B cells. Nat Protoc 11, 1908-1923,
   which are incorporated herein by reference. A further specific example is provided in the example section of the present specification.

Accordingly, the skilled person is well aware of methods to obtain the sequences of the heavy and light chain variable region (VH/VL) genes of a B cell. In some embodiments, at first VH/VL nucleic acids (of the B cell receptor (BCR) of the B cell) may be retrieved by reverse transcription, which may be followed by nested PCR reactions (and sequencing). Methods to amplify and sequence Ig variable regions are well-known in the art (e.g., Robbiani et al., 2017, Tiller et al., 2008 and von Boehmer et al., 2016). For example, a reverse transcription PCR (RT-PCR) may be performed, in particular to obtain cDNA of the heavy and light chain variable region (VH/VL) genes. In this context, primers specific for the constant regions of the heavy and light chains (in particular IgG), such as primers specific to the constant regions of IgG, Igκ, and Igλ, respectively, may be used. Sequences of the constant regions of human antibodies are well-known in the art, such that the skilled person can readily obtain respective primers (which are also commercially available). The obtained cDNA may then be amplified, for example by PCR (such as two-step PCR, e.g. using two sets of Ig-specific primers, one nested within the other) and optionally purified. Then, the (purified) cDNA may be sequenced. Accordingly, the method for identifying an antibody, or an antigen-binding fragment thereof, which binds to (the N-loop of) a human chemokine may include a step of obtaining the sequences of the heavy and light chain variable region (VH/VL) genes of a B-cell capable of producing said antibody.

In a further aspect, the present invention also provides a method for generating an expression vector encoding the heavy and/or light chain of an antibody, which binds to (the N-loop of) a human chemokine, the method comprising the following steps:
(1) identification of an antibody, or an antigen-binding fragment thereof, which binds to (the N-loop of) a human chemokine, in particular identification of the nucleic acid sequences encoding the heavy and light chain variable region (VH/VL) of the antibody, as described above; and
(2) cloning of the heavy and light chain variable region (VH/VL) of the antibody into an expression vector for expression of antibody heavy and light chains, respectively.

The above detailed description of the identification of nucleic acid sequences of the heavy and light chain variable region (VH/VL) of the antibody identified with the method of the invention applies accordingly to step (1) of the method for generating an expression vector encoding the heavy and/or light chain of the antibody.

A vector is usually a recombinant nucleic acid molecule, i.e. a nucleic acid molecule which does not occur in nature. Accordingly, the vector may comprise heterologous elements (i.e., sequence elements of different origin in nature). For example, the vector may comprise a multi cloning site, a heterologous promotor, a heterologous enhancer, a heterologous selection marker (to identify cells comprising said vector in comparison to cells not comprising said vector) and the like. A vector in the context of the present invention is suitable for incorporating or harboring a desired nucleic acid sequence. Thus, the vector may comprise a sequence corresponding, e.g., to (a heavy and/or light chain (variable region) of) the antibody. In particular, the vector is an expression vector. An expression vector may be used for production of expression products such as RNA, e.g. mRNA, or peptides, polypeptides or proteins. For example, an expression vector may comprise sequences needed for transcription of a sequence stretch of the vector, such as a (heterologous) promoter sequence. A vector in the context of the present invention may be, e.g., an RNA vector or a DNA vector. For example, a vector in the sense of the present application comprises a cloning site, a selection marker, such as an antibiotic resistance factor, and a sequence suitable for multiplication of the vector, such as an origin of replication. A vector in the context of the present application may be a plasmid vector.

Expression vectors for (recombinant) expression of (human) monoclonal antibodies are known in the art commercially available. Such expression vectors may already comprise the sequences of the (human) constant regions (e.g., for IgG-type antibodies), such that only the VH/VL sequences need to be inserted by common cloning techniques well-known in the art. In some embodiments, the heavy chain and the light chain of the antibody are encoded by distinct expression vectors (i.e. a plurality of vectors), such that the entire antibody is encoded by two expression vectors (one for the heavy chain and the other for the light chain). In this case, for expression of the antibody a host cell may be transfected with both vectors. In other embodiments, the same vector may encode the heavy chain and the light chain of the antibody, e.g. in a multicistronic (bicistronic) manner.

The cloning of the antibody in an expression vector may be carried out, for example, as described in Tiller et al. Efficient generation of monoclonal antibodies from single human B cells by single cell RT-PCR and expression vector cloning. J Immunol Methods. 2008; 329 (1-2):112-124. doi:10.1016/j.jim.2007.09.017, which is incorporated herein by reference.

In a further aspect, the present invention also provides a method for producing a recombinant cell expressing an antibody, which binds to (the N-loop of) a human chemokine, the method comprising the following steps:
(i) generating an expression vector encoding the heavy and/or light chain of the antibody, which binds to (the N-loop of) a human chemokine, as described above;
(ii) transfecting a host cell with said expression vector; and
(iii) optionally, culturing said host cell.

The detailed description of the method for generating an expression vector encoding the heavy and/or light chain of an antibody, which binds to (the N-loop of) a human chemokine, in particular of step (1) and step (2) described above, applies accordingly to step (i) of the method for producing a recombinant cell expressing the antibody, which binds to (the N-loop of) a human chemokine.

Examples of suitable cells include, but are not limited to, eukaryotic cells, e.g., yeast cells, animal cells or plant cells. Other examples of such cells include, but are not limited to, prokaryotic cells, in particular bacterial cells, e.g. E. coli. In some embodiments, the cells are mammalian cells, such as a mammalian cell line. Examples include human cells, CHO cells, HEK293T cells, PER. C6 cells, NS0 cells, human liver cells, myeloma cells or hybridoma cells. In some embodiments, the host cell is a 293T cell.

The cell may be transfected with the vector (or the plurality of vectors). The term “transfection” refers to the introduction of nucleic acid molecules, such as DNA or RNA (e.g. mRNA) molecules, into cells, e.g. into eukaryotic or prokaryotic cells. In the context of the present invention, the term “transfection” encompasses any method known to the skilled person for introducing nucleic acid molecules into cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In some embodiments, the introduction is non-viral.

The cells may be transfected stably or transiently with the vector, e.g. for expressing the antibody. In some embodiments, the cells are stably transfected with the vector (or the two vectors) encoding the antibody, which binds to (the N-loop of) a human chemokine. In other embodiments, the cells are transiently transfected with the vector (or the two vectors) encoding the antibody, which binds to (the N-loop of) a human chemokine. A stably transfected cell may be used to establish a cell line. A cell line is typically continuous (i.e., it can proliferate indefinitely), in particular due to tumor or artificial immortalization.

The host cell (and, optionally, its progeny; e.g., a cell line) may be cultured, in particular for expression of the encoded antibody. Suitable culture conditions for culturing host cells are well-known in the art, e.g. using a commercially available culture medium. The detailed conditions may be selected depending on the cell type of the host cell, as known by the person skilled in the art.

In a further aspect, the present invention also provides a method for producing an antibody, which binds to (the N-loop of) a human chemokine, the method comprising the following steps:
(a) producing a recombinant cell expressing the antibody, which binds to (the N-loop of) a human chemokine, as described above; and
(b) isolating the antibody, which binds to (the N-loop of) a human chemokine.

The detailed description of the method for producing a recombinant cell expressing an antibody, which binds to (the N-loop of) a human chemokine, in particular step (i), step (ii) and step (iii) as described above, applies accordingly to step (a) of the method for producing the antibody, which binds to (the N-loop of) a human chemokine. In general, the identification of the VH/VL sequences, the cloning of nucleic acids in expression vectors, the transfection of host cells, the culture of the transfected host cells and the isolation of the produced antibody can be done using any methods known to one of skill in the art.

In general, a host cell transfected with the expression vector (or a combination of expression vectors) encoding the antibody usually expresses said antibody, which can then be isolated from the supernatant of the host cell culture. Various methods are known in the art for isolation of an antibody from cell culture supernatant. Non-limiting examples include the use of protein A (a 42 kDa protein with high affinity for the Fc region of IgG), of alternative IgG binding proteins (e.g. protein G, protein L), of synthetic protein A mimics, or of bioengineered peptides or synthetic ligands, all of which may be coupled to a support (e.g., for chromatography), in order to capture the desired antibody. Thereafter, the antibody may be separated and, optionally, further purification steps may be performed. The antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art. For isolation and/or purification of the antibody, which binds to (the N-loop of) a human chemokine, tags introduced into the antibody (e.g. by means of the recombinant expression vector) may be used.

In a further aspect the present invention also provides a recombinant (fusion) peptide comprising or consisting of (i) an amino acid sequence according to any one of SEQ ID NOs 52-93, and (ii) an amino acid sequence unrelated to the human chemokine (from which the other part of the peptide, i.e. any one of SEQ ID NOs 52-93, is derived). Preferably, the additional sequence (other than the chemokine N-loop) is useful in the binding assay of the method of the invention, for example to attach the peptide to a support or to provide a label. In some embodiments, the peptides used in the method of the invention comprise or consist of (i) the N-loop of the (human) chemokine, in particular an amino acid sequence according to any one of SEQ ID NOs 52-93, and (ii) a linker, a tag or a label. Accordingly, the present invention also provides a recombinant (fusion) peptide comprising an amino acid sequence according to any one of SEQ ID NOs 52-93 and a linker, a tag or a label.

In general, the peptide of the invention may be of any length. Preferably, the length of the peptide does not exceed 100 amino acids. More preferably, the maximum length of the peptide does not exceed 50 amino acids, e.g., not more than 50, 49, 48, 47, 46, 45, 44, 43, 42, 41, 40, 39, 38, 37, 36, 35, 34, 33, 32, 31, 30, 29, 28, 27, 26 or 25 amino acids. In particular, the length of the peptide is preferably between 30 and 25 amino acids, e.g., 30, 29, 28, 27, 26 or 25 amino acids, with smaller peptides being even more preferred. Even more preferably, the length of the peptide is about 25 amino acids.

The peptide does preferably not include the C-terminus of the chemokines, i.e. the peptide may not include the C-terminal 1, 2, 3, 4, 5, 6, 7, 8, 9, 10 or more amino acids of the respective chemokine. As described above, the peptides are based on the N-loop of the chemokines.

In general, the detailed description provided above for the peptide(s) (comprising any one of SEQ ID NOs 52-93) as used in the method of the present invention, including the various embodiments of the peptide(s) as used in the method of the present invention, applies accordingly to the peptide of the invention. Likewise, the definitions, e.g. for the terms “linker”, “tag” and “label” apply accordingly. Accordingly, in some embodiments, the peptide consists of (i) an amino acid sequence according to any one of SEQ ID NOs 52-93 and (ii) a linker, in particular as described above; for example wherein the linker has a length of (no more than) 1-10 amino acids, preferably (no more than) 2-7 amino acids, more preferably (no more than) 2-5 amino acids, and still more preferably (no more than) 2-4 amino acids; e.g. a linker selected from GS, GGS, GGGS (SEQ ID NO: 94), or GGK. As described above, in some embodiments, the peptide may consist of an amino acid sequence according to any one of SEQ ID NOs 1 to 42. The peptide may be modified at its N-terminus and/or at its C-terminus, for example the peptide may be biotinylated at its N-terminus and/or amidated at its C-terminus.

In a further aspect, the present invention also provides a composition comprising the peptide of the invention. Optionally, the composition further comprises a diluent, a carrier or a vehicle. Such a composition may be useful for analytical purposes, e.g. in the context of the method of the invention as described above. Therefore, the composition may comprise a buffer, such as PBS (phosphate buffered saline). In some embodiments, the composition may be an aqueous composition.

In a further aspect, the present invention also provides a kit comprising at least two distinct peptides according to the invention. In particular, the kit may comprise
(i) a first peptide of the invention as described above, which comprises a first sequence selected from any one of SEQ ID NOs 52-93; and
(ii) a second peptide of the invention as described above, which comprises a second sequence selected from any one of SEQ ID NOs 52-93 (i.e., a different SEQ ID NO from the first).

Optionally, the kit may comprise further peptides of the invention (which differ from the first and second peptide—and from each other—in the same manner). For example, the kit may comprise 2, 3, 4, 5, 6, 7, 8, 9, 10 or more different peptides, wherein each peptide preferably comprises a different sequence selected from SEQ ID NOs 52-93.

Similarly, the above-described kits may optionally also comprise a fifth, sixth, seventh etc. peptide of the invention. In some embodiments, the different peptides having the different sequences according to SEQ ID NO: 52-93 may be selected/combined in the kit in view of a chemokine-auto-antibody profile for a specific disease, in particular as described herein below.

In some embodiments, the kit may further comprise (analytical) compositions and/or products, such as vessels, for performing a method of the invention, e.g. an ELISA. The kit may also comprise a leaflet of instructions for use, for example describing how to perform a method of the invention with the provided peptides.

### Antibodies Binding to Human Chemokines

By using the method of the invention as described herein above, the present inventors have identified various (human) antibodies (specifically) binding to different human chemokines.

Standard methods to assess binding of the antibody according to the present invention, or the antigen-binding fragment thereof, are known to those skilled in the art and described above. Such methods for testing antibody binding include, for example, ELISA (enzyme-linked immunosorbent assay). Thereby, the relative affinities of antibody binding may be determined by measuring the concentration of the antibody (EC₅₀) required to achieve 50% maximal binding at saturation. An exemplary standard ELISA may be performed as described above. A specific example of an ELISA, which may be used to assess binding of an antibody, is described in the example section of this specification.

In general, the antibody, or an antigen-binding fragment thereof, according to the present invention, may comprise (at least) three complementarity determining regions (CDRs) on a heavy chain and (at least) three CDRs on a light chain. In general, complementarity determining regions (CDRs) are the hypervariable regions present in heavy chain variable domains and light chain variable domains. Typically, the CDRs of a heavy chain and the connected light chain of an antibody together form the antigen receptor. Usually, the three CDRs (CDR1, CDR2, and CDR3) are arranged non-consecutively in the variable domain. Since antigen receptors are typically composed of two variable domains (on two different polypeptide chains, i.e. heavy and light chain: heavy chain variable region (VH) and light chain variable region (VL)), there are typically six CDRs for each antigen receptor (heavy chain: CDRH1, CDRH2, and CDRH3; light chain: CDRL1, CDRL2, and CDRL3). For example, a classical IgG antibody molecule usually has two antigen receptors and therefore contains twelve CDRs. The CDRs on the heavy and/or light chain may be separated by framework regions, whereby a framework region (FR) is a region in the variable domain which is less “variable” than the CDR. For example, a variable region (or each variable region, respectively) may be composed of four framework regions, separated by three CDR's.

The sequences of the heavy chains and light chains of exemplary antibodies of the invention, comprising three different CDRs on the heavy chain and three different CDRs on the light chain were determined. The position of the CDRs are defined according to the IMGT numbering system (IMGT: http://www.imgt.org/; cf. Lefranc, M.-P. et al. (2009) Nucleic Acids Res. 37, D1006-D1012).

Antibodies Binding to CCL8 (Chemokine (C-C motif) Ligand 8)

In some aspects, the present invention provides an antibody, or an antigen-binding fragment thereof, which binds to CCL8 (Chemokine (C-C motif) ligand 8), wherein the antibody, or the antigen-binding fragment thereof, comprises:
(i) heavy chain CDR1, CDR2, and CDR3 sequences having at least 70% sequence identity with the amino acid sequences of SEQ ID NO: 95, SEQ ID NO: 96, and SEQ ID NO: 97, respectively, and light chain CDR1, CDR2, and CDR3 sequences having at least 70% sequence identity with the amino acid sequences of SEQ ID NO: 98, SEQ ID NO: 99 (or 100), and SEQ ID NO: 101, respectively;
(ii) heavy chain CDR1, CDR2, and CDR3 sequences having at least 70% sequence identity with the amino acid sequences of SEQ ID NO: 105, SEQ ID NO: 106, and SEQ ID NO: 107, respectively, and light chain CDR1, CDR2, and CDR3 sequences having at least 70% sequence identity with the amino acid sequences of SEQ ID NO: 108, SEQ ID NO: 99 (or 109), and SEQ ID NO: 110, respectively;
(iii) heavy chain CDR1, CDR2, and CDR3 sequences having at least 70% sequence identity with the amino acid sequences of SEQ ID NO: 113, SEQ ID NO: 114, and SEQ ID NO: 115, respectively, and light chain CDR1, CDR2, and CDR3 sequences having at least 70% sequence identity with the amino acid sequences of SEQ ID NO: 98, SEQ ID NO: 99 (or 116), and SEQ ID NO: 117, respectively;
(iv) heavy chain CDR1, CDR2, and CDR3 sequences having at least 70% sequence identity with the amino acid sequences of SEQ ID NO: 105, SEQ ID NO: 122, and SEQ ID NO: 123, respectively, and light chain CDR1, CDR2, and CDR3 sequences having at least 70% sequence identity with the amino acid sequences of SEQ ID NO: 124, SEQ ID NO: 99 (or 109), and SEQ ID NO: 110, respectively;
(v) heavy chain CDR1, CDR2, and CDR3 sequences having at least 70% sequence identity with the amino acid sequences of SEQ ID NO: 127, SEQ ID NO: 128, and SEQ ID NO: 129, respectively, and light chain CDR1, CDR2, and CDR3 sequences having at least 70% sequence identity with the amino acid sequences of SEQ ID NO: 130, SEQ ID NO: 131 (or 132), and SEQ ID NO: 133, respectively;
(vi) heavy chain CDR1, CDR2, and CDR3 sequences having at least 70% sequence identity with the amino acid sequences of SEQ ID NO: 138, SEQ ID NO: 106, and SEQ ID NO: 107, respectively, and light chain CDR1, CDR2, and CDR3 sequences having at least 70% sequence identity with the amino acid sequences of SEQ ID NO: 108, SEQ ID NO: 99 (or 109), and SEQ ID NO: 110, respectively;
(vii) heavy chain CDR1, CDR2, and CDR3 sequences having at least 70% sequence identity with the amino acid sequences of SEQ ID NO: 141, SEQ ID NO: 142, and SEQ ID NO: 143, respectively, and light chain CDR1, CDR2, and CDR3 sequences having at least 70% sequence identity with the amino acid sequences of SEQ ID NO: 108, SEQ ID NO: 99 (or 109), and SEQ ID NO: 110, respectively;
(viii) heavy chain CDR1, CDR2, and CDR3 sequences having at least 70% sequence identity with the amino acid sequences of SEQ ID NO: 138, SEQ ID NO: 142, and SEQ ID NO: 107, respectively, and light chain CDR1, CDR2, and CDR3 sequences having at least 70% sequence identity with the amino acid sequences of SEQ ID NO: 108, SEQ ID NO: 99 (or 109), and SEQ ID NO: 110, respectively;
(ix) heavy chain CDR1, CDR2, and CDR3 sequences having at least 70% sequence identity with the amino acid sequences of SEQ ID NO: 148, SEQ ID NO: 149, and SEQ ID NO: 150, respectively, and light chain CDR1, CDR2, and CDR3 sequences having at least 70% sequence identity with the amino acid sequences of SEQ ID NO: 151, SEQ ID NO: 152 (or 153), and SEQ ID NO: 154, respectively;
(x) heavy chain CDR1, CDR2, and CDR3 sequences having at least 70% sequence identity with the amino acid sequences of SEQ ID NO: 157, SEQ ID NO: 158, and SEQ ID NO: 159, respectively, and light chain CDR1, CDR2, and CDR3 sequences having at least 70% sequence identity with the amino acid sequences of SEQ ID NO: 160, SEQ ID NO: 161 (or 162), and SEQ ID NO: 163, respectively;
(xi) heavy chain CDR1, CDR2, and CDR3 sequences having at least 70% sequence identity with the amino acid sequences of SEQ ID NO: 157, SEQ ID NO: 158, and SEQ ID NO: 159, respectively, and light chain CDR1, CDR2, and CDR3 sequences having at least 70% sequence identity with the amino acid sequences of SEQ ID NO: 166, SEQ ID NO: 99 (or 167), and SEQ ID NO: 168, respectively;
(xii) heavy chain CDR1, CDR2, and CDR3 sequences having at least 70% sequence identity with the amino acid sequences of SEQ ID NO: 171, SEQ ID NO: 172, and SEQ ID NO: 173, respectively, and light chain CDR1, CDR2, and CDR3 sequences having at least 70% sequence identity with the amino acid sequences of SEQ ID NO: 174, SEQ ID NO: 99 (or 167), and SEQ ID NO: 168, respectively; or
(xiii) heavy chain CDR1, CDR2, and CDR3 sequences having at least 70% sequence identity with the amino acid sequences of SEQ ID NO: 177, SEQ ID NO: 178, and SEQ ID NO: 179, respectively, and light chain CDR1, CDR2, and CDR3 sequences having at least 70% sequence identity with the amino acid sequences of SEQ ID NO: 180, SEQ ID NO: 99 (or 116), and SEQ ID NO: 168, respectively.

The general definition of sequence variants as described above applies accordingly to the sequence variants of the CDR sequences according to any one of (i)-(xiii) (see above) of the antibody, or an antigen-binding fragment thereof, which binds to CCL8. Likewise, the general definition of sequence variants as described above applies accordingly to the sequence variants of the VH and VL sequences according to any one of (i)-(xvi) (see below) of the antibody, or an antigen-binding fragment thereof, which binds to CCL8.

Preferably, the antibody, or an antigen-binding fragment thereof, which binds to CCL8, comprises
(i) heavy chain CDR1, CDR2, and CDR3 sequences according to SEQ ID NO: 95, SEQ ID NO: 96, and SEQ ID NO: 97, respectively, and light chain CDR1, CDR2, and CDR3 sequences according to SEQ ID NO: 98, SEQ ID NO: 99 (or 100), and SEQ ID NO: 101, respectively;
(ii) heavy chain CDR1, CDR2, and CDR3 sequences according to SEQ ID NO: 105, SEQ ID NO: 106, and SEQ ID NO: 107, respectively, and light chain CDR1, CDR2, and CDR3 sequences according to SEQ ID NO: 108, SEQ ID NO: 99 (or 109), and SEQ ID NO: 110, respectively;
(iii) heavy chain CDR1, CDR2, and CDR3 according to SEQ ID NO: 113, SEQ ID NO: 114, and SEQ ID NO: 115, respectively, and light chain CDR1, CDR2, and CDR3 according to SEQ ID NO: 98, SEQ ID NO: 99 (or 116), and SEQ ID NO: 117, respectively;
(iv) heavy chain CDR1, CDR2, and CDR3 according to SEQ ID NO: 105, SEQ ID NO: 122, and SEQ ID NO: 123, respectively, and light chain CDR1, CDR2, and CDR3 according to SEQ ID NO: 124, SEQ ID NO: 99 (or 109), and SEQ ID NO: 110, respectively;
(v) heavy chain CDR1, CDR2, and CDR3 according to SEQ ID NO: 127, SEQ ID NO: 128, and SEQ ID NO: 129, respectively, and light chain CDR1, CDR2, and CDR3 according to SEQ ID NO: 130, SEQ ID NO: 131 (or 132), and SEQ ID NO: 133, respectively;
(vi) heavy chain CDR1, CDR2, and CDR3 according to SEQ ID NO: 138, SEQ ID NO: 106, and SEQ ID NO: 107, respectively, and light chain CDR1, CDR2, and CDR3 according to SEQ ID NO: 108, SEQ ID NO: 99 (or 109), and SEQ ID NO: 110, respectively;
(vii) heavy chain CDR1, CDR2, and CDR3 according to SEQ ID NO: 141, SEQ ID NO: 142, and SEQ ID NO: 143, respectively, and light chain CDR1, CDR2, and CDR3 according to SEQ ID NO: 108, SEQ ID NO: 99 (or 109), and SEQ ID NO: 110, respectively;
(viii) heavy chain CDR1, CDR2, and CDR3 according to SEQ ID NO: 138, SEQ ID NO: 142, and SEQ ID NO: 107, respectively, and light chain CDR1, CDR2, and CDR3 according to SEQ ID NO: 108, SEQ ID NO: 99 (or 109), and SEQ ID NO: 110, respectively;
(ix) heavy chain CDR1, CDR2, and CDR3 according to SEQ ID NO: 148, SEQ ID NO: 149, and SEQ ID NO: 150, respectively, and light chain CDR1, CDR2, and CDR3 according to SEQ ID NO: 151, SEQ ID NO: 152 (or 153), and SEQ ID NO: 154, respectively;
(x) heavy chain CDR1, CDR2, and CDR3 according to SEQ ID NO: 157, SEQ ID NO: 158, and SEQ ID NO: 159, respectively, and light chain CDR1, CDR2, and CDR3 according to SEQ ID NO: 160, SEQ ID NO: 161 (or 162), and SEQ ID NO: 163, respectively;
(xi) heavy chain CDR1, CDR2, and CDR3 according to SEQ ID NO: 157, SEQ ID NO: 158, and SEQ ID NO: 159, respectively, and light chain CDR1, CDR2, and CDR3 according to SEQ ID NO: 166, SEQ ID NO: 99 (or 167), and SEQ ID NO: 168, respectively;
(xii) heavy chain CDR1, CDR2, and CDR3 according to SEQ ID NO: 171, SEQ ID NO: 172, and SEQ ID NO: 173, respectively, and light chain CDR1, CDR2, and CDR3 according to SEQ ID NO: 174, SEQ ID NO: 99 (or 167), and SEQ ID NO: 168, respectively; or
(xiii) heavy chain CDR1, CDR2, and CDR3 according to SEQ ID NO: 177, SEQ ID NO: 178, and SEQ ID NO: 179, respectively, and light chain CDR1, CDR2, and CDR3 according to SEQ ID NO: 180, SEQ ID NO: 99 (or 116), and SEQ ID NO: 168, respectively.

Accordingly, the antibody or the antigen-binding fragment thereof preferably comprises:
a heavy chain CDR1 sequence according to SEQ ID NO: 95;
a heavy chain CDR2 sequence according to SEQ ID NO: 96;
a heavy chain CDR3 sequence according to SEQ ID NO: 97;
a light chain CDR1 sequence according to SEQ ID NO: 98;
a light chain CDR2 sequence according to SEQ ID NO: 99 (or 100); and
a light chain CDR3 sequence according to SEQ ID NO: 101.

In some embodiments, the antibody or the antigen-binding fragment thereof preferably comprises:
a heavy chain CDR1 sequence according to SEQ ID NO: 105;
a heavy chain CDR2 sequence according to SEQ ID NO: 106;
a heavy chain CDR3 sequence according to SEQ ID NO: 107;
a light chain CDR1 sequence according to SEQ ID NO: 108;
a light chain CDR2 sequence according to SEQ ID NO: 99 (or 109); and
a light chain CDR3 sequence according to SEQ ID NO: 110.

In some embodiments, the antibody or the antigen-binding fragment thereof preferably comprises:
a heavy chain CDR1 sequence according to SEQ ID NO: 113;
a heavy chain CDR2 sequence according to SEQ ID NO: 114;
a heavy chain CDR3 sequence according to SEQ ID NO: 115;
a light chain CDR1 sequence according to SEQ ID NO: 98;
a light chain CDR2 sequence according to SEQ ID NO: 99 (or 116); and
a light chain CDR3 sequence according to SEQ ID NO: 117.

In some embodiments, the antibody or the antigen-binding fragment thereof preferably comprises:
a heavy chain CDR1 sequence according to SEQ ID NO: 105;
a heavy chain CDR2 sequence according to SEQ ID NO: 122;
a heavy chain CDR3 sequence according to SEQ ID NO: 123;
a light chain CDR1 sequence according to SEQ ID NO: 124;
a light chain CDR2 sequence according to SEQ ID NO: 99 (or 109); and
a light chain CDR3 sequence according to SEQ ID NO: 110.

In some embodiments, the antibody or the antigen-binding fragment thereof preferably comprises:
a heavy chain CDR1 sequence according to SEQ ID NO: 127;
a heavy chain CDR2 sequence according to SEQ ID NO: 128;
a heavy chain CDR3 sequence according to SEQ ID NO: 129;
a light chain CDR1 sequence according to SEQ ID NO: 130;
a light chain CDR2 sequence according to SEQ ID NO: 131 (or 132); and
a light chain CDR3 sequence according to SEQ ID NO: 133.

In some embodiments, the antibody or the antigen-binding fragment thereof preferably comprises:
a heavy chain CDR1 sequence according to SEQ ID NO: 138;
a heavy chain CDR2 sequence according to SEQ ID NO: 106;
a heavy chain CDR3 sequence according to SEQ ID NO: 107;
a light chain CDR1 sequence according to SEQ ID NO: 108;
a light chain CDR2 sequence according to SEQ ID NO: 99 (or 109); and
a light chain CDR3 sequence according to SEQ ID NO: 110.

In some embodiments, the antibody or the antigen-binding fragment thereof preferably comprises:
a heavy chain CDR1 sequence according to SEQ ID NO: 141;
a heavy chain CDR2 sequence according to SEQ ID NO: 142;
a heavy chain CDR3 sequence according to SEQ ID NO: 143;
a light chain CDR1 sequence according to SEQ ID NO: 108;
a light chain CDR2 sequence according to SEQ ID NO: 99 (or 109); and
a light chain CDR3 sequence according to SEQ ID NO: 110.

In some embodiments, the antibody or the antigen-binding fragment thereof preferably comprises:
a heavy chain CDR1 sequence according to SEQ ID NO: 138;
a heavy chain CDR2 sequence according to SEQ ID NO: 142;
a heavy chain CDR3 sequence according to SEQ ID NO: 107;
a light chain CDR1 sequence according to SEQ ID NO: 108;
a light chain CDR2 sequence according to SEQ ID NO: 99 (or 109); and
a light chain CDR3 sequence according to SEQ ID NO: 110.

In some embodiments, the antibody or the antigen-binding fragment thereof preferably comprises:
a heavy chain CDR1 sequence according to SEQ ID NO: 148;
a heavy chain CDR2 sequence according to SEQ ID NO: 149;
a heavy chain CDR3 sequence according to SEQ ID NO: 150;
a light chain CDR1 sequence according to SEQ ID NO: 151;
a light chain CDR2 sequence according to SEQ ID NO: 152 (or 153); and
a light chain CDR3 sequence according to SEQ ID NO: 154.

In some embodiments, the antibody or the antigen-binding fragment thereof preferably comprises:
a heavy chain CDR1 sequence according to SEQ ID NO: 157;
a heavy chain CDR2 sequence according to SEQ ID NO: 158;
a heavy chain CDR3 sequence according to SEQ ID NO: 159;
a light chain CDR1 sequence according to SEQ ID NO: 160;
a light chain CDR2 sequence according to SEQ ID NO: 161 (or 162); and
a light chain CDR3 sequence according to SEQ ID NO: 163.

In some embodiments, the antibody or the antigen-binding fragment thereof preferably comprises:
a heavy chain CDR1 sequence according to SEQ ID NO: 157;
a heavy chain CDR2 sequence according to SEQ ID NO: 158;
a heavy chain CDR3 sequence according to SEQ ID NO: 159;
a light chain CDR1 sequence according to SEQ ID NO: 166;
a light chain CDR2 sequence according to SEQ ID NO: 99 (or 167); and
a light chain CDR3 sequence according to SEQ ID NO: 168.

In some embodiments, the antibody or the antigen-binding fragment thereof preferably comprises:
a heavy chain CDR1 sequence according to SEQ ID NO: 171;
a heavy chain CDR2 sequence according to SEQ ID NO: 172;
a heavy chain CDR3 sequence according to SEQ ID NO: 173;
a light chain CDR1 sequence according to SEQ ID NO: 174;
a light chain CDR2 sequence according to SEQ ID NO: 99 (or 167); and
a light chain CDR3 sequence according to SEQ ID NO: 168.

In some embodiments, the antibody or the antigen-binding fragment thereof preferably comprises:
a heavy chain CDR1 sequence according to SEQ ID NO: 177;
a heavy chain CDR2 sequence according to SEQ ID NO: 178;
a heavy chain CDR3 sequence according to SEQ ID NO: 179;
a light chain CDR1 sequence according to SEQ ID NO: 180;
a light chain CDR2 sequence according to SEQ ID NO: 99 (or 116); and
a light chain CDR3 sequence according to SEQ ID NO: 168.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 102 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 103. Thereby, the CDR sequences as defined above (heavy chain CDR1, CDR2, and CDR3 sequences as set forth in SEQ ID NO: 95, SEQ ID NO: 96, and SEQ ID NO: 97, respectively; and light chain CDR1, CDR2, and CDR3 sequences as set forth in SEQ ID NO: 98, SEQ ID NO: 99 (or 100), and SEQ ID NO: 101, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 102 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 104. Thereby, the CDR sequences as defined above (heavy chain CDR1, CDR2, and CDR3 sequences as set forth in SEQ ID NO: 95, SEQ ID NO: 96, and SEQ ID NO: 97, respectively; and light chain CDR1, CDR2, and CDR3 sequences as set forth in SEQ ID NO: 98, SEQ ID NO: 99 (or 100), and SEQ ID NO: 101, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 111 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 112. Thereby, the CDR sequences as defined above (heavy chain CDR1, CDR2, and CDR3 sequences as set forth in SEQ ID NO: 105, SEQ ID NO: 106, and SEQ ID NO: 107, respectively; and light chain CDR1, CDR2, and CDR3 sequences as set forth in SEQ ID NO: 108, SEQ ID NO: 99 (or 109), and SEQ ID NO: 110, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 118 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 119. Thereby, the CDR sequences as defined above (heavy chain CDR1, CDR2, and CDR3 sequences as set forth in SEQ ID NO: 113, SEQ ID NO: 114, and SEQ ID NO: 115, respectively; and light chain CDR1, CDR2, and CDR3 sequences as set forth in SEQ ID NO: 98, SEQ ID NO: 99 (or 116), and SEQ ID NO: 117, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 120 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 121. Thereby, the CDR sequences as defined above (heavy chain CDR1, CDR2, and CDR3 sequences as set forth in SEQ ID NO: 113, SEQ ID NO: 114, and SEQ ID NO: 115, respectively; and light chain CDR1, CDR2, and CDR3 sequences as set forth in SEQ ID NO: 98, SEQ ID NO: 99 (or 116), and SEQ ID NO: 117, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 125 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 126. Thereby, the CDR sequences as defined above (heavy chain CDR1, CDR2, and CDR3 sequences as set forth in SEQ ID NO: 105, SEQ ID NO: 122, and SEQ ID NO: 123, respectively; and light chain CDR1, CDR2, and CDR3 sequences as set forth in SEQ ID NO: 124, SEQ ID NO: 99 (or 109), and SEQ ID NO: 110, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 134 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 135. Thereby, the CDR sequences as defined above (heavy chain CDR1, CDR2, and CDR3 sequences as set forth in SEQ ID NO: 127, SEQ ID NO: 128, and SEQ ID NO: 129, respectively; and light chain CDR1, CDR2, and CDR3 sequences as set forth in SEQ ID NO: 130, SEQ ID NO: 131 (or 132), and SEQ ID NO: 133, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 111 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 112. Thereby, the CDR sequences as defined above (heavy chain CDR1, CDR2, and CDR3 sequences as set forth in SEQ ID NO: 105, SEQ ID NO: 106, and SEQ ID NO: 107, respectively; and light chain CDR1, CDR2, and CDR3 sequences as set forth in SEQ ID NO: 108, SEQ ID NO: 99 (or 109), and SEQ ID NO: 110, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 136 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 137. Thereby, the CDR sequences as defined above (heavy chain CDR1, CDR2, and CDR3 sequences as set forth in SEQ ID NO: 105, SEQ ID NO: 106, and SEQ ID NO: 107, respectively; and light chain CDR1, CDR2, and CDR3 sequences as set forth in SEQ ID NO: 108, SEQ ID NO: 99 (or 109), and SEQ ID NO: 110, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 139 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 140. Thereby, the CDR sequences as defined above (heavy chain CDR1, CDR2, and CDR3 sequences as set forth in SEQ ID NO: 138, SEQ ID NO: 106, and SEQ ID NO: 107, respectively; and light chain CDR1, CDR2, and CDR3 sequences as set forth in SEQ ID NO: 108, SEQ ID NO: 99 (or 109), and SEQ ID NO: 110, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 144 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 145. Thereby, the CDR sequences as defined above (heavy chain CDR1, CDR2, and CDR3 sequences as set forth in SEQ ID NO: 141, SEQ ID NO: 142, and SEQ ID NO: 143, respectively; and light chain CDR1, CDR2, and CDR3 sequences as set forth in SEQ ID NO: 108, SEQ ID NO: 99 (or 109), and SEQ ID NO: 110, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 146 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 147. Thereby, the CDR sequences as defined above (heavy chain CDR1, CDR2, and CDR3 sequences as set forth in SEQ ID NO: 138, SEQ ID NO: 142, and SEQ ID NO: 107, respectively; and light chain CDR1, CDR2, and CDR3 sequences as set forth in SEQ ID NO: 108, SEQ ID NO: 99 (or 109), and SEQ ID NO: 110, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 155 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 156. Thereby, the CDR sequences as defined above (heavy chain CDR1, CDR2, and CDR3 sequences as set forth in SEQ ID NO: 148, SEQ ID NO: 149, and SEQ ID NO: 150, respectively; and light chain CDR1, CDR2, and CDR3 sequences as set forth in SEQ ID NO: 151, SEQ ID NO: 152 (or 153), and SEQ ID NO: 154, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 164 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 165. Thereby, the CDR sequences as defined above (heavy chain CDR1, CDR2, and CDR3 sequences as set forth in SEQ ID NO: 157, SEQ ID NO: 158, and SEQ ID NO: 159, respectively; and light chain CDR1, CDR2, and CDR3 sequences as set forth in SEQ ID NO: 160, SEQ ID NO: 161 (or 162), and SEQ ID NO: 163, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 169 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 170. Thereby, the CDR sequences as defined above (heavy chain CDR1, CDR2, and CDR3 sequences as set forth in SEQ ID NO: 157, SEQ ID NO: 158, and SEQ ID NO: 159, respectively; and light chain CDR1, CDR2, and CDR3 sequences as set forth in SEQ ID NO: 166, SEQ ID NO: 99 (or 167), and SEQ ID NO: 168, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 175 and a light chain variable region (VL) comprising an amino acid sequence having 70% or more (e.g., at least 70%, at least 710%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 176. Thereby, the CDR sequences as defined above (heavy chain CDR1, CDR2, and CDR3 sequences as set forth in SEQ ID NO: 171, SEQ ID NO: 172, and SEQ ID NO: 173, respectively; and light chain CDR1, CDR2, and CDR3 sequences as set forth in SEQ ID NO: 174, SEQ ID NO: 99 (or 167), and SEQ ID NO: 168, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 910%, at least 92%, at least 93%, at least 94%, at least 95%, at least 96%, at least 97%, at least 98% or at least 99%) identity to SEQ ID NO: 181 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 182. Thereby, the CDR sequences as defined above (heavy chain CDR1, CDR2, and CDR3 sequences as set forth in SEQ ID NO: 177, SEQ ID NO: 178, and SEQ ID NO: 179, respectively; and light chain CDR1, CDR2, and CDR3 sequences as set forth in SEQ ID NO: 180, SEQ ID NO: 99 (or 116), and SEQ ID NO: 168, respectively) are preferably maintained.

More preferably, the antibody, or an antigen-binding fragment thereof, preferably comprises a heavy chain variable region comprising or consisting of an amino acid sequence as set forth in SEQ ID NO: 102 and a light chain variable region comprising or consisting of an amino acid sequence as set forth in SEQ ID NO: 103.

In some embodiments, the antibody, or an antigen-binding fragment thereof, more preferably comprises a heavy chain variable region comprising or consisting of an amino acid sequence as set forth in SEQ ID NO: 102 and a light chain variable region comprising or consisting of an amino acid sequence as set forth in SEQ ID NO: 104.

In some embodiments, the antibody, or an antigen-binding fragment thereof, more preferably comprises a heavy chain variable region comprising or consisting of an amino acid sequence as set forth in SEQ ID NO: 111 and a light chain variable region comprising or consisting of an amino acid sequence as set forth in SEQ ID NO: 112.

In some embodiments, the antibody, or an antigen-binding fragment thereof, more preferably comprises a heavy chain variable region comprising or consisting of an amino acid sequence as set forth in SEQ ID NO: 118 and a light chain variable region comprising or consisting of an amino acid sequence as set forth in SEQ ID NO: 119.

In some embodiments, the antibody, or an antigen-binding fragment thereof, more preferably comprises a heavy chain variable region comprising or consisting of an amino acid sequence as set forth in SEQ ID NO: 120 and a light chain variable region comprising or consisting of an amino acid sequence as set forth in SEQ ID NO: 121.

In some embodiments, the antibody, or an antigen-binding fragment thereof, more preferably comprises a heavy chain variable region comprising or consisting of an amino acid sequence as set forth in SEQ ID NO: 125 and a light chain variable region comprising or consisting of an amino acid sequence as set forth in SEQ ID NO: 126.

In some embodiments, the antibody, or an antigen-binding fragment thereof, more preferably comprises a heavy chain variable region comprising or consisting of an amino acid sequence as set forth in SEQ ID NO: 134 and a light chain variable region comprising or consisting of an amino acid sequence as set forth in SEQ ID NO: 135.

In some embodiments, the antibody, or an antigen-binding fragment thereof, more preferably comprises a heavy chain variable region comprising or consisting of an amino acid sequence as set forth in SEQ ID NO: 136 and a light chain variable region comprising or consisting of an amino acid sequence as set forth in SEQ ID NO: 137.

In some embodiments, the antibody, or an antigen-binding fragment thereof, more preferably comprises a heavy chain variable region comprising or consisting of an amino acid sequence as set forth in SEQ ID NO: 139 and a light chain variable region comprising or consisting of an amino acid sequence as set forth in SEQ ID NO: 140.

In some embodiments, the antibody, or an antigen-binding fragment thereof, more preferably comprises a heavy chain variable region comprising or consisting of an amino acid sequence as set forth in SEQ ID NO: 144 and a light chain variable region comprising or consisting of an amino acid sequence as set forth in SEQ ID NO: 145.

In some embodiments, the antibody, or an antigen-binding fragment thereof, more preferably comprises a heavy chain variable region comprising or consisting of an amino acid sequence as set forth in SEQ ID NO: 146 and a light chain variable region comprising or consisting of an amino acid sequence as set forth in SEQ ID NO: 147.

In some embodiments, the antibody, or an antigen-binding fragment thereof, more preferably comprises a heavy chain variable region comprising or consisting of an amino acid sequence as set forth in SEQ ID NO: 155 and a light chain variable region comprising or consisting of an amino acid sequence as set forth in SEQ ID NO: 156.

In some embodiments, the antibody, or an antigen-binding fragment thereof, more preferably comprises a heavy chain variable region comprising or consisting of an amino acid sequence as set forth in SEQ ID NO: 164 and a light chain variable region comprising or consisting of an amino acid sequence as set forth in SEQ ID NO: 165.

In some embodiments, the antibody, or an antigen-binding fragment thereof, more preferably comprises a heavy chain variable region comprising or consisting of an amino acid sequence as set forth in SEQ ID NO: 169 and a light chain variable region comprising or consisting of an amino acid sequence as set forth in SEQ ID NO: 170.

In some embodiments, the antibody, or an antigen-binding fragment thereof, more preferably comprises a heavy chain variable region comprising or consisting of an amino acid sequence as set forth in SEQ ID NO: 175 and a light chain variable region comprising or consisting of an amino acid sequence as set forth in SEQ ID NO: 176.

In some embodiments, the antibody, or an antigen-binding fragment thereof, more preferably comprises a heavy chain variable region comprising or consisting of an amino acid sequence as set forth in SEQ ID NO: 181 and a light chain variable region comprising or consisting of an amino acid sequence as set forth in SEQ ID NO: 182.

The CDR and VH/VL sequences of exemplified antibodies of the invention binding to CCL8 are shown in Table 2 below.

TABLE 2
CDR and VH/VL sequences (SEQ ID NOs) of exemplified
antibodies of the invention binding to CCL8.

| Antibody | CDRH1 | CDRH2 | CDRH3 | CDRL1 | CDRL2 | CDRL3 | VH | VL |
|---|---|---|---|---|---|---|---|---|
| aCCL8.001 | 95 | 96 | 97 | 98 | 99/100 | 101 | 102 | 103 |
| aCCL8.001v1 | 95 | 96 | 97 | 98 | 99/100 | 101 | 102 | 104 |
| aCCL8.003 | 105 | 106 | 107 | 108 | 99/109 | 110 | 111 | 112 |
| aCCL8.004 | 113 | 114 | 115 | 98 | 99/116 | 117 | 118 | 119 |
| aCCL8.004v1 | 113 | 114 | 115 | 98 | 99/116 | 117 | 120 | 121 |
| aCCL8.005 | 105 | 122 | 123 | 124 | 99/109 | 110 | 125 | 126 |
| aCCL8.008 | 127 | 128 | 129 | 130 | 131/132 | 133 | 134 | 135 |
| aCCL8.008v1 | 127 | 128 | 129 | 130 | 131/132 | 133 | 136 | 137 |
| aCCL8_B5 | 138 | 106 | 107 | 108 | 99/109 | 110 | 139 | 140 |
| aCCL8_C4 | 141 | 142 | 143 | 108 | 99/109 | 110 | 144 | 145 |
| aCCL8_D9 | 138 | 142 | 107 | 108 | 99/109 | 110 | 146 | 147 |
| aCCL8_A2 | 148 | 149 | 150 | 151 | 152/153 | 154 | 155 | 156 |
| aCCL8_A5 | 157 | 158 | 159 | 160 | 161/162 | 163 | 164 | 165 |
| aCCL8_D2 | 157 | 158 | 159 | 166 | 99/167 | 168 | 169 | 170 |
| aCCL8_C1 | 171 | 172 | 173 | 174 | 99/167 | 168 | 175 | 176 |
| aCCL8_B11 | 177 | 178 | 179 | 180 | 99/116 | 168 | 181 | 182 |

Preferably, the antibody of the invention comprises the combination of six CDR sequences (optionally the VH and VL sequences) of the exemplified antibodies shown in Table 2, or respective sequence variants thereof, as defined herein above.

In some embodiments, the antibody, or the antigen-binding fragment thereof, binds to the N-loop of CCL8; but preferably not to the N-loop of CCL2, CCL7, CCL13 or CCL11.

In some embodiments, the antibody, or the antigen-binding fragment thereof, reduces or inhibits cell migration towards CCL8.

In some embodiments, the antibody, or the antigen-binding fragment thereof, reduces or inhibits CCR1-mediated chemotaxis (but not CCR2-mediated chemotaxis)

As demonstrated in the examples, exemplified antibodies of the invention, which bind to CCL8, exhibit such features. To assess any one of those features, methods as described for this purpose in the present examples may be used.

### Antibodies Binding to CCL20 (Chemokine (C-C Motif) Ligand 20)

In some aspects, the present invention provides an antibody, or an antigen-binding fragment thereof, which binds to CCL20 (Chemokine (C-C motif) ligand 20), wherein the antibody, or the antigen-binding fragment thereof, comprises heavy chain CDR1, CDR2, and CDR3 sequences having at least 70% sequence identity with the amino acid sequences SEQ ID NO: 183, SEQ ID NO: 184, and SEQ ID NO: 185, respectively, and light chain CDR1, CDR2, and CDR3 sequences having at least 70% sequence identity with the amino acid sequences of SEQ ID NO: 186, SEQ ID NO: 99 (or 187), and SEQ ID NO: 188, respectively.

The general definition of sequence variants as described above applies accordingly to the sequence variants of the above-defined specific CDR sequences of the antibody, or an antigen-binding fragment thereof, which binds to CCL20. Likewise, the general definition of sequence variants as described above applies accordingly to the sequence variants of the specific VH and VL sequences of the antibody, or an antigen-binding fragment thereof, which binds to CCL20 (as described below).

Preferably, the antibody, or an antigen-binding fragment thereof, which binds to CCL20, comprises heavy chain CDR1, CDR2, and CDR3 sequences according to SEQ ID NO: 183, SEQ ID NO: 184, and SEQ ID NO: 185, respectively, and light chain CDR1, CDR2, and CDR3 sequences according to SEQ ID NO: 186, SEQ ID NO: 99 (or 187), and SEQ ID NO: 188, respectively.

Accordingly, the antibody or the antigen-binding fragment thereof preferably comprises:
a heavy chain CDR1 sequence according to SEQ ID NO: 183;
a heavy chain CDR2 sequence according to SEQ ID NO: 184;
a heavy chain CDR3 sequence according to SEQ ID NO: 185;
a light chain CDR1 sequence according to SEQ ID NO: 186;
a light chain CDR2 sequence according to SEQ ID NO: 99 (or 187); and
a light chain CDR3 sequence according to SEQ ID NO: 188.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 189 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 190. Thereby, the CDR sequences as defined above (heavy chain CDR1, CDR2, and CDR3 sequences as set forth in SEQ ID NO: 183, SEQ ID NO: 184, and SEQ ID NO: 185, respectively; and light chain CDR1, CDR2, and CDR3 sequences as set forth in SEQ ID NO: 186, SEQ ID NO: 99 (or 187), and SEQ ID NO: 188, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 191 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 192. Thereby, the CDR sequences as defined above (heavy chain CDR1, CDR2, and CDR3 sequences as set forth in SEQ ID NO: 183, SEQ ID NO: 184, and SEQ ID NO: 185, respectively; and light chain CDR1, CDR2, and CDR3 sequences as set forth in SEQ ID NO: 186, SEQ ID NO: 99 (or 187), and SEQ ID NO: 188, respectively) are preferably maintained.

More preferably, the antibody, or an antigen-binding fragment thereof, preferably comprises a heavy chain variable region comprising or consisting of an amino acid sequence as set forth in SEQ ID NO: 190 and a light chain variable region comprising or consisting of an amino acid sequence as set forth in SEQ ID NO: 191.

In some embodiments, the antibody, or an antigen-binding fragment thereof, more preferably comprises a heavy chain variable region comprising or consisting of an amino acid sequence as set forth in SEQ ID NO: 191 and a light chain variable region comprising or consisting of an amino acid sequence as set forth in SEQ ID NO: 192.

The CDR and VH/VL sequences of exemplified antibodies of the invention binding to CCL20 are shown in Table 3 below.

TABLE 3
CDR and VH/VL sequences (SEQ ID NOs) of exemplified
antibodies of the invention binding to CCL20.

| Antibody | CDRH1 | CDRH2 | CDRH3 | CDRL1 | CDRL2 | CDRL3 | VH | VL |
|---|---|---|---|---|---|---|---|---|
| aCCL20.001 | 183 | 184 | 185 | 186 | 99/187 | 188 | 189 | 190 |
| aCCL20.001v1 | 183 | 184 | 185 | 186 | 99/187 | 188 | 191 | 192 |

Preferably, the antibody of the invention comprises the combination of six CDR sequences (optionally the VH and VL sequences) of the exemplified antibodies shown in Table 3, or respective sequence variants thereof, as defined herein above.

In some embodiments, the antibody, or the antigen-binding fragment thereof, binds to the N-loop of CCL20.

In some embodiments, the antibody, or the antigen-binding fragment thereof, reduces or inhibits cell migration towards CCL20.

As demonstrated in the examples, exemplified antibodies of the invention, which bind to CCL20, exhibit such features. To assess any one of those features, methods as described for this purpose in the present examples may be used.

### Antibodies Binding to CCL23 (Chemokine (C-C Motif) Ligand 23)

In some aspects, the present invention provides an antibody, or an antigen-binding fragment thereof, which binds to CCL23 (Chemokine (C-C motif) ligand 23), wherein the antibody, or the antigen-binding fragment thereof, comprises:
(i) heavy chain CDR1, CDR2, and CDR3 sequences having at least 70% sequence identity with the amino acid sequences of SEQ ID NO: 193, SEQ ID NO: 194, and SEQ ID NO: 195, respectively, and light chain CDR1, CDR2, and CDR3 sequences having at least 70% sequence identity with the amino acid sequences of SEQ ID NO: 196, SEQ ID NO: 197 (or 198), and SEQ ID NO: 199, respectively; or
(ii) heavy chain CDR1, CDR2, and CDR3 sequences having at least 70% sequence identity with the amino acid sequences of SEQ ID NO: 203, SEQ ID NO: 204, and SEQ ID NO: 205, respectively, and light chain CDR1, CDR2, and CDR3 sequences having at least 70% sequence identity with the amino acid sequences of SEQ ID NO: 206, SEQ ID NO: 197 (or 207), and SEQ ID NO: 208, respectively.

The general definition of sequence variants as described above applies accordingly to the sequence variants of the above-defined specific CDR sequences of the antibody, or an antigen-binding fragment thereof, which binds to CCL23. Likewise, the general definition of sequence variants as described above applies accordingly to the sequence variants of the specific VH and VL sequences of the antibody, or an antigen-binding fragment thereof, which binds to CCL23 (as described below).

Preferably, the antibody, or an antigen-binding fragment thereof, which binds to CCL23, comprises
(i) heavy chain CDR1, CDR2, and CDR3 sequences according to SEQ ID NO: 193, SEQ ID NO: 194, and SEQ ID NO: 195, respectively, and light chain CDR1, CDR2, and CDR3 sequences according to SEQ ID NO: 196, SEQ ID NO: 197 (or 198), and SEQ ID NO: 199, respectively; or
(ii) heavy chain CDR1, CDR2, and CDR3 sequences according to SEQ ID NO: 203, SEQ ID NO: 204, and SEQ ID NO: 205, respectively, and light chain CDR1, CDR2, and CDR3 sequences according to SEQ ID NO: 206, SEQ ID NO: 197 (or 207), and SEQ ID NO: 208, respectively.

Accordingly, the antibody or the antigen-binding fragment thereof preferably comprises:
a heavy chain CDR1 sequence according to SEQ ID NO: 193;
a heavy chain CDR2 sequence according to SEQ ID NO: 194;
a heavy chain CDR3 sequence according to SEQ ID NO: 195;
a light chain CDR1 sequence according to SEQ ID NO: 196;
a light chain CDR2 sequence according to SEQ ID NO: 197 (or 198); and
a light chain CDR3 sequence according to SEQ ID NO: 199.

In some embodiments, the antibody or the antigen-binding fragment thereof preferably comprises:
a heavy chain CDR1 sequence according to SEQ ID NO: 203;
a heavy chain CDR2 sequence according to SEQ ID NO: 204;
a heavy chain CDR3 sequence according to SEQ ID NO: 205;
a light chain CDR1 sequence according to SEQ ID NO: 206;
a light chain CDR2 sequence according to SEQ ID NO: 197 (or 207); and
a light chain CDR3 sequence according to SEQ ID NO: 208.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 200 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 201. Thereby, the CDR sequences as defined above (heavy chain CDR1, CDR2, and CDR3 sequences as set forth in SEQ ID NO: 193, SEQ ID NO: 194, and SEQ ID NO: 195, respectively; and light chain CDR1, CDR2, and CDR3 sequences as set forth in SEQ ID NO: 196, SEQ ID NO: 197 (or 198), and SEQ ID NO: 199, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 202 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 201. Thereby, the CDR sequences as defined above (heavy chain CDR1, CDR2, and CDR3 sequences as set forth in SEQ ID NO: 193, SEQ ID NO: 194, and SEQ ID NO: 195, respectively; and light chain CDR1, CDR2, and CDR3 sequences as set forth in SEQ ID NO: 196, SEQ ID NO: 197 (or 198), and SEQ ID NO: 199, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 209 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 210. Thereby, the CDR sequences as defined above (heavy chain CDR1, CDR2, and CDR3 sequences as set forth in SEQ ID NO: 203, SEQ ID NO: 204, and SEQ ID NO: 205, respectively; and light chain CDR1, CDR2, and CDR3 sequences as set forth in SEQ ID NO: 206, SEQ ID NO: 197 (or 207), and SEQ ID NO: 208, respectively) are preferably maintained.

More preferably, the antibody, or an antigen-binding fragment thereof, preferably comprises a heavy chain variable region comprising or consisting of an amino acid sequence as set forth in SEQ ID NO: 200 and a light chain variable region comprising or consisting of an amino acid sequence as set forth in SEQ ID NO: 201.

In some embodiments, the antibody, or an antigen-binding fragment thereof, more preferably comprises a heavy chain variable region comprising or consisting of an amino acid sequence as set forth in SEQ ID NO: 202 and a light chain variable region comprising or consisting of an amino acid sequence as set forth in SEQ ID NO: 201.

In some embodiments, the antibody, or an antigen-binding fragment thereof, more preferably comprises a heavy chain variable region comprising or consisting of an amino acid sequence as set forth in SEQ ID NO: 209 and a light chain variable region comprising or consisting of an amino acid sequence as set forth in SEQ ID NO: 210.

The CDR and VH/VL sequences of exemplified antibodies of the invention binding to CCL23 are shown in Table 4 below.

TABLE 4
CDR and VH/VL sequences (SEQ ID NOs) of exemplified
antibodies of the invention binding to CCL23.

| Antibody | CDRH1 | CDRH2 | CDRH3 | CDRL1 | CDRL2 | CDRL3 | VH | VL |
|---|---|---|---|---|---|---|---|---|
| aCCL23.001 | 193 | 194 | 195 | 196 | 197/198 | 199 | 200 | 201 |
| aCCL23.001v1 | 193 | 194 | 195 | 196 | 197/198 | 199 | 202 | 201 |
| aCCL23.002 | 203 | 204 | 205 | 206 | 197/207 | 208 | 209 | 210 |

Preferably, the antibody of the invention comprises the combination of six CDR sequences (optionally the VH and VL sequences) of the exemplified antibodies shown in Table 4, or respective sequence variants thereof, as defined herein above.

In some embodiments, the antibody, or the antigen-binding fragment thereof, binds to the N-loop of CCL23.

In some embodiments, the antibody, or the antigen-binding fragment thereof, reduces or inhibits cell migration towards CCL23.

### Antibodies Binding to CXCL13 (Chemokine (C-X-C Motif) Ligand 13)

In some aspects, the present invention provides an antibody, or an antigen-binding fragment thereof, which binds to CXCL13 (Chemokine (C-X-C motif) ligand 13), wherein the antibody, or the antigen-binding fragment thereof, comprises:
(i) heavy chain CDR1, CDR2, and CDR3 sequences having at least 70% sequence identity with the amino acid sequences of SEQ ID NO: 211, SEQ ID NO: 212, and SEQ ID NO: 213, respectively, and light chain CDR1, CDR2, and CDR3 sequences having at least 70% sequence identity with the amino acid sequences of SEQ ID NO: 214, SEQ ID NO: 215 (or 216), and SEQ ID NO: 217, respectively;
(ii) heavy chain CDR1, CDR2, and CDR3 sequences having at least 70% sequence identity with the amino acid sequences of SEQ ID NO: 221, SEQ ID NO: 222, and SEQ ID NO: 223, respectively, and light chain CDR1, CDR2, and CDR3 sequences having at least 70% sequence identity with the amino acid sequences of SEQ ID NO: 224, SEQ ID NO: 99 (or 109), and SEQ ID NO: 225, respectively; or
(iii) heavy chain CDR1, CDR2, and CDR3 sequences having at least 70% sequence identity with the amino acid sequences of SEQ ID NO: 229, SEQ ID NO: 230, and SEQ ID NO: 231, respectively, and light chain CDR1, CDR2, and CDR3 sequences having at least 70% sequence identity with the amino acid sequences of SEQ ID NO: 224, SEQ ID NO: 99 (or 109), and SEQ ID NO: 225, respectively.

The general definition of sequence variants as described above applies accordingly to the sequence variants of the above-defined specific CDR sequences of the antibody, or an antigen-binding fragment thereof, which binds to CXCL13. Likewise, the general definition of sequence variants as described above applies accordingly to the sequence variants of the specific VH and VL sequences of the antibody, or an antigen-binding fragment thereof, which binds to CXCL13 (as described below).

Preferably, the antibody, or an antigen-binding fragment thereof, which binds to CXCL13, comprises
(i) heavy chain CDR1, CDR2, and CDR3 sequences according to SEQ ID NO: 211, SEQ ID NO: 212, and SEQ ID NO: 213, respectively, and light chain CDR1, CDR2, and CDR3 sequences according to SEQ ID NO: 214, SEQ ID NO: 215 (or 216), and SEQ ID NO: 217, respectively;
(ii) heavy chain CDR1, CDR2, and CDR3 sequences according to SEQ ID NO: 221, SEQ ID NO: 222, and SEQ ID NO: 223, respectively, and light chain CDR1, CDR2, and CDR3 sequences according to SEQ ID NO: 224, SEQ ID NO: 99 (or 109), and SEQ ID NO: 225, respectively; or
(iii) heavy chain CDR1, CDR2, and CDR3 sequences according to SEQ ID NO: 229, SEQ ID NO: 230, and SEQ ID NO: 231, respectively, and light chain CDR1, CDR2, and CDR3 sequences according to SEQ ID NO: 224, SEQ ID NO: 99 (or 109), and SEQ ID NO: 225, respectively.

Accordingly, the antibody or the antigen-binding fragment thereof preferably comprises:
a heavy chain CDR1 sequence according to SEQ ID NO: 211;
a heavy chain CDR2 sequence according to SEQ ID NO: 212;
a heavy chain CDR3 sequence according to SEQ ID NO: 213;
a light chain CDR1 sequence according to SEQ ID NO: 214;
a light chain CDR2 sequence according to SEQ ID NO: 215 (or 216); and
a light chain CDR3 sequence according to SEQ ID NO: 217.

In some embodiments, the antibody or the antigen-binding fragment thereof preferably comprises:
a heavy chain CDR1 sequence according to SEQ ID NO: 221;
a heavy chain CDR2 sequence according to SEQ ID NO: 222;
a heavy chain CDR3 sequence according to SEQ ID NO: 223;
a light chain CDR1 sequence according to SEQ ID NO: 224;
a light chain CDR2 sequence according to SEQ ID NO: 99 (or 109); and
a light chain CDR3 sequence according to SEQ ID NO: 225.

In some embodiments, the antibody or the antigen-binding fragment thereof preferably comprises:
a heavy chain CDR1 sequence according to SEQ ID NO: 229;
a heavy chain CDR2 sequence according to SEQ ID NO: 230;
a heavy chain CDR3 sequence according to SEQ ID NO: 231;
a light chain CDR1 sequence according to SEQ ID NO: 224;
a light chain CDR2 sequence according to SEQ ID NO: 99 (or 109); and
a light chain CDR3 sequence according to SEQ ID NO: 225.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 218 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 219. Thereby, the CDR sequences as defined above (heavy chain CDR1, CDR2, and CDR3 sequences as set forth in SEQ ID NO: 211, SEQ ID NO: 212, and SEQ ID NO: 213, respectively; and light chain CDR1, CDR2, and CDR3 sequences as set forth in SEQ ID NO: 214, SEQ ID NO: 215 (or 216), and SEQ ID NO: 217, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 218 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 220. Thereby, the CDR sequences as defined above (heavy chain CDR1, CDR2, and CDR3 sequences as set forth in SEQ ID NO: 211, SEQ ID NO: 212, and SEQ ID NO: 213, respectively; and light chain CDR1, CDR2, and CDR3 sequences as set forth in SEQ ID NO: 214, SEQ ID NO: 215 (or 216), and SEQ ID NO: 217, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 226 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 227. Thereby, the CDR sequences as defined above (heavy chain CDR1, CDR2, and CDR3 sequences as set forth in SEQ ID NO: 221, SEQ ID NO: 222, and SEQ ID NO: 223, respectively; and light chain CDR1, CDR2, and CDR3 sequences as set forth in SEQ ID NO: 224, SEQ ID NO: 99 (or 109), and SEQ ID NO: 225, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 226 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 228. Thereby, the CDR sequences as defined above (heavy chain CDR1, CDR2, and CDR3 sequences as set forth in SEQ ID NO: 221, SEQ ID NO: 222, and SEQ ID NO: 223, respectively; and light chain CDR1, CDR2, and CDR3 sequences as set forth in SEQ ID NO: 224, SEQ ID NO: 99 (or 109), and SEQ ID NO: 225, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 232 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 233. Thereby, the CDR sequences as defined above (heavy chain CDR1, CDR2, and CDR3 sequences as set forth in SEQ ID NO: 229, SEQ ID NO: 230, and SEQ ID NO: 231, respectively; and light chain CDR1, CDR2, and CDR3 sequences as set forth in SEQ ID NO: 224, SEQ ID NO: 99 (or 109), and SEQ ID NO: 225, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 234 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 233. Thereby, the CDR sequences as defined above (heavy chain CDR1, CDR2, and CDR3 sequences as set forth in SEQ ID NO: 229, SEQ ID NO: 230, and SEQ ID NO: 231, respectively; and light chain CDR1, CDR2, and CDR3 sequences as set forth in SEQ ID NO: 224, SEQ ID NO: 99 (or 109), and SEQ ID NO: 225, respectively) are preferably maintained.

More preferably, the antibody, or an antigen-binding fragment thereof, preferably comprises a heavy chain variable region comprising or consisting of an amino acid sequence as set forth in SEQ ID NO: 218 and a light chain variable region comprising or consisting of an amino acid sequence as set forth in SEQ ID NO: 219.

In some embodiments, the antibody, or an antigen-binding fragment thereof, more preferably comprises a heavy chain variable region comprising or consisting of an amino acid sequence as set forth in SEQ ID NO: 218 and a light chain variable region comprising or consisting of an amino acid sequence as set forth in SEQ ID NO: 220.

In some embodiments, the antibody, or an antigen-binding fragment thereof, more preferably comprises a heavy chain variable region comprising or consisting of an amino acid sequence as set forth in SEQ ID NO: 226 and a light chain variable region comprising or consisting of an amino acid sequence as set forth in SEQ ID NO: 227.

In some embodiments, the antibody, or an antigen-binding fragment thereof, more preferably comprises a heavy chain variable region comprising or consisting of an amino acid sequence as set forth in SEQ ID NO: 226 and a light chain variable region comprising or consisting of an amino acid sequence as set forth in SEQ ID NO: 228.

In some embodiments, the antibody, or an antigen-binding fragment thereof, more preferably comprises a heavy chain variable region comprising or consisting of an amino acid sequence as set forth in SEQ ID NO: 232 and a light chain variable region comprising or consisting of an amino acid sequence as set forth in SEQ ID NO: 233.

In some embodiments, the antibody, or an antigen-binding fragment thereof, more preferably comprises a heavy chain variable region comprising or consisting of an amino acid sequence as set forth in SEQ ID NO: 234 and a light chain variable region comprising or consisting of an amino acid sequence as set forth in SEQ ID NO: 233.

The CDR and VH/VL sequences of exemplified antibodies of the invention binding to CXCL13 are shown in Table 5 below.

TABLE 5
CDR and VH/VL sequences (SEQ ID NOs) of exemplified
antibodies of the invention binding to CXCL13.

| Antibody | CDRH1 | CDRH2 | CDRH3 | CDRL1 | CDRL2 | CDRL3 | VH | VL |
|---|---|---|---|---|---|---|---|---|
| aCXCL13.001 | 211 | 212 | 213 | 214 | 215/216 | 217 | 218 | 219 |
| aCXCL13.001v1 | 211 | 212 | 213 | 214 | 215/216 | 217 | 218 | 220 |
| aCXCL13.002 | 221 | 222 | 223 | 224 | 99/109 | 225 | 226 | 227 |
| aCXCL13.002v1 | 221 | 222 | 223 | 224 | 99/109 | 225 | 226 | 228 |
| aCXCL13.003 | 229 | 230 | 231 | 224 | 99/109 | 225 | 232 | 233 |
| aCXCL13.003v1 | 229 | 230 | 231 | 224 | 99/109 | 225 | 234 | 233 |

Preferably, the antibody of the invention comprises the combination of six CDR sequences (optionally the VH and VL sequences) of the exemplified antibodies shown in Table 5, or respective sequence variants thereof, as defined herein above.

In some embodiments, the antibody, or the antigen-binding fragment thereof, binds to the N-loop of CXCL13.

In some embodiments, the antibody, or the antigen-binding fragment thereof, reduces or inhibits cell migration towards CXCL13.

As demonstrated in the examples, exemplified antibodies of the invention, which bind to CXCL13, exhibit such features. To assess any one of those features, methods as described for this purpose in the present examples may be used.

Antibodies binding to CXCL16 (Chemokine (C-X-C motif) ligand 16) In some aspects, the present invention provides an antibody, or an antigen-binding fragment thereof, which binds to CXCL16 (Chemokine (C-X-C motif) ligand 16), wherein the antibody, or the antigen-binding fragment thereof, comprises:
(i) heavy chain CDR1, CDR2, and CDR3 sequences having at least 70% sequence identity with the amino acid sequences of SEQ ID NO: 235, SEQ ID NO: 236, and SEQ ID NO: 237, respectively, and light chain CDR1, CDR2, and CDR3 sequences having at least 70% sequence identity with the amino acid sequences of SEQ ID NO: 238, SEQ ID NO: 239 (or 240), and SEQ ID NO: 241, respectively;
(ii) heavy chain CDR1, CDR2, and CDR3 sequences having at least 70% sequence identity with the amino acid sequences of SEQ ID NO: 246, SEQ ID NO: 247, and SEQ ID NO: 248, respectively, and light chain CDR1, CDR2, and CDR3 sequences having at least 70% sequence identity with the amino acid sequences of SEQ ID NO: 249, SEQ ID NO: 197 (or 250), and SEQ ID NO: 251, respectively;
(iii) heavy chain CDR1, CDR2, and CDR3 sequences having at least 70% sequence identity with the amino acid sequences of SEQ ID NO: 246, SEQ ID NO: 247, and SEQ ID NO: 248, respectively, and light chain CDR1, CDR2, and CDR3 sequences having at least 70% sequence identity with the amino acid sequences of SEQ ID NO: 249, SEQ ID NO: 197 (or 254), and SEQ ID NO: 251, respectively; or
(iv) heavy chain CDR1, CDR2, and CDR3 sequences having at least 70% sequence identity with the amino acid sequences of SEQ ID NO: 257, SEQ ID NO: 258, and SEQ ID NO: 259, respectively, and light chain CDR1, CDR2, and CDR3 sequences having at least 70% sequence identity with the amino acid sequences of SEQ ID NO: 260, SEQ ID NO: 261 (or 262), and SEQ ID NO: 263, respectively.

The general definition of sequence variants as described above applies accordingly to the sequence variants of the above-defined specific CDR sequences of the antibody, or an antigen-binding fragment thereof, which binds to CXCL16. Likewise, the general definition of sequence variants as described above applies accordingly to the sequence variants of the specific VH and VL sequences of the antibody, or an antigen-binding fragment thereof, which binds to CXCL16 (as described below).

Preferably, the antibody, or an antigen-binding fragment thereof, which binds to CXCL16, comprises
(i) heavy chain CDR1, CDR2, and CDR3 sequences according to SEQ ID NO: 235, SEQ ID NO: 236, and SEQ ID NO: 237, respectively, and light chain CDR1, CDR2, and CDR3 sequences according to of SEQ ID NO: 238, SEQ ID NO: 239 (or 240), and SEQ ID NO: 241, respectively;
(ii) heavy chain CDR1, CDR2, and CDR3 sequences according to SEQ ID NO: 246, SEQ ID NO: 247, and SEQ ID NO: 248, respectively, and light chain CDR1, CDR2, and CDR3 sequences according to SEQ ID NO: 249, SEQ ID NO: 197 (or 250), and SEQ ID NO: 251, respectively;
(iii) heavy chain CDR1, CDR2, and CDR3 sequences according to SEQ ID NO: 246, SEQ ID NO: 247, and SEQ ID NO: 248, respectively, and light chain CDR1, CDR2, and CDR3 sequences according to SEQ ID NO: 249, SEQ ID NO: 197 (or 254), and SEQ ID NO: 251, respectively; or
(iv) heavy chain CDR1, CDR2, and CDR3 sequences according to SEQ ID NO: 257, SEQ ID NO: 258, and SEQ ID NO: 259, respectively, and light chain CDR1, CDR2, and CDR3 sequences according to SEQ ID NO: 260, SEQ ID NO: 261 (or 262), and SEQ ID NO: 263, respectively.

Accordingly, the antibody or the antigen-binding fragment thereof preferably comprises:
a heavy chain CDR1 sequence according to SEQ ID NO: 235;
a heavy chain CDR2 sequence according to SEQ ID NO: 236;
a heavy chain CDR3 sequence according to SEQ ID NO: 237;
a light chain CDR1 sequence according to SEQ ID NO: 238;
a light chain CDR2 sequence according to SEQ ID NO: 239 (or 240); and
a light chain CDR3 sequence according to SEQ ID NO: 241.

In some embodiments, the antibody or the antigen-binding fragment thereof preferably comprises:
a heavy chain CDR1 sequence according to SEQ ID NO: 246;
a heavy chain CDR2 sequence according to SEQ ID NO: 247;
a heavy chain CDR3 sequence according to SEQ ID NO: 248;
a light chain CDR1 sequence according to SEQ ID NO: 249;
a light chain CDR2 sequence according to SEQ ID NO: 197 (or 250); and
a light chain CDR3 sequence according to SEQ ID NO: 251.

In some embodiments, the antibody or the antigen-binding fragment thereof preferably comprises:
a heavy chain CDR1 sequence according to SEQ ID NO: 246;
a heavy chain CDR2 sequence according to SEQ ID NO: 247;
a heavy chain CDR3 sequence according to SEQ ID NO: 248;
a light chain CDR1 sequence according to SEQ ID NO: 249;
a light chain CDR2 sequence according to SEQ ID NO: 197 (or 254); and
a light chain CDR3 sequence according to SEQ ID NO: 251.

In some embodiments, the antibody or the antigen-binding fragment thereof preferably comprises:
a heavy chain CDR1 sequence according to SEQ ID NO: 257;
a heavy chain CDR2 sequence according to SEQ ID NO: 258;
a heavy chain CDR3 sequence according to SEQ ID NO: 259;
a light chain CDR1 sequence according to SEQ ID NO: 260;
a light chain CDR2 sequence according to SEQ ID NO: 261 (or 262); and
a light chain CDR3 sequence according to SEQ ID NO: 263.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 242 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 243. Thereby, the CDR sequences as defined above (heavy chain CDR1, CDR2, and CDR3 sequences as set forth in SEQ ID NO: 235, SEQ ID NO: 236, and SEQ ID NO: 237, respectively; and light chain CDR1, CDR2, and CDR3 sequences as set forth in SEQ ID NO: 238, SEQ ID NO: 239 (or 240), and SEQ ID NO: 241, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 244 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 245. Thereby, the CDR sequences as defined above (heavy chain CDR1, CDR2, and CDR3 sequences as set forth in SEQ ID NO: 235, SEQ ID NO: 236, and SEQ ID NO: 237, respectively; and light chain CDR1, CDR2, and CDR3 sequences as set forth in SEQ ID NO: 238, SEQ ID NO: 239 (or 240), and SEQ ID NO: 241, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 252 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 253. Thereby, the CDR sequences as defined above (heavy chain CDR1, CDR2, and CDR3 sequences as set forth in SEQ ID NO: 246, SEQ ID NO: 247, and SEQ ID NO: 248, respectively; and light chain CDR1, CDR2, and CDR3 sequences as set forth in SEQ ID NO: 249, SEQ ID NO: 197 (or 250), and SEQ ID NO: 251, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 255 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 256. Thereby, the CDR sequences as defined above (heavy chain CDR1, CDR2, and CDR3 sequences as set forth in SEQ ID NO: 246, SEQ ID NO: 247, and SEQ ID NO: 248, respectively; and light chain CDR1, CDR2, and CDR3 sequences as set forth in SEQ ID NO: 249, SEQ ID NO: 197 (or 254), and SEQ ID NO: 251, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 264 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 265. Thereby, the CDR sequences as defined above (heavy chain CDR1, CDR2, and CDR3 sequences as set forth in SEQ ID NO: 257, SEQ ID NO: 258, and SEQ ID NO: 259, respectively; and light chain CDR1, CDR2, and CDR3 sequences as set forth in SEQ ID NO: 260, SEQ ID NO: 261 (or 262), and SEQ ID NO: 263, respectively) are preferably maintained.

In some embodiments, the antibody of the invention, or the antigen-binding fragment thereof, comprises (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 266 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 265. Thereby, the CDR sequences as defined above (heavy chain CDR1, CDR2, and CDR3 sequences as set forth in SEQ ID NO: 257, SEQ ID NO: 258, and SEQ ID NO: 259, respectively; and light chain CDR1, CDR2, and CDR3 sequences as set forth in SEQ ID NO: 260, SEQ ID NO: 261 (or 262), and SEQ ID NO: 263, respectively) are preferably maintained.

More preferably, the antibody, or an antigen-binding fragment thereof, preferably comprises a heavy chain variable region comprising or consisting of an amino acid sequence as set forth in SEQ ID NO: 242 and a light chain variable region comprising or consisting of an amino acid sequence as set forth in SEQ ID NO: 243.

In some embodiments, the antibody, or an antigen-binding fragment thereof, more preferably comprises a heavy chain variable region comprising or consisting of an amino acid sequence as set forth in SEQ ID NO: 244 and a light chain variable region comprising or consisting of an amino acid sequence as set forth in SEQ ID NO: 245.

In some embodiments, the antibody, or an antigen-binding fragment thereof, more preferably comprises a heavy chain variable region comprising or consisting of an amino acid sequence as set forth in SEQ ID NO: 252 and a light chain variable region comprising or consisting of an amino acid sequence as set forth in SEQ ID NO: 253.

In some embodiments, the antibody, or an antigen-binding fragment thereof, more preferably comprises a heavy chain variable region comprising or consisting of an amino acid sequence as set forth in SEQ ID NO: 255 and a light chain variable region comprising or consisting of an amino acid sequence as set forth in SEQ ID NO: 256.

In some embodiments, the antibody, or an antigen-binding fragment thereof, more preferably comprises a heavy chain variable region comprising or consisting of an amino acid sequence as set forth in SEQ ID NO: 264 and a light chain variable region comprising or consisting of an amino acid sequence as set forth in SEQ ID NO: 265.

In some embodiments, the antibody, or an antigen-binding fragment thereof, more preferably comprises a heavy chain variable region comprising or consisting of an amino acid sequence as set forth in SEQ ID NO: 266 and a light chain variable region comprising or consisting of an amino acid sequence as set forth in SEQ ID NO: 265.

The CDR and VH/VL sequences of exemplified antibodies of the invention binding to CXCL16 are shown in Table 6 below.

TABLE 6
CDR and VH/VL sequences (SEQ ID NOs) of exemplified
antibodies of the invention binding to CXCL16.

| Antibody | CDRH1 | CDRH2 | CDRH3 | CDRL1 | CDRL2 | CDRL3 | VH | VL |
|---|---|---|---|---|---|---|---|---|
| aCXCL16.001 | 235 | 236 | 237 | 238 | 239/240 | 241 | 242 | 243 |
| aCXCL16.001v1 | 235 | 236 | 237 | 238 | 239/240 | 241 | 244 | 245 |
| aCXCL16.002 | 246 | 247 | 248 | 249 | 197/250 | 251 | 252 | 253 |
| aCXCL16.002v1 | 246 | 247 | 248 | 249 | 197/254 | 251 | 255 | 256 |
| aCXCL16.003 | 257 | 258 | 259 | 260 | 261/262 | 263 | 264 | 265 |
| aCXCL16.003v1 | 257 | 258 | 259 | 260 | 261/262 | 263 | 266 | 265 |

Preferably, the antibody of the invention comprises the combination of six CDR sequences (optionally the VH and VL sequences) of the exemplified antibodies shown in Table 6, or respective sequence variants thereof, as defined herein above.

In some embodiments, the antibody, or the antigen-binding fragment thereof, binds to the N-loop of CXCL16.

In some embodiments, the antibody, or the antigen-binding fragment thereof, reduces or inhibits cell migration towards CXCL16.

As demonstrated in the examples, exemplified antibodies of the invention, which bind to CXCL16, exhibit such features. To assess any one of those features, methods as described for this purpose in the present examples may be used.

### Further Features of the Antibodies of the Invention

The above-described antibodies according to the present invention bind to different human chemokines, as indicated above, in particular to the N-loop of said chemokines. Thereby, the antibodies of the invention may reduce or inhibit migration of immune cells, as demonstrated in the Examples of the present application. In particular, the antibodies may reduce or inhibit cell migration towards the specific chemokine, to which they bind.

In some embodiments, the antibody of the invention is a human antibody. In some embodiments, the antibody of the invention is a monoclonal antibody. For example, the antibody of the invention may be a human monoclonal antibody.

In some embodiments, the antibody according to the present invention, or an antigen binding fragment thereof, comprises an Fc moiety. The Fc moiety may be derived from human origin, e.g. from human IgG1, IgG2, IgG3, and/or IgG4, such as human IgG1.

As used herein, the term “Fc moiety” refers to a sequence derived from the portion of an immunoglobulin heavy chain beginning in the hinge region just upstream of the papain cleavage site (e.g., residue 216 in native IgG, taking the first residue of heavy chain constant region to be 114) and ending at the C-terminus of the immunoglobulin heavy chain. Accordingly, an Fc moiety may be a complete Fc moiety or a portion (e.g., a domain) thereof. A complete Fc moiety comprises at least a hinge domain, a CH2 domain, and a CH3 domain (e.g., EU amino acid positions 216-446). An additional lysine residue (K) is sometimes present at the extreme C-terminus of the Fc moiety, but is often cleaved from a mature antibody.

Each of the amino acid positions within an Fc moiety have been numbered herein according to the art-recognized EU numbering system of Kabat, see e.g., by Kabat et al., in “Sequences of Proteins of Immunological Interest”, U.S. Dept. Health and Human Services, 1983 and 1987. The EU index or EU index as in Kabat or EU numbering refers to the numbering of the EU antibody (Edelman G M, Cunningham B A, Gall W E, Gottlieb P D, Rutishauser U, Waxdal M J. The covalent structure of an entire gammaG immunoglobulin molecule. Proc Natl Acad Sci USA. 1969; 63(1):78-85; Kabat E. A., National Institutes of Health (U.S.) Office of the Director, “Sequences of Proteins of Immunological Interest”, 5^{th} edition, Bethesda, MD: U.S. Dept. of Health and Human Services, Public Health Service, National Institutes of Health, 1991, hereby entirely incorporated by reference).

In some embodiments, in the context of the present invention an Fc moiety comprises at least one of: a hinge (e.g., upper, middle, and/or lower hinge region) domain, a CH2 domain, a CH3 domain, or a variant, portion, or fragment thereof. An Fc moiety may comprise at least a hinge domain, a CH2 domain or a CH3 domain. The Fc moiety may be a complete Fc moiety. The Fc moiety may also comprises one or more amino acid insertions, deletions, or substitutions relative to a naturally-occurring Fc moiety. For example, at least one of a hinge domain, CH2 domain or CH3 domain (or portion thereof) may be deleted. For example, an Fc moiety may comprise or consist of: (i) hinge domain (or portion thereof) fused to a CH2 domain (or portion thereof), (ii) a hinge domain (or portion thereof) fused to a CH3 domain (or portion thereof), (iii) a CH2 domain (or portion thereof) fused to a CH3 domain (or portion thereof), (iv) a hinge domain (or portion thereof), (v) a CH2 domain (or portion thereof), or (vi) a CH3 domain or portion thereof.

It will be understood by one of ordinary skill in the art that the Fc moiety may be modified such that it varies in amino acid sequence from the complete Fc moiety of a naturally occurring immunoglobulin molecule, while retaining at least one desirable function conferred by the naturally-occurring Fc moiety. Such functions include Fc receptor (FcR) binding, antibody half-life modulation, ADCC function, protein A binding, protein G binding, and complement binding. The portions of naturally occurring Fc moieties, which are responsible and/or essential for such functions are well known by those skilled in the art. In some embodiments, the antibody according to the present invention comprises a (complete) Fc moiety/Fc region, wherein the interaction/binding with FcR is not compromised.

In general, binding of the antibody to an Fc receptor may be assessed by various methods known to the skilled person, such as ELISA (Hessell A J, Hangartner L, Hunter M, Havenith C E G, Beurskens F J, Bakker J M, Lanigan C M S, Landucci G, Forthal D N, Parren PWHI, et al.: Fc receptor but not complement binding is important in antibody protection against HIV. Nature 2007, 449:101-104; Grevys A, Bern M, Foss S, Bratlie D B, Moen A, Gunnarsen K S, Aase A, Michaelsen T E, Sandlie I, Andersen J T: Fc Engineering of Human IgG1 for Altered Binding to the Neonatal Fc Receptor Affects Fc Effector Functions. 2015, 194:5497-5508) or flow-cytometry (Perez L G, Costa M R, Todd C A, Haynes B F, Montefiori D C: Utilization of immunoglobulin G Fc receptors by human immunodeficiency virus type 1: a specific role for antibodies against the membrane-proximal external region of gp41. J Virol 2009, 83:7397-7410; Piccoli L, Campo I, Fregni C S, Rodriguez B M F, Minola A, Sallusto F, Luisetti M, Corti D, Lanzavecchia A: Neutralization and clearance of GM-CSF by autoantibodies in pulmonary alveolar proteinosis. Nat Commun 2015, 6:1-9).

In some embodiments, the antibody, or antigen binding fragment thereof, according to the present invention comprises an Fc region. As used herein, the term “Fc region” refers to the portion of an immunoglobulin formed by two or more Fc moieties of antibody heavy chains. For example, the Fc region may be monomeric or “single-chain” Fc region (i.e., a scFc region). Single chain Fc regions are comprised of Fc moieties linked within a single polypeptide chain (e.g., encoded in a single contiguous nucleic acid sequence). Exemplary scFc regions are disclosed in WO 2008/143954 A2. The Fc region may be dimeric. A “dimeric Fc region” or “dcFc” refers to the dimer formed by the Fc moieties of two separate immunoglobulin heavy chains. The dimeric Fc region may be a homodimer of two identical Fc moieties (e.g., an Fc region of a naturally occurring immunoglobulin) or a heterodimer of two non-identical Fc moieties.

In some embodiments, the Fc moiety, or the Fc region, comprises or consists of an amino acid sequence derived from a human immunoglobulin sequence (e.g., from an Fc region or Fc moiety from a human IgG molecule). However, the Fc moiety, or the Fc region, may comprise one or more amino acids from another mammalian species. For example, a primate Fc moiety or a primate binding site may be included in the antibody, or antigen-binding fragment. Alternatively, one or more murine amino acids may be present in the Fc moiety or in the Fc region.

The Fc moieties of the Fc region may be of the same or different class and/or subclass. For example, the Fc moieties may be derived from an immunoglobulin (e.g., a human immunoglobulin) of an IgG1, IgG2, IgG3 or IgG4 subclass.

Accordingly, antibodies of the invention can be of any isotype (e.g., IgA, IgG, IgM i.e. an a, γ or μ heavy chain). Preferably, the antibody may be of the IgG type. Within the IgG isotype, antibodies may be IgG1, IgG2, IgG3 or IgG4 subclass, for example IgG1. Antibodies of the invention may have a κ or λ light chain. In some embodiments, the antibody is of IgG1 type and has a lambda or kappa light chain.

In some embodiments, the antibody is of the human IgG1 type. The antibody may be of any allotype. The term “allotype” refers to the allelic variation found among the IgG subclasses. For example, the antibody may be of the G1 m1 (or G1 m(a)) allotype, of the G1 m2 (or G1 m(x)) allotype, of the G1 m3 (or G1 m(f)) allotype, and/or of the G1 m17 (or Gm(z)) allotype. The G1 m3 and G1 m17 allotypes are located at the same position in the CH1 domain (position 214 according to EU numbering). G1m3 corresponds to R214 (EU), while G1m17 corresponds to K214 (EU). The G1 m1 allotype is located in the CH3 domain (at positions 356 and 358 (EU)) and refers to the replacements E356D and M358L. The G1m2 allotype refers to a replacement of the alanine in position 431 (EU) by a glycine. The G1 m1 allotype may be combined, for example, with the G1 m3 or the G1 m17 allotype. In some embodiments, the antibody is of the allotype G1m3 with no G1 m1 (G1m3,-1). In some embodiments, the antibody is of the G1m17,1 allotype. In some embodiments, the antibody is of the G1m3,1 allotype. In some embodiments, the antibody is of the allotype G1m17 with no G1 m1 (G1 m17,-1). Optionally, these allotypes may be combined (or not combined) with the G1 m2, G1 m27 or G1 m28 allotype. For example, the antibody may be of the G1 m17,1,2 allotype.

In general, the antibody, or antigen-binding fragment, according to the present invention may be glycosylated. N-linked glycans attached to the CH2 domain of a heavy chain, for instance, can influence C1q and FcR binding, with glycosylated antibodies having lower affinity for these receptors. Accordingly, the CH2 domain of the Fc moiety of the antibody according to the present invention may comprise one or more mutations, in which a glycosylated residue is substituted by a non-glycosylated residue. For example, the antibody's glycans do not lead to a human immunogenic response after administration.

As outlined above, the present invention encompasses antigen-binding fragments. An antigen-binding fragment may or may not comprise an Fc moiety, in particular a portion of a complete Fc region. In some embodiments, the antibody, or antigen-binding fragment thereof, is selected from Fab, Fab′, F(ab′)2, Fv or scFv. For example, F(ab′)2 (which may be obtained by pepsin cleavage or recombinant expression) as well as Fab′ (which can be obtained from F(ab′)2 or by recombinant expression) usually includes the hinge region.

In some embodiments, the antibody, or antigen-binding fragment, may be a single-chain antibody (or fragment). The single-chain antibody (or fragment) may encode the complete set of six CDRs, i.e. include the three heavy chain CDRs as well as the three light chain CDRs. More specifically, the single-chain antibody (or fragment) may include a heavy chain variable region (VH) as well as a light chain variable region (VL), for example including the VH and VL sequences as described above.

Antibodies of the invention also include hybrid antibody molecules that comprise the six CDRs from an antibody of the invention as defined above and one or more CDRs from another antibody to an antigen. For example, the antibody may be bispecific.

Variant antibodies are also included within the scope of the invention. Thus, variants of the sequences recited in the application are also included within the scope of the invention. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Antibodies of the invention, or antigen-binding fragments thereof, may be provided in purified form. Typically, the antibody, or antigen-binding fragment, will be present in a composition that is substantially free of other polypeptides e.g., where less than 90% (by weight), usually less than 60% and more usually less than 50% of the composition is made up of other polypeptides.

Antibodies of the invention may be immunogenic in non-human (or heterologous) hosts e.g., in mice. In particular, the antibodies may have an idiotope that is immunogenic in non-human hosts, but not in a human host. In particular, antibodies of the invention for human use include those that cannot be easily isolated from hosts such as mice, goats, rabbits, rats, non-primate mammals, etc. and cannot generally be obtained by humanization or from xeno-mice.

### Nucleic Acids

In another aspect, the invention also provides a nucleic acid molecule comprising a polynucleotide encoding the antibody according to the present invention, or an antigen-binding fragment thereof, as described above.

A nucleic acid molecule is a molecule comprising nucleic acid components. The term nucleic acid molecule usually refers to DNA or RNA molecules. It may be used synonymous with the term “polynucleotide”, i.e. the nucleic acid molecule may consist of a polynucleotide encoding the antibody. Alternatively, the nucleic acid molecule may also comprise further elements in addition to the polynucleotide encoding the antibody. Typically, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules.

Examples of nucleic acid molecules and/or polynucleotides include, e.g., a recombinant polynucleotide, a vector, an oligonucleotide, an RNA molecule such as an rRNA, an mRNA, an miRNA, an siRNA, or a tRNA, or a DNA molecule such as a cDNA. Nucleic acids may encode the light chain and/or the heavy chain of an antibody. In other words, the light chain and the heavy chain of the antibody may be encoded by the same nucleic acid molecule (e.g., in bicistronic manner). Alternatively, the light chain and the heavy chain of the antibody may be encoded by distinct nucleic acid molecules.

In some embodiments, the nucleic acid molecule comprises one or more polynucleotide(s) encoding the exemplified antibodies of the invention (e.g., as described in Tables 2-6 above), or a sequence variant thereof as described herein (e.g., having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity as described above).

Due to the redundancy of the genetic code, the present invention also comprises sequence variants of nucleic acid sequences, which encode the same amino acid sequences. The polynucleotide encoding the antibody (or the complete nucleic acid molecule) may be optimized for expression of the antibody. For example, codon optimization of the nucleotide sequence may be used to improve the efficiency of translation in expression systems for the production of the antibody. Moreover, the nucleic acid molecule may comprise heterologous elements (i.e., elements, which in nature do not occur on the same nucleic acid molecule as the coding sequence for the (heavy or light chain of) an antibody. For example, a nucleic acid molecule may comprise a heterologous promotor, a heterologous enhancer, a heterologous UTR (e.g., for optimal translation/expression), a heterologous Poly-A-tail, and the like.

In general, the nucleic acid molecule may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the antibody's amino acid sequence. For example, such point mutations can introduce amino acids for the attachment of covalent groups (e.g., labels) or can introduce tags (e.g., for purification purposes). In some embodiments, a mutation in a nucleic acid sequence may be “silent”, i.e. not reflected in the amino acid sequence due to the redundancy of the genetic code. In general, mutations can be introduced in specific sites or can be introduced at random, followed by selection (e.g., molecular evolution). For instance, one or more nucleic acids encoding any of the light or heavy chains of an (exemplary) antibody can be randomly or directionally mutated to introduce different properties in the encoded amino acids. Such changes can be the result of an iterative process wherein initial changes are retained and new changes at other nucleotide positions are introduced. Further, changes achieved in independent steps may be combined.

In some embodiments, the polynucleotide encoding the antibody, or an antigen-binding fragment thereof, (or the (complete) nucleic acid molecule) may be codon-optimized. The skilled artisan is aware of various tools for codon optimization, such as those described in: Ju Xin Chin, Bevan Kai-Sheng Chung, Dong-Yup Lee, Codon Optimization OnLine (COOL): a web-based multi-objective optimization platform for synthetic gene design, Bioinformatics, Volume 30, Issue 15, 1 Aug. 2014, Pages 2210-2212; or in: Grote A, Hiller K, Scheer M, Munch R, Nortemann B, Hempel D C, Jahn D, JCat: a novel tool to adapt codon usage of a target gene to its potential expression host. Nucleic Acids Res. 2005 Jul. 1; 33 (Web Server issue):W526-31; or, for example, Genscript's OptimumGene™ algorithm (as described in US 2011/0081708 A1).

The present invention also provides a combination of a first and a second nucleic acid molecule, wherein the first nucleic acid molecule comprises a polynucleotide encoding the heavy chain of the antibody, or an antigen-binding fragment thereof, of the present invention; and the second nucleic acid molecule comprises a polynucleotide encoding the corresponding light chain of the same antibody, or the same antigen-binding fragment thereof. The above description regarding the (general) features of the nucleic acid molecule of the invention applies accordingly to the first and second nucleic acid molecule of the combination. Accordingly, one or both of the polynucleotides encoding the heavy and/or light chain(s) of the antibody, or an antigen-binding fragment thereof, may be codon-optimized.

### Vector

Further included within the scope of the invention are vectors, for example, expression vectors, comprising a nucleic acid molecule according to the present invention. Usually, a vector comprises a nucleic acid molecule as described above.

The present invention also provides a combination of a first and a second vector, wherein the first vector comprises a first nucleic acid molecule as described above (for the combination of nucleic acid molecules) and the second vector comprises a second nucleic acid molecule as described above (for the combination of nucleic acid molecules).

A vector is usually a recombinant nucleic acid molecule, i.e. a nucleic acid molecule which does not occur in nature. Accordingly, the vector may comprise heterologous elements (i.e., sequence elements of different origin in nature). For example, the vector may comprise a multi cloning site, a heterologous promotor, a heterologous enhancer, a heterologous selection marker (to identify cells comprising said vector in comparison to cells not comprising said vector) and the like. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a (heavy and/or light chain of a) desired antibody according to the present invention. An expression vector may be used for production of expression products such as RNA, e.g. mRNA, or peptides, polypeptides or proteins. For example, an expression vector may comprise sequences needed for transcription of a sequence stretch of the vector, such as a (heterologous) promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. For example, a vector in the sense of the present application comprises a cloning site, a selection marker, such as an antibiotic resistance factor, and a sequence suitable for multiplication of the vector, such as an origin of replication. A vector in the context of the present application may be a plasmid vector.

### Cells

In a further aspect, the present invention also provides a cell expressing the antibody according to the present invention, or an antigen-binding fragment thereof; and/or comprising the vector (or the combination of vectors) according the present invention.

Examples of such cells include but are not limited to, eukaryotic cells, e.g., yeast cells, animal cells or plant cells. Other examples of such cells include but are not limited, to prokaryotic cells, e.g. E. coli. In some embodiments, the cells are mammalian cells, such as a mammalian cell line. Examples include human cells, CHO cells, HEK293T cells, PER. C6 cells, NS0 cells, human liver cells, myeloma cells or hybridoma cells.

The cell may be transfected with a vector according to the present invention, for example with an expression vector. The term “transfection” refers to the introduction of nucleic acid molecules, such as DNA or RNA (e.g. mRNA) molecules, into cells, e.g. into eukaryotic or prokaryotic cells. In the context of the present invention, the term “transfection” encompasses any method known to the skilled person for introducing nucleic acid molecules into cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In some embodiments, the introduction is non-viral.

Moreover, the cells of the present invention may be transfected stably or transiently with the vector according to the present invention, e.g. for expressing the antibody according to the present invention. In some embodiments, the cells are stably transfected with the vector according to the present invention encoding the antibody according to the present invention. In other embodiments, the cells are transiently transfected with the vector according to the present invention encoding the antibody according to the present invention.

Accordingly, the present invention also provides a recombinant host cell, which heterologously expresses the antibody of the invention or the antigen-binding fragment thereof. For example, the cell may be of another species than the antibody (e.g., CHO cells expressing human antibodies). In some embodiments, the cell type of the cell does not express (such) antibodies in nature. Moreover, the host cell may impart a post-translational modification (PTM; e.g., glycosylation) on the antibody that is not present in their native state. Such a PTM may result in a functional difference (e.g., reduced immunogenicity). Accordingly, the antibody of the invention, or the antigen-binding fragment thereof, may have a post-translational modification, which is distinct from the naturally produced antibody (e.g., an antibody of an immune response in a human).

### Production of Antibodies

Antibodies according to the invention can be made by any method known in the art. For example, the general methodology for making monoclonal antibodies using hybridoma technology is well known (Kohler, G. and Milstein, C., 1975; Kozbar et al. 1983).

Standard techniques of molecular biology may be used to prepare DNA sequences encoding the antibodies or antigen-binding fragments of the present invention. Desired DNA sequences may be synthesized completely or in part, e.g., using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present invention. Eukaryotic, e.g., mammalian, host cell expression systems may be used for production of antibody molecules, such as complete antibody molecules. Suitable mammalian host cells include, but are not limited to, CHO, HEK293, PER. C6, NS0, myeloma or hybridoma cells. Also, prokaryotic, e.g. bacterial host cell expression systems may be used for the production of antibody molecules, such as complete antibody molecules. Suitable bacterial host cells include, but are not limited to, E. coli cells.

Accordingly, the present invention provides a method for preparing the antibody, or an antigen-binding fragment or an immunoglobulin chain(s) thereof, according to the present invention, said method comprising
(i) culturing the host cell as described above; and
(ii) isolating the antibody or immunoglobulin chain(s) thereof from the culture.

In other words, the present invention also provides a process for the production of an antibody molecule according to the present invention comprising culturing a (heterologous) host cell comprising a vector encoding a nucleic acid of the present invention, in particular under conditions suitable for expression of protein from DNA encoding the antibody molecule of the present invention, and isolating the antibody molecule.

For production of the antibody comprising both heavy and light chains, a host cell, such as a cell line, may be transfected with two vectors, a first vector encoding a light chain polypeptide and a second vector encoding a heavy chain polypeptide, e.g. as described above. Alternatively, a single vector may be used, the vector including sequences encoding light chain and heavy chain polypeptides (e.g. for single chain antibodies or in a bicistronic manner).

Thus, the invention also provides a method for preparing a recombinant cell, comprising the steps of: (i) providing one or more nucleic acids that encode(s) the antibody of the invention; (ii) inserting the nucleic acid into an expression vector and (iii) transfecting the vector into a (heterologous) host cell in order to permit expression of the antibody of interest in that host cell. The nucleic acid of step (i) may, but need not, be manipulated to introduce restriction sites, to change codon usage, and/or to optimize transcription and/or translation regulatory sequences.

Furthermore, the invention also provides a method of preparing a transfected host cell, comprising the step of transfecting a host cell with one or more nucleic acids that encode an antibody of interest. Thus the procedures for first preparing the nucleic acid(s) and then using it to transfect a host cell can be performed at different times by different people in different places (e.g., in different countries).

These recombinant cells of the invention can then be used for expression and culture purposes. They are particularly useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

The transfected host cell may be a eukaryotic cell, including yeast and animal cells, particularly mammalian cells (e.g., CHO cells, NS0 cells, human cells such as PER. C6, HEK293 or HKB-11 cells, myeloma cells, or a human liver cell), as well as plant cells. In some embodiments, the transfected host cell is a mammalian cell, such as a human cell. In some embodiments, expression hosts can glycosylate the antibody of the invention, particularly with carbohydrate structures that are not themselves immunogenic in humans. In some embodiments the transfected host cell may be able to grow in serum-free media. In further embodiments the transfected host cell may be able to grow in culture without the presence of animal-derived products. The transfected host cell may also be cultured to give a cell line.

The invention also provides a method of preparing the antibody of interest comprising the steps of: culturing or sub-culturing a transfected host cell population, e.g. a stably transfected host cell population, under conditions where the antibody of interest is expressed and, optionally, purifying the antibody of interest. The transfected host cell population may be prepared by (i) providing nucleic acid(s) encoding a selected antibody of interest, (ii) inserting the nucleic acid(s) into an expression vector, (iii) transfecting the vector in a host cell that can express the antibody of interest, and (iv) culturing or sub-culturing the transfected host cell comprising the inserted nucleic acids to produce the antibody of interest.

In some embodiments, antibodies according to the invention may be produced by (i) expressing a nucleic acid sequence according to the invention in a host cell, e.g. by use of a vector (or host cell) according to the present invention, and (ii) isolating the expressed antibody product. Additionally, the method may include (iii) purifying the isolated antibody.

Accordingly, after production, the antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

### Compositions

The present invention also provides a composition comprising one or more of:
(i) the antibody of the present invention, or an antigen-binding fragment thereof;
(ii) the nucleic acid or the combination of nucleic acids of the present invention;
(iii) the vector or the combination of vectors of the present invention; and/or
(iv) the cell expressing the antibody according to the present invention or comprising the vector according to the present invention.

In some embodiments, the composition is a pharmaceutical composition, which may further comprise a pharmaceutically acceptable excipient, diluent or carrier. In other words, the present invention also provides a pharmaceutical composition comprising the antibody according to the present invention, the nucleic acid according to the present invention, the vector according to the present invention and/or the cell according to the present invention.

The pharmaceutical composition may optionally also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. A thorough discussion of pharmaceutically acceptable carriers is available in Gennaro (2000) Remington: The Science and Practice of Pharmacy, 20th edition, ISBN: 0683306472.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutically acceptable salts can be used, for example mineral acid salts, such as hydrochlorides, hydrobromides, phosphates and sulphates, or salts of organic acids, such as acetates, propionates, malonates and benzoates.

A vehicle is typically understood to be a material that is suitable for storing, transporting, and/or administering a compound, such as a pharmaceutically active compound, in particular the antibodies according to the present invention. For example, the vehicle may be a physiologically acceptable liquid, which is suitable for storing, transporting, and/or administering a pharmaceutically active compound, in particular the antibodies according to the present invention. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Once formulated, the compositions of the invention can be administered directly to the subject. In some embodiments the compositions are adapted for administration to mammalian, e.g., human subjects.

Pharmaceutical compositions may include an antimicrobial, particularly if packaged in a multiple dose format. They may comprise detergent e.g., a Tween (polysorbate), such as Tween 80. Detergents are generally present at low levels e.g., less than 0.01%. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

In some embodiments, the pharmaceutically acceptable carrier, vehicle, diluent and/or excipient in the pharmaceutical composition according to the present invention is not an active component in respect to chemokines, in particular in respect to the chemokine to which the antibody comprised in the composition binds to. In some embodiments, the (only) active ingredient in the composition is the antibody according to the present invention. As such, it may be susceptible to degradation in the gastrointestinal tract. Thus, if the composition is to be administered by a route using the gastrointestinal tract, the composition may contain agents which protect the antibody from degradation but which release the antibody once it has been absorbed from the gastrointestinal tract.

Pharmaceutical compositions of the invention may generally have a pH between 5.5 and 8.5, in some embodiments this may be between 6 and 8, for example about 7. The pH may be maintained by the use of a buffer. The composition may be sterile and/or pyrogen free. The composition may be isotonic with respect to humans. In some embodiments pharmaceutical compositions of the invention are supplied in hermetically-sealed containers.

Whether it is an antibody, a peptide, a nucleic acid molecule, or another pharmaceutically useful compound according to the present invention that is to be given to an individual, administration is usually in an “effective amount”, e.g. in a “prophylactically effective amount” or a “therapeutically effective amount” (as the case may be), this being sufficient to show benefit to the individual. The actual amount administered, and rate and time-course of administration, may depend upon the subject's size, weight, and health, the nature and extent of the condition, and the therapeutics or combination of therapeutics selected for administration. The effective amount for a given situation is usually determined by routine experimentation and is within the judgment of a clinician. For purposes of the present invention, an effective dose may generally be from about 0.005 to about 100 mg/kg, for example from about 0.0075 to about 50 mg/kg or from about 0.01 to about 10 mg/kg. In some embodiments, the effective dose will be from about 0.02 to about 5 mg/kg, of the antibody of the present invention (e.g. amount of the antibody in the pharmaceutical composition) in relation to the bodyweight (e.g., in kg) of the individual to which it is administered.

Moreover, the pharmaceutical composition according to the present invention may also comprise an additional active component, which may be a further antibody or a component, which is not an antibody. Accordingly, the pharmaceutical composition according to the present invention may comprise one or more of the additional active components. In some embodiments, the pharmaceutical composition comprises two distinct antibodies or antigen-binding fragments, in particular two distinct antibodies or antigen-binding fragments (specifically) binding to a chemokine. In some embodiments, the each of the two distinct antibodies, or antigen-binding fragments thereof, is an antibody according to the present invention as described above.

The antibody according to the present invention can be present either in the same pharmaceutical composition as the additional active component (e.g., a second antibody as described above) or, alternatively, the antibody according to the present invention is comprised in a first pharmaceutical composition and the additional active component (e.g., a second antibody as described above) is comprised in a second pharmaceutical composition different from the first pharmaceutical composition. Accordingly, if more than one additional active component is envisaged, each additional active component (e.g., a second antibody as described above) and the antibody according to the present invention may be comprised in a different pharmaceutical composition. Such different pharmaceutical compositions may be administered either combined/simultaneously or at separate times or at separate locations (e.g. separate parts of the body).

Accordingly, the present invention also provides a combination of two (or more, e.g., 3, 4, 5, 6, 7, 8, 9, or 10) distinct antibodies, or antigen-binding fragments thereof, wherein each of the two (or more, e.g., 3, 4, 5, 6, 7, 8, 9, or 10) distinct antibodies, or antigen-binding fragments thereof, is an antibody according to the present invention as described above, preferably for use in medicine as described in more detail below.

Furthermore, the present invention also provides a kit of parts comprising two (or more, e.g., 3, 4, 5, 6, 7, 8, 9, or 10) distinct antibodies, or antigen-binding fragments thereof, wherein each of the two (or more, e.g., 3, 4, 5, 6, 7, 8, 9, or 10) distinct antibodies, or antigen-binding fragments thereof, is an antibody according to the present invention as described above. In the kit of parts, the two distinct antibodies may be provided in distinct vessels (e.g., in distinct pharmaceutical compositions).

The antibody according to the present invention and the additional active component (e.g., a second antibody as described above) may provide an additive therapeutic effect, such as a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

In some embodiments, a composition of the invention may include antibodies of the invention, wherein the antibodies may make up at least 50% by weight (e.g., 60%, 70%, 75%, 80%, 85%, 90%, 95%, 96%, 97%, 98%, 99% or more) of the total protein in the composition. In the composition of the invention, the antibodies may be in purified form.

The present invention also provides a method of preparing a pharmaceutical composition comprising the steps of: (i) preparing an antibody of the invention; and (ii) admixing the purified antibody with one or more pharmaceutically acceptable excipients, diluents or carriers.

### Medical Treatments Using the Antibodies of the Invention

In a further aspect, the present invention provides the use of the antibody according to the present invention, or an antigen-binding fragment thereof, the nucleic acid molecule (or the combination of nucleic acid molecules) according to the present invention, the vector (or the combination of vectors) according to the present invention, the cell according to the present invention, the pharmaceutical composition according to the present invention, the combination according to the present invention or the kit of parts according to the present invention as a medicament.

In particular, the antibody according to the present invention binding to CXCL13 or CXCL16, or an antigen-binding fragment thereof, the nucleic acid molecule (or the combination of nucleic acid molecules) according to the present invention encoding said antibody, the respective vector (or the combination of vectors) according to the present invention, the respective cell according to the present invention, the respective pharmaceutical composition according to the present invention, the combination according to the present invention or the kit of parts according to the present invention may be used in prophylaxis and/or treatment of Long COVID.

Accordingly, the present invention also provides a method of ameliorating or reducing the symptoms of Long COVID, or lowering the risk of developing Long COVID, comprising: administering to a subject in need thereof, a therapeutically effective amount of the antibody, or an antigen-binding fragment thereof, according to the present invention, the nucleic acid molecule (or the combination of nucleic acid molecules) according to the present invention, the vector (or the combination of vectors) according to the present invention, the cell according to the present invention or the pharmaceutical composition according to the present invention. Moreover, the present invention also provides the use of the antibody according to the present invention binding to CXCL13 or CXCL16, or an antigen-binding fragment thereof, the nucleic acid molecule (or the combination of nucleic acid molecules) according to the present invention, the vector (or the combination of vectors) according to the present invention, the cell according to the present invention or the pharmaceutical composition according to the present invention in the manufacture of a medicament for prophylaxis, treatment or attenuation of Long COVID.

In a further aspect, the present invention also provides a combination of antibodies binding to CCL21, CXCL13 and CXCL16, or antigen-binding fragments thereof, for use in the prophylaxis or treatment of long-term symptoms of COVID-19. Such a combination may include (i) an antibody binding to CXCL13 according to the invention as described above, or an antigen-binding fragment thereof; and/or an antibody binding to CXCL16 according to the invention as described above, or an antigen-binding fragment thereof.

In some embodiments the subject may be a human. One way of checking efficacy of therapeutic treatment involves monitoring disease symptoms after administration of the composition of the invention. Treatment can be a single dose schedule or a multiple dose schedule. In one embodiment, an antibody, antibody fragment, nucleic acid, vector, cell or composition according to the invention is administered to a subject in need of such treatment. In some embodiments, the subject is a mammal, preferably a human.

### Use of Anti-Chemokine Antibodies

In a further aspect, the present invention also provides a (in-vitro) method of evaluating the COVID-19 status in a subject, wherein the method comprises determining the level (amount or concentration) of antibodies binding to CCL19, CCL22 and CXCL17 in a (isolated) sample of said subject.

The present inventors have surprisingly found that the level of auto-antibodies binding to human chemokines CCL19, CCL22 and CXCL17 correlates with the COVID-19 status in a subject. As shown in the experimental data below, antibodies to the three chemokines CCL19, CCL22 and CXCL17 (“COVID-19 signature”) clustered together with p<10⁻⁻⁴, and by themselves were sufficient to correctly assign uninfected controls and COVID-19 convalescents with accuracies >95%. Thus, COVID-19 is associated with a specific pattern of anti-chemokine antibodies.

Accordingly, the COVID-19 status of a subject, in particular whether (i) the subject is or has been suffering from COVID-19 and/or (ii) the subject is or has been infected with SARS-CoV-2 can be determined based on the levels of auto-antibodies to human chemokines CCL19, CCL22 and CXCL17.

To this end, a sample, in particular a body fluid sample (such as blood), may be obtained (isolated) from said subject and the levels of antibodies to CCL19, CCL22 and CXCL17 may be determined in said sample. In some embodiments, the sample may be obtained from the subject about 1 day to 24 months after the onset of COVID-19 symptoms or infection with SARS-CoV-2, preferably about 1 week to 18 months after the onset of COVID-19 symptoms or infection with SARS-CoV-2, more preferably about 3 months to 18 months after the onset of COVID-19 symptoms or infection with SARS-CoV-2.

The level (amount or concentration) of antibodies in said sample may be determined by using the method of the invention for determining binding of an antibody to a chemokine as described above, the (N-loop chemokine) peptide according to the invention as described above, the composition containing said peptide the invention as described above or the kit the invention as described above. For example, the kit may contain peptides relating to each of the three human chemokines CCL19, CCL22 and CXCL17. Namely, a peptide as described above comprising the amino acid sequence of SEQ ID NO: 15 or 66 may be used to determine the level of antibodies binding to CCL19; a peptide as described above comprising the amino acid sequence of SEQ ID NO: 18 or 69 may be used to determine the level of antibodies binding to CCL22; and/or a peptide as described above comprising the amino acid sequence of SEQ ID NO: 40 or 91 may be used to determine the level of antibodies binding to CXCL17. Likewise the respective peptide(s) (or composition) may be used, e.g. in the method of the invention.

In some embodiments, the method of the invention for evaluating the COVID-19 status further comprises a step of comparing the level (amount or concentration) of said antibodies to the corresponding antibody levels in a reference group of subjects without COVID-19 or uninfected subjects. Namely, it may be determined whether or not the levels of antibodies binding to CCL19, CCL22 and CXCL17 are increased compared to subjects without COVID-19 or uninfected subjects. The increase of said antibody levels in a statistically significant manner, e.g. p<10⁻⁻⁴ or p<10⁻⁻³, indicates whether (i) the subject is or has been suffering from COVID-19 and/or (ii) whether the subject is or has been infected with SARS-CoV-2. The experimental data described herein below provide an exemplified data set regarding the levels of antibodies binding to CCL19, CCL22 and CXCL17 in uninfected subjects. If this data set is used as reference, the antibody levels in the test sample are preferably determined with the same method, i.e. as described herein below. Alternatively, for example, the levels of antibodies binding to CCL19, CCL22 and CXCL17 may be determined (i) for the test subject; and (ii) for a plurality (e.g., at least 5, such as 5, 6, 7, 8, 9, 10, 11, 12, 13, 14, 15, 16, 17, 18, 19, 20 or more) of subjects known to be uninfected; and the respective levels may be compared.

In a further aspect, the present invention also provides a (in-vitro) method of evaluating the severity of COVID-19 in a subject, wherein the method comprises determining the level (amount or concentration) of antibodies binding to CXCL5, CXCL8 and CCL25 in a (isolated) sample of said subject.

The present inventors have surprisingly found that the level of auto-antibodies binding to human chemokines CXCL5, CXCL8 and CCL25 correlates with the severity of COVID-19 in a subject. As shown in the experimental data below, antibodies to the three chemokines CXCL5, CXCL8 and CCL25 highly significantly distinguished outpatients from hospitalized subjects (p<10⁻⁻⁴): antibodies against CXCL5, CXCL8 and CCL25 were all lower in individuals with severe illness requiring hospitalization. The combination of antibody values against these three chemokines alone could correctly assign hospitalized and outpatient individuals with an accuracy of 77.5%, which was improved to 98.6% by including anti-CCL2 antibodies (“COVID-19 hospitalization signature”). Accordingly, it is preferred that in the method of the invention also the level (amount or concentration) of antibodies binding to CCL2 is determined in the sample of said subject.

Accordingly, the severity of COVID-19 in a subject, in particular whether the subject needs to be/is/was hospitalized (due to the severity of COVID-19 symptoms) can be determined based on the levels of auto-antibodies to human chemokines CXCL5, CXCL8 and CCL25.

To this end, a sample, in particular a body fluid sample (such as blood), may be obtained (isolated) from said subject and the levels of antibodies to CXCL5, CXCL8 and CCL25 may be determined in said sample. In some embodiments, the sample may be obtained from the subject about 1 day to 24 months after the onset of COVID-19 symptoms or infection with SARS-CoV-2, or preferably about 1 day to 1 month after the onset of COVID-19 symptoms or infection with SARS-CoV-2.

The level (amount or concentration) of antibodies in said sample may be determined by using the method of the invention for determining binding of an antibody to a chemokine as described above, the (N-loop chemokine) peptide according to the invention as described above, the composition containing said peptide the invention as described above or the kit the invention as described above. For example, the kit may contain peptides relating to each of the three human chemokines CXCL5, CXCL8 and CCL25. Namely, a peptide as described above comprising the amino acid sequence of SEQ ID NO: 29 or 80 may be used to determine the level of antibodies binding to CXCL5; a peptide as described above comprising the amino acid sequence of SEQ ID NO: 32 or 83 may be used to determine the level of antibodies binding to CXCL8; and/or a peptide as described above comprising the amino acid sequence of SEQ ID NO: 21 or 72 may be used to determine the level of antibodies binding to CCL25. Likewise the respective peptide(s) (or composition) may be used, e.g. in the method of the invention.

In some embodiments, the method of the invention for evaluating the severity of COVID-19 further comprises a step of comparing the level (amount or concentration) of said antibodies to the corresponding antibody levels in a reference group of subjects without hospitalization due to COVID-19. Namely, it may be determined whether or not the levels of antibodies binding to CXCL5, CXCL8 and CCL25 are decreased compared to subjects without COVID-19 hospitalization. The decrease of said antibody levels in a statistically significant manner, p<10⁻⁴ or p<10⁻¹, indicates severe illness. The experimental data described herein below provide an exemplified data set regarding the levels of antibodies binding to CXCL5, CXCL8 and CCL25 in respective outpatients. If this data set is used as reference, the antibody levels in the test sample are preferably determined with the same method, i.e. as described herein below. Alternatively, for example, the levels of antibodies binding to CXCL5, CXCL8 and CCL25 may be determined (i) for the test subject; and (ii) for a plurality (e.g., at least 5, such as 5, 6, 7, 8, 9, 10, 11, 12, 13, 14, 15, 16, 17, 18, 19, 20 or more) of subjects known to be outpatients; and the respective levels may be compared.

In a further aspect, the present invention also provides a method of evaluating the occurrence of long-term COVID-19 symptoms in a subject, wherein the method comprises determining the level (amount or concentration) of antibodies binding to CCL21, CXCL13 and CXCL16 in a (isolated) sample of said subject.

The present inventors have surprisingly found that the level of auto-antibodies binding to human chemokines CCL21, CXCL13 and CXCL16 correlates with the occurrence of long-term COVID-19 symptoms in a subject. As shown in the experimental data below, antibodies to the three chemokines CCL21, CXCL13 and CXCL16 distinguished patients with the occurrence of long-term COVID-19 symptoms from former COVID-19 patients without long-term symptoms with high significance: CCL21 (p=0.0001), CXCL13 (p=0.0010) and CXCL16 (p=0.0011) (“COVID-19 persisting symptoms signature”). Logistic regression analysis using the antibody values for these three chemokines predicted long-term persistence of symptoms with accuracies of 77.8%. These results suggest that specific patterns of anti-chemokine antibodies can predict the occurrence of long-term COVID-19 symptoms.

Accordingly, the occurrence of long-term COVID-19 symptoms in a subject, can be determined based on the levels of auto-antibodies to human chemokines CCL21, CXCL13 and CXCL16. Examples of long-term symptoms of COVID-19 include extreme fatigue, long lasting cough, muscle weakness, low grade fever, inability to concentrate (brain fog), memory lapses, mental health problems (such as changes in mood or depression), sleep difficulties, headaches, joint pain, needle pains in arms and legs, diarrhea, bouts of vomiting, loss or changes in sense of taste, loss or changes in sense of smell (clinical Parosmia or Anosmia), sore throat and or difficulties swallowing, blood disorders (including new onset's of diabetes and hypertension), heartburn (gastroesophageal reflux disease), skin rash, shortness of breath, chest pains, palpitations, kidney problems (including, acute kidney injury and chronic kidney disease), changes in oral health (teeth, saliva, gums), tinnitus, and blood clotting (including deep vein thrombosis and pulmonary embolism). The expression “long-term” with regard to the COVID-19 symptoms refers in particular to COVID-19-related symptoms occurring at about 9 months or more, preferably at about 10 months or more, more preferably at about 11 months or more, even more preferably at about one year or more, for example at about 1.5 years or even more after the onset of COVID-19 symptoms or infection with SARS-CoV-2.

To this end, a sample, in particular a body fluid sample (such as blood), may be obtained (isolated) from said subject and the levels of antibodies to CCL21, CXCL13 and CXCL16 may be determined in said sample. In some embodiments, the sample may be obtained from the subject about 3 to 9 months, preferably about 4 to 8 months, more preferably about 5 to 7 months and even more preferably about 6 months after the onset of COVID-19 symptoms or infection with SARS-CoV-2. In some embodiments such samples may be used to predict the occurrence of COVID-19-related symptoms in a subject at about 9 months or more, preferably at about 10 months or more, more preferably at about 11 months or more, even more preferably at about one year or more, for example at about 1.5 years or even more after the onset of COVID-19 symptoms or infection with SARS-CoV-2.

Accordingly, the present invention also provides a method of predicting the occurrence of long-term symptoms of COVID-19 in a patient at about 9 months or more, preferably at about months or more, more preferably at about 11 months or more, even more preferably at about one year or more, for example at about 12 months, after onset of COVID-19 or infection with SARS-CoV-2, wherein the method comprises determining the level (amount or concentration) of antibodies binding to CCL21, CXCL13 and CXCL16 in a (isolated) sample of said subject, wherein the sample was obtained from the subject about 3 to 9 months, preferably about 4 to 8 months, more preferably about 5 to 7 months and even more preferably about 6 months after the onset of COVID-19 symptoms or infection with SARS-CoV-2.

The level (amount or concentration) of antibodies in said sample may be determined by using the method of the invention for determining binding of an antibody to a chemokine as described above, the (N-loop chemokine) peptide according to the invention as described above, the composition containing said peptide the invention as described above or the kit the invention as described above. For example, the kit may contain peptides relating to each of the three human chemokines CCL21, CXCL13 and CXCL16. Namely, a peptide as described above comprising the amino acid sequence of SEQ ID NO: 17 or 68 may be used to determine the level of antibodies binding to CCL21; a peptide as described above comprising the amino acid sequence of SEQ ID NO: 37 or 88 may be used to determine the level of antibodies binding to CXCL13; and/or a peptide as described above comprising the amino acid sequence of SEQ ID NO: 39 or 90 may be used to determine the level of antibodies binding to CXCL16. Likewise the respective peptide(s) (or composition) may be used, e.g. in the method of the invention.

In some embodiments, the method of the invention for evaluating the occurrence of long-term COVID-19 symptoms and the method of the invention for predicting the occurrence of long-term symptoms of COVID-19 further comprises a step of comparing the level (amount or concentration) of said antibodies to the corresponding antibody levels in a reference group of subjects without long-term COVID-19 symptoms, in particular subjects previously infected with SARS-CoV-2 and without long-term COVID-19 symptoms. Namely, it may be determined whether or not the levels of antibodies binding to CCL21, CXCL13 and CXCL16 are decreased compared to subjects without long-term COVID-19 symptoms. The decrease of said antibody levels in a statistically significant manner, e.g. p<10⁻⁴ or p<10⁻³ or p<10⁻¹, may allow indicating long-term persistence of symptoms. The experimental data described herein below provide an exemplified data set regarding the levels of antibodies binding to CCL21, CXCL13 and CXCL16 in subjects without long-term COVID-19 symptoms. If this data set is used as reference, the antibody levels in the test sample are preferably determined with the same method, i.e. as described herein below. Alternatively, for example, the levels of antibodies binding to CCL21, CXCL13 and CXCL16 may be determined (i) for the test subject; and (ii) for a plurality (e.g., at least 5, such as 5, 6, 7, 8, 9, 10, 11, 12, 13, 14, 15, 16, 17, 18, 19, 20 or more) of subjects without long-term COVID-19 symptoms; and the respective levels may be compared.

In a further aspect, the present invention also provides a (in-vitro) method for assisting in diagnosis of Long COVID, wherein the method comprises determining the level (amount or concentration) of antibodies binding to CCL21, CXCL13 and CXCL16. As some symptoms of long-term COVID-19 (see examples of above), may not be sufficiently specific for diagnosing Long-COVID, for example due to an overlap with other disorders, the specific anti-chemokine antibody signature found by the present inventors for Long COVID, i.e. lower levels of antibodies against chemokines CCL21, CXCL13 and CXCL16 as described above, may assist in the diagnosis of Long COVID, in particular as it can be easily measured, e.g. as described herein above. Accordingly, determining the (decreased) levels of auto-antibodies against the three chemokines CCL21, CXCL13 and CXCL16 may be a useful tool, in particular when combined with (“classical”) diagnosis of Long COVID, e.g., based on one or more COVID-19-related long-term symptom(s), such as a symptom selected from the group consisting of extreme fatigue, long lasting cough, muscle weakness, low grade fever, inability to concentrate (brain fog), memory lapses, mental health problems (such as changes in mood or depression), sleep difficulties, headaches, joint pain, needle pains in arms and legs, diarrhea, bouts of vomiting, loss or changes in sense of taste, loss or changes in sense of smell (clinical Parosmial or Anosmia), sore throat and or difficulties swallowing, blood disorders (including new onset's of diabetes and hypertension), heartburn (gastroesophageal reflux disease), skin rash, shortness of breath, chest pains, palpitations, kidney problems (including, acute kidney injury and chronic kidney disease), changes in oral health (teeth, saliva, gums), tinnitus, blood clotting (including deep vein thrombosis and pulmonary embolism) and combinations thereof. The detailed description above with regard to determining the level (amount or concentration) of antibodies binding to CCL21, CXCL13 and CXCL16 applies likewise to the method for assisting in diagnosis of Long COVID.

The presence of decreased levels of auto-antibodies against the three human chemokines CCL21, CXCL13 and CXCL16 observed by the present inventors in patients developing COVID-19-related long-term symptoms (Long COVID) indicates a beneficial effect of the antibodies against CCL21, CXCL13 and CXCL16 in prophylaxis and/or treatment of long-term symptoms of COVID-19.

Accordingly, the present invention also provides a (combination of) antibodies, or antigen-binding fragments thereof, which bind to CCL21, CXCL13 and CXCL16, for use in the prophylaxis or treatment of long-term symptoms of COVID-19. The present invention also provides a method for prophylaxis or treatment of long-term symptoms of COVID-19 comprising administering to a subject in need thereof antibodies binding to CCL21, CXCL13 and CXCL16. In other words, for prophylaxis or treatment of Long COVID, an antibody, or antigen-binding fragments thereof, which binds to CCL21, an antibody, or antigen-binding fragments thereof, which binds to CXCL13 and an antibody, or antigen-binding fragments thereof, which binds to CXCL16, may be combined (i.e. administered in combination, e.g. in the same treatment schedule). Administration of the antibodies is usually in a “prophylactically effective amount” or a “therapeutically effective amount” (as the case may be), this being sufficient to show benefit to the individual. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician.

In particular, the antibodies of the present invention as described above, which bind to CCL21, CXCL13 and CXCL16, may be used in the prophylaxis or treatment of long-term symptoms of COVID-19. That is, the antibody, or an antigen-binding fragment thereof, binding to CCL21 of the present invention as described above, the antibody, or an antigen-binding fragment thereof, binding to CXCL13 of the present invention as described above, and the antibody, or an antigen-binding fragment thereof, binding to CXCL16 of the present invention as described above, the respective nucleic acids encoding such antibodies as described above, the combination of nucleic acids encoding such antibodies as described above, the respective vectors as described above, the combination of vectors as described above, the respective cell(s) as described above, or the respective composition(s) as described above may be used in the prophylaxis or treatment of Long COVID.

In a further aspect, the present invention also provides a (in-vitro) method of evaluating the status of HIV infection in a subject, wherein the method comprises determining the level (amount or concentration) of antibodies binding to CCL4, CCL2, CXCL9 and CXCL12 in a (isolated) sample of said subject. In some embodiments, the method comprises determining the level (amount or concentration) of antibodies binding to one or more further chemokines selected from the group CCL3, CCL5, CCL20, CCL21, CCL22, CCL23, CCL27, CCL28, CXCL7 and CXCL8. In some embodiments, the method comprises determining the level (amount or concentration) of antibodies binding to each of the chemokines CCL2, CCL3, CCL4, CCL5, CCL20, CCL21, CCL22, CCL23, CCL27, CCL28, CXCL7, CXCL8, CXCL9 and CXCL12.

The present inventors have surprisingly found that the level of auto-antibodies binding to human chemokines CCL4, CCL2, CXCL9 and CXCL12 correlates with the status of HIV infection in a subject. As shown in the experimental data below, the levels of antibodies to the four chemokines CCL4, CCL2, CXCL9 and CXCL12 were significantly increased in HIV-infected subjects. In addition also antibody levels of antibodies binding to other chemokines were increased in HIV-infected subjects. Namely, antibodies against 14 chemokines were increased with high confidence: CCL2, CCL3, CCL4, CCL5, CCL20, CCL21, CCL22, CCL23, CCL27, CCL28, CXCL7, CXCL8, CXCL9 and CXCL12 (p<10⁻⁴ for all). Thus, HIV infection is associated with a specific pattern of anti-chemokine antibodies.

To this end, a sample, in particular a body fluid sample (such as blood), may be obtained (isolated) from said subject and the levels of antibodies to CCL4, CCL2, CXCL9 and CXCL12 may be determined in said sample. In some embodiments, in addition the levels (amount or concentration) of antibodies binding to one or more further chemokines selected from the group CCL3, CCL5, CCL20, CCL21, CCL22, CCL23, CCL27, CCL28, CXCL7 and CXCL8 may be determined in said sample. For example, the levels of antibodies binding to each of the chemokines CCL2, CCL3, CCL4, CCL5, CCL20, CCL21, CCL22, CCL23, CCL27, CCL28, CXCL7, CXCL8, CXCL9 and CXCL12 may be determined. In some embodiments, the sample may be obtained from the subject 1, 2, 3, 4, 5, 6, 7, 8, 9, 10 or more days or weeks after the onset of HIV symptoms or infection with HIV.

The level (amount or concentration) of antibodies in said sample may be determined by using the method of the invention for determining binding of an antibody to a chemokine as described above, the (N-loop chemokine) peptide according to the invention as described above, the composition containing said peptide the invention as described above or the kit the invention as described above. For example, the kit may contain peptides relating to each of the four human chemokines CCL4, CCL2, CXCL9 and CXCL12. In some embodiments, the kit may contain one or more additional peptide(s) relating to one or more further chemokines selected from the group consisting of CCL3, CCL5, CCL20, CCL21, CCL22, CCL23, CCL27, CCL28, CXCL7 and CXCL8. In some embodiments, the kit may contain peptides relating to each of the chemokines CCL2, CCL3, CCL4, CCL5, CCL20, CCL21, CCL22, CCL23, CCL27, CCL28, CXCL7, CXCL8, CXCL9 and CXCL12.

Namely, a peptide as described above comprising the amino acid sequence of SEQ ID NO: 2 or 53 may be used to determine the level of antibodies binding to CCL2; a peptide as described above comprising the amino acid sequence of SEQ ID NO: 3 or 54 may be used to determine the level of antibodies binding to CCL3; a peptide as described above comprising the amino acid sequence of SEQ ID NO: 4 or 55 may be used to determine the level of antibodies binding to CCL4; a peptide as described above comprising the amino acid sequence of SEQ ID NO: 5 or 56 may be used to determine the level of antibodies binding to CCL5; a peptide as described above comprising the amino acid sequence of SEQ ID NO: 16 or 67 may be used to determine the level of antibodies binding to CCL20; a peptide as described above comprising the amino acid sequence of SEQ ID NO: 17 or 68 may be used to determine the level of antibodies binding to CCL21; a peptide as described above comprising the amino acid sequence of SEQ ID NO: 18 or 69 may be used to determine the level of antibodies binding to CCL22; a peptide as described above comprising the amino acid sequence of SEQ ID NO: 19 or 70 may be used to determine the level of antibodies binding to CCL23; a peptide as described above comprising the amino acid sequence of SEQ ID NO: 23 or 74 may be used to determine the level of antibodies binding to CCL27; a peptide as described above comprising the amino acid sequence of SEQ ID NO: 24 or 75 may be used to determine the level of antibodies binding to CCL28; a peptide as described above comprising the amino acid sequence of SEQ ID NO: 31 or 82 may be used to determine the level of antibodies binding to CXCL7; a peptide as described above comprising the amino acid sequence of SEQ ID NO: 32 or 83 may be used to determine the level of antibodies binding to CXCL8; a peptide as described above comprising the amino acid sequence of SEQ ID NO: 33 or 84 may be used to determine the level of antibodies binding to CXCL9; and a peptide as described above comprising the amino acid sequence of SEQ ID NO: 36 or 87 may be used to determine the level of antibodies binding to CXCL12. Likewise the respective peptide(s) (or composition) may be used, e.g. in the method of the invention.

In some embodiments, the method of the invention for evaluating the HIV status further comprises a step of comparing the level (amount or concentration) of said antibodies to the corresponding antibody levels in a reference group of subjects without HIV infection (uninfected subjects). Namely, it may be determined whether or not the levels of antibodies binding to CCL4, CCL2, CXCL9 and CXCL12 (and, optionally, one or more further antibodies binding to one or more chemokine selected from the group consisting of CCL3, CCL5, CCL20, CCL21, CCL22, CCL23, CCL27, CCL28, CXCL7 and CXCL8) are increased compared to subjects without HIV. The experimental data described herein below provide an exemplified data set regarding the levels of antibodies binding to CCL2, CCL3, CCL4, CCL5, CCL20, CCL21, CCL22, CCL23, CCL27, CCL28, CXCL7, CXCL8, CXCL9 and CXCL12 in uninfected subjects. If this data set is used as reference, the antibody levels in the test sample are preferably determined with the same method, i.e. as described herein below. Alternatively, for example, the levels of antibodies may be determined (i) for the test subject; and (ii) for a plurality (e.g., at least 5, such as 5, 6, 7, 8, 9, 10, 11, 12, 13, 14, 15, 16, 17, 18, 19, 20 or more) of subjects known to be uninfected; and the respective levels may be compared.

In a further aspect, the present invention also provides a (in-vitro) method of evaluating the status of an autoimmune disorder in a subject, wherein the method comprises determining the level (amount or concentration) of antibodies binding to CCL4, CCL19, CCL25 and CXCL9 in a (isolated) sample of said subject.

The present inventors have surprisingly found that the level of auto-antibodies binding to human chemokines CCL4, CCL19, CCL25 and CXCL9 correlates with the status of various autoimmune disorders in a subject. As shown in the experimental data below, the levels of antibodies to the four chemokines CCL4, CCL19, CCL25 and CXCL9 were significantly increased in subjects suffering from any autoimmune disorder selected from ankylosing spondylitis, rheumatoid arthritis and Sjögren syndrome. Thus, autoimmune disorders are associated with a specific pattern of anti-chemokine antibodies.

Moreover, the present inventors also found that the level of auto-antibodies binding to human chemokines CCL4, CCL19, CCL25, CXCL9, CCL13, CXCL7 and CXCL8 correlates with the status of ankylosing spondylitis and/or rheumatoid arthritis in a subject. Accordingly, the method may be used to determine the status of ankylosing spondylitis and/or rheumatoid arthritis and, to this end, the method may further comprise a step of determining the level (amount or concentration) of antibodies binding to a chemokine selected from the group consisting of CCL13, CXCL7 and CXCL8 (in addition to the levels of antibodies to the four chemokines CCL4, CCL19, CCL25 and CXCL9). Accordingly, the present invention also provides a (in-vitro) method for determining the status of ankylosing spondylitis and/or rheumatoid arthritis in a subject, wherein the method comprises determining the level (amount or concentration) of antibodies binding to CCL4, CCL19, CCL25, CXCL9, CCL13, CXCL7 and CXCL8 in a (isolated) sample of said subject.

In addition, the present inventors have found that the level of auto-antibodies binding to human chemokines CCL2, CCL5 CCL8, CCL11, CCL14, CCL16, CCL17, CCL18 and CXCL12 correlates with the status of ankylosing spondylitis (but not with rheumatoid arthritis or Sjögren syndrome) in a subject. Accordingly, the method may be used to determine the status of ankylosing spondylitis and, to this end, the method may further comprise a step of determining the level (amount or concentration) of antibodies binding to a chemokine selected from the group consisting of CCL2, CCL5 CCL8, CCL11, CCL14, CCL16, CCL17, CCL18 and CXCL12 (in addition to the levels of antibodies to the four chemokines CCL4, CCL19, CCL25 and CXCL9; and, optionally, CCL13, CXCL7 and CXCL8). In particular, determining the level (amount or concentration) of antibodies binding to a chemokine selected from the group consisting of CCL2, CCL5 CCL8, CCL11, CCL14, CCL16, CCL17, CCL18 and CXCL12 may be used to distinguish ankylosing spondylitis from other autoimmune disorders, such as rheumatoid arthritis or Sjögren syndrome. Accordingly, the present invention also provides a (in-vitro) method for determining the status of ankylosing spondylitis and/or rheumatoid arthritis in a subject, wherein the method comprises determining the level (amount or concentration) of antibodies binding to CCL4, CCL19, CCL25, CXCL9, CCL13, CXCL7, CXCL8, CCL2, CCL5 CCL8, CCL11, CCL14, CCL16, CCL17, CCL18 and/or CXCL12 in a (isolated) sample of said subject.

In addition, the present inventors have found that the level of auto-antibodies binding to the human chemokine CCL1 correlates with the status of Sjögren syndrome (but not with rheumatoid arthritis or ankylosing spondylitis) in a subject. Accordingly, the method may be used to determine the status of Sjögren syndrome and, to this end, the method may further comprise a step of determining the level (amount or concentration) of antibodies binding to the chemokine CCL1 (in addition to the levels of antibodies to the four chemokines CCL4, CCL19, CCL25 and CXCL9). In particular, determining the level (amount or concentration) of antibodies binding to the chemokine CCL1 may be used to distinguish Sjögren syndrome from other autoimmune disorders, such as ankylosing spondylitis or rheumatoid arthritis. Accordingly, the present invention also provides a (in-vitro) method for determining the status of Sjögren syndrome in a subject, wherein the method comprises determining the level (amount or concentration) of antibodies binding to CCL4, CCL19, CCL25, CXCL9 and CCL1 in a (isolated) sample of said subject.

To determine the status of an autoimmune disorder, such as ankylosing spondylitis, rheumatoid arthritis or Sjögren syndrome, a sample, in particular a body fluid sample (such as blood), may be obtained (isolated) from said subject and the levels of antibodies to the chemokines as described above for autoimmune disorders, such as ankylosing spondylitis, rheumatoid arthritis or Sjögren syndrome, may be determined in said sample.

The level (amount or concentration) of antibodies in said sample may be determined by using the method of the invention for determining binding of an antibody to a chemokine as described above, the (N-loop chemokine) peptide according to the invention as described above, the composition containing said peptide the invention as described above or the kit the invention as described above. For example, the kit may contain peptides relating to each of the four human chemokines CCL4, CCL9, CCL25 and CXCL9. In some embodiments, the kit may contain one or more additional peptide(s) relating to one or more further chemokines selected from the group consisting of CCL13, CXCL7 and CXCL8. In some embodiments, the kit may contain one or more additional peptide(s) relating to one or more further chemokines selected from the group consisting of CCL2, CCL5 CCL8, CCL11, CCL14, CCL16, CCL17, CCL18 and CXCL12. In some embodiments, the kit may contain an additional peptide relating to the chemokine CCL1. In some embodiments, the kit may contain peptides relating to each of the chemokines CCL4, CCL19, CCL25, CXCL9, CCL13, CXCL7 and CXCL8. In some embodiments, the kit may contain peptides relating to each of the chemokines CCL4, CCL19, CCL25, CXCL9, CCL13, CXCL7, CXCL8, CCL2, CCL5 CCL8, CCL11, CCL14, CCL16, CCL17, CCL18 and/or CXCL12. In some embodiments, the kit may contain peptides relating to each of the chemokines CCL4, CCL19, CCL25, CXCL9 and CCL1.

Namely, a peptide as described above comprising the amino acid sequence of SEQ ID NO: 1 or 52 may be used to determine the level of antibodies binding to CCL1; a peptide as described above comprising the amino acid sequence of SEQ ID NO: 2 or 53 may be used to determine the level of antibodies binding to CCL2; a peptide as described above comprising the amino acid sequence of SEQ ID NO: 4 or 55 may be used to determine the level of antibodies binding to CCL4; a peptide as described above comprising the amino acid sequence of SEQ ID NO: 5 or 56 may be used to determine the level of antibodies binding to CCL5; a peptide as described above comprising the amino acid sequence of SEQ ID NO: 7 or 58 may be used to determine the level of antibodies binding to CCL8; a peptide as described above comprising the amino acid sequence of SEQ ID NO: 8 or 59 may be used to determine the level of antibodies binding to CCL11; a peptide as described above comprising the amino acid sequence of SEQ ID NO: 9 or 60 may be used to determine the level of antibodies binding to CCL13; a peptide as described above comprising the amino acid sequence of SEQ ID NO: 10 or 61 may be used to determine the level of antibodies binding to CCL14; a peptide as described above comprising the amino acid sequence of SEQ ID NO: 12 or 63 may be used to determine the level of antibodies binding to CCL16; a peptide as described above comprising the amino acid sequence of SEQ ID NO: 13 or 64 may be used to determine the level of antibodies binding to CCL17; a peptide as described above comprising the amino acid sequence of SEQ ID NO: 14 or 65 may be used to determine the level of antibodies binding to CCL18; a peptide as described above comprising the amino acid sequence of SEQ ID NO: 15 or 66 may be used to determine the level of antibodies binding to CCL19; a peptide as described above comprising the amino acid sequence of SEQ ID NO: 21 or 72 may be used to determine the level of antibodies binding to CCL25; a peptide as described above comprising the amino acid sequence of SEQ ID NO: 12 or 63 may be used to determine the level of antibodies binding to CCL; a peptide as described above comprising the amino acid sequence of SEQ ID NO: 31 or 82 may be used to determine the level of antibodies binding to CXCL7; a peptide as described above comprising the amino acid sequence of SEQ ID NO: 32 or 83 may be used to determine the level of antibodies binding to CXCL8; a peptide as described above comprising the amino acid sequence of SEQ ID NO: 33 or 84 may be used to determine the level of antibodies binding to CXCL9; and a peptide as described above comprising the amino acid sequence of SEQ ID NO: 36 or 87 may be used to determine the level of antibodies binding to CXCL12. Likewise the respective peptide(s) (or composition) may be used, e.g. in the method of the invention.

In some embodiments, the method of the invention for evaluating the status of an autoimmune disorder, such as ankylosing spondylitis, rheumatoid arthritis or Sjögren syndrome, further comprises a step of comparing the level (amount or concentration) of said antibodies to the corresponding antibody levels in a reference group of subjects without autoimmune disorder, such as ankylosing spondylitis, rheumatoid arthritis or Sjögren syndrome. Namely, it may be determined whether or not the levels of antibodies binding to CCL4, CCL19, CCL25 and CXCL9 (and, optionally, one or more further antibodies binding to one or more chemokine selected from the group consisting of CCL1, CCL13, CXCL7, CXCL8, CCL2, CCL5 CCL8, CCL11, CCL14, CCL6, CCL17, CCL18 and/or CXCL12) are increased compared to subjects without autoimmune disorder, such as ankylosing spondylitis, rheumatoid arthritis or Sjögren syndrome. The experimental data described herein below provide an exemplified data set regarding the levels of antibodies binding to CCL4, CCL19, CCL25, CXCL9, CCL13, CXCL7, CXCL8, CCL2, CCL5 CCL8, CCL11, CCL14, CCL16, CCL17, CCL18 and/or CXCL12 in subjects without autoimmune disorder, such as ankylosing spondylitis, rheumatoid arthritis or Sjögren syndrome. If this data set is used as reference, the antibody levels in the test sample are preferably determined with the same method, i.e. as described herein below. Alternatively, for example, the levels of antibodies may be determined (i) for the test subject; and (ii) for a plurality (e.g., at least 5, such as 5, 6, 7, 8, 9, 10, 11, 12, 13, 14, 15, 16, 17, 18, 19, 20 or more) of subjects known to be uninfected; and the respective levels may be compared.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1. Human monoclonal antibodies that impede CCL8 chemotaxis
(A) Characteristics of the COVID-19 cohort.
(B) Model of the interaction between a chemokine and its receptor. Arrows point to the area of putative interaction between the N-terminus of the receptor and the chemokine N-loop (shown by spheres). Chemokine is magenta and chemokine receptor is cyan.
(C) Identification of individuals with high anti-CCL8 antibodies. Top, optical density (OD₄₅₀) shows plasma IgG reactivity to the CCL8 N-loop peptide (SEQ ID NO: 7), as determined by ELISA. Bottom, area under the curve (AUC) of the data in top panel. Average of two independent experiments. COVID-19 convalescents (n=71); controls (n=23). Horizontal bars indicate median values.
(D) Human B cells specific for CCL8. Flow cytometry plots identify human B cells binding to the CCL8 N-loop peptide (gate). The frequency of antigen-specific B cells is shown.
(E) Pie chart summarizing the 23 anti-CCL8 antibodies sequences. Black represents clonally related VH4-39/VK4-1 antibodies. White represents singlets.
(F) Number of Ig Variable Heavy and Light chain gene somatic mutations in VH4-39/VK4-1 antibodies. Horizontal bars indicate median values.
(G) VH4-39/VK4-1 antibodies bind to the CCL8 N-loop. ELISA binding curves of representative VH4-39/VK4-1 antibodies. Average of two independent experiments (Mean+SEM).
(H) Chemotaxis of human monocytes towards CCL8 is inhibited by VH4-39/VK4-1 antibodies. Mean±SEM of migrated cells in 5 high-power fields (HPF). At least 3 independent experiments with cells from different donors. Up-pointing triangle is antibody alone, and down-pointing triangle is buffer control. Two-way RM ANOVA followed by Sdak's multiple comparisons test.
(I) VH4-39/VK4-1 antibodies selectively inhibit CCL8 chemotaxis to CCR1. Relative cell migration to CCL8 in the presence of antibodies compared to no antibody control with cells uniquely expressing CCR1 or CCR2 (see Methods). Mean±SEM of at least 3 independent experiments. Two-tailed Mann-Whitney U-tests.

FIG. 2. Identification and specificity of anti-CCL8 antibodies
(A) IgGs from COVID-19 convalescents inhibit CCL8 chemotaxis. Chemotaxis of CCR2 expressing preB 300.19 cells towards the indicated chemokines was measured in the presence of plasma IgGs from COVID-19 convalescents (n=24) or controls (n=8). Technical triplicates (Mean±SEM) of migrated cells in 5 high-power fields (HPF). Two-tailed Mann-Whitney U-tests.
(B) Detection of anti-IFNα2 IgGs. AUC of optical density (OD₄₅₀) measurements obtained in ELISA from serial plasma dilutions against an IFNα2 peptide. Average AUC from two independent experiments.
(C) Gating strategy for sorting CCL8 N-loop specific B cells by flow cytometry.
(D) VH4-39/VK4-1 antibodies bind the CCL8 N-loop specifically. Similarity analysis of all human chemokines N-loops by Neighbour joining-BLOSUM62 identifies those that are most similar to CCL8 (red in the right panel). VH4-39/VK4-1 antibodies only bind to CCL8 in ELISA (left panel). Dashed lines indicate the signal of a positive control plasma sample with broad reactivity (CLM70). Average of two independent experiments (Mean+SEM). (E) VH4-39/VK4-1 antibodies do not bind to the Spike protein of SARS-CoV-2. C135 is positive control (Robbiani et al., 2020). Average of two independent experiments (Mean+SEM).
(F) Chemotaxis of primary human monocytes towards CCL8 is inhibited by the aCCL8.003 and aCCL8.004 VH4-39/VK4-1 antibodies. Mean±SEM of migrated cells in 5 HPF from at least 3 independent experiments performed with cells from different donors. Two-way RM ANOVA followed by Sidak's multiple comparisons test.

FIG. 3. Antibodies to CCL19, CCL22 and CXCL17 distinguish COVID-19
(A) Anti-chemokine antibodies 6 months after COVID-19. Heatmap representing plasma IgG binding to 42 peptides comprising the N-loop sequence of all 43 human chemokines (SEQ ID NOs 1-42), as determined by ELISA (AUC, average of two independent experiments). Samples are ranked according to the level of anti-SARS-CoV-2-RBD. Anti-chemokine IgG signals are ordered by unsupervised clustering analysis. SARS-CoV-2 pseudovirus neutralizing activity (NT₅₀) and IgG binding to peptides corresponding to negative control, IFNα2 (SEQ ID NO: 50) and SARS-CoV-2 nucleocapsid protein (N) (SEQ ID NO: 51) are shown. COVID-19 convalescents (n=71); controls (n=23).
(B) t-SNE distribution of COVID-19 convalescents and controls, as determined with the 42 datasets combined.
(C) Pearson correlations of antibodies to the N-loop (SEQ ID NOs 7, 12, 22, 25, 36, 37 and 39) and C-term peptides (SEQ ID NOs 43-49) of the same chemokine. ELISA was performed in a cohort subset (Controls, n=5; COVID-19, n=31). Average of two independent experiments.
(D) Differences in anti-chemokine antibodies between groups. Summary circle plot: circle size indicates significance; greyscale tone shows the Log₂ fold-change (FC); #indicates a negative FC in the COVID-19 group over controls. Two-tailed Mann-Whitney U-tests.
(E) Difference in antibodies to CCL19, CCL22 and CXCL17 (COVID-19 signature). Horizontal bars indicate median values. Two-tailed Mann-Whitney U-tests.
(F) Assignment of COVID-19 convalescents and controls based on the COVID-19 signature antibodies by logistic regression analysis. Dots on grey background are correctly assigned.

FIG. 4. Anti-chemokine N-loop antibodies in COVID-19

The amount of plasma IgG antibodies against each chemokine N-loop was determined by ELISA for COVID-19 convalescents (n=71) and controls (n=23). Average optical density (OD₄₅₀) of two independent experiments.

FIG. 5. Anti-chemokine antibodies clustering and correlation analyses
(A) Pearson correlations of antibodies to the N-loop and C-term peptides of the same chemokine. ELISA was performed in a cohort subset (Controls, n=5; COVID-19, n=31). Average AUC of two independent experiments.
(B) Antibodies to CCL19, CCL22 and CXCL17 correctly classify COVID-19 convalescents versus controls. Unsupervised hierarchical clustering analysis with the COVID-19 signature antibodies. The distribution of the groups within each cluster is also shown. Fisher's exact test.
(C) Anti-RBD IgG antibodies in the cohort samples. Top, optical density reactivity (OD₄₅₀) of serial plasma dilutions to the receptor binding domain (RBD) of SARS-CoV-2 Spike, as determined by ELISA. Bottom, AUC of the data in the top panel. COVID-19 convalescents (n=71); controls (n=23). Average of two independent experiments. Horizontal bars indicate median values. Two-tailed Mann-Whitney U-tests.
(D) Plasma neutralizing activity against SARS-CoV-2 pseudovirus. Top, relative luciferase units (RLU) normalized to no plasma control. Bottom, half-maximal neutralizing titers (NT₅₀) based on the data in the top panel. COVID-19 convalescents (n=71); controls (n=9). Average of two independent experiments. Horizontal bars indicate median values. Two-tailed Mann-Whitney U-tests.
(E) Pearson correlations of anti-RBD IgG and NT₅₀ values to each other and with age. Average of two independent experiments.
(F) Pearson correlations of anti-chemokine IgG with anti-RBD IgG, NT₅₀ values and age. COVID-19 signature antibodies individually, and cumulative signal of the IgGs against the peptides for all 43 chemokines.
(G) Analysis of anti-signature chemokines IgG by gender. Data are shown as average AUC of two independent experiments. Horizontal bars indicate median values. Kruskal-Wallis test followed by Dunn's multiple comparison test.

FIG. 6. Anti-chemokine antibodies over time and upon COVID-19 vaccination
(A) Diagram of the time points of blood collection after onset of COVID-19 symptoms.
(B) Anti-RBD IgG antibodies at 6 and 12 months in vaccinated and non-vaccinated convalescents, as determined by ELISA. Average AUC from two independent experiments. Wilcoxon signed-rank test.
(C) Anti-chemokine IgG antibodies at 6 and 12 months in convalescents. AUC from two independent experiments. Wilcoxon signed-rank test.
(D) Diagram of the time points of blood collection after onset of COVID-19 symptoms in a subset of COVID-19 hospitalized individuals.
(E) Anti-chemokine IgG antibodies at 15 days (Acute), 6, 12 and 18 months after onset of COVID-19 symptoms. Average optical density (OD₄₅₀) values from two independent experiments. One-way ANOVA test followed by Tukey's multiple comparison test. Data are shown as median±range.
(F) Anti-chemokine IgG antibodies before and approximately 4 months after COVID-19 mRNA vaccination of uninfected individuals (n=16). AUC from two independent experiments. Pink lines represent the signal of a positive control plasma sample with broad reactivity (CLM70). Anti-RBD IgG is shown alongside as control (right panel). Wilcoxon signed-rank test with false discovery rate (FDR) approach.

FIG. 7. Distinct patterns of anti-chemokine antibodies in COVID-19 outpatient and hospitalized individuals at six months after infection
(A) Difference in anti-chemokine antibodies between COVID-19 groups and controls. Summary circle plot: circle size indicates significance; greyscale tone shows the Log₂ fold-change (FC); #indicates a negative FC over controls. Kruskal-Wallis test followed by Dunn's multiple comparison test.
(B) Difference in antibodies to chemokines that most significantly distinguish across COVID-19 and control groups. Horizontal bars indicate median values. Kruskal-Wallis test followed by Dunn's multiple comparison test.
(C) Difference in total anti-chemokine antibodies. Cumulative signal of the IgGs against the 42 peptides comprising the N-loop sequence of all 43 human chemokines (SEQ ID NOs 1-42). Horizontal bars indicate median values. Kruskal-Wallis test followed by Dunn's multiple comparison test.
(D) t-SNE distribution of COVID-19 outpatient and hospitalized individuals, as determined with the 42 datasets combined.
(E) Difference in anti-chemokine antibodies between COVID-19 outpatient and hospitalized individuals. Summary circle plot: circle size indicates significance; greyscale tone shows the Log₂ fold-change (FC); #indicates a negative FC in the COVID-19 group over outpatients. Kruskal-Wallis test followed by Dunn's multiple comparison test.
(F) Assignment of COVID-19 outpatient and hospitalized individuals based on the COVID-19 signature antibodies by logistic regression analysis. Dots on grey background are correctly assigned.

FIG. 8. Anti-chemokine autoantibodies in COVID-19 outpatient and hospitalized individuals at six months after infection
(A) Difference in anti-chemokine antibodies in outpatient versus hospitalized individuals at 6 months. Average AUC of two independent experiments. Horizontal bars indicate median values. Kruskal-Wallis test followed by Dunn's multiple comparison test.
(B) Group assignment based on the antibodies against CXCL8, CCL25 and CXCL5, by logistic regression analysis. Dots on grey background are correctly assigned.
(C) Pearson correlations of anti-chemokine IgGs with anti-RBD IgG, NT₅₀ values and age. COVID-19 hospitalization signature antibodies individually. Average of two independent experiments.
(D) Analysis of anti-signature chemokine IgGs by gender. Data are shown as average AUC of two independent experiments. Horizontal bars indicate median values. Kruskal-Wallis test followed by Dunn's multiple comparison test.
(E) Analysis of anti-RBD IgG and NT₅₀ values by group and by gender. Average of two independent experiments. Horizontal bars indicate median values. Two-tailed Mann-Whitney U-tests.

FIG. 9. Anti-chemokine antibodies and long-term COVID-19 symptoms
(A) Characteristics of the COVID-19 convalescent cohort at 12 months.
(B-D) Persisting symptoms (Sx) at 12 months and anti-chemokine IgG (cumulative; B), anti-RBD IgG (C), and NT₅₀ (D) values at 6 months. Left panels are any symptoms, right panels are symptoms by category. Horizontal bars indicate median values. Average AUC from two independent experiments. Kruskal-Wallis test followed by Dunn's multiple comparison test.
(E) Difference in antibodies to CCL21, CXCL13 and CXCL16 (COVID-19 persisting symptoms signature). Horizontal bars indicate median values. Average AUC from two independent experiments. Two-tailed Mann-Whitney U-tests.
(F) Group assignment based on the COVID-19 persisting symptoms signature antibodies at 6 months against CCL21, CXCL13 and CXCL16, by logistic regression analysis. Dots on grey background are correctly assigned.

FIG. 10. Anti-chemokine antibodies and long-term COVID-19 symptoms
(A) Classification of long-term COVID-19 symptoms at 12 months (t=12 m).
(B) Incidence of symptoms at 12 months. Participants are grouped in outpatient and hospitalized individuals.
(C) Analysis of age (left), gender distribution (middle) and time from COVID-19 onset to 2^{nd} visit (t=12 m; right panel). Horizontal bars indicate median values. Two-tailed Mann-Whitney U-tests.
(D,E) Difference in cumulative anti-chemokine antibodies according to the presence or absence of symptoms at 12 months in disease severity groups (D) or by gender (E). Data are shown as average AUC of two independent experiments. Horizontal bars indicate median values. Two-tailed Mann-Whitney U-tests.
(F) Pearson correlation of anti-chemokine antibodies and the number of symptoms at 12 months. Average of two independent experiments.
(G) Difference in anti-chemokines antibodies at 6 months and the presence or absence of symptoms at 12 month. Data are shown as average AUC of two independent experiments. Horizontal bars indicate median values. Two-tailed Mann-Whitney U-tests. The exact P-value is given for the 3 chemokines displaying the highest significance.

FIG. 11. Distinct patterns of anti-chemokine antibodies in COVID-19, HIV-1 or autoimmune diseases
(A) Difference in anti-chemokine antibodies between disease groups and controls. Summary circle plot: circle size indicates significance; greyscale tone shows the Log₂ fold-change (FC); #indicates a negative FC in the COVID-19 group over controls. Kruskal-Wallis test followed by Dunn's multiple comparison test.
(B) Difference in antibodies to CCL19, CCL4, CCL2, CXCL9 and CXCL12 across groups. Controls (n=23), COVID-19 (n=71), HIV-1 (n=24), Ankylosing Spondylitis (AS, n=13), Rheumatoid Arthritis (RA, n=13), and Sjögren's syndrome (SjS, n=13). Horizontal bars indicate median values. Average AUC from two independent experiments. Kruskal-Wallis test followed by Dunn's multiple comparison test over rank of the control group.
(C) Venn diagram of the chemokines targeted by autoantibodies across the autoimmune disorders AS, RA and SjS. Red and blue colors indicate either an increase or decrease over controls with p<10⁻⁴.
(D) Anti-chemokine antibodies correctly classify COVID-19 convalescents, HIV-1-infected, and patients with autoimmune disorders. Heatmap representing the normalized plasma IgG binding to 42 peptides comprising the N-loop sequence of all 43 human chemokines (SEQ ID NOs 1-42). Unsupervised hierarchical clustering analysis. The distribution of the groups within each cluster is shown.
(E) t-SNE distribution of the different disease groups, as determined with the 42 datasets combined.

FIG. 12. Anti-chemokine N-loop antibodies in HIV-1 and autoimmune diseases

The amount of plasma IgG antibodies against each chemokine N-loop was determined by ELISA for HIV-1 infected (n=24, blue) and autoimmune patients (n=39, orange). Autoimmune patients were subdivided in Ankylosing Spondylitis (AS, n=13), Rheumatoid Arthritis (RA, n=13), and Sjögren's syndrome (SjS, n=13). Values from controls (n=23, black), and COVID-19 convalescents (n=71, green) are shown alongside. Average AUC of two independent experiments. Horizontal bars indicate median values. Statistical significance was determined using Kruskal-Wallis test followed by Dunn's multiple comparison test over rank of the control group.

FIG. 13. Anti-CXCL13, -CXCL16 and-CCL20 N-loop chemotaxis-blocking antibodies in COVID-19 convalescents
(A) Human B cells specific for CXCL16. Representative flow cytometry plots identifying human B cells binding to the CXCL16 N-loop peptide (gate). The frequency of antigen-specific B cells is shown.
(B) Anti-CXCL16 antibodies binding to the CXCL16 N-loop in ELISA. Average of two independent experiments (Mean+SEM).
(C) Anti-CXCL16 N-loop antibodies inhibit CXCL16 chemotaxis to CXCR6. Relative cell migration towards CXCL16 by cells uniquely expressing CXCR6 (see Methods). Mean+SEM of 3 independent experiments. Kruskal-Wallis test followed by Dunn's multiple comparison test.
(D) Human B cells specific for CXCL13. Representative flow cytometry plots identifying human B cells binding to the CXCL13 N-loop peptide (gate). The frequency of antigen-specific B cells is shown.
(E) Anti-CXCL13 antibodies binding to the CXCL13 N-loop in ELISA. Average of two independent experiments (Mean+SEM).
(F) The anti-CXCL13 N-loop antibody aCXCL13.001 inhibits CXCL13 chemotaxis of primary CD19′ human B cells. Mean±SEM of migrated cells in 5 high-power fields (HPF). The average of 3 independent experiments with cells from different donors is shown. Up-pointing triangles indicate antibody alone, and down-pointing triangle is buffer control. Two-way RM ANOVA followed by Ŝidák's multiple comparisons test.
(G) Identification of individuals with high anti-CCL20 antibodies. Right, optical density (OD₄₅₀) shows plasma IgG reactivity to the CCL20 N-loop peptide, as determined by ELISA. Left, area under the curve (AUC) of the data in top panel. Average of two independent experiments. COVID-19 convalescents (n=71); controls (n=23). Horizontal bars indicate median values.
(H) ELISA Monoclonal antibodies to the CCL20 N-loop. ELISA binding curves of representative antibodies. Average of two independent experiments (Mean+SEM).
(I) Anti-CCL20 N-loop antibodies inhibit CCL20 chemotaxis to CCR6. Relative cell migration towards CCL20. Mean+SEM of at least 3 independent experiments. Two-tailed Mann-Whitney U-tests.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Methods

### Study Participants and Ethical Approvals

COVID-19 cohort: 71 participants, who were diagnosed with COVID-19 at the Clinica Luganese Moncucco (CLM, Switzerland) between 08.03.2020 and 22.11.2020, were enrolled in the study and divided into two groups, according to the severity of the acute disease. The hospitalized group included 50 participants; the outpatient group included 21 close contacts of the hospitalized group, who only received at-home care. Inclusion criteria for the hospitalized group were a SARS-CoV-2 positive nasopharyngeal swab test by real-time reverse transcription-polymerase chain reaction (RT-PCR) and age ≥18 years. Inclusion criteria for the outpatient group were being a symptomatic close contact (living in the same household) of an individual enrolled in the hospitalized group and age ≥18 years. At the 12-month visits, participants were asked to indicate the presence or absence of persisting symptoms related to COVID-19 according to a questionnaire. The study was performed in compliance with all relevant ethical regulations and the study protocols were approved by the Ethical Committee of the Canton Ticino (ECCT): CE-3428 and CE-3960.

Control cohort: 15 adult participants (18 years) with self-reported absence of prior SARS-CoV-2 infection or vaccination (confirmed by negative serologic test, FIG. 3A) were enrolled between November 2020 and June 2021. Additional 8 pre-pandemic samples were obtained from blood bank donors (ECCT: CE-3428).

Vaccination cohort: 16 adult participants (≥18 years) with self-reported absence of prior SARS-CoV-2 infection (confirmed by negative serologic test, FIG. 6F) and who received two doses of mRNA-based COVID-19 vaccine (Baden, L. R., El Sahly, H. M., Essink, B., Kotloff, K., Frey, S., Novak, R., Diemert, D., Spector, S. A., Rouphael, N., Creech, C. B., et al. (2021). Efficacy and Safety of the mRNA-1273 SARS-CoV-2 Vaccine. N Engl J Med 384, 403-416; Polack, F. P., Thomas, S. J., Kitchin, N., Absalon, J., Gurtman, A., Lockhart, S., Perez, J. L., Perez Marc, G., Moreira, E. D., Zerbini, C., et al. (2020). Safety and Efficacy of the BNT162b2 mRNA Covid-19 Vaccine. N Engl J Med 383, 2603-2615), were enrolled on the day of first vaccine dose or earlier, between November 2020 and October 2021. (ECCT: CE-3428) HIV-1 and autoimmune diseases cohorts: Pre-pandemic plasma samples were obtained from the following participants: 24 HIV-1 positive (ECCT: CE-813) (Cecchinato, V., Bernasconi, E., Speck, R. F., Proietti, M., Sauermann, U., D'Agostino, G., Danelon, G., Rezzonico Jost, T., Grassi, F., Raeli, L., et al. (2017). Impairment of CCR6+ and CXCR3+Th Cell Migration in HIV-1 Infection Is Rescued by Modulating Actin Polymerization. J Immunol 198, 184-195), 13 each with Ankylosing Spondylitis, Rheumatoid Arthritis (ECCT: CE-3065, and Ethical Committee of the Canton Zurich: EK-515), or Sjögren's syndrome (IRCCS Policlinico San Matteo Foundation Ethics Committee n.20070001302).

Written informed consent was obtained from all participants, and all samples were coded to remove identifiers at the time of blood withdrawal.

### Blood Collection, Processing, and Storage

Blood was collected by venipuncture at approximately 6 months intervals and the peripheral blood mononuclear cells (PBMCs) were isolated using Histopaque density centrifugation. Total PBMCs were aliquoted and frozen in liquid nitrogen in the presence of fetal calf serum and DMSO. Plasma was aliquoted and stored at −20° C. or less. Before use, plasma aliquots were heat-inactivated (56° C. for 1 h) and then stored at 4° C. For chemotaxis assays, CD14⁺ monocytes and CD19⁺ B cells were enriched from fresh PBMCs derived from blood donors (Swiss Red Cross Laboratory; ECCT: CE-3428) through positive immunoselection (130-050-201 and 130-050-301, respectively [Miltenyi Biotec, Bergisch Gladbach, Germany]) according to the manufacturer's instructions. After isolation, CD19⁺ B cells were rested overnight in RPMI-1640 medium supplemented with 10% (v/v) fetal bovine serum (FBS), 1% (v/v) non-essential amino acids, 1 mM sodium pyruvate, 2 mM GlutaMAX, 50 μM β-Mercaptoethanol and 50 U/ml penicillin/streptomycin (all from Gibco) before being used in chemotactic assays.

### Reagents

Peptides: Synthetic peptides containing the N-loop (SEQ ID NOs 1-42) or the C-terminal sequence (SEQ ID NOs 43-49) of human chemokines were designed and obtained (>75% purity) from GenScript (Hong Kong). All peptides are biotinylated (biotin-Ahx) at the N-terminus and amidated at the C-terminus. In addition, the first 2-4 amino acids of each peptide (GS, GGS, GGGS, or GGK depending on the length of the N-loop/C-terminus of the chemokine) consist of a linker between the biotin and the chemokine sequence. Peptides are generally 25 amino acids long, or between 22-25 amino acids when synthesis was problematic. The sequence of the IFNα2 peptide (aa7-28), as shown in SEQ ID NO: 50, was based on a previously described immunoreactive epitope in myasthenia gravis patients Bello-Rivero, I., Cervantes, M., Torres, Y., Ferrero, J., Rodriguez, E., Perez, J., Garcia, I., Diaz, G., and Lopez-Saura, P. (2004). Characterization of the immunoreactivity of anti-interferon alpha antibodies in myasthenia gravis patients. Epitope mapping. J Autoimmun 23, 63-73). The sequence from the SARS-CoV-2 nucleocapsid protein (N) peptide (157-178), as shown in SEQ ID NO: 51, was described in Shrock, E., Fujimura, E., Kula, T., Timms, R. T., Lee, I. H., Leng, Y., Robinson, M. L., Sie, B. M., Li, M. Z., Chen, Y., et al. (2020). Viral epitope profiling of COVID-19 patients reveals cross-reactivity and correlates of severity. Science 370. An irrelevant peptide was used as negative control.

Proteins: CCL2, CCL7, CCL20 and CXCL13 were synthesized using tBoc solid-phase chemistry (Clark-Lewis, I., Vo, L., Owen, P., and Anderson, J. (1997). Chemical synthesis, purification, and folding of C-X-C and C-C chemokines. Methods Enzymol 287, 233-250). CCL8 and CXCL16 were obtained from Peprotech (Cat #300-15 and Cat #300-55, respectively) or produced and purified in house. Briefly, recombinant chemokines were expressed in E. coli, purified from inclusion bodies by immobilized-metal affinity chromatography, and folded under N₂ protection in an arginine-containing buffer (80 mM Tris-Cl [pH 8.51, 100 mM NaCl, 0.8 M arginine, 2 mM EDTA, 1 mM cysteine, 0.2 mM cystine) as previously described (Moepps, B., and Thelen, M. (2016). Monitoring Scavenging Activity of Chemokine Receptors. Methods Enzymol 570, 87-118). After recovery and concentration, the purification tag was cleaved with enterokinase, and the processed chemokine was purified by C₁₈ reverse phase chromatography. The SARS-CoV-2 Spike (S) protein and the S receptor binding domain (RBD) were produced and purified as described (De Gasparo, R., Pedotti, M., Simonelli, L., Nickl, P., Muecksch, F., Cassaniti, I., Percivalle, E., Lorenzi, J. C. C., Mazzola, F., Magri, D., et al. (2021). Bispecific IgG neutralizes SARS-CoV-2 variants and prevents escape in mice. Nature 593, 424-428).

### Chemotaxis

The migration of primary human monocytes isolated from buffy coats, or of murine preB 300.19 cells stably expressing the human chemokine receptors CCR1 or CCR2 (Ogilvie, P., Bardi, G., Clark-Lewis, I., Baggiolini, M., and Uguccioni, M. (2001). Eotaxin is a natural antagonist for CCR2 and an agonist for CCR5. Blood 97, 1920-1924), or CCR6 and CXCR6 (Loetscher, M., Amara, A., Oberlin, E., Brass, N., Legler, D., Loetscher, P., D'Apuzzo, M., Meese, E., Rousset, D., Virelizier, J. L., et al. (1997). TYMSTR, a putative chemokine receptor selectively expressed in activated T cells, exhibits HIV-1 coreceptor function. Curr Biol 7, 652-660) was assayed using 48-well Boyden chambers (Neuro Probe, Cabin John, MD) with polyvinylpyrrolidone-free polycarbonate membranes with pore size of 3 μm for primary human B cells and 5 μm for the other cell types, as previously described (Uguccioni, M., D'Apuzzo, M., Loetscher, M., Dewald, B., and Baggiolini, M. (1995). Actions of the chemotactic cytokines MCP-1, MCP-2, MCP-3, RANTES, MIP-1 alpha and MIP-1 beta on human monocytes. Eur J Immunol 25, 64-68). Briefly, 10⁵ primary human B cells or 5×10⁴ primary human monocytes and murine preB 300.19 cells were diluted in RPMI-1640 supplemented with 20 mM Hepes, pH7.4, and 1% pasteurized plasma protein solution (5% PPL SRK; Swiss Red Cross Laboratory, Bern, Switzerland). Cells were then added to the upper wells and the chemokine (with or without antibodies) to the bottom wells. After 120 min of incubation for primary human B cells and 90 min for the other cell types, the membrane was removed, washed on the upper side with phosphate-buffered saline (PBS), fixed, and stained with DiffQuik. All assays were done in triplicate, and for each well the migrated cells were counted at 100-fold magnification in 5 randomly selected high-power fields (5HPF).

Inhibition of chemotaxis by plasma purified IgGs (FIG. 2A): IgGs were purified from a subset of samples of the COVID-19 and uninfected control cohorts using Protein G Sepharose 4 Fast Flow (Cytiva) according to manufacturer's instructions (plasma:resuspended beads at a 5:4 [v/v] ratio), buffer-exchanged and concentrated in PBS by Amicon Ultra-4 centrifugal filters (30 kDa cutoff, Millipore). Chemotaxis was performed with preB 300.19 expressing CCR2, at a final IgG concentration of 200 μg/ml, in the presence of the chemokine concentration resulting in peak migration when no antibodies were added (CCL2 [10 nM], CCL7 [100 nM], CCL8 [100 nM]).

Inhibition of chemotaxis by monoclonal antibodies (FIGS. 1H, I. and 2F; FIGS. 13C, F and I): Experiments were performed with monoclonal antibodies at a final concentration of μg/ml (FIGS. 1H, I, and 2F) or 50 μg/ml (FIGS. 13C, F and I). Baseline migration was determined in the absence of chemoattractant (buffer control).

### ELISA

To evaluate the antibodies' binding to chemokine peptides, 96-well plates (ThermoFisher, 442404) were coated with 50 μl per well of a 2 μg/ml Neutravidin (Life Technologies, 31000) solution in PBS, overnight at room temperature. Plates were washed 4 times with washing buffer (PBS+0.05% Tween-20 [Sigma-Aldrich]) and incubated with 50 μl per well of a 50 nM biotinylated peptide solution in PBS for 1 h at room temperature. After washing 4 times with washing buffer, plates were incubated with 200 μl per well of blocking buffer (PBS+2% BSA+0.05% Tween-20) for 2 h at room temperature. Plates were then washed 4 times with washing buffer, and serial dilutions of monoclonal antibodies or plasma were added in PBS +0.05% Tween-20 and incubated for 1 h at room temperature. To screen for the presence of anti-chemokine IgGs, plasma samples were assayed (unless otherwise stated) at 1:50 starting dilution followed by 3 fourfold serial dilutions (1:200, 1:800, 1:3200). Monoclonal antibodies were tested at 5 μg/ml starting concentration followed by 11 threefold serial dilutions. Plates were subsequently washed 4 times with washing buffer and incubated with anti-human IgG secondary antibody conjugated to horseradish peroxidase (HRP) (GE Healthcare, NA933) at a 1:5000 dilution in PBS+0.05% Tween-20. Finally, after washing 4 times with washing buffer, plates were developed by the addition of 50 μl per well of the HRP substrate TMB (ThermoFisher, 34021) for 9 min. The developing reaction was stopped with 50 μl per well of a 1 M H₂SO₄ solution, and absorbance was measured at 450 nm with an ELISA microplate reader (BioTek) with Gen5 software. A positive control (broadly reactive plasma from donor CLM70) and a negative control (uninfected participant) samples were included in each experiment. The Area Under the Curve (AUC) was obtained from two independent experiments and plotted with GraphPad Prism. The main findings were further confirmed by assaying subsets of samples belonging to the different groups, side-by-side on the same plates (data not shown).

Reactivity at 6 versus 12 months (FIGS. 6B and C): Experiments were performed with the samples side-by-side on the same plate. In FIG. 6B, plasma samples were assayed at a 1:50 starting dilution, followed by 4 additional fivefold dilutions. Anti-RBD IgG levels are shown in COVID-19 convalescents, who have not received a COVID-19-mRNA vaccine between first and second visit (no vaccination) or in individuals with at least one dose of vaccine at least 10 days before blood sampling at the second visit (FIG. 6B).

Kinetic of signature anti-chemokine IgG antibodies (FIG. 6E): Experiments were performed with plasma samples assayed at a 1:50 dilution side-by-side on the same plate, and the average optical density at 450 nm obtained from two independent experiments was plotted with GraphPad Prism.

Antibodies' binding to SARS-CoV-2 RBD or S (FIGS. 3A, 6B, 9C, 2E, 5C, 5E and 8C): Experiments were performed with 96-well plates coated with 50 μl per well of a 5 μg/ml protein solution in PBS overnight at room temperature, and subsequently blocked and treated as described above. In this case, plasma samples were assayed at a 1:50 starting dilution, followed by 7 additional threefold serial dilutions.

### Single Cell Sorting by Flow Cytometry

B cells were enriched from PBMCs of COVID-19 convalescent individual CLM9 and from a uninfected control using the pan-B-cell isolation kit according to manufacturer's instructions (Miltenyi Biotec, 130-101-638). The enriched B cells were subsequently stained in FACS buffer (PBS+2% FCS+1 mM EDTA) with the following antibodies/reagents (all 1:200 diluted) for 30 min on ice: anti-CD20-PE-Cy7 (BD Biosciences, 335828), anti-CD14-APC-eFluor 780 (Thermo Fischer Scientific, 47-0149-42), anti-CD16-APC-eFluor 780 (Thermo Fischer Scientific, 47-0168-41), anti-CD3-APC-eFluor 780 (Thermo Fischer Scientific, 47-0037-41), anti-CD8-APC-eFluor 780 (Invitrogen, 47-0086-42), Zombie NIR (BioLegend, 423105), as well as fluorophore-labeled ovalbumin (Ova) and CCL8 peptides. Live single Zombie-NIR⁻CD14⁻CD16⁻CD3⁻CD8⁻CD20⁺Ova⁻CCL8-N-loop-PE⁻CCL8-N-loop-AF647⁻ B cells were single-cell sorted into 96-well plates containing 4 μl of lysis buffer (0.5× PBS, 10 mM DTT, 3,000 units/ml RNasin Ribonuclease Inhibitors [Promega, N2615]) per well using a FACS Aria Ill, and the analysis was performed with FlowJo software. The sorted cells were frozen on dry ice and stored at −80° C.

### Antibody Sequencing, Cloning, Production and Purification

Antibody genes were sequenced, cloned and expressed as previously reported (Robbiani, D. F., Bozzacco, L., Keeffe, J. R., Khouri, R., Olsen, P. C., Gazumyan, A., Schaefer-Babajew, D., Avila-Rios, S., Nogueira, L., Patel, R., et al. (2017). Recurrent Potent Human Neutralizing Antibodies to Zika Virus in Brazil and Mexico. Cell 169, 597-609 e511; Tiller, T., Meffre, E., Yurasov, S., Tsuiji, M., Nussenzweig, M. C., and Wardemann, H. (2008). Efficient generation of monoclonal antibodies from single human B cells by single cell RT-PCR and expression vector cloning. J Immunol Methods 329, 112-124; von Boehmer, L., Liu, C., Ackerman, S., Gitlin, A. D., Wang, Q., Gazumyan, A., and Nussenzweig, M. C. (2016). Sequencing and cloning of antigen-specific antibodies from mouse memory B cells. Nat Protoc 11, 1908-1923). Briefly, reverse-transcription of RNA from FACS-sorted single cells was performed to obtain cDNA, which was then used for amplification of the immunoglobulin IGH, IGK and IGL genes by nested PCR. Amplicons from this first PCR reaction served as templates for sequence and ligation independent cloning (SLIC) into human IgG1 antibody expression vectors. Monoclonal antibodies were produced by transiently transfecting Expi293F cells cultured in Freestyle-293 Expression Medium (ThermoFisher) with equal amounts of immunoglobulin heavy and light chain expression vectors using polyethylenimine Max (PEI-MAX, Polysciences) as a transfection reagent. After 6-7 days of culture, cell supernatants were filtered through 0.22 μm Millex-GP filters (Merck Millipore), and antibodies were purified using Protein G Sepharose 4 Fast Flow (Cytiva) according to manufacturer's instructions and buffer-exchanged and concentrated in PBS by Amicon Ultra-4 centrifugal filters (30 kDa cutoff, Millipore). Where indicated, the anti-Zika virus monoclonal antibody Z021 (Robbiani, D. F., Bozzacco, L., Keeffe, J. R., Khouri, R., Olsen, P. C., Gazumyan, A., Schaefer-Babajew, D., Avila-Rios, S., Nogueira, L., Patel, R., et al. (2017). Recurrent Potent Human Neutralizing Antibodies to Zika Virus in Brazil and Mexico. Cell 169, 597-609 e511) was used as an isotype control.

### Computational Analysis of Antibody Sequences

Antibody sequences were analyzed using a collection of Perl and R scripts provided by IgPipeline and publicly available on GitHub (https://github.com/stratust/igpipeline) (Robbiani, D. F., Gaebler, C., Muecksch, F., Lorenzi, J. C. C., Wang, Z., Cho, A., Agudelo, M., Barnes, C. O., Gazumyan, A., Finkin, S., et al. (2020). Convergent antibody responses to SARS-CoV-2 in convalescent individuals. Nature 584, 437-442). In brief, sequences where annotated using IgBlast (Ye, J., Ma, N., Madden, T. L., and Ostell, J. M. (2013). IgBLAST: an immunoglobulin variable domain sequence analysis tool. Nucleic Acids Res 41, W34-40) v 1.14.0 with IMGT domain delineation system and the Change-O toolkit v 0.4.5 (Gupta, N. T., Vander Heiden, J. A., Uduman, M., Gadala-Maria, D., Yaari, G., and Kleinstein, S. H. (2015). Change-O: a toolkit for analyzing large-scale B cell immunoglobulin repertoire sequencing data. Bioinformatics 31, 3356-3358). Nucleotide somatic hypermutation and CDR3 were determined by aligning the IGHV and IGLV nucleotide sequence against their closest germlines using the blastn function of IgBlast. Differences outside CDR3 were considered as mutations.

t-SNE and Clustering

t-SNE analysis was performed using the Rtsne R package v 0.15 using the AUC values for all chemokines. The theta parameter for the accuracy of the mapping was set to zero in all cases for exact TSNE. Hierarchical clustering was created using the hclust R function v 4.1.1. Clustering analysis was performed using the correlation as distance and WPGMA (Weighted Pair Group Method with Arithmetic Mean) as agglomeration method. Heatmaps were created with either GraphPad Prism (FIGS. 3A and 5B) or the Pretty Heatmaps (pheatmap) R package v 1.0.12 (FIG. 11D). In FIG. 11D, each column containing a distinct chemokine was scaled with the scaling function provided by R, which sets the mean and the standard deviation to 0 and 1, respectively.

### Logistic Regression

Logistic regression was performed using the glm (generalized linear models) function provided by the R package v 4.1.1. To identify which variables to include in the analysis, AUCs were ranked according to the p-value obtained with a Mann-Whitney parametric test. The first N variables minimizing the AIC (Akaike information criterion) were then used in the fitting. In each plot, values from 0 to 0.5 and from 0.5 to 1 on the y-axis represent the assignment of individuals to the A and B groups (of a Prediction A versus B; see grey backgrounds), respectively. On the x axis, samples are divided into the two groups and subsequently ordered according to sample ID. Dots in the grey area represent individuals that are assigned to the correct group.

### SARS-CoV-2 Pseudotyped Reporter Virus and Neutralization Assay

To generate (HIV-1/NanoLuc2AEGFP)-SARS-CoV-2 particles, HEK293T cells were co-transfected with the three plasmids pHIV_{N}LGagPol, pCCNanoLuc2AEGFP, and SARS-CoV-2 S as described elsewhere (Robbiani, D. F., Gaebler, C., Muecksch, F., Lorenzi, J. C. C., Wang, Z., Cho, A., Agudelo, M., Barnes, C. O., Gazumyan, A., Finkin, S., et al. (2020). Convergent antibody responses to SARS-CoV-2 in convalescent individuals. Nature 584, 437-442; Schmidt, F., Weisblum, Y., Muecksch, F., Hoffmann, H. H., Michailidis, E., Lorenzi, J. C. C., Mendoza, P., Rutkowska, M., Bednarski, E., Gaebler, C., et al. (2020). Measuring SARS-CoV-2 neutralizing antibody activity using pseudotyped and chimeric viruses. J Exp Med 217). Supernatants containing virions were collected 48 h after transfection, and virion infectivity was determined by titration on 293T_{ACE2} cells. The plasma neutralizing activity was measured as previously reported (Robbiani et al., 2020; Schmidt et al., 2020). Briefly, threefold serially diluted plasma samples (from 1:50 to 1:328′050) were incubated with SARS-CoV-2 pseudotyped virus for 1 h at 37° C., and the virus-plasma mixture was subsequently incubated with 293T_{ACE2} cells for 48 h. Cells were then washed with PBS and lysed with Luciferase Cell Culture Lysis 5× reagent (Promega). Nanoluc Luciferase activity in cell lysates was measured using the Nano-Glo Luciferase Assay System (Promega) with Modulus II Microplate Reader User interface (TURNER BioSystems). The obtained relative luminescence units were normalized to those derived from cells infected with SARS-CoV-2 pseudotyped virus in the absence of plasma. The NT₅₀ values were determined using four-parameter nonlinear regression with bottom and top constrains equal to 0 and 1, respectively (GraphPad Prism). The dotted line (NT₅₀=5) in the plots represents the lower limit of detection of the assay.

### Model Interaction Between Chemokine and Chemokine Receptor

The illustrative model in FIG. 1 was generated from the structure of inactive CCR2 (PDB code: 5T1A) (Zheng, Y., Qin, L., Zacarias, N. V., de Vries, H., Han, G. W., Gustavsson, M., Dabros, M., Zhao, C., Cherney, R. J., Carter, P., et al. (2016). Structure of CC chemokine receptor 2 with orthosteric and allosteric antagonists. Nature 540, 458-461), together with the electron microscopy structures of CCR5 and CCR6 (PDB codes: 6MEO and 6WWZ, respectively (Shaik, M. M., Peng, H., Lu, J., Rits-Volloch, S., Xu, C., Liao, M., and Chen, B. (2019). Structural basis of coreceptor recognition by HIV-1 envelope spike. Nature 565, 318-323; Wasilko, D. J., Johnson, Z. L., Ammirati, M., Che, Y., Griffor, M. C., Han, S., and Wu, H. (2020). Structural basis for chemokine receptor CCR6 activation by the endogenous protein ligand CCL20. Nat Commun 11, 3031) by using SWISS-MODEL (Waterhouse, A., Bertoni, M., Bienert, S., Studer, G., Tauriello, G., Gumienny, R., Heer, F. T., de Beer, T. A. P., Rempfer, C., Bordoli, L., et al. (2018). SWISS-MODEL: homology modelling of protein structures and complexes. Nucleic Acids Res 46, W296-W303) server and the molecular graphics program PyMOL 2.5.0 for modeling the N- and C-terminus of the receptor. The crystal structure of CCL8 (MCP-2) (PDB code: 1 ESR) (Blaszczyk, J., Coillie, E. V., Proost, P., Damme, J. V., Opdenakker, G., Bujacz, G. D., Wang, J. M., and Ji, X. (2000). Complete crystal structure of monocyte chemotactic protein-2, a CC chemokine that interacts with multiple receptors. Biochemistry 39, 14075-14081), and the electron microscopy structure of CCR6 (Wasilko, D. J., Johnson, Z. L., Ammirati, M., Che, Y., Griffor, M. C., Han, S., and Wu, H. (2020). Structural basis for chemokine receptor CCR6 activation by the endogenous protein ligand CCL20. Nat Commun 11, 3031) were used to model the complex. The intracellular residues were removed for clarity.

### Statistical Analysis

Statistical significance between two groups was determined using non-parametric two-tailed Mann-Whitney U-tests, or Wilcoxon signed-rank test, for unpaired or paired samples, respectively. Upon testing of parametric assumptions, statistical significance between more than two groups was evaluated using Kruskal-Wallis test (followed by Dunn multiple comparisons), one-way ANOVA (followed by Tukey multiple comparisons), or two-way Repeated Measures ANOVA (followed by Ŝidák multiple comparisons), as described in the figure legends. Statistical significance of the signature chemokines (CCL19, CCL22, CXCL17, CXCL8, CCL25, CXCL5, CCL21, CXCL13 and CXCL16) was also confirmed by applying the Bonferroni criterion in order to guarantee a familywise level of significance equal to 0.05. Statistical significance from a 2×2 contingency table was determined with Fisher's exact test. Correlations were assessed using Pearson correlation analysis. A p-value of less than 0.05 was considered statistically significant. In the figures, significance is shown as follow: ns p≥0.05 (not significant), *p<0.05, **p<0.01, ***p<0.001 and ****p<0.0001. Data and statistical analyses were performed with GraphPad Prism.

### Example 1: CCL8-Blocking Antibodies from COVID-19 Convalescent Individuals

To determine whether SARS-CoV-2 infection induces antibodies that interfere with immune cell migration, cells expressing the CC chemokine receptor 2 (CCR2), a key mediator of monocyte chemotaxis were analyzed. In vitro chemotaxis assays were performed with three CCR2 agonists (CCL2, CCL7 and CCL8) in the presence of IgGs purified from the plasma of COVID-19 convalescent individuals or uninfected controls (n=24 and n=8, respectively; see Methods). Cell migration to CCL8 was significantly impaired (26.0% reduction; p=0.0039), suggesting the presence of antibodies specific to CCL8 following COVID-19 (FIG. 2A).

To corroborate this possibility, a larger COVID-19 convalescent cohort that includes previously hospitalized (n=50) and non-hospitalized (outpatient) individuals (n=21), of which a fraction went on to develop long term symptoms (FIG. 1A), was evaluated. Without being bound to any theory, it was assumed that antibodies impacting cell migration would likely target the N-terminal loop (N-loop) of the chemokine, because this region is required for receptor binding (FIG. 1B). Therefore, a peptide comprising the N-loop of CCL8 was synthesized (SEQ ID NO: 7) for use in enzyme-linked immunosorbent assays (ELISA). Analysis of serial plasma dilutions obtained in average at 6 months (t=6 m) after disease onset revealed high levels of anti-CCL8 IgGs in two out of 71 COVID-19 convalescents (FIG. 1C). This was similar to the analysis of antibodies against Interferon a2 (IFNα2), which showed a single strongly reactive sample (FIG. 2B).

To examine the molecular features of anti-CCL8 antibodies, peripheral blood mononuclear cells were obtained from the donor with the highest reactivity in ELISA (CLM9) and memory B cells that bound to the CCL8 N-loop were isolated for antibody sequencing (FIGS. 1D and 2C; see Methods). Out of 23 IgG sequences, 20 were clonally related VH4-39/VK4-1 antibodies with variable numbers of V gene somatic mutations (FIGS. 1E and F). Exemplified antibodies binding to CCL8 with their CDR and VH/VL sequences are the antibodies described in Table 2 above

Four VH4-39/VK4-1 antibodies were cloned and recombinantly expressed (aCCL8.001, aCCL8.003, aCCL8.004, aCCL8.005; see Table 2 above). All of them similarly bound to the CCL8 N-loop with half-maximal effective concentrations (EC₅₀) between 11-16 ng/mL (FIG. 1G).

Anti-CCL8 antibodies were specific, because they failed to bind to the most similar chemokine N-loops or to the Spike protein of SARS-CoV-2 (FIGS. 2D and E). Thus, antibodies to CCL8 can be found after COVID-19.

To evaluate whether anti-CCL8 antibodies are biologically active, chemotaxis assays with primary human monocytes were performed. All four antibodies inhibited cell migration towards CCL8 (FIGS. 1H and 2F). Since blood-derived classical monocytes express two CCL8 receptors (CCR1 and CCR2), to determine whether the blocking effect is receptor-specific, migration of cells expressing either receptor alone was measured (see Methods). Antibodies aCCL8.001 and aCCL8.005 inhibited CCR1—but not CCR2-mediated chemotaxis (FIG. 1I). This indicates that some COVID-19 convalescents have biologically active antibodies targeting CCL8, which interfere with immune cell migration in a receptor-specific manner.

### Example 2: Antibodies Against Chemokines after COVID-19

Having validated the CCL8 N-loop as target of antibodies that interfere with cell migration, peptides corresponding to the N-loop of all known human chemokines were designed, with the goal to comprehensively examine anti-chemokine antibodies in COVID-19 (FIG. 3). The following peptides corresponding to the N-loop of all known human chemokines were designed:

TABLE 7

| | | SEQ |
|---|---|---|
| Chemokine | Sequence N-loop peptides | ID NO. |
| CCL1 | GGKFSFAEQEIPLRAILCYRNT | 1 |
| CCL2 | GGKYNFTNRKISVQRLASYRRIT | 2 |
| CCL3 | GGSFSYTSRQIPQNFIADYFETSSQ | 3 |
| CCL4 | GGSFSYTARKLPRNFVVDYYETSSL | 4 |
| CCL5 | GGSFAYIARPLPRAHIKEYFYTSGK | 5 |
| CCL7 | GGSYRFINKKIPKQRLESYRRTTSS | 6 |
| CCL8 | GGSFNVINRKIPIQRLESYTRITNI | 7 |
| CCL11 | GGSFNLANRKIPLQRLESYRRITSG | 8 |
| CCL13 | GGSFTFSSKKISLQRLKSYVITTSR | 9 |
| CCL14 | GGSFTYTTYKIPRQRIMDYYETNSQ | 10 |
| CCL15 | GGSTSYISQSIPCSLMKSYFETSSE | 11 |
| CCL16 | GGGSLKYYEKVLPRRLVVGYRKALN | 12 |
| CCL17 | GGSLEYFKGAIPLRKLKTWYQTSED | 13 |
| CCL18 | GGSLVYTSWQIPQKFIVDYSETSPQ | 14 |
| CCL19 | GGSLSVTQKPIPGYIVRNFHYLLIK | 15 |
| CCL20 | GGSLGYTDRILHPKFIVGFTRQLAN | 16 |
| CCL21 | GGSLKYSQRKIPAKVVRSYRKQEPS | 17 |
| CCL22 | GGSRDYVRYRLPLRVVKHFYWTSDS | 18 |
| CCL23 | GGSISYTPRSIPCSLLESYFETNSE | 19 |
| CCL24 | GGSMFFVSKRIPENRVVSYQLSSRS | 20 |
| CCL25 | GGSLAYHYPIGWAVLRRAWTYRIQE | 21 |
| CCL26 | GGSFQYSHKPLPWTWVRSYEFTSNS | 22 |
| CCL27 | GGSTQLYRKPLSDKLLRKVIQVELQ | 23 |
| CCL28 | GGSTEVSHHISRRLLERVNMCRIQR | 24 |
| CXCL1 | GGSLQTLQGIHPKNIQSVNVKSPGP | 25 |
| CXCL2 | GGSLQTLQGIHLKNIQSVKVKSPGP | 26 |
| CXCL3 | GSLQTLQGIHLKNIQSVNVRSPGPH | 27 |
| CXCL4 | GGSVKTTSQVRPRHITSLEVIKAGP | 28 |
| CXCL5 | GGSLQTTQGVHPKMISNLQVFAIGP | 29 |
| CXCL6 | GGSLRVTLRVNPKTIGKLQVFPAGP | 30 |
| CXCL7 | GGSIKTTSGIHPKNIQSLEVIGKGT | 31 |
| CXCL8 | GGSIKTYSKPFHPKFIKELRVIESG | 32 |
| CXCL9 | GGSISTNQGTIHLQSLKDLKQFAPS | 33 |
| CXCL10 | GGSISISNQPVNPRSLEKLEIIPAS | 34 |
| CXCL11 | GGSIGPGVKAVKVADIEKASIMYPS | 35 |
| CXCL12 | GGSRFFESHVARANVKHLKILNTPN | 36 |
| CXCL13 | GGSVQESSVFIPRRFIDRIQILPRG | 37 |
| CXCL14 | GGSSRKGPKIRYSDVKKLEMKPKYP | 38 |
| CXCL16 | GGSGKRISSDSPPSVQFMNRLRKHL | 39 |
| CXCL17 | GGSKDWFLRAPRRKFMTVSGLPKKQ | 40 |
| XCL1/XCL2 | GGSVSLTTQRLPVSRIKTYTITEGS | 41 |
| CX3CL1 | GGGSSKMTSKIPVALLIHYQQNQAS | 42 |

For using the peptides in an ELISA, the peptides were prepared as described above, in particular biotinylated at their N-terminus. Specifically, the following biotinylated peptides were used in an ELISA:

TABLE 8

| | | SEQ ID NO. of |
|---|---|---|
| | | peptide (without |
| Chemokine | N-loop peptide | biotin) |
| CCL1 | Biotin-Ahx-GGKFSFAEQEIPLRAILCYRNT | 1 |
| CCL2 | Biotin-Ahx-GGKYNFTNRKISVQRLASYRRIT | 2 |
| CCL3 | Biotin-Ahx-GGSFSYTSRQIPQNFIADYFETSSQ | 3 |
| CCL4 | Biotin-Ahx-GGSFSYTARKLPRNFVVDYYETSSL | 4 |
| CCL5 | Biotin-Ahx-GGSFAYIARPLPRAHIKEYFYTSGK | 5 |
| CCL7 | Biotin-Ahx-GGSYRFINKKIPKQRLESYRRTTSS | 6 |
| CCL8 | Biotin-Ahx-GGSFNVINRKIPIQRLESYTRITNI | 7 |
| CCL11 | Biotin-Ahx-GGSFNLANRKIPLQRLESYRRITSG | 8 |
| CCL13 | Biotin-Ahx-GGSFTFSSKKISLQRLKSYVITTSR | 9 |
| CCL14 | Biotin-Ahx-GGSFTYTTYKIPRQRIMDYYETNSQ | 10 |
| CCL15 | Biotin-Ahx-GGSTSYISQSIPCSLMKSYFETSSE | 11 |
| CCL16 | Biotin-Ahx-GGGSLKYYEKVLPRRLVVGYRKALN | 12 |
| CCL17 | Biotin-Ahx-GGSLEYFKGAIPLRKLKTWYQTSED | 13 |
| CCL18 | Biotin-Ahx-GGSLVYTSWQIPQKFIVDYSETSPQ | 14 |
| CCL19 | Biotin-Ahx-GGSLSVTQKPIPGYIVRNFHYLLIK | 15 |
| CCL20 | Biotin-Ahx-GGSLGYTDRILHPKFIVGFTRQLAN | 16 |
| CCL21 | Biotin-Ahx-GGSLKYSQRKIPAKVVRSYRKQEPS | 17 |
| CCL22 | Biotin-Ahx-GGSRDYVRYRLPLRVVKHFYWTSDS | 18 |
| CCL23 | Biotin-Ahx-GGSISYTPRSIPCSLLESYFETNSE | 19 |
| CCL24 | Biotin-Ahx-GGSMFFVSKRIPENRVVSYQLSSRS | 20 |
| CCL25 | Biotin-Ahx-GGSLAYHYPIGWAVLRRAWTYRIQE | 21 |
| CCL26 | Biotin-Ahx-GGSFQYSHKPLPWTWVRSYEFTSNS | 22 |
| CCL27 | Biotin-Ahx-GGSTQLYRKPLSDKLLRKVIQVELQ | 23 |
| CCL28 | Biotin-Ahx-GGSTEVSHHISRRLLERVNMCRIQR | 24 |
| CXCL1 | Biotin-Ahx-GGSLQTLQGIHPKNIQSVNVKSPGP | 25 |
| CXCL2 | Biotin-Ahx-GGSLQTLQGIHLKNIQSVKVKSPGP | 26 |
| CXCL3 | Biotin-Ahx-GSLQTLQGIHLKNIQSVNVRSPGPH | 27 |
| CXCL4 | Biotin-Ahx-GGSVKTTSQVRPRHITSLEVIKAGP | 28 |
| CXCL5 | Biotin-Ahx-GGSLQTTQGVHPKMISNLQVFAIGP | 29 |
| CXCL6 | Biotin-Ahx-GGSLRVTLRVNPKTIGKLQVFPAGP | 30 |
| CXCL7 | Biotin-Ahx-GGSIKTTSGIHPKNIQSLEVIGKGT | 31 |
| CXCL8 | Biotin-Ahx-GGSIKTYSKPFHPKFIKELRVIESG | 32 |
| CXCL9 | Biotin-Ahx-GGSISTNQGTIHLQSLKDLKQFAPS | 33 |
| CXCL10 | Biotin-Ahx-GGSISISNQPVNPRSLEKLEIIPAS | 34 |
| CXCL11 | Biotin-Ahx-GGSIGPGVKAVKVADIEKASIMYPS | 35 |
| CXCL12 | Biotin-Ahx-GGSRFFESHVARANVKHLKILNTPN | 36 |
| CXCL13 | Biotin-Ahx-GGSVQESSVFIPRRFIDRIQILPRG | 37 |
| CXCL14 | Biotin-Ahx-GGSSRKGPKIRYSDVKKLEMKPKYP | 38 |
| CXCL16 | Biotin-Ahx-GGSGKRISSDSPPSVQFMNRLRKHL | 39 |
| CXCL17 | Biotin-Ahx-GGSKDWFLRAPRRKFMTVSGLPKKQ | 40 |
| XCL1/XCL2 | Biotin-Ahx-GGSVSLTTQRLPVSRIKTYTITEGS | 41 |
| CX3CL1 | Biotin-Ahx-GGGSSKMTSKIPVALLIHYQQNQAS | 42 |

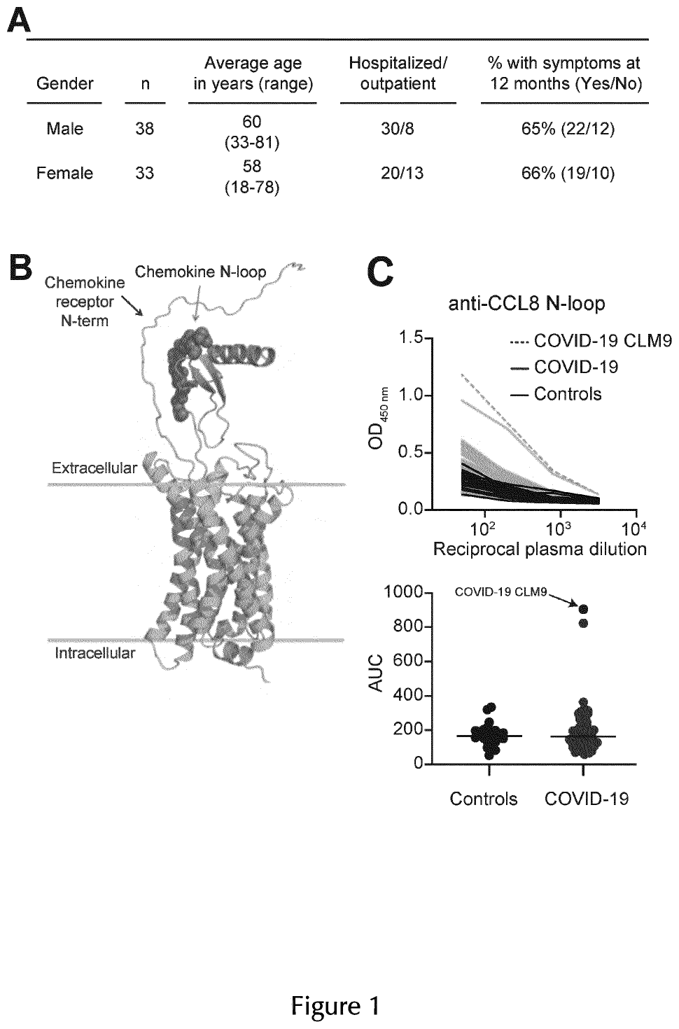

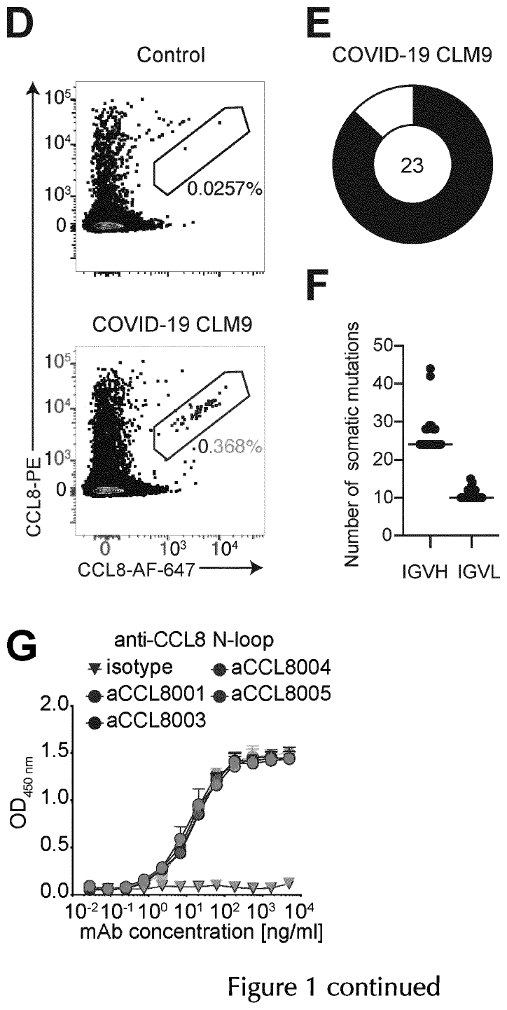

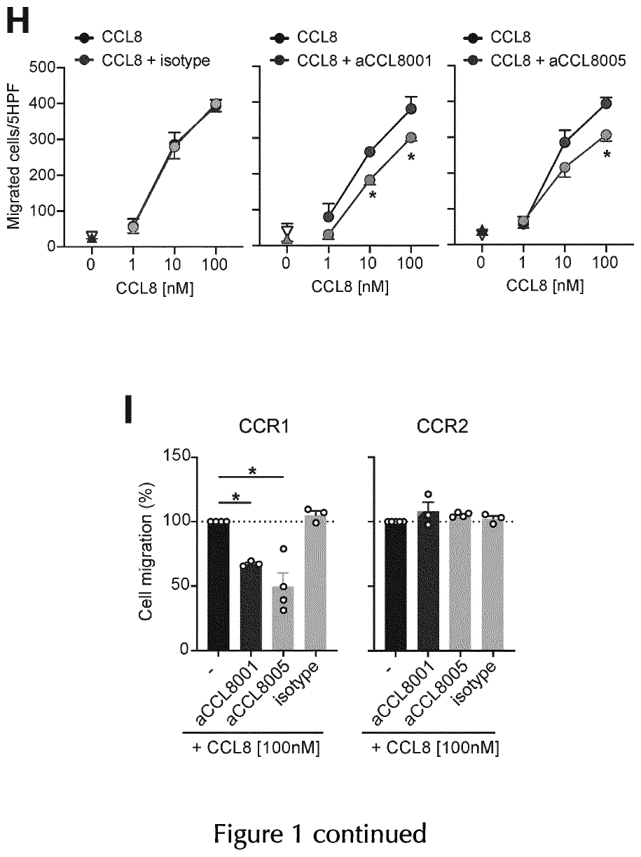

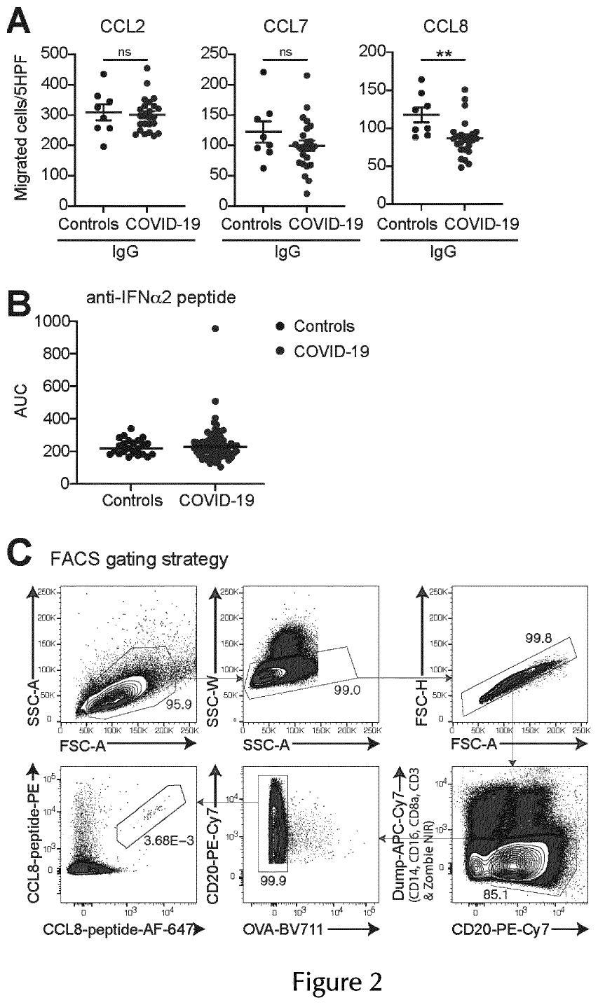

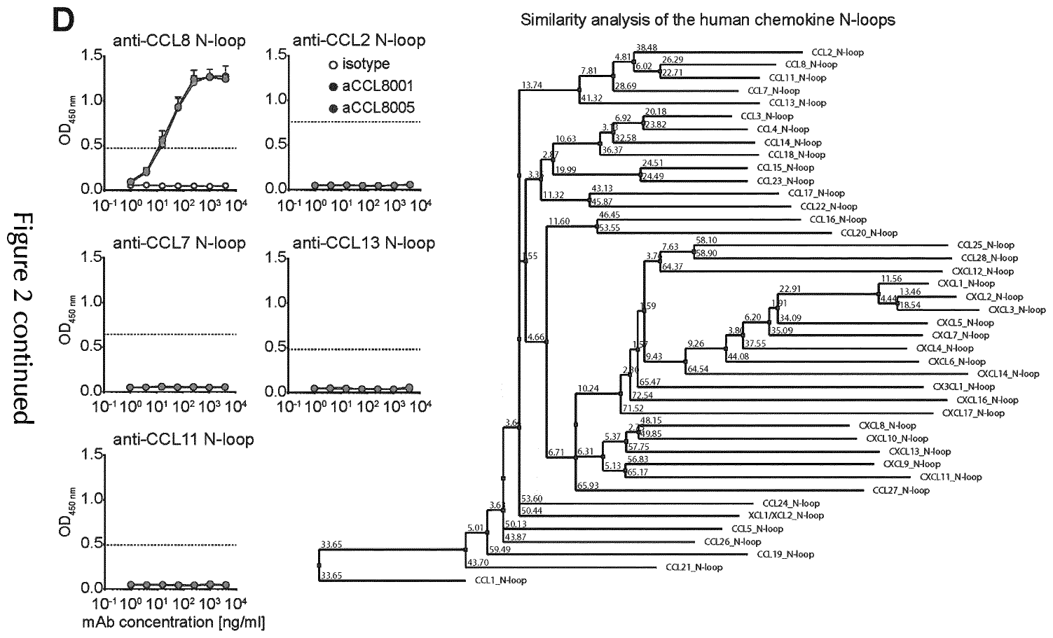

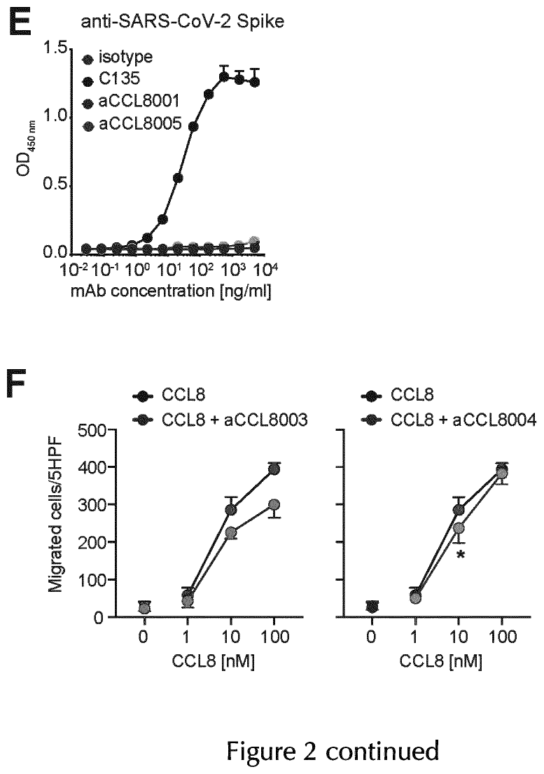

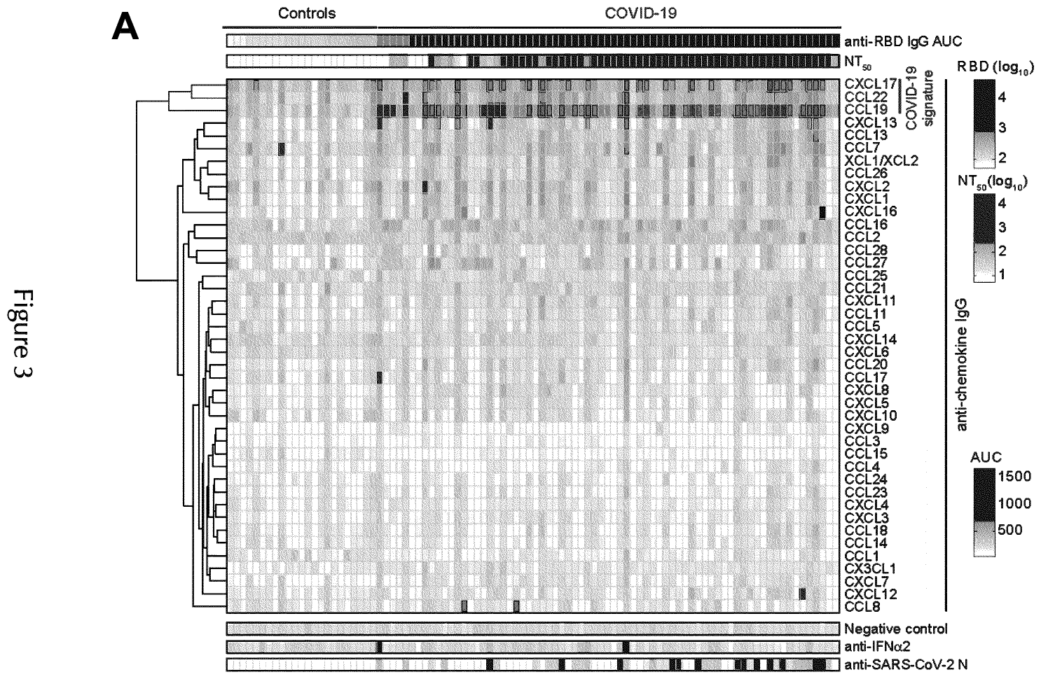

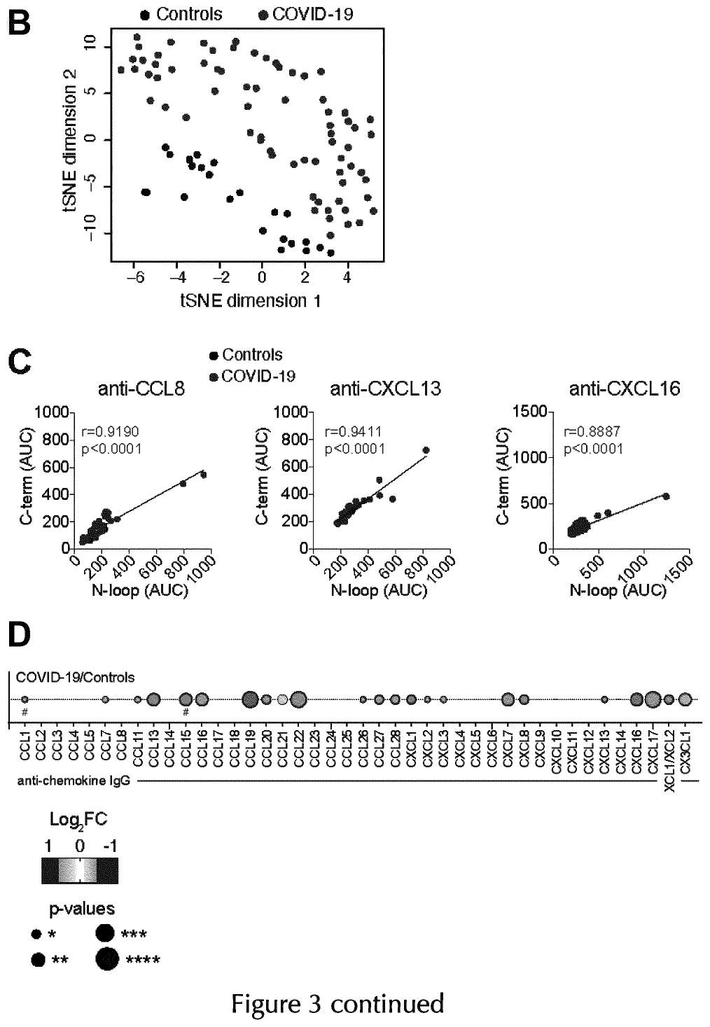

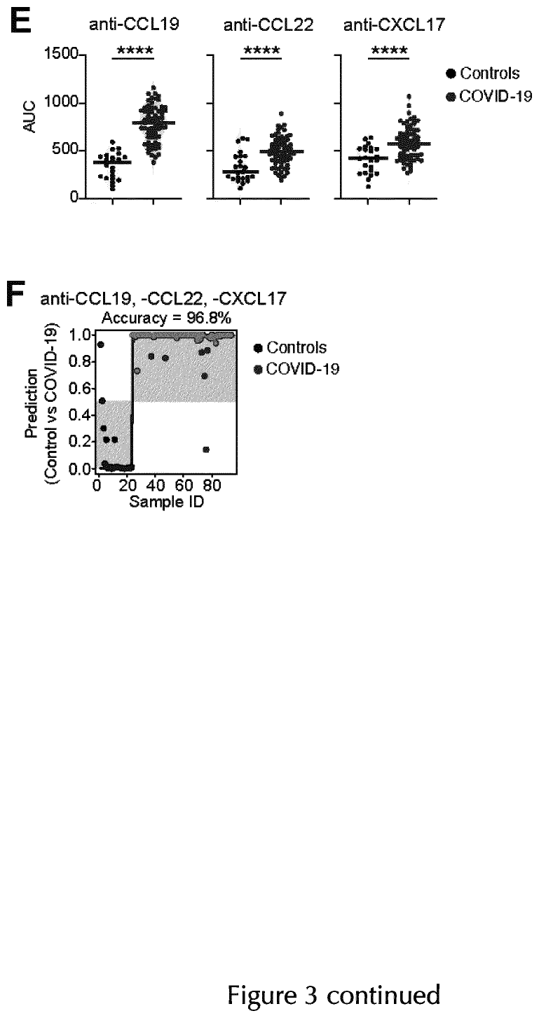

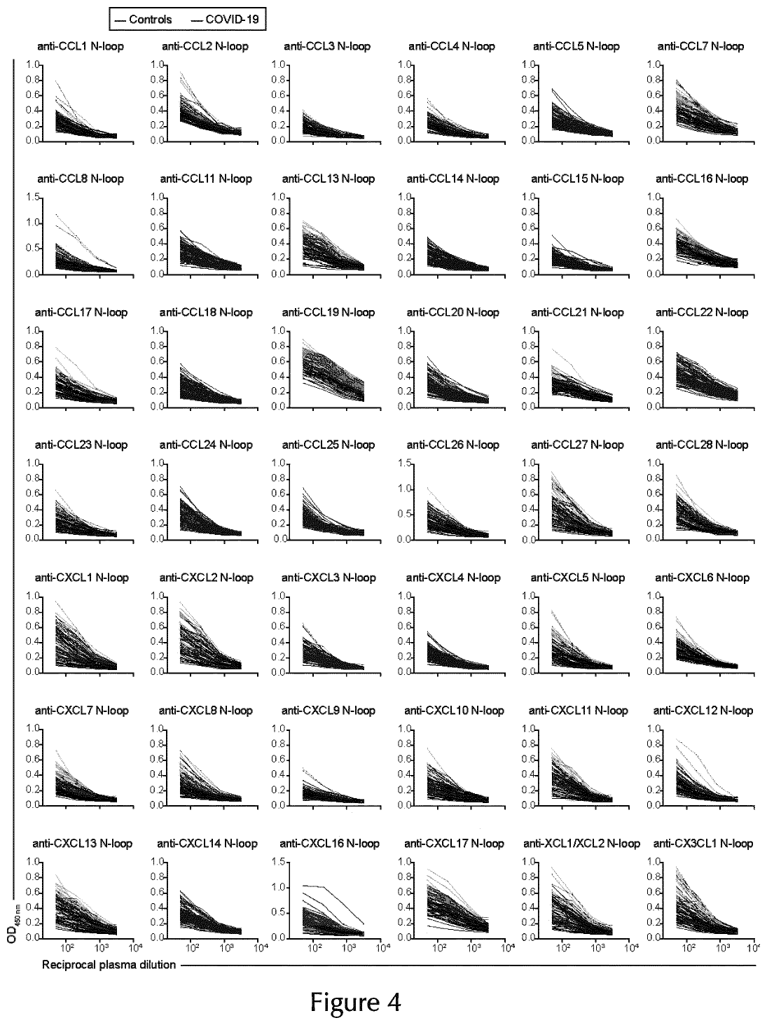

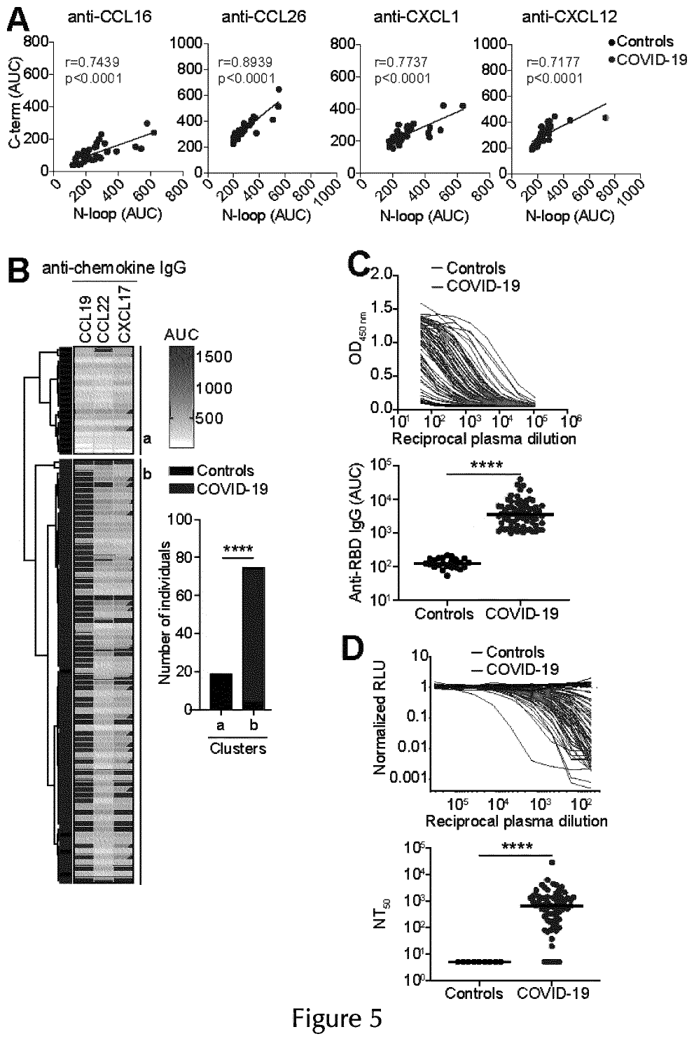

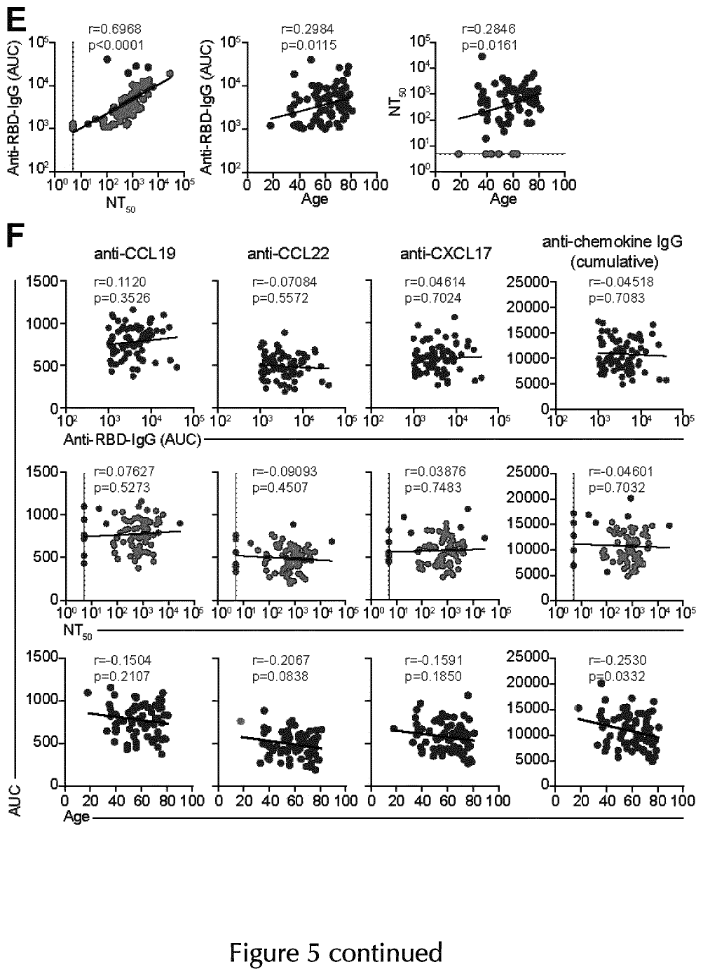

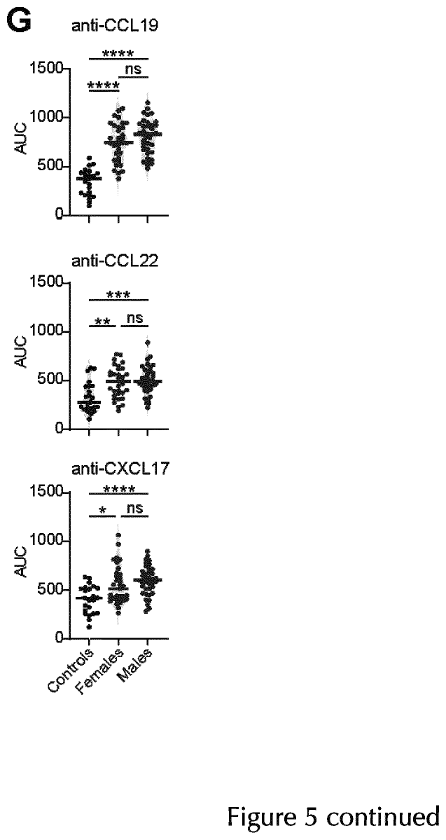

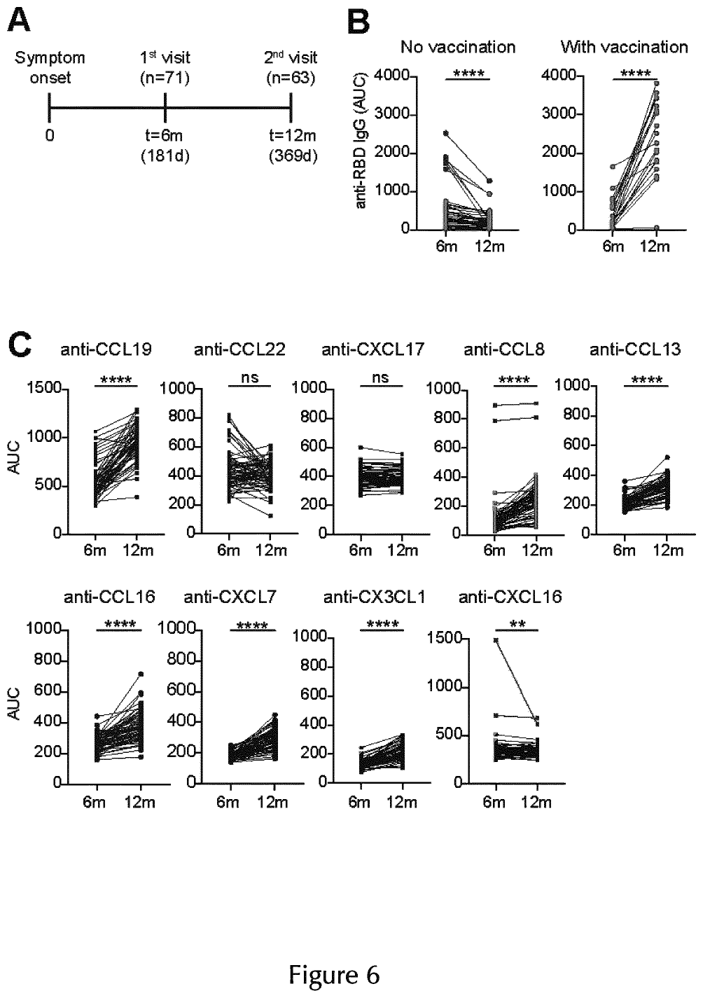

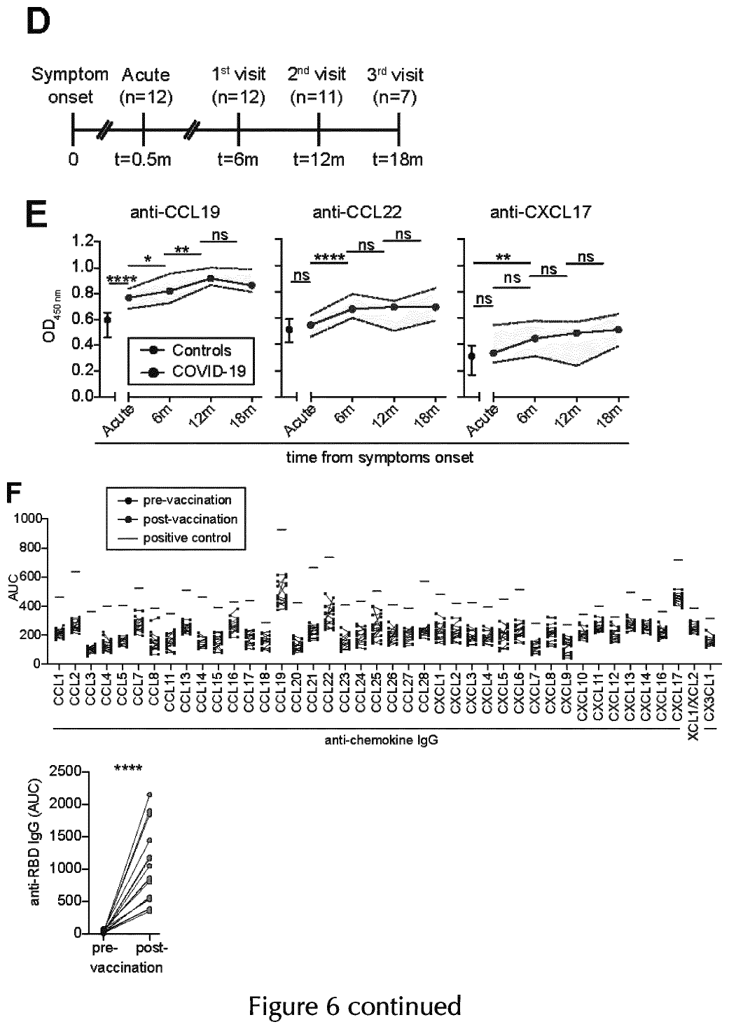

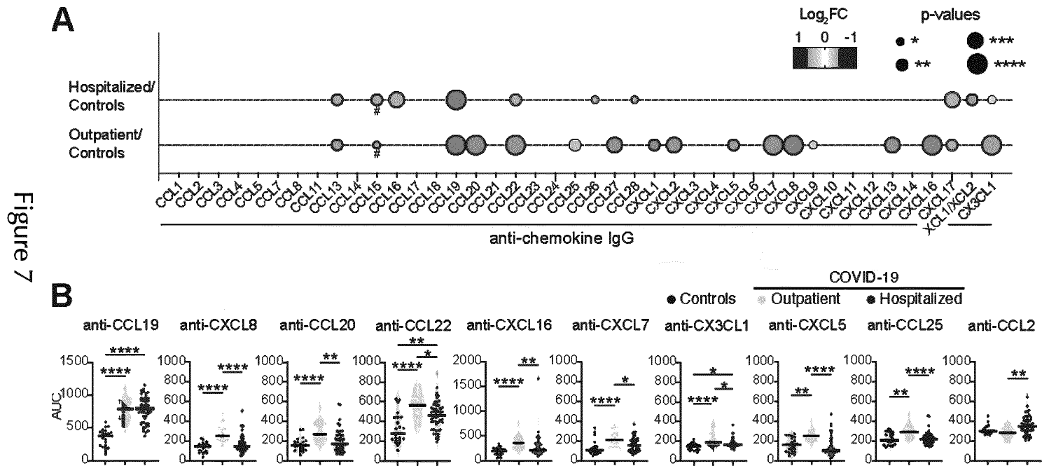

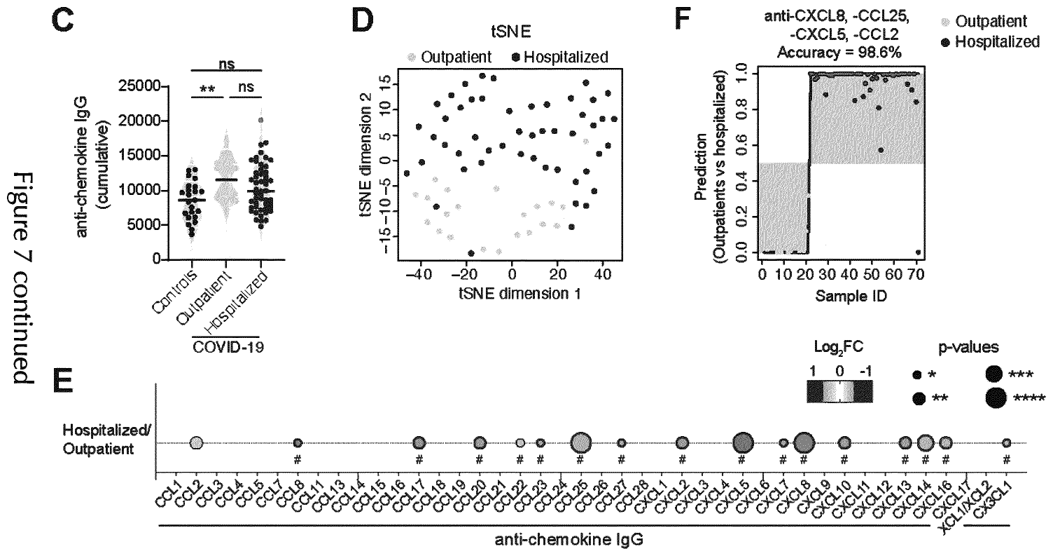

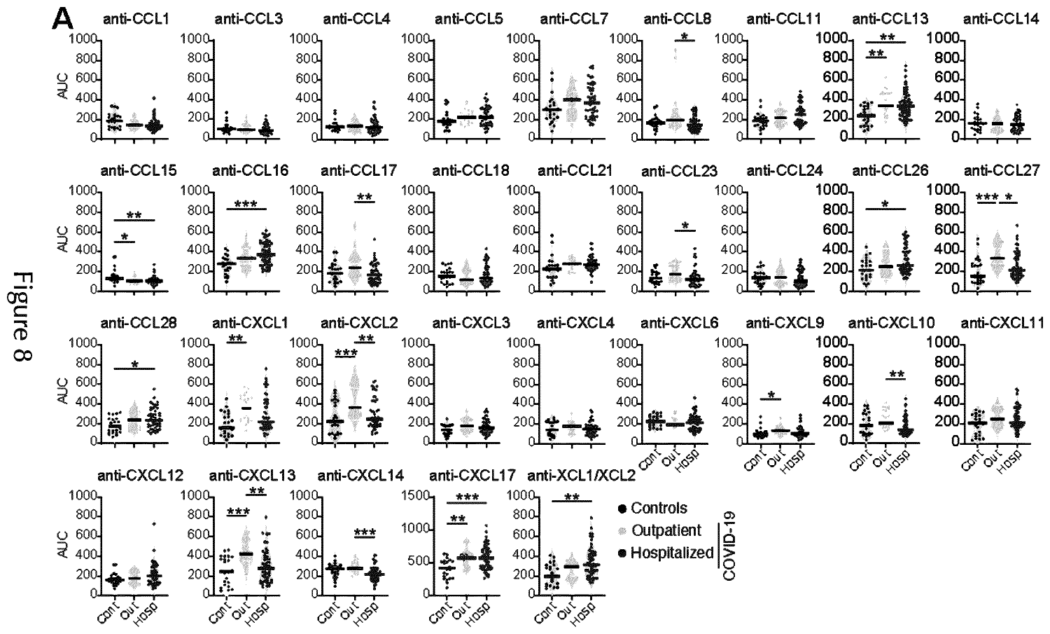

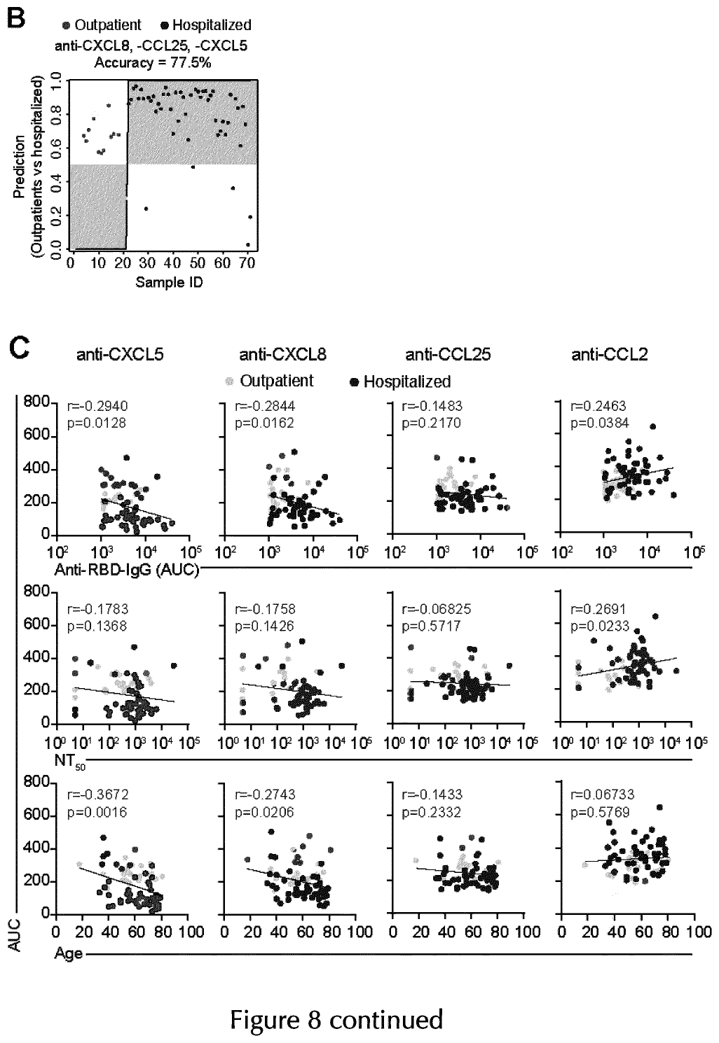

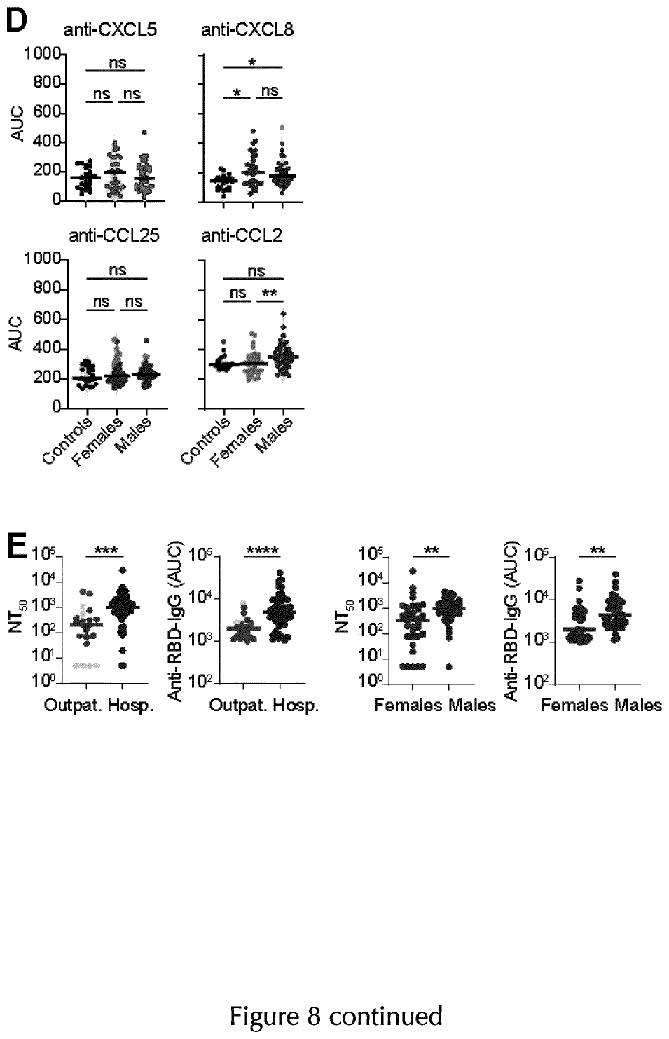

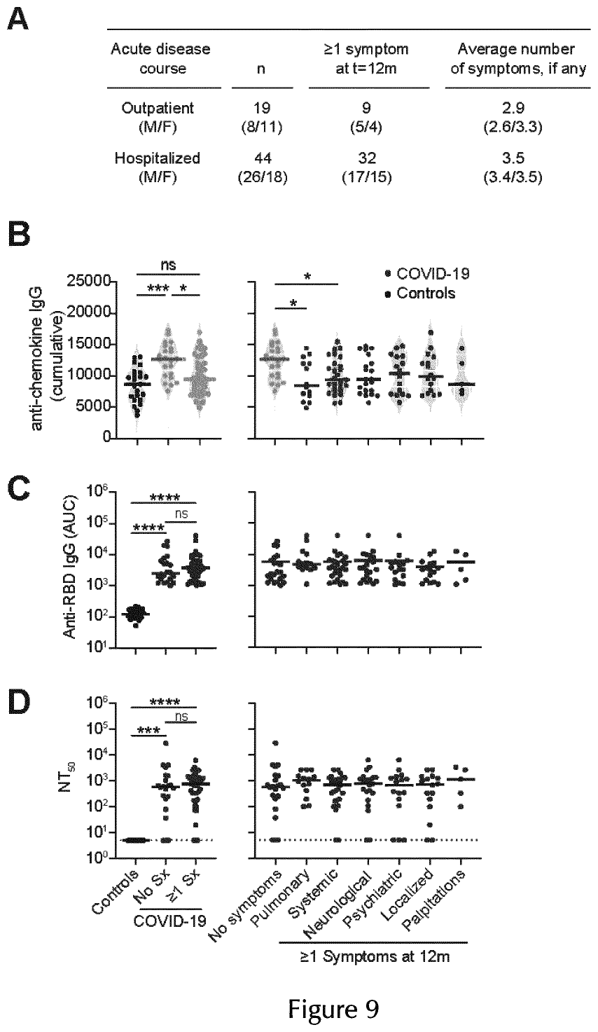

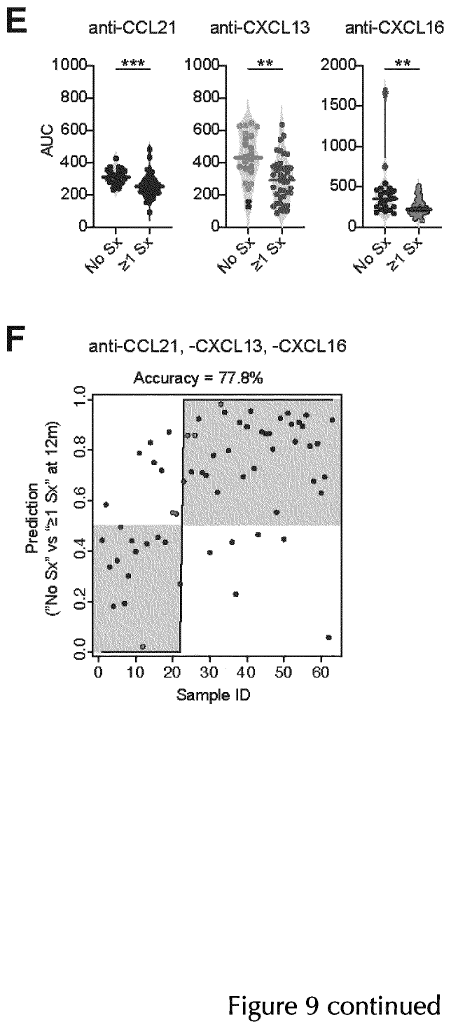

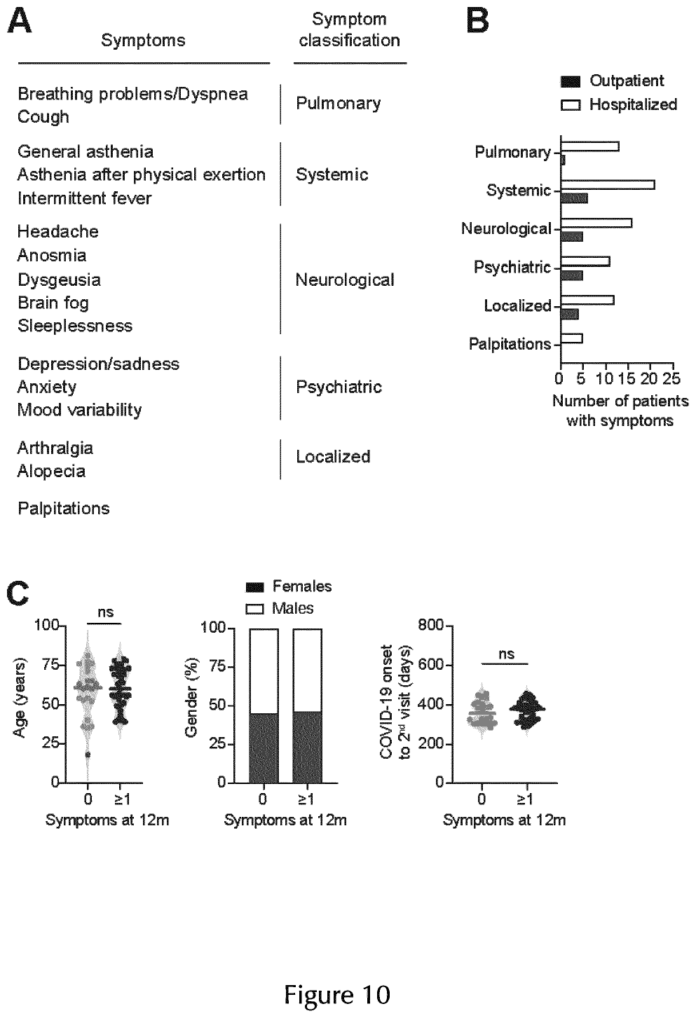

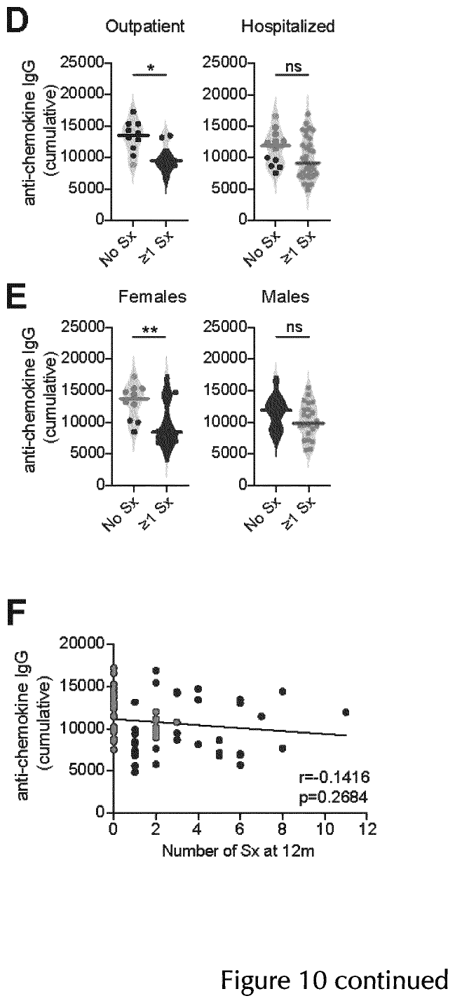

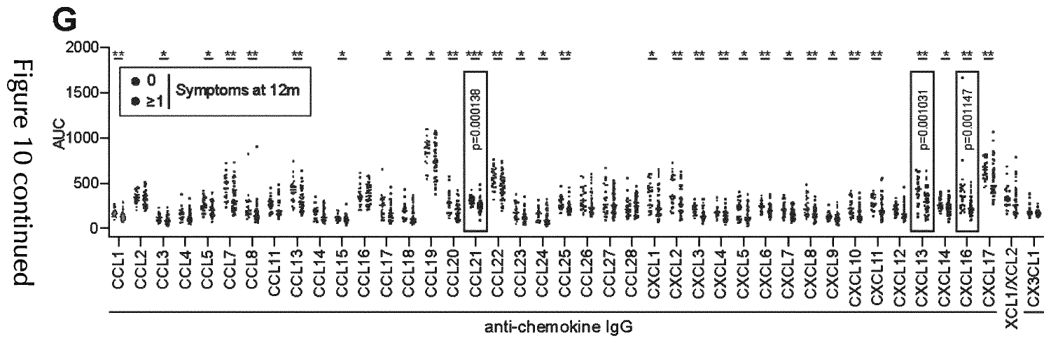

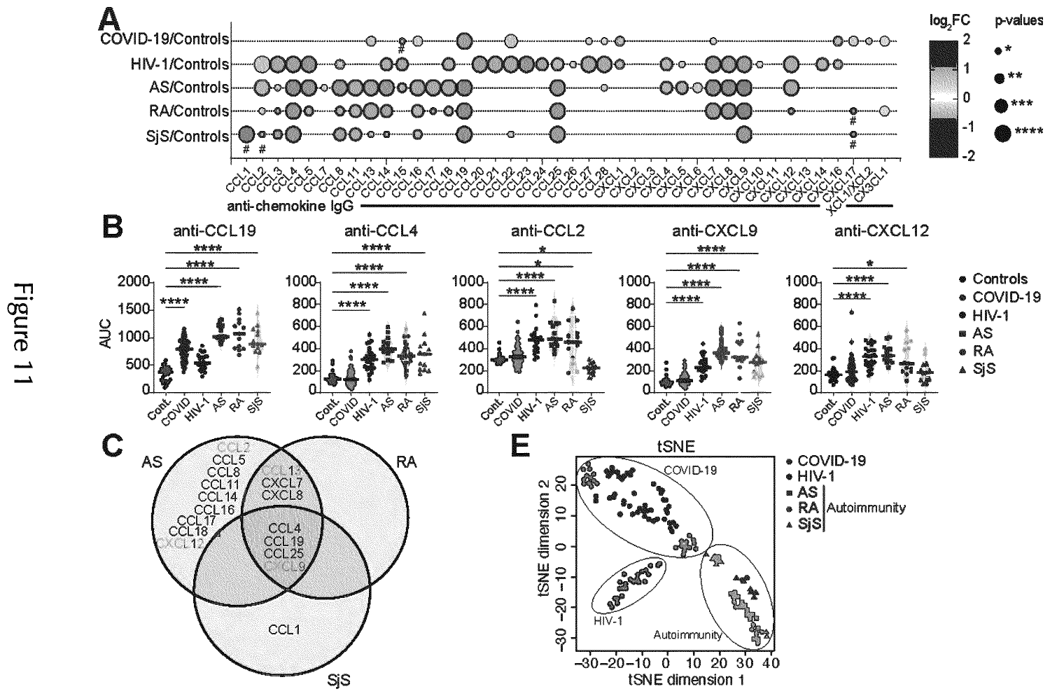

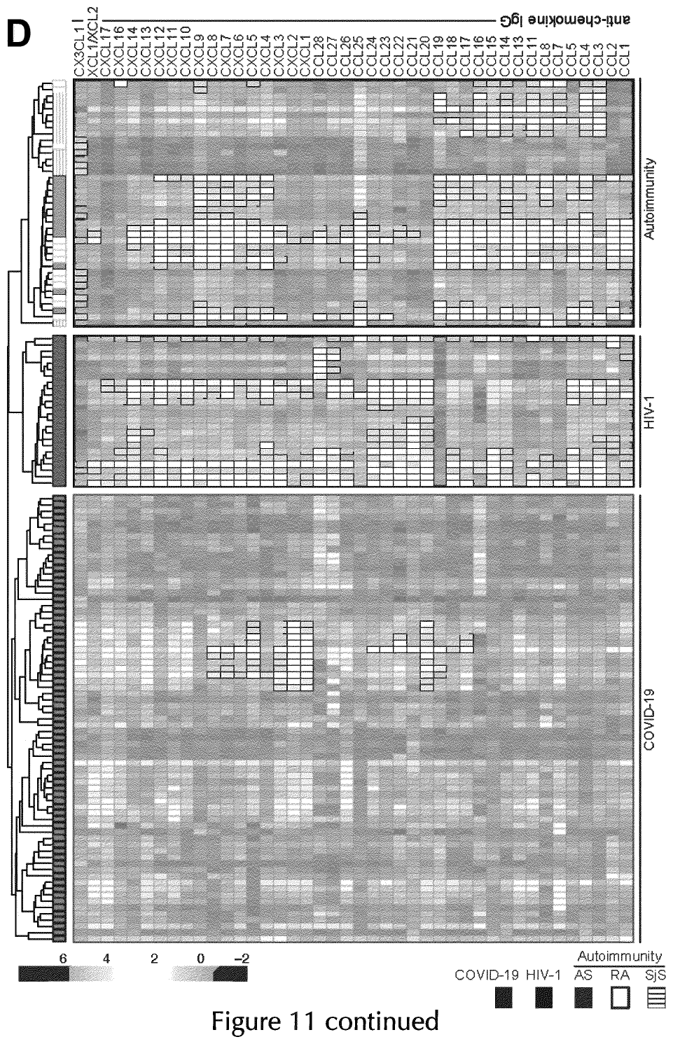

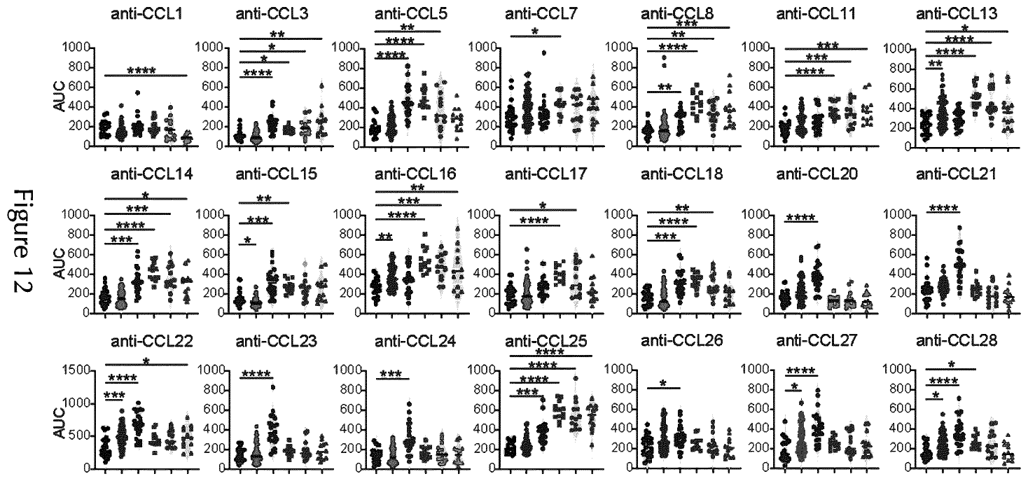

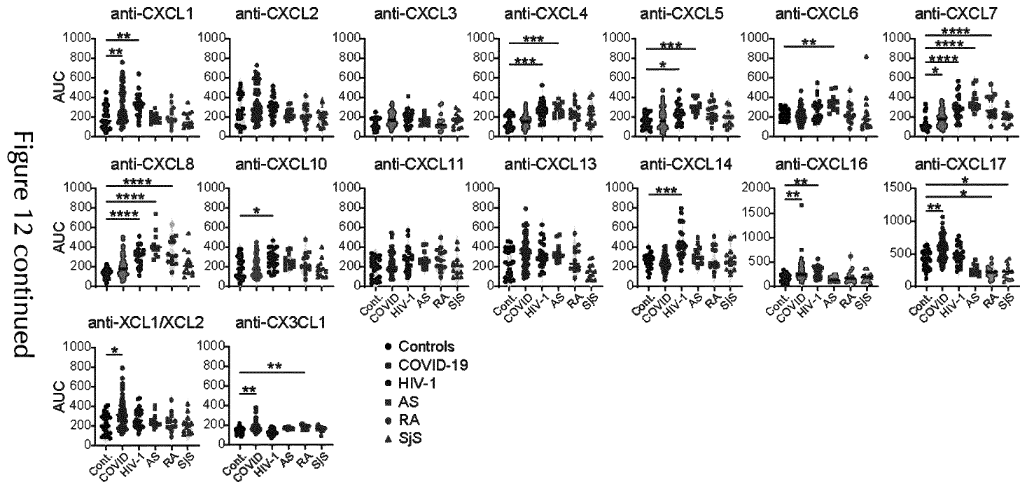

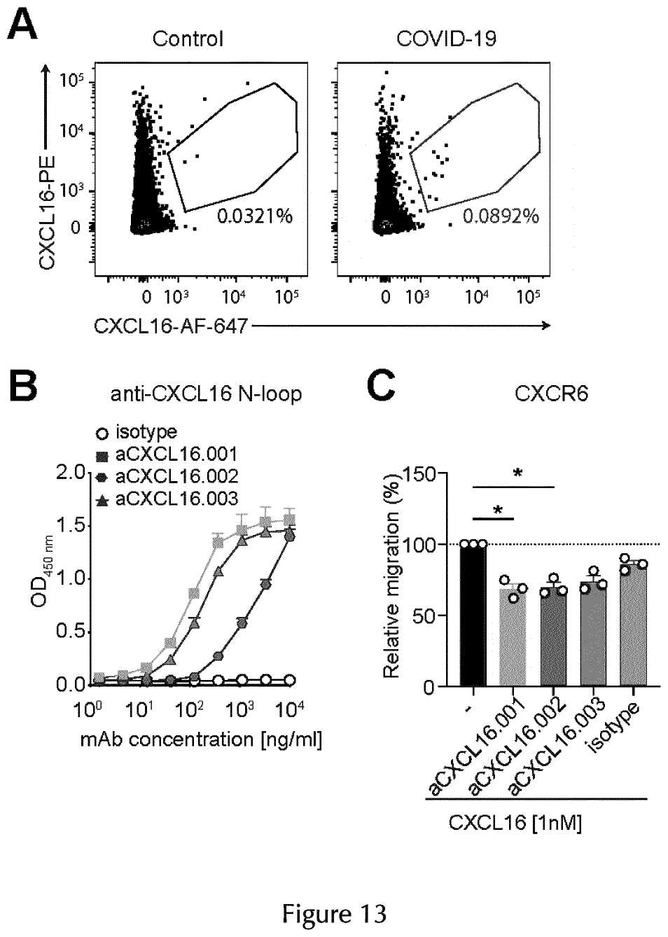

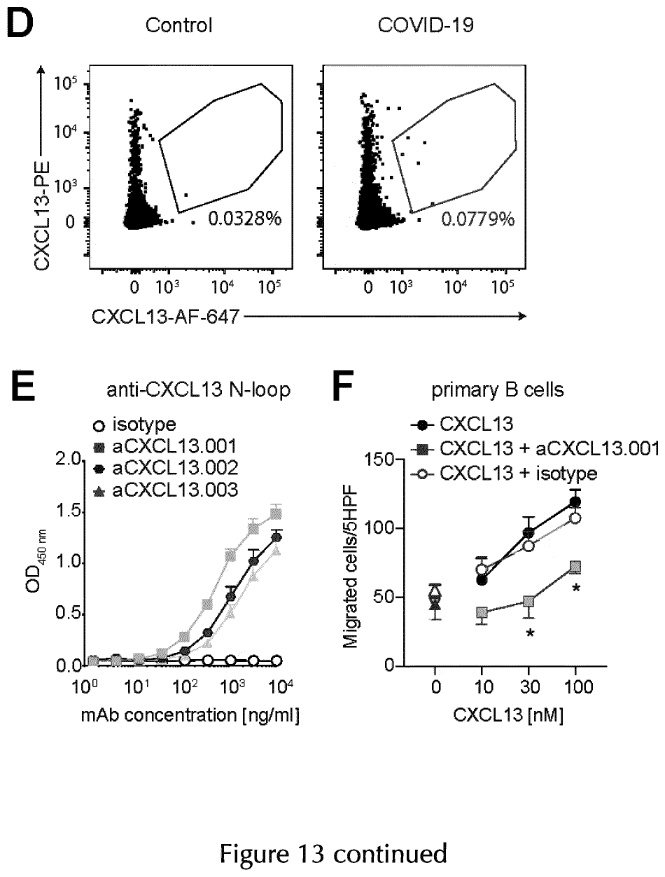

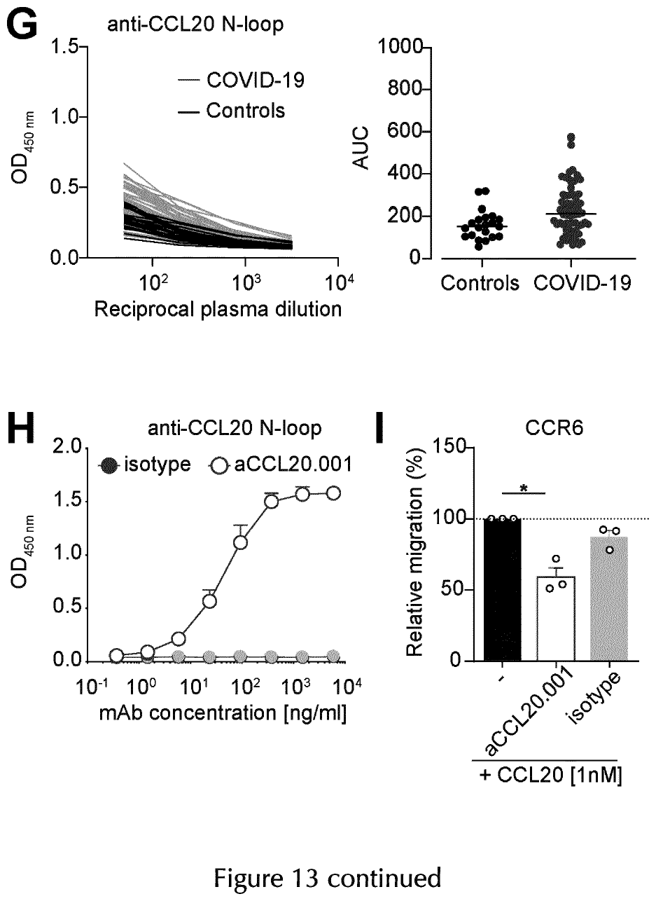

Antibody levels were measured by ELISA of serial plasma dilutions and the signal plotted as heatmap (FIGS. 3A and 4). Analysis of all parameters by nonlinear dimensionality reduction with t-distributed stochastic neighbor embedding (t-SNE) revealed a clear separation between controls and COVID-19 convalescents (FIG. 3B). Similar to CCL8, some individual convalescent plasma revealed high levels of IgGs to certain chemokines (for example CXCL13 and CXCL16). For these chemokines, antibody levels to the N-loop significantly correlated with those against the C-terminal region of the same chemokine, suggesting that, when present, antibodies formed against multiple chemokine epitopes (FIGS. 3A,C and 5A). When considering antibodies against each chemokine individually, a significant difference in reactivity over uninfected control was observed for peptides corresponding to 23 of the 43 chemokines (FIG. 3D). Antibodies to the three chemokines with p<104 (CCL19, CCL22 and CXCL17; “COVID-19 signature”) clustered together, and by themselves were sufficient to correctly assign uninfected controls and COVID-19 convalescents with accuracies >95% (FIGS. 3A, 3D-F and 5B; see Methods). Thus, COVID-19 is associated with a specific pattern of anti-chemokine antibodies.

To examine the relationship between anti-chemokine antibodies and other serologic features of the COVID-19 cohort, ELISA and pseudovirus-based neutralization assays were used to measure binding and neutralizing capacity of antibodies against SARS-CoV-2 (Robbiani et al., 2020). In agreement with previous studies, IgG binding to SARS-CoV-2 Spike receptor binding domain (RBD) and plasma half-maximal neutralizing titers (NT₅₀) against SARS-CoV-2 were variable but positively correlated with each other and with age (FIG. 5C-E) (Robbiani et al., 2020). Instead, there was no correlation between NT₅₀ or anti-RBD IgGs and the levels of antibodies to the signature chemokines CCL19, CCL22 and CXCL17, or to the sum of all anti-chemokine IgG reactivities (FIG. 5F). There was a weak negative correlation between age and the sum of all anti-chemokine IgG reactivities (cumulative area under the curve [AUC]; FIG. 5F), and no differences in the levels of the antibodies to the signature chemokines between males and females (FIG. 5G). This indicates that after COVID-19, antibodies against specific chemokines are not correlated with those against SARS-CoV-2.

### Example 3: Anti-Chemokine Antibodies Overtime and Upon Vaccination

To document the temporal evolution of anti-chemokine antibodies following COVID-19, the reactivities to a subset of chemokines in plasma collected from the same cohort at approximately 6 months (t=6 m) and 12 months (t=12 m) from symptom onset were compared side-by-side (FIG. 6A). Antibodies to the virus RBD significantly decreased in unvaccinated COVID-19 convalescents, while they increased in those receiving at least one dose of mRNA-based COVID-19 vaccine (FIG. 6B). In contrast, and regardless of vaccination status (data not shown), antibodies to the COVID-19 signature chemokine CCL9 significantly increased (2.1-fold, p<0.0001), those to CCL22 remained generally stable, and those to CXCL17 followed variable kinetics (FIG. 6C). Similar to CCL9, antibody levels to CCL8, CCL13, CCL16, CXCL7 and CX3CL1 were also augmented at 12 months, while a reduction was observed for CXCL16 (FIG. 6C). To further investigate the kinetic of COVID-19 signature antibodies, cohort individuals for which acute and later samples were available were analyzed (n=12; t=Acute; FIG. 6D). During acute COVID-19, IgG antibodies to CCL19, but not to CCL22 or CXCL17, were already higher than in uninfected control, and continued to increase until 12 months (FIG. 6E).

In contrast to natural infection, no significant change in antibody reactivity to any of the chemokines was observed upon COVID-19 mRNA vaccination of SARS-CoV-2 naïve individuals after about 4 months (130 days on average; n=16; FIG. 6F). Therefore, unlike the antibodies to SARS-CoV-2 RBD, which decrease over time, the levels of some anti-chemokine antibodies that are present upon COVID-19 increase over one year of observation.

### Example 4: Anti-Chemokine Antibodies Distinguish COVID-19 Outpatients from Hospitalized Individuals

To evaluate the relationship between the severity of acute COVID-19 and convalescent anti-chemokine IgGs, individuals in the cohort, who because of the infection were either previously hospitalized (n=50) or outpatients (n=21) were compared (FIG. 7). No significant difference in age distribution was observed between groups (age [years]: mean±SD; 60±14 in hospitalized, 57±15 in outpatients; p=0.3487), while a higher proportion of males was observed among hospitalized but not outpatients (60% and 38.1%, respectively).

When the most significant differences in antibody levels were taken into account (p<10⁻⁴), only the antibodies against CCL19 were higher in hospitalized individuals over uninfected controls, while antibodies against 7 chemokines (CXCL8, CCL20, CCL22, CXCL16, CXCL7, CX3CL1, in addition to CCL19) were increased in outpatients (FIGS. 7A,B and 8A). Consistent with this finding, outpatients, but not the hospitalized individuals displayed significantly higher cumulative anti-chemokine reactivity (p=0.0038; FIG. 7C). Thus, a broader pattern and higher overall amounts of anti-chemokine antibodies are observed in those COVID-19 convalescents that 6 months earlier were outpatients during the acute phase of the disease.

Direct comparison of previously hospitalized and outpatient individuals by t-SNE analysis of all anti-chemokine datasets separated the two groups (FIG. 7D). Antibodies against three chemokines highly significantly distinguished outpatients from hospitalized subjects (p<10⁻⁴): antibodies against CXCL5, CXCL8 and CCL25 were all lower in individuals with severe illness requiring hospitalization (FIGS. 7B, E and 8A). The combination of antibody values against these three chemokines alone could correctly assign formerly hospitalized and outpatient individuals with an accuracy of 77.5%, which was improved to 98.6% by including anti-CCL2 antibodies (“COVID-19 hospitalization signature”; FIGS. 7F and 8B). Among the COVID-19 hospitalization signature antibodies, those against CCL2 positively correlated with anti-RBD IgG and NT₅₀, while those to CXCL5 and CXCL8 were negatively correlated with anti-RBD IgG and age (FIG. 8C). Antibodies against CCL2 were increased in males (FIG. 8D). Consistent with previous work (Robbiani et al., 2020), both anti-RBD IgG and NT₅₀ values were significantly higher in hospitalized individuals compared to outpatients and in males compared to females of both groups (FIG. 8E). This indicates that the anti-chemokine antibody signature that distinguishes uninfected from COVID-19 convalescents (CCL19, CCL22 and CXCL17; FIG. 3) is different from the signature associated with different severity of COVID-19 disease (CXCL5, CXCL8, CCL25 and CCL2; FIG. 7).

### Example 5: Anti-Chemokine Antibodies and Long-Term COVID-19 Symptoms

A fraction of individuals who recovered from SARS-CoV-2 infection experience long-term sequelae. To determine whether a specific pattern of anti-chemokine antibodies at t=6 m is associated with the persistence of symptoms, this information was collected from the cohort at t=12 m (FIG. 9). 65.1% of all participants reported persistence of at least one symptom related to COVID-19. Among these, the average number of long-term symptoms was 3.3, and they were more frequent among formerly hospitalized individuals than outpatients (72.7% versus 47.4%; FIGS. 9A and 10A and B). No differences in age, gender distribution or time from disease onset to second visit were observed between individuals with and without protracted symptoms (FIG. 10C).

Convalescents with long-term sequelae showed significantly lower cumulative levels of anti-chemokine antibodies compared to those without symptoms (p=0.0135; FIG. 9B). This was particularly true for outpatients and among females (FIGS. 10D and E). In contrast, anti-RBD IgG and NT₅₀ values were comparable between the two groups (FIGS. 9C and D). The total levels of anti-chemokine antibodies did not correlate with the number of symptoms (FIG. 10F). These data indicate that overall higher levels of anti-chemokine antibodies at 6 months after COVID-19 are associated with absence of long-term symptoms at 12 months.

IgG antibodies against three chemokines distinguished the groups with high significance: CCL21 (p=0.0001), CXCL13 (p=0.0010) and CXCL16 (p=0.0011; FIGS. 9E and 10G; “COVID-19 persisting symptoms signature”). Logistic regression analysis using the antibody values for these 3 chemokines predicted long-term persistence of symptoms with accuracies of 77.8% (FIG. 9F). These results suggest that specific patterns of anti-chemokine antibodies at 6 months may predict the longer-term persistence of symptoms after COVID-19. The findings thus correlate the occurrence of patterns of autoantibodies targeting chemokines and the persistence of symptoms after a COVID-19 infection.

### Example 6: Anti-Chemokine Antibodies in HIV-1 and Autoimmune Diseases

To examine the relevance of anti-chemokine antibodies beyond COVID-19, we measured their presence in plasma from patients chronically infected with HIV-1 (n=24), and from individuals affected by Ankylosing Spondylitis (AS, n=13), Rheumatoid Arthritis (RA, n=13) and Sjögren Syndrome (SjS, n=13; FIG. 11, see Methods). While antibodies against a single chemokine distinguished with high confidence COVID-19 from uninfected controls (CCL19, p<10⁻⁴), in HIV-1, antibodies against 14 chemokines (that did not include CCL19) were increased with high confidence: CCL2, CCL3, CCL4, CCL5, CCL20, CCL21, CCL22, CCL23, CCL27, CCL28, CXCL7, CXCL8, CXCL9 and CXCL12 (p<10⁻⁴ for all; FIGS. 11A and B and FIG. 12). Similarly, AS, RA and SjS shared autoantibodies against 4 chemokines:CCL4, CCL19, CCL25 and CXCL9 (p<10⁻⁴ for all; FIGS. 11A-C and FIG. 12). Unsupervised clustering analysis with all anti-chemokine antibodies values correctly categorized all COVID-19 and HIV-1 samples with 100% accuracy, while the autoimmune diseases all clustered with each other (FIG. 11D; see Methods). A similar result was obtained by t-SNE analysis (FIG. 11E). Thus, patterns of anti-chemokine antibodies not only distinguish different COVID-19 trajectories, but also characterize other infections and autoimmune disorders.

### Example 7: Other Chemokine-Blocking Antibodies from COVID-19 Convalescent Individuals

Since anti-chemokine antibodies to CXCL13 and CXCL16 are associated with favorable long COVID outcome, we next derived corresponding memory B cell antibodies from available PBMC samples. Three N-loop binding monoclonal antibodies were obtained for CXCL16, which blocked migration of a cell line expressing the cognate receptor (CXCR6; FIGS. 13A-C). Similarly, 3 anti-CXCL13 N-loop antibodies bound in ELISA and inhibited chemotaxis of primary CD19⁺ human B cells to CXCL13 (FIGS. 13D-F). By the same approach, we discovered chemotaxis-blocking antibodies specific for CCL20 and for CCL23 (FIGS. 13G-1). Exemplified antibodies binding to CCL20, CCL23, CXCL13 and CXCL16 with their CDR and VH/VL sequences are the antibodies described in Table 3, 4, 5 and 6 above. Unlike the prior art, e.g. Wang et al. (Nature, Vol. 595, No. 7866; pp. 283-288, 2021), these data show that, like the antibodies against CCL8, antibodies that bind to the N-loop of CXCL13, CXCL16 and CCL20 interfere with cell migration.

TABLE OF SEQUENCES AND SEQ ID NUMBERS (SEQUENCE LISTING):

| SEQ ID NO | Sequence | Remarks |
|---|---|---|
| SEQ ID NO: 1 | GGKFSFAEQEIPLRAILCYRNT | CCL1 N-loop peptide |
| SEQ ID NO: 2 | GGKYNFTNRKISVQRLASYRRIT | CCL2 N-loop peptide |
| SEQ ID NO: 3 | GGSFSYTSRQIPQNFIADYFETSSQ | CCL3 N-loop peptide |
| SEQ ID NO: 4 | GGSFSYTARKLPRNFVVDYYETSSL | CCL4 N-loop peptide |
| SEQ ID NO: 5 | GGSFAYIARPLPRAHIKEYFYTSGK | CCL5 N-loop peptide |
| SEQ ID NO: 6 | GGSYRFINKKIPKQRLESYRRTTSS | CCL7 N-loop peptide |
| SEQ ID NO: 7 | GGSFNVINRKIPIQRLESYTRITNI | CCL8 N-loop peptide |
| SEQ ID NO: 8 | GGSFNLANRKIPLQRLESYRRITSG | CCL11 N-loop peptide |
| SEQ ID NO: 9 | GGSFTFSSKKISLQRLKSYVITTSR | CCL13 N-loop peptide |
| SEQ ID NO: 10 | GGSFTYTTYKIPRQRIMDYYETNSQ | CCL14 N-loop peptide |
| SEQ ID NO: 11 | GGSTSYISQSIPCSLMKSYFETSSE | CCL15 N-loop peptide |
| SEQ ID NO: 12 | GGGSLKYYEKVLPRRLVVGYRKALN | CCL16 N-loop peptide |
| SEQ ID NO: 13 | GGSLEYFKGAIPLRKLKTWYQTSED | CCL17 N-loop peptide |
| SEQ ID NO: 14 | GGSLVYTSWQIPQKFIVDYSETSPQ | CCL18 N-loop peptide |
| SEQ ID NO: 15 | GGSLSVTQKPIPGYIVRNFHYLLIK | CCL19 N-loop peptide |
| SEQ ID NO: 16 | GGSLGYTDRILHPKFIVGFTRQLAN | CCL20 N-loop peptide |
| SEQ ID NO: 17 | GGSLKYSQRKIPAKVVRSYRKQEPS | CCL21 N-loop peptide |
| SEQ ID NO: 18 | GGSRDYVRYRLPLRVVKHFYWTSDS | CCL22 N-loop peptide |
| SEQ ID NO: 19 | GGSISYTPRSIPCSLLESYFETNSE | CCL23 N-loop peptide |
| SEQ ID NO: 20 | GGSMFFVSKRIPENRVVSYQLSSRS | CCL24 N-loop peptide |
| SEQ ID NO: 21 | GGSLAYHYPIGWAVLRRAWTYRIQE | CCL25 N-loop peptide |
| SEQ ID NO: 22 | GGSFQYSHKPLPWTWVRSYEFTSNS | CCL26 N-loop peptide |
| SEQ ID NO: 23 | GGSTQLYRKPLSDKLLRKVIQVELQ | CCL27 N-loop peptide |
| SEQ ID NO: 24 | GGSTEVSHHISRRLLERVNMCRIQR | CCL28 N-loop peptide |
| SEQ ID NO: 25 | GGSLQTLQGIHPKNIQSVNVKSPGP | CXCL1 N-loop peptide |
| SEQ ID NO: 26 | GGSLQTLQGIHLKNIQSVKVKSPGP | CXCL2 N-loop peptide |
| SEQ ID NO: 27 | GSLQTLQGIHLKNIQSVNVRSPGPH | CXCL3 N-loop peptide |
| SEQ ID NO: 28 | GGSVKTTSQVRPRHITSLEVIKAGP | CXCL4 N-loop peptide |
| SEQ ID NO: 29 | GGSLQTTQGVHPKMISNLQVFAIGP | CXCL5 N-loop peptide |
| SEQ ID NO: 30 | GGSLRVTLRVNPKTIGKLQVFPAGP | CXCL6 N-loop peptide |
| SEQ ID NO: 31 | GGSIKTTSGIHPKNIQSLEVIGKGT | CXCL7 N-loop peptide |
| SEQ ID NO: 32 | GGSIKTYSKPFHPKFIKELRVIESG | CXCL8 N-loop peptide |
| SEQ ID NO: 33 | GGSISTNQGTIHLQSLKDLKQFAPS | CXCL9 N-loop peptide |
| SEQ ID NO: 34 | GGSISISNQPVNPRSLEKLEIIPAS | CXCL10 N-loop peptide |
| SEQ ID NO: 35 | GGSIGPGVKAVKVADIEKASIMYPS | CXCL11 N-loop peptide |
| SEQ ID NO: 36 | GGSRFFESHVARANVKHLKILNTPN | CXCL12 N-loop peptide |
| SEQ ID NO: 37 | GGSVQESSVFIPRRFIDRIQILPRG | CXCL13 N-loop peptide |
| SEQ ID NO: 38 | GGSSRKGPKIRYSDVKKLEMKPKYP | CXCL14 N-loop peptide |
| SEQ ID NO: 39 | GGSGKRISSDSPPSVQFMNRLRKHL | CXCL16 N-loop peptide |
| SEQ ID NO: 40 | GGSKDWFLRAPRRKFMTVSGLPKKQ | CXCL17 N-loop peptide |
| SEQ ID NO: 41 | GGSVSLTTQRLPVSRIKTYTITEGS | XCL1/XCL2 N-loop |
| | | peptide |
| SEQ ID NO: 42 | GGGSSKMTSKIPVALLIHYQQNQAS | CX3CL1 N-loop peptide |
| SEQ ID NO: 43 | GGGSKERWVRDSMKHLDQIFQNLKP | CCL8 C-terminal peptide |
| SEQ ID NO: 44 | GGSNDDWVQEYIKDPNLPLLPTRNL | CCL16 C-terminal |
| | | peptide |
| SEQ ID NO: 45 | GGGSTHPRKKWVQKYISLLKTPKQL | CCL26 C-terminal |
| | | peptide |
| SEQ ID NO: 46 | GGSLNPASPIVKKIIEKMLNSDKSN | CXCL1 C-terminal |
| | | peptide |
| SEQ ID NO: 47 | GGSIDPKLKWIQEYLEKALNKRFKM | CXCL12 C-terminal |
| | | peptide |
| SEQ ID NO: 48 | GGSQAEWIQRMMEVLRKRSSSTLPV | CXCL13 C-terminal |
| | | peptide |
| SEQ ID NO: 49 | GGSKDPWVQELMSCLDLKECGHAYS | CXCL16 C-terminal |
| | | peptide |
| SEQ ID NO: 50 | GGSHSLGSRRTLMLLAQMRKISLFS | IFN-a2 (aa 7-28) |
| SEQ ID NO: 51 | GGSIVLQLPQGTTLPKGFYAEGSRG | SARS-CoV-2 |
| | | Nucleocapsid |
| | | (aa 157-178) |
| SEQ ID NO: 52 | FSFAEQEIPLRAILCYRNT | CCL1 N-loop peptide |
| SEQ ID NO: 53 | YNFTNRKISVQRLASYRRIT | CCL2 N-loop peptide |
| SEQ ID NO: 54 | FSYTSRQIPQNFIADYFETSSQ | CCL3 N-loop peptide |
| SEQ ID NO: 55 | FSYTARKLPRNFVVDYYETSSL | CCL4 N-loop peptide |
| SEQ ID NO: 56 | FAYIARPLPRAHIKEYFYTSGK | CCL5 N-loop peptide |
| SEQ ID NO: 57 | YRFINKKIPKQRLESYRRTTSS | CCL7 N-loop peptide |
| SEQ ID NO: 58 | FNVINRKIPIQRLESYTRITNI | CCL8 N-loop peptide |
| SEQ ID NO: 59 | FNLANRKIPLQRLESYRRITSG | CCL11 N-loop peptide |
| SEQ ID NO: 60 | FTFSSKKISLQRLKSYVITTSR | CCL13 N-loop peptide |
| SEQ ID NO: 61 | FTYTTYKIPRQRIMDYYETNSQ | CCL14 N-loop peptide |
| SEQ ID NO: 62 | TSYISQSIPCSLMKSYFETSSE | CCL15 N-loop peptide |
| SEQ ID NO: 63 | LKYYEKVLPRRLVVGYRKALN | CCL16 N-loop peptide |
| SEQ ID NO: 64 | LEYFKGAIPLRKLKTWYQTSED | CCL17 N-loop peptide |
| SEQ ID NO: 65 | LVYTSWQIPQKFIVDYSETSPQ | CCL18 N-loop peptide |
| SEQ ID NO: 66 | LSVTQKPIPGYIVRNFHYLLIK | CCL19 N-loop peptide |
| SEQ ID NO: 67 | LGYTDRILHPKFIVGFTRQLAN | CCL20 N-loop peptide |
| SEQ ID NO: 68 | LKYSQRKIPAKVVRSYRKQEPS | CCL21 N-loop peptide |
| SEQ ID NO: 69 | RDYVRYRLPLRVVKHFYWTSDS | CCL22 N-loop peptide |
| SEQ ID NO: 70 | ISYTPRSIPCSLLESYFETNSE | CCL23 N-loop peptide |
| SEQ ID NO: 71 | MFFVSKRIPENRVVSYQLSSRS | CCL24 N-loop peptide |
| SEQ ID NO: 72 | LAYHYPIGWAVLRRAWTYRIQE | CCL25 N-loop peptide |
| SEQ ID NO: 73 | FQYSHKPLPWTWVRSYEFTSNS | CCL26 N-loop peptide |
| SEQ ID NO: 74 | TQLYRKPLSDKLLRKVIQVELQ | CCL27 N-loop peptide |
| SEQ ID NO: 75 | TEVSHHISRRLLERVNMCRIQR | CCL28 N-loop peptide |
| SEQ ID NO: 76 | LQTLQGIHPKNIQSVNVKSPGP | CXCL1 N-loop peptide |
| SEQ ID NO: 77 | LQTLQGIHLKNIQSVKVKSPGP | CXCL2 N-loop peptide |
| SEQ ID NO: 78 | LQTLQGIHLKNIQSVNVRSPGPH | CXCL3 N-loop peptide |
| SEQ ID NO: 79 | VKTTSQVRPRHITSLEVIKAGP | CXCL4 N-loop peptide |
| SEQ ID NO: 80 | LQTTQGVHPKMISNLQVFAIGP | CXCL5 N-loop peptide |
| SEQ ID NO: 81 | LRVTLRVNPKTIGKLQVFPAGP | CXCL6 N-loop peptide |
| SEQ ID NO: 82 | IKTTSGIHPKNIQSLEVIGKGT | CXCL7 N-loop peptide |
| SEQ ID NO: 83 | IKTYSKPFHPKFIKELRVIESG | CXCL8 N-loop peptide |
| SEQ ID NO: 84 | ISTNQGTIHLQSLKDLKQFAPS | CXCL9 N-loop peptide |
| SEQ ID NO: 85 | ISISNQPVNPRSLEKLEIIPAS | CXCL10 N-loop peptide |
| SEQ ID NO: 86 | IGPGVKAVKVADIEKASIMYPS | CXCL11 N-loop peptide |
| SEQ ID NO: 87 | RFFESHVARANVKHLKILNTPN | CXCL12 N-loop peptide |
| SEQ ID NO: 88 | VQESSVFIPRRFIDRIQILPRG | CXCL13 N-loop peptide |
| SEQ ID NO: 89 | SRKGPKIRYSDVKKLEMKPKYP | CXCL14 N-loop peptide |
| SEQ ID NO: 90 | GKRISSDSPPSVQFMNRLRKHL | CXCL16 N-loop peptide |
| SEQ ID NO: 91 | KDWFLRAPRRKFMTVSGLPKKQ | CXCL17 N-loop peptide |
| SEQ ID NO: 92 | VSLTTQRLPVSRIKTYTITEGS | XCL1/XCL2 N-loop |
| | | peptide |
| SEQ ID NO: 93 | SKMTSKIPVALLIHYQQNQAS | CX3CL1 N-loop peptide |
| SEQ ID NO: 94 | GGGS | linker |

Antibodies amino acid sequences
aCCL8.001

| | | |
|---|---|---|
| SEQ ID NO: 95 | GDSVSSESYF | CDRH1 |
| SEQ ID NO: 96 | VWYNGDT | CDRH2 |
| SEQ ID NO: 97 | ATEHCIDEICSPASDS | CDRH3 |
| SEQ ID NO: 98 | QSVFFSSNNENC | CDRL1 |
| SEQ ID NO: 99 | WAS | CDRL2 |
| SEQ ID NO: 100 | LIHWASTRA | CDRL2 long |
| SEQ ID NO: 101 | HQYLKAPRT | CDRL3 |
| SEQ ID NO: 102 | QVQLQESGPGLVKPAETLSLTCTVSGDSVSSES | VH |
| | YFWGWIRQSPRKGLEWLGAVWYNGDTYYNP | |
| | SVGGRVTISMDTSMNEISLRLSSVTAADTAVYF | |
| | CATEHCIDEICSPASDSWGQGTLVTVSS | |
| SEQ ID NO: 103 | DIVMTQSPDSLAVSLGERATINCKSSQSVFFSSN | VL |
| | NENCLAWFQQKPGQPPKLLIHWASTRASGVP | |
| | DRFSGSGSGTDFTLTISSLQAEDVAVYYCHQYL | |
| | KAPRTFGQGTKVEIK | |

aCCL8.001v1

| | | |
|---|---|---|
| SEQ ID NO: 104 | DIVMTQSPDSLAVSLGERATINCKSSQSVFFSSN | VL |
| | NENCLAWFQQKPGQPPKLLIHWASTRASGVP | |
| | DRFSGSGSGTDFTLTISSLQAEDVAVYYCHQYL | |
| | KAPRTFGQGTKVEI | |

aCCL8.003

| | | |
|---|---|---|
| SEQ ID NO: 105 | GDSVSSSFYF | CDRH1 |
| SEQ ID NO: 106 | IYYTGDT | CDRH2 |
| SEQ ID NO: 107 | ATEFCTSGICHPASDS | CDRH3 |
| SEQ ID NO: 108 | QSVLLSANNKNC | CDRL1 |
| SEQ ID NO: 109 | LIYWASTRE | CDRL2 long |
| SEQ ID NO: 110 | QQYYKAPRT | CDRL3 |
| SEQ ID NO: 111 | QLQLQESGPGLVKPSETLSLTCTVSGDSVSSSFY | VH |
| | FWGWVRQPPGKGLEWIGSIYYTGDTYYKPSLK | |
| | SRVTISMNTSKNQFSLRLSSVTAADTAVYYCATE | |
| | FCTSGICHPASDSWGQGTLVTVSS | |
| SEQ ID NO: 112 | DIVMTQSPDSLAVSLGERATINCKSSQSVLLSA | VL |
| | NNKNCLAWFQQKPGQPPKLLIYWASTRESGV | |
| | PDRFSGSGSGTDFTLTISSLQAEDVAIYYCQQY | |
| | YKAPRTFGQGTKVEIK | |

aCCL8.004

| | | |
|---|---|---|
| SEQ ID NO: 113 | GDSVSSPSYF | CDRH1 |
| SEQ ID NO: 114 | TWFNGDT | CDRH2 |
| SEQ ID NO: 115 | ATEHCFDEICSPASDS | CDRH3 |
| SEQ ID NO: 116 | LIYWASTRA | CDRL2 long |
| SEQ ID NO: 117 | QQYLKAPRT | CDRL3 |
| SEQ ID NO: 118 | QVQLQESGPGLVKPAETLSLTCIVSGDSVSSPSY | VH |
| | FWGWIRQPPGKGLEWIGATWFNGDTYYNPSL | |
| | QSRVSISMDMSLNRIYLSLNSVTAADTAVYFCA | |
| | TEHCFDEICSPASDSWGQGTLVTVSS | |
| SEQ ID NO: 119 | DIVMTQSPDSLAVSLGERATISCMSSQSVFFSSN | VL |
| | NENCLAWFQQKPGQPPKLLIYWASTRASGVP | |
| | DRFSGSGSGTDFTLTISSLQAEDVAVYYCQQYL | |
| | KAPRTFGQGTKVEIK | |

aCCL8.004v1

| | | |
|---|---|---|
| SEQ ID NO: 120 | QLQLQESGPGLVKPAETLSLTCIVSGDSVSSPSY | VH |
| | FWGWIRQPPGKGLEWIGATWFNGDTYYNPSL | |
| | QSRVSISMDMSLNRIYLSLNSVTAADTAVYFCA | |
| | TEHCFDEICSPASDSWGQGTLVTVSS | |
| SEQ ID NO: 121 | DIVMTQSPDSLAVSLGERATISCMSSQSVFFSSN | VL |
| | NENCLAWFQQKPGQPPKLLIYWASTRASGVP | |
| | DRFSGSGSGTDFTLTISSLQAEDVAVYYCQQYL | |
| | KAPRTFGQGTKVELK | |

aCCL8.005

| | | |
|---|---|---|
| SEQ ID NO: 122 | TYYTGDT | CDRH2 |
| SEQ ID NO: 123 | ATEFCINGICRPASDS | CDRH3 |
| SEQ ID NO: 124 | QSVLLSSNNKNC | CDRL1 |
| SEQ ID NO: 125 | QLQLQESGPGLVKPSETLSLTCTVSGDSVSSSFY | VH |
| | FWGWVRQPPGKGLEWIGGTYYTGDTYHNPS | |
| | LKSRLTISMDTSKNQFSLRLSSVTAADTAVYYCA | |
| | TEFCINGICRPASDSWGQGTLVTVSS | |
| SEQ ID NO: 126 | DIVMTQSPDSLAVSLGERATINCKSSQSVLLSSN | VL |
| | NKNCLAWFQQRPGQPPKLLIYWASTRESGVP | |
| | DRFSGSGSGTDFTLTISSLQAEDVAIYYCQQYY | |
| | KAPRTFGQGTKVEIK | |

aCCL8.008

| | | |
|---|---|---|
| SEQ ID NO: 127 | GYQFTDYC | CDRH1 |
| SEQ ID NO: 128 | IFPDTFDI | CDRH2 |
| SEQ ID NO: 129 | ARIERVPYGSEGEYLNVGCDS | CDRH3 |
| SEQ ID NO: 130 | QNINSW | CDRL1 |
| SEQ ID NO: 131 | MAS | CDRL2 |
| SEQ ID NO: 132 | LIYMASTLQ | CDRL2 long |
| SEQ ID NO: 133 | QHFNKSPYT | CDRL3 |
| SEQ ID NO: 134 | EVQLVQSRAEVKKPGQSLTISCEASGYQFTDYC | VH |
| | VAWVRQVPGKGLEFVAIIFPDTFDIKYSPSFQG | |
| | HVTISADKSITTVFLHWTSLETSDTAMYFCARIE | |
| | RVPYGSEGEYLNVGCDSWGQGTLVTVSS | |
| SEQ ID NO: 135 | DIQMTQSPSSLSASVGGRVTITCRASQNINSWL | VL |
| | AWYQQKPGKTPKLLIYMASTLQPGVPSRFSGS | |
| | GFGTDFTLTISGLQPDDFATYYCQHENKSPYTF | |
| | GQGTKVEIK | |

aCCL8.008v1

| | | |
|---|---|---|
| SEQ ID NO: 136 | EVQLVQSGAEVKKPGQSLTISCEASGYQFTDYC | VH |
| | VAWVRQVPGKGLEFVAIIFPDTFDIKYSPSFQG | |
| | HVTISADKSITTVFLHWTSLETSDTAMYFCARIE | |
| | RVPYGSEGEYLNVGCDSWGQGTQVIVSS | |
| SEQ ID NO: 137 | DIQMTQSPSTLSASVGGRVTITCRASQNINSWL | VL |
| | AWYQQKPGKTPKLLIYMASTLQPGVPSRFSGS | |
| | GFGTDFTLTISGLQPDDFATYYCQHENKSPYTF | |
| | GQGTLVEVK | |

aCCL8_B5

| | | |
|---|---|---|
| SEQ ID NO: 138 | GDSVSSSSYF | CDRH1 |
| SEQ ID NO: 139 | QLQLQESGPGLVKPSETLSLTCTVSGDSVSSSSY | VH |
| | FWGWVRQPPGKGLEWIGSIYYTGDTYYKPSLK | |
| | SRVTISMNTSKNQISLRLSSVTAADTAVYYCATE | |
| | FCTSGICHPASDSWGQGTLVTVSS | |
| SEQ ID NO: 140 | DIVMTQSPDSLAMSLGERATINCKSSQSVLLSA | VL |
| | NNKNCLAWIQQKPGQPPKVLIYWASTRESGV | |
| | PDRFSGSGSGTDFTLTISSLQAEDVAIYYCQQY | |
| | YKAPRTFGQGTKVEIK | |

aCCL8_C4

| | | |
|---|---|---|
| SEQ ID NO: 141 | GDSVSTSSYF | CDRH1 |
| SEQ ID NO: 142 | IFYTGDT | CDRH2 |
| SEQ ID NO: 143 | ATEFCTSGICHPASDS | CDRH3 |
| SEQ ID NO: 144 | QLQLQESGPGLLKPSETLSLTCTVSGDSVSTSSY | VH |
| | FWGWVRQPPGKGLEWIGSIFYTGDTYYKPSLK | |
| | SRVTISMNTSKNQISLRLSSVTAADTAVYYCATE | |
| | FCTSGICHPASDSWGQGTLVTVSS | |
| SEQ ID NO: 145 | DIVMTQSPDSLAVSLGERATINCKSSQSVLLSA | VL |
| | NNKNCLAWIQQKPGQPPKVLIYWASTRESGV | |
| | PDRFSGSGSGTDFTLTISSLQAEDVAIYYCQQY | |
| | YKAPRTFGQGTKVEIK | |

aCCL8_D9

| | | |
|---|---|---|
| SEQ ID NO: 146 | QLQLQESGPGLLKPSETLSLTCTVSGDSVSSSSY | VH |
| | FWGWVRQPPGKGLEWIGSIFYTGDTYYKPSLK | |
| | SRVTISMNTSKNQISLRLSSVTAADTAVYYCATE | |
| | FCTSGICHPASDSWGQGTLVTVSS | |
| SEQ ID NO: 147 | DIVMTQSPDSLAVSLGERATINCKSSQSVLLSA | VL |
| | NNKNCLAWIQQKPGQPPKVLIYWASTRESGV | |
| | PDRFSGSGSGTDFTLTISSLQAEDVAIYYCQQY | |
| | YKAPRTFGQGTKVEIK | |

aCCL8_A2

| | | |
|---|---|---|
| SEQ ID NO: 148 | GSRVTEYY | CDRH1 |
| SEQ ID NO: 149 | IRPATLDI | CDRH2 |
| SEQ ID NO: 150 | ARIKVMPYGSEGNYWNVGCDS | CDRH3 |
| SEQ ID NO: 151 | QNIYSW | CDRL1 |
| SEQ ID NO: 152 | LAS | CDRL2 |
| SEQ ID NO: 153 | LIYLASDLQ | CDRL2 long |
| SEQ ID NO: 154 | QHYNKSPYT | CDRL3 |
| SEQ ID NO: 155 | EVQLVQSGPEVKKPGESLTISCEASGSRVTEYYV | VH |
| | AWVRQMPGRGLEYVGIIRPATLDIRYDPSFQG | |
| | HVTMSADESSTTATDRSRTTVFLHWTSLETSDT | |
| | AIYYCARIKVMPYGSEGNYWNVGCDSWGQG | |
| | TQVIVSS | |
| SEQ ID NO: 156 | DIQMTQSPSTLSASVGDRVTITCRASQNIYSWL | VL |
| | AWYQQKPGKTPKLLIYLASDLQPAVPSRFSGSG | |
| | FGTYFTLTINDLQPDDFATYYCQHYNKSPYTF | |
| | GQGTRVEI | |

aCCL8_A5

| | | |
|---|---|---|
| SEQ ID NO: 157 | GFIFSNYA | CDRH1 |
| SEQ ID NO: 158 | ISFDARNK | CDRH2 |
| SEQ ID NO: 159 | ARDLFLSDYDNGVYDPTRGGFDY | CDRH3 |
| SEQ ID NO: 160 | QSVSDW | CDRL1 |
| SEQ ID NO: 161 | KAS | CDRL2 |
| SEQ ID NO: 162 | LIYKASILE | CDRL2 long |
| SEQ ID NO: 163 | QKYNKSPYT | CDRL3 |
| SEQ ID NO: 164 | QVQLVESGGGVVQPGRPLRLSCSGSGFIFSNY | VH |
| | AMHWVRQAPGKGLEWVAIISFDARNKRYADS | |
| | VEGRFTISRDNSKNILYLQMNSLRLEDSAVYFCA | |
| | RDLFLSDYDNGVYDPTRGGFDYWGQGTLVT | |
| | VSS | |
| SEQ ID NO: 165 | DIQMTQSPXSLSASVGDRVIITCRTSQSVSDWL | VL |
| | AWYQQKPGRTPKLLIYKASILEAGVPSRFRGSG | |
| | YGTEFTLTINSLQPDDFATYYCQKYNKSPYTFG | |
| | QGTRLEL | |

aCCL8_D2

| | | |
|---|---|---|
| SEQ ID NO: 166 | QSVLFGSNNENC | CDRL1 |
| SEQ ID NO: 167 | LISWASTRA | CDRL2 long |
| SEQ ID NO: 168 | QQYRKAPRT | CDRL3 |
| SEQ ID NO: 169 | QVQLVESGGGVVQPGRPLRLSCSGSGFIFSNY | VH |
| | AMHWVRQAPGKGLEWVAIISFDARNKRYADS | |
| | VEGRFTISRDNSKNILYLQMNSLRLEDSAVYFCA | |
| | RDLFLSDYDNGVYDPTRGGFDYWGQGTLVT | |
| | VSS | |
| SEQ ID NO: 170 | DIVMTQSPDSLAVPLGERATISCKSSQSVLFGSN | VL |
| | NENCLAWFQQRPGQPPKLLISWASTRASGVP | |
| | DRFSGSGSETDFTLTISSLQPEDVAIYYCQQYRK | |
| | APRTFGQGTRLEIK | |

aCCL8_C1

| | | |
|---|---|---|
| SEQ ID NO: 171 | GASISRGDYA | CDRH1 |
| SEQ ID NO: 172 | VYHSGTT | CDRH2 |
| SEQ ID NO: 173 | REGRATAWEKNNFFDS | CDRH3 |
| SEQ ID NO: 174 | QSVLFGSNNENC | CDRL1 |
| SEQ ID NO: 175 | QLQLQESGSGLVKPSQTLSLTCIVSGASISRGDY | VH |
| | AWSWFRQPPGKGLEWIGYVYHSGTTYYSPSLK | |
| | NRVTISVDRSRNQFSVRLTSVTAADTAVYYCAR | |
| | EGRATAWEKNNFFDSWGQGTLVTVS | |
| SEQ ID NO: 176 | DIVMTQSPDSLAVSLGERATISCRSSQSVLFGSN | VL |
| | NENCLAWFQQRPGQPPKLLISWASTRASGVP | |
| | DRFSGSGSETDFTLTISSLQPEDVAIYYCQQYRK | |
| | APRTFGQGTRLEIK | |

aCCL8_B11

| | | |
|---|---|---|
| SEQ ID NO: 177 | GASVNSDAYF | CDRH1 |
| SEQ ID NO: 178 | IYYTGET | CDRH2 |
| SEQ ID NO: 179 | ATESCIDGLCSPASDS | CDRH3 |
| SEQ ID NO: 180 | QSVLFSSDNDNENC | CDRL1 |
| SEQ ID NO: 181 | QLQLQESGPGLVKPWETLSLTCSVSGASVNSD | VH |
| | AYFWGWVRQPPGQGLEWIGSIYYTGETHYNP | |
| | SLKSRVSISMDTSENQLSLILNSVTAADTAVYYC | |
| | ATESCIDGLCSPASDSWGQGTLVTVSA | |
| SEQ ID NO: 182 | DIVMTQSPXSLAVSLGERATISCKSSQSVLFSSD | VL |
| | NDNENCLAWFQQKPGQPPKLLIYWASTRASG | |
| | VPDRFSGSGSGTDFTLTISSLQPEDVAVYYCQQ | |
| | YRKAPRTFGQGTRLEIK | |

aCCL20.001

| | | |
|---|---|---|
| SEQ ID NO: 183 | IGYTFTNHY | CDRH1 |
| SEQ ID NO: 184 | IVNPSGGNT | CDRH2 |
| SEQ ID NO: 185 | ARDGSHYDDDGGYYGDAFDI | CDRH3 |
| SEQ ID NO: 186 | QSVLYNSNNKNF | CDRL1 |
| SEQ ID NO: 187 | LMYWASTRE | CDRL2 long |
| SEQ ID NO: 188 | QQYYSSPFT | CDRL3 |
| SEQ ID NO: 189 | QVQLVQSGAEVKKPGASVKISCKASGYTFTNH | VH |
| | YMHWVRQAPGVGLEWMGIVNPSGGNTSYA | |
| | RNFQGRIIMTRDMSTRTIYMELGSLRSEDTAVY | |
| | YCARDGSHYDDDGGYYGDAFDIWGQGTMV | |
| | TVSS | |
| SEQ ID NO: 190 | DIVMTQSPDSLAVSLGERATINCKSSQSVLYNS | VL |
| | NNKNFLAWYQQRPGQPPKLLMYWASTRESG | |
| | VPDRFSGSGSATDFTLTISSLQAEDVAVYYCQQ | |
| | YYSSPFTFGPGTKVDIK | |

aCCL20.001v1

| | | |
|---|---|---|
| SEQ ID NO: 191 | QVQLVQSGAEVKKPGASVKISCKASGYTFTNH | VH |
| | YMHWVRQAPGVGLEWMGIVNPSGGNTSYA | |
| | RNFQGRIIMTRDMSTRTIYMELGSLRSEDTAVY | |
| | YCARDGSHYDDDGGYYGDAFDIWGQGTVV | |
| | TVSS | |
| SEQ ID NO: 192 | DIVMTQSPDSLAVSLGERATINCKSSQSVLYNS | VL |
| | NNKNFLAWYQQRPGQPPKLLMYWASTRESG | |
| | VPDRFSGSGSATDFTLTISSLQAEDVAVYYCQQ | |
| | YYSSPFTFGPGTKVDLK | |

aCCL23.001

| | | |
|---|---|---|
| SEQ ID NO: 193 | GFTFSNYS | CDRH1 |
| SEQ ID NO: 194 | ISSRSSYI | CDRH2 |
| SEQ ID NO: 195 | ARVEVIRGVRFDY | CDRH3 |
| SEQ ID NO: 196 | QGISSY | CDRL1 |
| SEQ ID NO: 197 | GAS | CDRL2 |
| SEQ ID NO: 198 | LIYGASTLQ | CDRL2 long |
| SEQ ID NO: 199 | QQLHSSPPS | CDRL3 |
| SEQ ID NO: 200 | QVQLVESGGGLVKPGGSLRLSCAASGFTFSNY | VH |
| | SMNWVRQAPGKGLEWVSSISSRSSYIYYADSV | |
| | KGRFTISRDNAKKSLFLQMNSLRVEDTAVYYCA | |
| | RVEVIRGVRFDYWGQGTLVTVSS | |
| SEQ ID NO: 201 | DIQLTQSPSFLSASVGDRVTITCRASQGISSYLA | VL |
| | WYQQKPGKAPKLLIYGASTLQGGVPSTFSGSG | |
| | SGTDFTLTISSLQPEDSATYYCQQLHSSPPSFGQ | |
| | GTKLEIK | |

aCCL23.001v1

| | | |
|---|---|---|
| SEQ ID NO: 202 | EVQLVESGGGLVKPGGSLRLSCAASGFTFSNYS | VH |
| | MNWVRQAPGKGLEWVSSISSRSSYIYYADSVK | |
| | GRFTISRDNAKKSLFLQMNSLRVEDTAVYYCAR | |
| | VEVIRGVRFDYWGQGTLVTVSS | |

aCCL23.002

| | | |
|---|---|---|
| SEQ ID NO: 203 | GFTFSSYA | CDRH1 |
| SEQ ID NO: 204 | ISGSGGGT | CDRH2 |
| SEQ ID NO: 205 | ARVKGEVGGVIYGMDV | CDRH3 |
| SEQ ID NO: 206 | QSVTSSY | CDRL1 |
| SEQ ID NO: 207 | LIYGASTRA | CDRL2 long |
| SEQ ID NO: 208 | QHRGFT | CDRL3 |
| SEQ ID NO: 209 | EVQLLESGGGLVQPGGSLRVSCAASGFTFSSYA | VH |
| | MSWVRQAPGKGLEWVSAISGSGGGTYYAHSV | |
| | KGRITISRDNSKNTLYLQMNSLRAEDTAVYHCA | |
| | RVKGEVGGVIYGMDVWGQGTTVTVSS | |
| SEQ ID NO: 210 | EIVLTQSPGTLSLSPGERATLSCRASQSVTSSYLA | VL |
| | WYQQKPGQAPRLLIYGASTRATGIPDRFSGSG | |
| | SGTDFTLTISRLEPEDFAVYYCQHRGFTFGPGTK | |
| | VDIK | |

aCXCL13.001

| | | |
|---|---|---|
| SEQ ID NO: 211 | GFMFSDYY | CDRH1 |
| SEQ ID NO: 212 | GNSDAST | CDRH2 |
| SEQ ID NO: 213 | ARDGYSHSRGYSYGRKDAFDI | CDRH3 |
| SEQ ID NO: 214 | QSVSSY | CDRL1 |
| SEQ ID NO: 215 | AAS | CDRL2 |
| SEQ ID NO: 216 | LIYAASQRA | CDRL2 long |
| SEQ ID NO: 217 | QQRGNWPGT | CDRL3 |
| SEQ ID NO: 218 | QVQLVESGGGLVKPGGSLRLSCAASGFMFSDY | VH |
| | YMSWIRQAPGKGLEWISYIGNSDASTFYADSV | |
| | KGRFAISRDNAKNLLFLQMNSLRAEDTAVYYC | |
| | ARDGYSHSRGYSYGRKDAFDIWGRGTMVTVS | |
| | S | |
| SEQ ID NO: 219 | EIVLTQSPATLSLSPGERATLSCRASQSVSSYLAW | VL |
| | YQQKPGQAPRLLIYAASQRATGIPARFSGTGS | |
| | GTDFTLTISSLEPEDSAVYYCQQRGNWPGTFG | |
| | QGTKVEIK | |

aCXCL13.001v1

| | | |
|---|---|---|
| SEQ ID NO: 220 | EIVLTQSPATLSLSPGERATLSCRASQSVSSYLAW | VL |
| | YQQKPGQAPRLLIYAASQRATGIPARFSGTGX | |
| | GTDFTLTISSLEPEDXAVYYCQQRGNWPGTFG | |
| | QGTKLEIK | |

aCXCL13.002

| | | |
|---|---|---|
| SEQ ID NO: 221 | GFTFSSYS | CDRH1 |
| SEQ ID NO: 222 | ISSSSSYI | CDRH2 |
| SEQ ID NO: 223 | ARDGIYDYAPVDY | CDRH3 |
| SEQ ID NO: 224 | QSVLYSSNNKNY | CDRL1 |
| SEQ ID NO: 225 | QQYYSTPFT | CDRL3 |
| SEQ ID NO: 226 | EVQLVESGGGLVKPGGSLRLSCAASGFTFSSYS | VH |
| | MNWVRQAPGKGLEWVSSISSSSSYIYYADSVK | |
| | GRFTISRDNAKNSLYLQMNSLRAEDTAVYYCA | |
| | RDGIYDYAPVDYWGQGTLVTVSS | |
| SEQ ID NO: 227 | DILMTQSPDSLAVSLGERATINCKSSQSVLYSSN | VL |
| | NKNYLAWYQQKPGQPPKLLIYWASTRESGVP | |
| | DRFSGSGSGTDFTLTISSLQAEDVAVYYCQQYY | |
| | STPFTFGGGTKVEIK | |

aCXCL13.002v1

| | | |
|---|---|---|
| SEQ ID NO: 228 | DIVMTQSPDSLAVSLGERATINCKSSQSVLYSSN | VL |
| | NKNYLAWYQQKPGQPPKLLIYWASTRESGVP | |
| | DRFSGSGSGTDFTLTISSLQAEDVAVYYCQQYY | |
| | STPFTFGGGTKVEIK | |

aCXCL13.003

| | | |
|---|---|---|
| SEQ ID NO: 229 | GGSFSGYY | CDRH1 |
| SEQ ID NO: 230 | INHSGST | CDRH2 |
| SEQ ID NO: 231 | ARAGRSYGDYGAFDI | CDRH3 |
| SEQ ID NO: 232 | QVQLQEWGAGLLKPSETLSLTCAVYGGSFSGY | VH |
| | YWSWIRQPPGKGLEWIGEINHSGSTNYNPSLK | |
| | SRVTISVDTSKNQFSLKLSSVTAADTAVYYCARA | |
| | GRSYGDYGAFDIWGQGTMVTVSS | |
| SEQ ID NO: 233 | DIVMTQSPDSLAVSLGERATINCKSSQSVLYSSN | VL |
| | NKNYLAWYQQKPGQPPKLLIYWASTRESGVP | |
| | DRFSGSGSGTDFTLTISSLQAEDVAVYYCQQYY | |
| | STPFTFGPGTKVDIK | |

aCXCL13.003v1

| | | |
|---|---|---|
| SEQ ID NO: 234 | QVQLQQWGAGLLKPSETLSLTCAVYGGSFSG | VH |
| | YYWSWIRQPPGKGLEWIGEINHSGSTNYNPSL | |
| | KSRVTISVDTSKNQFSLKLSSVTAADTAVYYCAR | |
| | AGRSYGDYGAFDIWGQGTMVTVSS | |

aCXCL16.001

| | | |
|---|---|---|
| SEQ ID NO: 235 | GYPFANAW | CDRH1 |
| SEQ ID NO: 236 | IKTKSEGETT | CDRH2 |
| SEQ ID NO: 237 | TTVFF | CDRH3 |
| SEQ ID NO: 238 | ALSQQY | CDRL1 |
| SEQ ID NO: 239 | KDT | CDRL2 |
| SEQ ID NO: 240 | VISKDTERP | CDRL2 long |
| SEQ ID NO: 241 | HSTDISDSYWV | CDRL3 |
| SEQ ID NO: 242 | EVQLVESGGGLVKPGESLTLSCVASGYPFANA | VH |
| | WMSWVRQAPGKGLEWVGRIKTKSEGETTDYA | |
| | PLVRGRFTISRDDSKTTVFLQMNSLKTEDTGMY | |
| | YCTTVFFWGRGTLVTVSS | |
| SEQ ID NO: 243 | SYELTQPPSVSVSPGQTARITCSGDALSQQYLF | VL |
| | WHQQRPGQAPKVVISKDTERPSGIPERFSGSIS | |
| | GTTGTLTISGVQTEDEADYYCHSTDISDSYWVF | |
| | GGGTKLTVL | |

aCXCL16.001v1

| | | |
|---|---|---|
| SEQ ID NO: 244 | EVQLVESGGGLVKPGESLTLSCVASGYPFANA | VH |
| | WMSWVRQAPGKGLEWVGRIKTKSEGETTDYA | |
| | PLVRGRFTISRDDSKTTVFLQMNSLKTEDTGMY | |
| | YCTTVFFWGRGTLVGVSS | |
| SEQ ID NO: 245 | SYELTQPPSVSVSPGQTARITCSGDALSQQYLF | VL |
| | WHQQRPGQAPKVVISKDTERPSGIPERFSGSIS | |
| | GTTGTLTISGVQTEDEADYYCHSTDISDSYWVF | |
| | GGGTKLT | |

aCXCL16.002

| | | |
|---|---|---|
| SEQ ID NO: 246 | GLTVTRNY | CDRH1 |
| SEQ ID NO: 247 | LWTDGQK | CDRH2 |
| SEQ ID NO: 248 | TRDKVVPVRITPDYHYYGMDV | CDRH3 |
| SEQ ID NO: 249 | QGVRRY | CDRL1 |
| SEQ ID NO: 250 | LIYGASSLH | CDRL2 long |
| SEQ ID NO: 251 | QQLDTYPLT | CDRL3 |
| SEQ ID NO: 252 | EVQLVESGGGLSQPGWSLRLSCAASGLTVTRN | VH |
| | YMSWVRQAPGKGLEWVSTLWTDGQKFYADS | |
| | VKGRFTVSRDSYTNTLNLEMSGLRAEDTAVYFC | |
| | TRDKVVPVRITPDYHYYGMDVWGQGTTVTVS | |
| | S | |
| SEQ ID NO: 253 | DIQLTQSPSFLSASVGDRVTITCRASQGVRRYL | VL |
| | AWYQQKPGKAPKFLIYGASSLHTGVPSRFSGG | |
| | GSDTEFTLTISNLQTEDFATYYCQQLDTYPLTF | |
| | GGGTKVEIK | |

aCXCL16.002v1

| | | |
|---|---|---|
| SEQ ID NO: 254 | XIYGASSLH | CDRL2 long |
| SEQ ID NO: 255 | EVQLVESGGGLSQPGWSLRLSCAASGLTVTRN | VH |
| | YMSWVRQAPGKGLEWVSTLWTDGQKFYADS | |
| | VKGRFTVSRDSYTNTLNLEMSGLRAEDTAVYFC | |
| | TRDKVVPVRITPDYHYYGMDVWGQGTTVIVS | |
| | S | |
| SEQ ID NO: 256 | DIQLTQSPSFLSASVGDRVTITCRASQGVRRYL | VL |
| | AWYQQKPGKAPKFXIYGASSLHTGVPSRFSGG | |
| | GXDTEFTLTISNLQTEDFATYYCQQLDTYPLTF | |
| | GGGTKVEIK | |

aCXCL16.003

| | | |
|---|---|---|
| SEQ ID NO: 257 | GYSFTSYW | CDRH1 |
| SEQ ID NO: 258 | YPGDSDT | CDRH2 |
| SEQ ID NO: 259 | ARLHGDLYYFDY | CDRH3 |
| SEQ ID NO: 260 | SNNVGSYA | CDRL1 |
| SEQ ID NO: 261 | GNS | CDRL2 |
| SEQ ID NO: 262 | VMFGNSLPS | CDRL2 long |
| SEQ ID NO: 263 | STWDYSLSAQV | CDRL3 |
| SEQ ID NO: 264 | EVQLVQSGAEVKKPGESLKISCKGSGYSFTSYWI | VH |
| | GWVRQMPGKGLEWMGIIYPGDSDTRYSPSFQ | |
| | GQVTISADKSISTAYLQWSSLKASDTAMYYCAR | |
| | LHGDLYYFDYWGQGTLVTVSS | |
| SEQ ID NO: 265 | QSVLTQEASVSGTVGQKVTLSCTGNSNNVGS | VL |
| | YAVGWYQQISHGAPKTVMFGNSLPSGIPDRFS | |
| | GSKSGTTASLTISGLQPEDEADYYCSTWDYSLS | |
| | AQVFGGGTKLTVL | |

aCXCL16.003v1

| | | |
|---|---|---|
| SEQ ID NO: 266 | EVALVQSGAEVKKPGESLKISCKGSGYSFTSYWI | VH |
| | GWVRQMPGKGLEWMGIIYPGDSDTRYSPSFQ | |
| | GQVTISADKSISTAYLQWSSLKASDTAMYYCAR | |
| | LHGDLYYFDYWGQGTLVT | |

1. A (in vitro) method for determining binding of an antibody, or an antigen binding fragment thereof, to a human chemokine comprising the step of determining binding of the antibody, or the antigen binding fragment thereof, to a peptide comprising an amino acid sequence according to any one of SEQ ID NOs 52-93.

2. The method according to claim 1, wherein the human chemokine is selected from the group consisting of CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL16, CXCL17, XCL1, XCL2 and CX3CL1, or any combination thereof.

3. The method according to claim 1 or 2, wherein the peptide consists of (i) an amino acid sequence according to any one of SEQ ID NOs 52-93 and (ii) a linker.

4. The method according to claim 3, wherein the linker is positioned at the N-terminus of the amino acid sequence according to any one of SEQ ID NOs 52-93.

5. The method according to claim 3 or 4, wherein the linker has a length of (no more than) 1-10 amino acids, preferably (no more than) 2-7 amino acids, more preferably (no more than) 2-5 amino acids, and still more preferably (no more than) 2-4 amino acids.

6. The method according to any one of claims 3 to 5, wherein the linker is selected from GS, GGS, GGGS (SEQ ID NO: 94), or GGK.

7. The method according to any one of the previous claims, wherein the peptide consists of an amino acid sequence according to any one of SEQ ID NOs 1 to 42.

8. The method according to any one of the previous claims, wherein the peptide is modified at its N-terminus and/or at its C-terminus.

9. The method according to claim 51, wherein the peptide is biotinylated at its N-terminus and/or amidated at its C-terminus.

10. The method according to any one of the previous claims, wherein the binding of the antibody, or the antigen binding fragment thereof, to the peptide is determined in an enzyme-linked immunoassay (ELISA).

11. A method for identifying an antibody, or an antigen-binding fragment thereof, which binds to a human chemokine, in a sample, the method comprising:
performing the method as defined in any one of claims 1 to 10, wherein the sample is tested for binding to the peptide as defined in any one of claims 1 to 9.

12. The method according to claim 11, wherein the sample is selected from whole blood, plasma or serum.

13. The method according to claim 11 or 12, wherein the method includes a step of obtaining the sequences of the heavy and light chain variable region (VH/VL) genes of a B-cell capable of producing said antibody.

14. A method for generating an expression vector encoding the heavy and/or light chain of an antibody, which binds to a human chemokine, the method comprising the following steps:
(1) identification of nucleic acid sequences encoding the heavy and light chain variable region (VH/VL) of an antibody, which binds to a human chemokine, according to claim 13; and
(2) cloning the nucleic acid sequences encoding the heavy and light chain variable region (VH/VL) of the antibody into an expression vector for expression of antibody heavy and light chains, respectively.

15. A method for producing a recombinant cell expressing an antibody, which binds to a human chemokine, the method comprising the following steps:
(i) generating an expression vector encoding the heavy and/or light chain of the antibody, which binds to a human chemokine, according to claim 15;
(ii) transfecting a host cell with said expression vector; and
(iii) optionally, culturing said host cell.

16. A method for producing an antibody, which binds to a human chemokine, the method comprising the following steps:
(a) producing a recombinant cell expressing the antibody, which binds to a human chemokine, as described above; and
(b) isolating the antibody, which binds to a human chemokine.

17. A recombinant peptide comprising an amino acid sequence according to any one of SEQ ID NOs 52-93 and a linker, a tag, or a label.

18. The peptide according to claim 17, wherein the peptide is as defined in any one of claims 3 to 9.

19. The peptide according to claim 17 or 18, wherein the peptide consists of (i) an amino acid sequence according to any one of SEQ ID NOs 52-93 and (ii) a linker.

20. The peptide according to any one of claims 17 to 19, wherein the peptide consists of an amino acid sequence according to any one of SEQ ID NOs 1 to 42.

21. A composition comprising the peptide according to any one of claims 17 to 20.

22. A kit comprising at least two distinct peptides according to any one of claims 17 to 20.

23. An antibody, or an antigen-binding fragment thereof, which binds to CCL8 (Chemokine (C-C motif) ligand 8), wherein the antibody, or the antigen-binding fragment thereof, comprises:
(i) heavy chain CDR1, CDR2, and CDR3 sequences having at least 70% sequence identity with the amino acid sequences of SEQ ID NO: 95, SEQ ID NO: 96, and SEQ ID NO: 97, respectively, and light chain CDR1, CDR2, and CDR3 sequences having at least 70% sequence identity with the amino acid sequences of SEQ ID NO: 98, SEQ ID NO: 99 (or 100), and SEQ ID NO: 101, respectively;
(ii) heavy chain CDR1, CDR2, and CDR3 sequences having at least 70% sequence identity with the amino acid sequences of SEQ ID NO: 105, SEQ ID NO: 106, and SEQ ID NO: 107, respectively, and light chain CDR1, CDR2, and CDR3 sequences having at least 70% sequence identity with the amino acid sequences of SEQ ID NO: 108, SEQ ID NO: 99 (or 109), and SEQ ID NO: 110, respectively;
(iii) heavy chain CDR1, CDR2, and CDR3 sequences having at least 70% sequence identity with the amino acid sequences of SEQ ID NO: 113, SEQ ID NO: 114, and SEQ ID NO: 115, respectively, and light chain CDR1, CDR2, and CDR3 sequences having at least 70% sequence identity with the amino acid sequences of SEQ ID NO: 98, SEQ ID NO: 99 (or 116), and SEQ ID NO: 117, respectively;
(iv) heavy chain CDR1, CDR2, and CDR3 sequences having at least 70% sequence identity with the amino acid sequences of SEQ ID NO: 105, SEQ ID NO: 122, and SEQ ID NO: 123, respectively, and light chain CDR1, CDR2, and CDR3 sequences having at least 70% sequence identity with the amino acid sequences of SEQ ID NO: 124, SEQ ID NO: 99 (or 109), and SEQ ID NO: 110, respectively;
(v) heavy chain CDR1, CDR2, and CDR3 sequences having at least 70% sequence identity with the amino acid sequences of SEQ ID NO: 127, SEQ ID NO: 128, and SEQ ID NO: 129, respectively, and light chain CDR1, CDR2, and CDR3 sequences having at least 70% sequence identity with the amino acid sequences of SEQ ID NO: 130, SEQ ID NO: 131 (or 132), and SEQ ID NO: 133, respectively;
(vi) heavy chain CDR1, CDR2, and CDR3 sequences having at least 70% sequence identity with the amino acid sequences of SEQ ID NO: 138, SEQ ID NO: 106, and SEQ ID NO: 107, respectively, and light chain CDR1, CDR2, and CDR3 sequences having at least 70% sequence identity with the amino acid sequences of SEQ ID NO: 108, SEQ ID NO: 99 (or 109), and SEQ ID NO: 110, respectively;
(vii) heavy chain CDR1, CDR2, and CDR3 sequences having at least 70% sequence identity with the amino acid sequences of SEQ ID NO: 141, SEQ ID NO: 142, and SEQ ID NO: 143, respectively, and light chain CDR1, CDR2, and CDR3 sequences having at least 70% sequence identity with the amino acid sequences of SEQ ID NO: 108, SEQ ID NO: 99 (or 109), and SEQ ID NO: 110, respectively;
(viii) heavy chain CDR1, CDR2, and CDR3 sequences having at least 70% sequence identity with the amino acid sequences of SEQ ID NO: 138, SEQ ID NO: 142, and SEQ ID NO: 107, respectively, and light chain CDR1, CDR2, and CDR3 sequences having at least 70% sequence identity with the amino acid sequences of SEQ ID NO: 108, SEQ ID NO: 99 (or 109), and SEQ ID NO: 110, respectively;
(ix) heavy chain CDR1, CDR2, and CDR3 sequences having at least 70% sequence identity with the amino acid sequences of SEQ ID NO: 148, SEQ ID NO: 149, and SEQ ID NO: 150, respectively, and light chain CDR1, CDR2, and CDR3 sequences having at least 70% sequence identity with the amino acid sequences of SEQ ID NO: 151, SEQ ID NO: 152 (or 153), and SEQ ID NO: 154, respectively;
(x) heavy chain CDR1, CDR2, and CDR3 sequences having at least 70% sequence identity with the amino acid sequences of SEQ ID NO: 157, SEQ ID NO: 158, and SEQ ID NO: 159, respectively, and light chain CDR1, CDR2, and CDR3 sequences having at least 70% sequence identity with the amino acid sequences of SEQ ID NO: 160, SEQ ID NO: 161 (or 162), and SEQ ID NO: 163, respectively;
(xi) heavy chain CDR1, CDR2, and CDR3 sequences having at least 70% sequence identity with the amino acid sequences of SEQ ID NO: 157, SEQ ID NO: 158, and SEQ ID NO: 159, respectively, and light chain CDR1, CDR2, and CDR3 sequences having at least 70% sequence identity with the amino acid sequences of SEQ ID NO: 166, SEQ ID NO: 99 (or 167), and SEQ ID NO: 168, respectively;
(xii) heavy chain CDR1, CDR2, and CDR3 sequences having at least 70% sequence identity with the amino acid sequences of SEQ ID NO: 171, SEQ ID NO: 172, and SEQ ID NO: 173, respectively, and light chain CDR1, CDR2, and CDR3 sequences having at least 70% sequence identity with the amino acid sequences of SEQ ID NO: 174, SEQ ID NO: 99 (or 167), and SEQ ID NO: 168, respectively; or
(xiii) heavy chain CDR1, CDR2, and CDR3 sequences having at least 70% sequence identity with the amino acid sequences of SEQ ID NO: 177, SEQ ID NO: 178, and SEQ ID NO: 179, respectively, and light chain CDR1, CDR2, and CDR3 sequences having at least 70% sequence identity with the amino acid sequences of SEQ ID NO: 180, SEQ ID NO: 99 (or 116), and SEQ ID NO: 168, respectively.

24. The antibody, or an antigen-binding fragment thereof, according to claim 23, wherein the antibody, or the antigen-binding fragment thereof, comprises:
(i) heavy chain CDR1, CDR2, and CDR3 sequences according to SEQ ID NO: 95, SEQ ID NO: 96, and SEQ ID NO: 97, respectively, and light chain CDR1, CDR2, and CDR3 sequences according to SEQ ID NO: 98, SEQ ID NO: 99 (or 100), and SEQ ID NO: 101, respectively;
(ii) heavy chain CDR1, CDR2, and CDR3 sequences according to SEQ ID NO: 105, SEQ ID NO: 106, and SEQ ID NO: 107, respectively, and light chain CDR1, CDR2, and CDR3 sequences according to SEQ ID NO: 108, SEQ ID NO: 99 (or 109), and SEQ ID NO: 110, respectively;
(iii) heavy chain CDR1, CDR2, and CDR3 according to SEQ ID NO: 113, SEQ ID NO: 114, and SEQ ID NO: 115, respectively, and light chain CDR1, CDR2, and CDR3 according to SEQ ID NO: 98, SEQ ID NO: 99 (or 116), and SEQ ID NO: 117, respectively;
(iv) heavy chain CDR1, CDR2, and CDR3 according to SEQ ID NO: 105, SEQ ID NO: 122, and SEQ ID NO: 123, respectively, and light chain CDR1, CDR2, and CDR3 according to SEQ ID NO: 124, SEQ ID NO: 99 (or 109), and SEQ ID NO: 110, respectively;
(v) heavy chain CDR1, CDR2, and CDR3 according to SEQ ID NO: 127, SEQ ID NO: 128, and SEQ ID NO: 129, respectively, and light chain CDR1, CDR2, and CDR3 according to SEQ ID NO: 130, SEQ ID NO: 131 (or 132), and SEQ ID NO: 133, respectively;
(vi) heavy chain CDR1, CDR2, and CDR3 according to SEQ ID NO: 138, SEQ ID NO: 106, and SEQ ID NO: 107, respectively, and light chain CDR1, CDR2, and CDR3 according to SEQ ID NO: 108, SEQ ID NO: 99 (or 109), and SEQ ID NO: 110, respectively;
(vii) heavy chain CDR1, CDR2, and CDR3 according to SEQ ID NO: 141, SEQ ID NO: 142, and SEQ ID NO: 143, respectively, and light chain CDR1, CDR2, and CDR3 according to SEQ ID NO: 108, SEQ ID NO: 99 (or 109), and SEQ ID NO: 110, respectively;
(viii) heavy chain CDR1, CDR2, and CDR3 according to SEQ ID NO: 138, SEQ ID NO: 142, and SEQ ID NO: 107, respectively, and light chain CDR1, CDR2, and CDR3 according to SEQ ID NO: 108, SEQ ID NO: 99 (or 109), and SEQ ID NO: 110, respectively;
(ix) heavy chain CDR1, CDR2, and CDR3 according to SEQ ID NO: 148, SEQ ID NO: 149, and SEQ ID NO: 150, respectively, and light chain CDR1, CDR2, and CDR3 according to SEQ ID NO: 151, SEQ ID NO: 152 (or 153), and SEQ ID NO: 154, respectively;
(x) heavy chain CDR1, CDR2, and CDR3 according to SEQ ID NO: 157, SEQ ID NO: 158, and SEQ ID NO: 159, respectively, and light chain CDR1, CDR2, and CDR3 according to SEQ ID NO: 160, SEQ ID NO: 161 (or 162), and SEQ ID NO: 163, respectively;
(xi) heavy chain CDR1, CDR2, and CDR3 according to SEQ ID NO: 157, SEQ ID NO: 158, and SEQ ID NO: 159, respectively, and light chain CDR1, CDR2, and CDR3 according to SEQ ID NO: 166, SEQ ID NO: 99 (or 167), and SEQ ID NO: 168, respectively;
(xii) heavy chain CDR1, CDR2, and CDR3 according to SEQ ID NO: 171, SEQ ID NO: 172, and SEQ ID NO: 173, respectively, and light chain CDR1, CDR2, and CDR3 according to SEQ ID NO: 174, SEQ ID NO: 99 (or 167), and SEQ ID NO: 168, respectively; or
(xiii) heavy chain CDR1, CDR2, and CDR3 according to SEQ ID NO: 177, SEQ ID NO: 178, and SEQ ID NO: 179, respectively, and light chain CDR1, CDR2, and CDR3 according to SEQ ID NO: 180, SEQ ID NO: 99 (or 116), and SEQ ID NO: 168, respectively.

25. The antibody, or an antigen-binding fragment thereof, according to claim 23 or 24, wherein the antibody or the antigen-binding fragment thereof comprises:
(i) a heavy chain variable region comprising an amino acid sequence having at least 70% identity to SEQ ID NO: 102 and a light chain variable region comprising an amino acid sequence having at least 70% identity to SEQ ID NO: 103;
(ii) a heavy chain variable region comprising an amino acid sequence having at least 70% identity to SEQ ID NO: 102 and a light chain variable region comprising an amino acid sequence having at least 70% identity to SEQ ID NO: 104;
(iii) a heavy chain variable region comprising an amino acid sequence having at least 70% identity to SEQ ID NO: 111 and a light chain variable region comprising an amino acid sequence having at least 70% identity to SEQ ID NO: 112;
(iv) a heavy chain variable region comprising an amino acid sequence having at least 70% identity to SEQ ID NO: 118 and a light chain variable region comprising an amino acid sequence having at least 70% identity to SEQ ID NO: 119;
(v) a heavy chain variable region comprising an amino acid sequence having at least 70% identity to SEQ ID NO: 120 and a light chain variable region comprising an amino acid sequence having at least 70% identity to SEQ ID NO: 121;
(vi) a heavy chain variable region comprising an amino acid sequence having at least 70% identity to SEQ ID NO: 125 and a light chain variable region comprising an amino acid sequence having at least 70% identity to SEQ ID NO: 126;
(vii) a heavy chain variable region comprising an amino acid sequence having at least 70% identity to SEQ ID NO: 134 and a light chain variable region comprising an amino acid sequence having at least 70% identity to SEQ ID NO: 135;
(viii) a heavy chain variable region comprising an amino acid sequence having at least 70% identity to SEQ ID NO: 136 and a light chain variable region comprising an amino acid sequence having at least 70% identity to SEQ ID NO: 137;
(ix) a heavy chain variable region comprising an amino acid sequence having at least 70% identity to SEQ ID NO: 139 and a light chain variable region comprising an amino acid sequence having at least 70% identity to SEQ ID NO: 140;
(x) a heavy chain variable region comprising an amino acid sequence having at least 70% identity to SEQ ID NO: 144 and a light chain variable region comprising an amino acid sequence having at least 70% identity to SEQ ID NO: 145;
(xi) a heavy chain variable region comprising an amino acid sequence having at least 70% identity to SEQ ID NO: 146 and a light chain variable region comprising an amino acid sequence having at least 70% identity to SEQ ID NO: 147;
(xii) a heavy chain variable region comprising an amino acid sequence having at least 70% identity to SEQ ID NO: 155 and a light chain variable region comprising an amino acid sequence having at least 70% identity to SEQ ID NO: 156;
(xiii) a heavy chain variable region comprising an amino acid sequence having at least 70% identity to SEQ ID NO: 164 and a light chain variable region comprising an amino acid sequence having at least 70% identity to SEQ ID NO: 165;
(xiv) a heavy chain variable region comprising an amino acid sequence having at least 70% identity to SEQ ID NO: 169 and a light chain variable region comprising an amino acid sequence having at least 70% identity to SEQ ID NO: 170;
(xv) a heavy chain variable region comprising an amino acid sequence having at least 70% identity to SEQ ID NO: 175 and a light chain variable region comprising an amino acid sequence having at least 70% identity to SEQ ID NO: 176; or
(xvi) a heavy chain variable region comprising an amino acid sequence having at least 70% identity to SEQ ID NO: 181 and a light chain variable region comprising an amino acid sequence having at least 70% identity to SEQ ID NO: 182.

26. The antibody, or an antigen-binding fragment thereof, according to claim 25, wherein the antibody or the antigen-binding fragment thereof comprises:
(i) a heavy chain variable region comprising the amino acid sequence according to SEQ ID NO: 102 and a light chain variable region comprising the amino acid sequence according to SEQ ID NO: 103;
(ii) a heavy chain variable region comprising the amino acid sequence according to SEQ ID NO: 102 and a light chain variable region comprising the amino acid sequence according to SEQ ID NO: 104;
(iii) a heavy chain variable region comprising the amino acid sequence according to SEQ ID NO: 111 and a light chain variable region comprising the amino acid sequence according to SEQ ID NO: 112;
(iv) a heavy chain variable region comprising the amino acid sequence according to SEQ ID NO: 118 and a light chain variable region comprising the amino acid sequence according to SEQ ID NO: 119;
(v) a heavy chain variable region comprising the amino acid sequence according to SEQ ID NO: 120 and a light chain variable region comprising the amino acid sequence according to SEQ ID NO: 121;
(vi) a heavy chain variable region comprising the amino acid sequence according to SEQ ID NO: 125 and a light chain variable region comprising the amino acid sequence according to SEQ ID NO: 126;
(vii) a heavy chain variable region comprising the amino acid sequence according to SEQ ID NO: 134 and a light chain variable region comprising the amino acid sequence according to SEQ ID NO: 135;
(viii) a heavy chain variable region comprising the amino acid sequence according to SEQ ID NO: 136 and a light chain variable region comprising the amino acid sequence according to SEQ ID NO: 137;
(ix) a heavy chain variable region comprising the amino acid sequence according to SEQ ID NO: 139 and a light chain variable region comprising the amino acid sequence according to SEQ ID NO: 140;
(x) a heavy chain variable region comprising the amino acid sequence according to SEQ ID NO: 144 and a light chain variable region comprising the amino acid sequence according to SEQ ID NO: 145;
(xi) a heavy chain variable region comprising the amino acid sequence according to SEQ ID NO: 146 and a light chain variable region comprising the amino acid sequence according to SEQ ID NO: 147;
(xii) a heavy chain variable region comprising the amino acid sequence according to SEQ ID NO: 155 and a light chain variable region comprising the amino acid sequence according to SEQ ID NO: 156;
(xiii) a heavy chain variable region comprising the amino acid sequence according to SEQ ID NO: 164 and a light chain variable region comprising the amino acid sequence according to SEQ ID NO: 165;
(xiv) a heavy chain variable region comprising the amino acid sequence according to SEQ ID NO: 169 and a light chain variable region comprising the amino acid sequence according to SEQ ID NO: 170;
(xv) a heavy chain variable region comprising the amino acid sequence according to SEQ ID NO: 175 and a light chain variable region comprising the amino acid sequence according to SEQ ID NO: 176; or
(xvi) a heavy chain variable region comprising the amino acid sequence according to SEQ ID NO: 181 and a light chain variable region comprising the amino acid sequence according to SEQ ID NO: 182.

27. The antibody, or an antigen-binding fragment thereof, according to any one of claims 23 to 26, wherein the antibody, or the antigen-binding fragment thereof, binds to the N-loop of CCL8; but preferably not to the N-loop of CCL2, CCL7, CCL13 or CCL11.

28. The antibody, or an antigen-binding fragment thereof, according to any one of claims 23 to 27, wherein the antibody, or the antigen-binding fragment thereof, reduces or inhibits cell migration towards CCL8.

29. The antibody, or an antigen-binding fragment thereof, according to any one of claims 23 to 28, wherein the antibody, or the antigen-binding fragment thereof, reduces or inhibits CCR1-mediated chemotaxis (but not CCR2-mediated chemotaxis).

30. An antibody, or an antigen-binding fragment thereof, which binds to CCL20 (Chemokine (C-C motif) ligand 20), wherein the antibody, or the antigen-binding fragment thereof, comprises heavy chain CDR1, CDR2, and CDR3 sequences having at least 70% sequence identity with the amino acid sequences of SEQ ID NO: 183, SEQ ID NO: 184, and SEQ ID NO: 185, respectively, and light chain CDR1, CDR2, and CDR3 sequences having at least 70% sequence identity with the amino acid sequences of SEQ ID NO: 186, SEQ ID NO: 99 (or 187), and SEQ ID NO: 188, respectively.

31. The antibody, or an antigen-binding fragment thereof, according to claim 30, wherein the antibody, or the antigen-binding fragment thereof, comprises heavy chain CDR1, CDR2, and CDR3 sequences according to SEQ ID NO: 183, SEQ ID NO: 184, and SEQ ID NO: 185, respectively, and light chain CDR1, CDR2, and CDR3 sequences according to SEQ ID NO: 186, SEQ ID NO: 99 (or 187), and SEQ ID NO: 188, respectively.

32. The antibody, or an antigen-binding fragment thereof, according to claim 30 or 31, wherein the antibody or the antigen-binding fragment thereof comprises:
(i) a heavy chain variable region comprising an amino acid sequence having at least 70% identity to SEQ ID NO: 189 and a light chain variable region comprising an amino acid sequence having at least 70% identity to SEQ ID NO: 190; or
(ii) a heavy chain variable region comprising an amino acid sequence having at least 70% identity to SEQ ID NO: 191 and a light chain variable region comprising an amino acid sequence having at least 70% identity to SEQ ID NO: 192.

33. The antibody, or an antigen-binding fragment thereof, according to claim 32, wherein the antibody or the antigen-binding fragment thereof comprises:
(i) a heavy chain variable region comprising the amino acid sequence according to SEQ ID NO: 189 and a light chain variable region comprising the amino acid sequence according to SEQ ID NO: 190; or
(ii) a heavy chain variable region comprising the amino acid sequence according to SEQ ID NO: 191 and a light chain variable region comprising the amino acid sequence according to SEQ ID NO: 192.

34. The antibody, or an antigen-binding fragment thereof, according to any one of claims to 33, wherein the antibody, or the antigen-binding fragment thereof, binds to the N-loop of CCL20.

35. The antibody, or an antigen-binding fragment thereof, according to any one of claims to 34, wherein the antibody, or the antigen-binding fragment thereof, reduces or inhibits cell migration towards CCL20.

36. An antibody, or an antigen-binding fragment thereof, which binds to CCL23 (Chemokine (C-C motif) ligand 23), wherein the antibody, or the antigen-binding fragment thereof, comprises:
(i) heavy chain CDR1, CDR2, and CDR3 sequences having at least 70% sequence identity with the amino acid sequences of SEQ ID NO: 193, SEQ ID NO: 194, and SEQ ID NO: 195, respectively, and light chain CDR1, CDR2, and CDR3 sequences having at least 70% sequence identity with the amino acid sequences of SEQ ID NO: 196, SEQ ID NO: 197 (or 198), and SEQ ID NO: 199, respectively; or
(ii) heavy chain CDR1, CDR2, and CDR3 sequences having at least 70% sequence identity with the amino acid sequences of SEQ ID NO: 203, SEQ ID NO: 204, and SEQ ID NO: 205, respectively, and light chain CDR1, CDR2, and CDR3 sequences having at least 70% sequence identity with the amino acid sequences of SEQ ID NO: 206, SEQ ID NO: 197 (or 207), and SEQ ID NO: 208, respectively.

37. The antibody, or an antigen-binding fragment thereof, according to claim 36, wherein the antibody, or the antigen-binding fragment thereof, comprises:
(i) heavy chain CDR1, CDR2, and CDR3 sequences according to SEQ ID NO: 193, SEQ ID NO: 194, and SEQ ID NO: 195, respectively, and light chain CDR1, CDR2, and CDR3 sequences according to SEQ ID NO: 196, SEQ ID NO: 197 (or 198), and SEQ ID NO: 199, respectively; or
(ii) heavy chain CDR1, CDR2, and CDR3 sequences according to SEQ ID NO: 203, SEQ ID NO: 204, and SEQ ID NO: 205, respectively, and light chain CDR1, CDR2, and CDR3 sequences according to SEQ ID NO: 206, SEQ ID NO: 197 (or 207), and SEQ ID NO: 208, respectively.

38. The antibody, or an antigen-binding fragment thereof, according to claim 36 or 37, wherein the antibody or the antigen-binding fragment thereof comprises:
(i) a heavy chain variable region comprising an amino acid sequence having at least 70% identity to SEQ ID NO: 200 and a light chain variable region comprising an amino acid sequence having at least 70% identity to SEQ ID NO: 201;
(ii) a heavy chain variable region comprising an amino acid sequence having at least 70% identity to SEQ ID NO: 202 and a light chain variable region comprising an amino acid sequence having at least 70% identity to SEQ ID NO: 201; or
(iii) a heavy chain variable region comprising an amino acid sequence having at least 70% identity to SEQ ID NO: 209 and a light chain variable region comprising an amino acid sequence having at least 70% identity to SEQ ID NO: 210.

39. The antibody, or an antigen-binding fragment thereof, according to claim 38, wherein the antibody or the antigen-binding fragment thereof comprises:
(i) a heavy chain variable region comprising the amino acid sequence according to SEQ ID NO: 200 and a light chain variable region comprising the amino acid sequence according to SEQ ID NO: 201;
(ii) a heavy chain variable region comprising the amino acid sequence according to SEQ ID NO: 202 and a light chain variable region comprising the amino acid sequence according to SEQ ID NO: 201; or
(iii) a heavy chain variable region comprising the amino acid sequence according to SEQ ID NO: 209 and a light chain variable region comprising the amino acid sequence according to SEQ ID NO: 210.

40. The antibody, or an antigen-binding fragment thereof, according to any one of claims 36 to 39, wherein the antibody, or the antigen-binding fragment thereof, binds to the N-loop of CCL23.

41. The antibody, or an antigen-binding fragment thereof, according to any one of claims 36 to 40, wherein the antibody, or the antigen-binding fragment thereof, reduces or inhibits cell migration towards CCL23.

42. An antibody, or an antigen-binding fragment thereof, which binds to CXCL13 (Chemokine (C-X-C motif) ligand 13), wherein the antibody, or the antigen-binding fragment thereof, comprises:
(i) heavy chain CDR1, CDR2, and CDR3 sequences having at least 70% sequence identity with the amino acid sequences of SEQ ID NO: 211, SEQ ID NO: 212, and SEQ ID NO: 213, respectively, and light chain CDR1, CDR2, and CDR3 sequences having at least 70% sequence identity with the amino acid sequences of SEQ ID NO: 214, SEQ ID NO: 215 (or 216), and SEQ ID NO: 217, respectively;
(ii) heavy chain CDR1, CDR2, and CDR3 sequences having at least 70% sequence identity with the amino acid sequences of SEQ ID NO: 221, SEQ ID NO: 222, and SEQ ID NO: 223, respectively, and light chain CDR1, CDR2, and CDR3 sequences having at least 70% sequence identity with the amino acid sequences of SEQ ID NO: 224, SEQ ID NO: 99 (or 109), and SEQ ID NO: 225, respectively; or
(iii) heavy chain CDR1, CDR2, and CDR3 sequences having at least 70% sequence identity with the amino acid sequences of SEQ ID NO: 229, SEQ ID NO: 230, and SEQ ID NO: 231, respectively, and light chain CDR1, CDR2, and CDR3 sequences having at least 70% sequence identity with the amino acid sequences of SEQ ID NO: 224, SEQ ID NO: 99 (or 109), and SEQ ID NO: 225, respectively.

43. The antibody, or an antigen-binding fragment thereof, according to claim 36, wherein the antibody, or the antigen-binding fragment thereof, comprises:
(i) heavy chain CDR1, CDR2, and CDR3 sequences according to SEQ ID NO: 211, SEQ ID NO: 212, and SEQ ID NO: 213, respectively, and light chain CDR1, CDR2, and CDR3 sequences according to SEQ ID NO: 214, SEQ ID NO: 215 (or 216), and SEQ ID NO: 217, respectively;
(ii) heavy chain CDR1, CDR2, and CDR3 sequences according to SEQ ID NO: 221, SEQ ID NO: 222, and SEQ ID NO: 223, respectively, and light chain CDR1, CDR2, and CDR3 sequences according to SEQ ID NO: 224, SEQ ID NO: 99 (or 109), and SEQ ID NO: 225, respectively; or
(iii) heavy chain CDR1, CDR2, and CDR3 sequences according to SEQ ID NO: 229, SEQ ID NO: 230, and SEQ ID NO: 231, respectively, and light chain CDR1, CDR2, and CDR3 sequences according to SEQ ID NO: 224, SEQ ID NO: 99 (or 109), and SEQ ID NO: 225, respectively.

44. The antibody, or an antigen-binding fragment thereof, according to claim 36 or 37, wherein the antibody or the antigen-binding fragment thereof comprises:
(i) a heavy chain variable region comprising an amino acid sequence having at least 70% identity to SEQ ID NO: 218 and a light chain variable region comprising an amino acid sequence having at least 70% identity to SEQ ID NO: 219;
(ii) a heavy chain variable region comprising an amino acid sequence having at least 70% identity to SEQ ID NO: 218 and a light chain variable region comprising an amino acid sequence having at least 70% identity to SEQ ID NO: 220;
(iii) a heavy chain variable region comprising an amino acid sequence having at least 70% identity to SEQ ID NO: 226 and a light chain variable region comprising an amino acid sequence having at least 70% identity to SEQ ID NO: 227;
(iv) a heavy chain variable region comprising an amino acid sequence having at least 70% identity to SEQ ID NO: 226 and a light chain variable region comprising an amino acid sequence having at least 70% identity to SEQ ID NO: 228;
(v) a heavy chain variable region comprising an amino acid sequence having at least 70% identity to SEQ ID NO: 232 and a light chain variable region comprising an amino acid sequence having at least 70% identity to SEQ ID NO: 233; or
(vi) a heavy chain variable region comprising an amino acid sequence having at least 70% identity to SEQ ID NO: 234 and a light chain variable region comprising an amino acid sequence having at least 70% identity to SEQ ID NO: 233.

45. The antibody, or an antigen-binding fragment thereof, according to claim 38, wherein the antibody or the antigen-binding fragment thereof comprises:
(i) a heavy chain variable region comprising the amino acid sequence according to SEQ ID NO: 218 and a light chain variable region comprising the amino acid sequence according to SEQ ID NO: 219;
(ii) a heavy chain variable region comprising the amino acid sequence according to SEQ ID NO: 218 and a light chain variable region comprising the amino acid sequence according to SEQ ID NO: 220;
(iii) a heavy chain variable region comprising the amino acid sequence according to SEQ ID NO: 226 and a light chain variable region comprising the amino acid sequence according to SEQ ID NO: 227;
(iv) a heavy chain variable region comprising the amino acid sequence according to SEQ ID NO: 226 and a light chain variable region comprising the amino acid sequence according to SEQ ID NO: 228;
(v) a heavy chain variable region comprising the amino acid sequence according to SEQ ID NO: 232 and a light chain variable region comprising the amino acid sequence according to SEQ ID NO: 233; or
(vi) a heavy chain variable region comprising the amino acid sequence according to SEQ ID NO: 234 and a light chain variable region comprising the amino acid sequence according to SEQ ID NO: 233.

46. The antibody, or an antigen-binding fragment thereof, according to any one of claims 42 to 45, wherein the antibody, or the antigen-binding fragment thereof, binds to the N-loop of CXCL13.

47. The antibody, or an antigen-binding fragment thereof, according to any one of claims 42 to 46, wherein the antibody, or the antigen-binding fragment thereof, reduces or inhibits cell migration towards CXCL13.

48. An antibody, or an antigen-binding fragment thereof, which binds to CXCL16 (Chemokine (C-X-C motif) ligand 16), wherein the antibody, or the antigen-binding fragment thereof, comprises:
(i) heavy chain CDR1, CDR2, and CDR3 sequences having at least 70% sequence identity with the amino acid sequences of SEQ ID NO: 235, SEQ ID NO: 236, and SEQ ID NO: 237, respectively, and light chain CDR1, CDR2, and CDR3 sequences having at least 70% sequence identity with the amino acid sequences of SEQ ID NO: 238, SEQ ID NO: 239 (or 240), and SEQ ID NO: 241, respectively;
(ii) heavy chain CDR1, CDR2, and CDR3 sequences having at least 70% sequence identity with the amino acid sequences of SEQ ID NO: 246, SEQ ID NO: 247, and SEQ ID NO: 248, respectively, and light chain CDR1, CDR2, and CDR3 sequences having at least 70% sequence identity with the amino acid sequences of SEQ ID NO: 249, SEQ ID NO: 197 (or 250), and SEQ ID NO: 251, respectively;
(iii) heavy chain CDR1, CDR2, and CDR3 sequences having at least 70% sequence identity with the amino acid sequences of SEQ ID NO: 246, SEQ ID NO: 247, and SEQ ID NO: 248, respectively, and light chain CDR1, CDR2, and CDR3 sequences having at least 70% sequence identity with the amino acid sequences of SEQ ID NO: 249, SEQ ID NO: 197 (or 254), and SEQ ID NO: 251, respectively; or
(iv) heavy chain CDR1, CDR2, and CDR3 sequences having at least 70% sequence identity with the amino acid sequences of SEQ ID NO: 257, SEQ ID NO: 258, and SEQ ID NO: 259, respectively, and light chain CDR1, CDR2, and CDR3 sequences having at least 70% sequence identity with the amino acid sequences of SEQ ID NO: 260, SEQ ID NO: 261 (or 262), and SEQ ID NO: 263, respectively.

49. The antibody, or an antigen-binding fragment thereof, according to claim 36, wherein the antibody, or the antigen-binding fragment thereof, comprises:
(i) heavy chain CDR1, CDR2, and CDR3 sequences according to SEQ ID NO: 235, SEQ ID NO: 236, and SEQ ID NO: 237, respectively, and light chain CDR1, CDR2, and CDR3 sequences according to of SEQ ID NO: 238, SEQ ID NO: 239 (or 240), and SEQ ID NO: 241, respectively;
(ii) heavy chain CDR1, CDR2, and CDR3 sequences according to SEQ ID NO: 246, SEQ ID NO: 247, and SEQ ID NO: 248, respectively, and light chain CDR1, CDR2, and CDR3 sequences according to SEQ ID NO: 249, SEQ ID NO: 197 (or 250), and SEQ ID NO: 251, respectively;
(iii) heavy chain CDR1, CDR2, and CDR3 sequences according to SEQ ID NO: 246, SEQ ID NO: 247, and SEQ ID NO: 248, respectively, and light chain CDR1, CDR2, and CDR3 sequences according to SEQ ID NO: 249, SEQ ID NO: 197 (or 254), and SEQ ID NO: 251, respectively; or
(iv) heavy chain CDR1, CDR2, and CDR3 sequences according to SEQ ID NO: 257, SEQ ID NO: 258, and SEQ ID NO: 259, respectively, and light chain CDR1, CDR2, and CDR3 sequences according to SEQ ID NO: 260, SEQ ID NO: 261 (or 262), and SEQ ID NO: 263, respectively.

50. The antibody, or an antigen-binding fragment thereof, according to claim 36 or 37, wherein the antibody or the antigen-binding fragment thereof comprises:
(i) a heavy chain variable region comprising an amino acid sequence having at least 70% identity to SEQ ID NO: 242 and a light chain variable region comprising an amino acid sequence having at least 70% identity to SEQ ID NO: 243;
(ii) a heavy chain variable region comprising an amino acid sequence having at least 70% identity to SEQ ID NO: 244 and a light chain variable region comprising an amino acid sequence having at least 70% identity to SEQ ID NO: 245;
(iii) a heavy chain variable region comprising an amino acid sequence having at least 70% identity to SEQ ID NO: 252 and a light chain variable region comprising an amino acid sequence having at least 70% identity to SEQ ID NO: 253;
(iv) a heavy chain variable region comprising an amino acid sequence having at least 70% identity to SEQ ID NO: 255 and a light chain variable region comprising an amino acid sequence having at least 70% identity to SEQ ID NO: 256;
(v) a heavy chain variable region comprising an amino acid sequence having at least 70% identity to SEQ ID NO: 264 and a light chain variable region comprising an amino acid sequence having at least 70% identity to SEQ ID NO: 265; or
(vi) a heavy chain variable region comprising an amino acid sequence having at least 70% identity to SEQ ID NO: 266 and a light chain variable region comprising an amino acid sequence having at least 70% identity to SEQ ID NO: 265.

51. The antibody, or an antigen-binding fragment thereof, according to claim 38, wherein the antibody or the antigen-binding fragment thereof comprises:
(i) a heavy chain variable region comprising the amino acid sequence according to SEQ ID NO: 242 and a light chain variable region comprising the amino acid sequence according to SEQ ID NO: 243;
(ii) a heavy chain variable region comprising the amino acid sequence according to SEQ ID NO: 244 and a light chain variable region comprising the amino acid sequence according to SEQ ID NO: 245;
(iii) a heavy chain variable region comprising the amino acid sequence according to SEQ ID NO: 252 and a light chain variable region comprising the amino acid sequence according to SEQ ID NO: 253;
(iv) a heavy chain variable region comprising the amino acid sequence according to SEQ ID NO: 255 and a light chain variable region comprising the amino acid sequence according to SEQ ID NO: 256;
(v) a heavy chain variable region comprising the amino acid sequence according to SEQ ID NO: 264 and a light chain variable region comprising the amino acid sequence according to SEQ ID NO: 265; or
(vi) a heavy chain variable region comprising the amino acid sequence according to SEQ ID NO: 266 and a light chain variable region comprising the amino acid sequence according to SEQ ID NO: 265.

52. The antibody, or an antigen-binding fragment thereof, according to any one of claims 48 to 51, wherein the antibody, or the antigen-binding fragment thereof, binds to the N-loop of CXCL16.

53. The antibody, or an antigen-binding fragment thereof, according to any one of claims 48 to 52, wherein the antibody, or the antigen-binding fragment thereof, reduces or inhibits cell migration towards CXCL16.

54. The antibody, or an antigen-binding fragment thereof, according to any one of claims 23 to 53, wherein the antibody or the antigen-binding fragment thereof reduces or inhibits migration of immune cells.

55. The antibody, or an antigen-binding fragment thereof, according to any one of claims 23 to 54, wherein the antibody or the antigen-binding fragment thereof is a human antibody.

56. The antibody, or an antigen-binding fragment thereof, according to any one of claims 23 to 55, wherein the antibody, or an antigen-binding fragment thereof, is a monoclonal antibody.

57. The antibody according to any one of claims 23 to 56, wherein the antibody comprises an Fc moiety.

58. The antibody according to any one of claims 23 to 57, wherein the antibody is of the IgG type.

59. The antibody, or an antigen-binding fragment thereof, according to any one of claims 23 to 58, wherein the antibody, or the antigen-binding fragment thereof, is purified.

60. The antibody, or an antigen-binding fragment thereof, according to any one of claims 23 to 59, wherein the antibody, or the antigen-binding fragment thereof, is a single-chain antibody.

61. The antibody, or an antigen-binding fragment thereof, according to any one of claims 23 to 60, wherein the antibody, or the antigen-binding fragment thereof, is selected from Fab, Fab′, F(ab′)2, Fv or scFv.

62. A nucleic acid molecule comprising a polynucleotide encoding the antibody, or an antigen-binding fragment thereof, according to any one of claims 23 to 61.

63. A combination of a first and a second nucleic acid molecule, wherein the first nucleic acid molecule comprises a polynucleotide encoding the heavy chain of the antibody, or an antigen-binding fragment thereof, according to any one of claims 23 to 61; and the second nucleic acid molecule comprises a polynucleotide encoding the corresponding light chain of the same antibody, or the same antigen-binding fragment thereof.

64. The nucleic acid molecule of claim 62, or the combination of nucleic acid molecules of claim 63, wherein one or both of the polynucleotides encoding the heavy and/or light chain(s) of the antibody, or an antigen-binding fragment thereof, is/are codon-optimized.

65. A vector comprising the nucleic acid molecule or the combination of nucleic acid molecules of any one of claims 62 to 64.

66. A combination of a first and a second vector, wherein the first vector comprises a first nucleic acid molecule as defined in any one of claims 63 to 64 and the second vector comprises the corresponding second nucleic acid molecule as defined in any one of claims 63 to 64.

67. A cell expressing the antibody, or an antigen-binding fragment thereof, of any one of claims 23 to 61, or comprising the vector of claim 65 or the combination of vectors of claim 66.

68. A composition comprising the antibody, or an antigen-binding fragment thereof, of any one of claims 23 to 61, the nucleic acid or the combination of nucleic acids of any one of claims 62 to 64, the vector of claim 65, the combination of vectors of claim 66 or the cell of claim 67, and, optionally, a pharmaceutically acceptable excipient, diluent or carrier.

69. The antibody, or an antigen-binding fragment thereof, according to any one of claims 23 to 61 for use as a medicament.

70. The antibody, or an antigen-binding fragment thereof, according to any one of claims 42 to 61 for use in prophylaxis or treatment of Long COVID.

71. A combination of antibodies binding to CCL21, CXCL13 and CXCL16, or antigen-binding fragments thereof, for use in the prophylaxis or treatment of long-term symptoms of COVID-19.

72. The combination according to claim 71, comprising an antibody, or an antigen-binding fragment thereof, as defined in any one of claims 42 to 47 and 54 to 61.

73. The combination according to claim 71 or 72, comprising an antibody, or an antigen-binding fragment thereof, as defined in any one of claims 48 to 61.

74. The antibody, or an antigen-binding fragment thereof, of any one of claims 42 to 61, the nucleic acid or the combination of nucleic acids of any one of claims 62 to 64, the vector of claim 65, the combination of vectors of claim 66, the cell of claim 67, or the composition of claim 68 for use in the prophylaxis or treatment of Long COVID.

75. A method of evaluating the COVID-19 status in a subject, wherein the method comprises determining the level of antibodies binding to CCL19, CCL22 and CXCL17 in a (isolated) sample of said subject.

76. The method according to claim 75, wherein the step of determining the levels of antibodies binding to CCL19, CCL22 and CXCL17 includes determination of whether said antibody levels are increased compared to subjects without COVID-19 or uninfected subjects, the increase of said antibody levels indicating whether (i) the subject is or has been suffering from COVID-19 and/or (ii) whether the subject is or has been infected with SARS-CoV-2.

77. A method of evaluating the severity of COVID-19 in a subject, wherein the method comprises determining the level of antibodies binding to CXCL5, CXCL8 and CCL25 in a (isolated) sample of said subject.

78. The method according to claim 77, wherein the step of determining the levels of antibodies binding to CXCL5, CXCL8 and CCL25 includes determination of whether said antibody levels are decreased compared to subjects without COVID-19 hospitalization, the decrease of said antibody levels indicating severe illness.

79. The method according to claim 77 or 78, wherein the level of antibodies binding to CCL2 is also determined in the sample of said subject.

80. A method of evaluating the occurrence of long-term COVID-19 symptoms in a subject, wherein the method comprises determining the level of antibodies binding to CCL21, CXCL13 and CXCL16 in a (isolated) sample of said subject.

81. A method of predicting the occurrence of long-term symptoms of COVID-19 in a subject at about 9 months or more after onset of COVID-19, wherein the method comprises determining the level of antibodies binding to CCL21, CXCL13 and CXCL16 in a (isolated) sample of said subject, wherein the sample was obtained about 3 to 9 months after onset of COVID-19 or infection with SARS-CoV-2.

82. The method according to claim 80 or 81, wherein the step of determining the levels of antibodies binding to CCL21, CXCL13 and CXCL16 includes determination of whether said antibody levels are decreased compared to subjects without long-term COVID-19 symptoms, the decrease of said antibody levels indicating long-term persistence of symptoms.

83. A (in-vitro) method for assisting in diagnosis of Long COVID, wherein the method comprises determining the level of antibodies binding to CCL21, CXCL13 and CXCL16.

84. A method of evaluating the status of HIV infection in a subject, wherein the method comprises determining the level of antibodies binding to CCL4, CCL2, CXCL9 and CXCL12 in a (isolated) sample of said subject.

85. A method of evaluating the status of an autoimmune disorder in a subject, wherein the method comprises determining the level of antibodies binding to CCL4, CCL19, CCL25 and CXCL9 in a (isolated) sample of said subject.

86. The method according to claim 85, wherein the status of ankylosing spondylitis is evaluated and wherein the method further comprises determining the level of antibodies binding to a chemokine selected from the group consisting of CCL2, CCL5 CCL8, CCL11, CCL14, CCL16, CCL17, CCL18 and CXCL12.

87. The method according to claim 85 or 86, wherein the status of ankylosing spondylitis and/or rheumatoid arthritis is evaluated and wherein the method further comprises determining the level of antibodies binding to a chemokine selected from the group consisting of CCL13, CXCL7 and CXCL8.

88. The method according to claim 85, wherein the status of Sjögren syndrome is evaluated and wherein the method further comprises determining the level of antibodies binding to CCL1.

89. The method according to any one of claims 75 to 88, wherein the level of antibodies is determined using a method according to any one of claims 1 to 10, a peptide according to any one of claims 13-20, a composition according to claim 21 or a kit according to claim 22.
